(19) United States
(12) Patent Application Publication
(10) Pub. No.: US 2016/0040215 A1
(43) Pub. Date: Feb. 11, 2016

(54) Methods for Pathogen Detection and Enrichment from Materials and Compositions

(71) Applicant: SERES THERAPEUTICS, INC., Cambridge, MA (US)

(72) Inventors: Matthew R. Henn, Somerville, MA (US); John Grant Aunins, Doylestown, PA (US); David Arthur Berry, Brookline, MA (US); David N. Cook, Brooklyn, NY (US)

(21) Appl. No.: 14/776,676

(22) PCT Filed: Mar. 14, 2014
(86) PCT No.: PCT/US14/29539

§ 371 Date: Sep. 14, 2015

Related U.S. Application Data
(60) Provisional application No. 61/781,854, filed on Mar. 14, 2013.

(51) Int. Cl. C12Q 1/68; G01N 33/569

(52) U.S. Cl. CPC C12Q 1/689; G01N 33/56911

(57) ABSTRACT

Provided are methods and compositions for characterization of bacterial compositions for the maintenance or restoration of a healthy microbiota in the gastrointestinal tract of a mammalian subject, and the resulting characterized compositions. Provided are methods of characterizing bacterial compositions including subjecting the compositions to various detecting processes.

### RELATED APPLICATIONS

This application is related to U.S. Provisional Application No. 61/781,854, filed Mar. 14, 2013, which is incorporated by reference in its entirety.

### REFERENCE TO A SEQUENCE LISTING

This application includes a Sequence Listing submitted electronically as a text file named 26335PCT_SEQUENCELISTING.TXT, created on Mar. 14, 2014 with a size of 4,196,119 bytes. The sequence listing is incorporated by reference.

### BACKGROUND

Mammals are colonized by microbes in the gastrointestinal (GI) tract, on the skin, and in other epithelial and tissue niches such as the oral cavity, eye surface and vagina. In particular, the gastrointestinal tract harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth to ultimately form a functionally mature microbial population by about 3 years of age. Interactions between constituents of these populations, between them and surrounding environmental components, and between microbes and the host, e.g. the host immune system, shape the community structure, with availability of and competition for resources affecting the distribution of microbes. Such resources may be food, location and the availability of space to grow or a physical structure to which the microbe may attach. For example, host diet is involved in shaping the GI tract flora. The situation is similar with respect to other human microbial niches, e.g. skin, eye, ear, nose, throat, etc.

A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. In settings of ‘dysbiosis’ or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, the microbiota plays a significant role in the pathogenesis of many diseases and disorders. This includes a variety of pathogenic infections of the gut. For instance, subjects become more susceptible to pathogenic infections when the normal intestinal microbiota has been disturbed due to use of broad-spectrum antibiotics. Many of these diseases and disorders are chronic conditions that significantly decrease a subject's quality of life and can be ultimately fatal.

Manufacturers of probiotics have asserted that their preparations of bacteria promote mammalian health by preserving the natural microflora in the GI tract and reinforcing the normal controls on aberrant immune responses. See, e.g., U.S. Pat. No. 8,034,601. Probiotics, however, have been limited to a very narrow group of genera and a correspondingly limited number of species; they also tend to be limited in the number of species provided in a given probiotic product. As such, they do not adequately replace or encourage replacement of the missing natural microflora of the GI tract in many situations. For example, despite routine inoculation with Bifidobacterium, Lactobacillus, Lactococcus, and Streptococcus species, significant changes in the bacterial species composition of monozygotic twin pairs were not observed (McNulty et al. (2011) Sci. Transl. Med. 3(106):106.

Thus practitioners have a need for a method of populating a subject's gastrointestinal tract, and other niches, with a diverse and useful selection of microbiota in order to alter a dysbiosis. In response to the need for durable, efficient, and effective compositions and methods for treatment of diseases, restoring or enhancing microbiota functions by providing a multi-component bacterial composition with a diverse and/or complex microbial composition is a solution. Assessing multivalent compositions to verify their safety, identity, viability, potency and purity for the treatment of mammalian subjects is required to assure the compositions are of the appropriate quality and consistency to meet global regulatory standards. A particular challenge for multi-component compositions is the detection of microbial contaminants at low levels in the composition (e.g. see Temmerman et al 2003 Identification of antibiotic susceptibility of bacterial isolates from probiotic products. Int J. of Food Microbiology 81:1-10 and Temmerman et al 2003 Development and Validation of a nested-PCR-denaturing gradient gel electrophoresis method for taxonomic characterization of bifidobacterial communities). Due to the complex nature of the microbial compositions there is a lack of techniques to appropriately characterize a beneficial microbial composition for therapeutic and other health uses.

### SUMMARY OF THE INVENTION

Methods of the invention are provided for characterizing a therapeutic composition, comprising the steps of: (a) providing a therapeutic composition comprising at least one desired bacterial strain and optionally comprising at least one undesired bacterial strain; (b) subjecting the therapeutic composition to a first detection step and a second detection step, wherein the first detection step comprises attempting to culture at least one undesired bacterial strain, and wherein the second detection step comprises attempting to amplify at least one target nucleic acid sequence not present in the desired bacterial strain, thereby characterizing the therapeutic composition.

In one embodiment, the desired bacterial strain comprises a plurality of desired bacterial strains. In another embodiment, the result of the attempt to culture the at least one undesired bacterial strain is that the undesired bacterial strain is not detectably cultured. In other embodiments, the undesired bacterial strain is not known to be present in the therapeutic composition. In yet another embodiment, the undesired bacterial strain is a contaminating bacterial strain derived from the manufacturing environment or process. In some embodiments, the result of the attempt to amplify the at least one target nucleic acid sequence is that the target nucleic acid sequence is not detectably amplified. In one embodiment, the target nucleic acid sequence is present in i) a bacterial strain derived from a fecal culture, and/or ii) a fecal material.

In one aspect, the first detection step has a sensitivity for the undesired bacterial strain of at least 1×10⁻³, and wherein the second detection step has a sensitivity for the undesired bacterial strain of at least 1×10⁻³. In another aspect, the first detection step has a sensitivity for the undesired bacterial strain of at least 1×10⁻⁴, and wherein the second detection step has a sensitivity for the undesired bacterial strain of at least 1×10⁻⁴. In some aspects, the first detection step has a sensitivity for the undesired bacterial strain of at least 1×10⁻⁵, and wherein the second detection step has a sensitivity for the undesired bacterial strain of at least 1×10⁻⁵. In another aspect, the method includes the step of detecting, or attempting to detect, a non-bacterial microbial contaminant in the therapeutic composition. In some aspects, the non-bacterial microbial contaminant comprises a phage, virus, or eukaryotic contaminant.

In other aspects, the first detection step is performed prior to the second detection step. In another aspect, the first detection step is performed after the second detection step. In certain aspects, the first detection step and the second detection step are performed concurrently. In one embodiment, the second detection step is carried out using a product of the first detection step, the first detection step is carried out using a product of the second detection step. In another embodiment, the therapeutic composition is validated to detect a contaminant in a background of 1×10⁵ CFU of the product bacteria. In yet another embodiment, the method includes the step of attempting to enrich at least one undesired bacterial strain in the therapeutic composition.

In some embodiments, the invention includes a validated therapeutic composition provided by the method described above.

In other embodiments, a method is provided of characterizing a therapeutic composition, comprising the steps of: (a) providing a therapeutic composition comprising at least one desired entity and optionally comprising at least one undesired entity; (b) subjecting the therapeutic composition to an enrichment step wherein the at least one undesired entity or component thereof, if present in the therapeutic composition, is enriched; and (c) subjecting the enriched therapeutic composition to a first detection step and a second detection step, wherein the first detection step comprises attempting to detect the undesired entity at a concentration of about less than or equal to 1×10⁻³ the concentration of the desired entity, and wherein the second detection step comprises attempting to detect the undesired entity at a concentration of about less than or equal to 1×10⁻³ the concentration of the desired entity, wherein the first detection step and the second detection step are not identical, thereby characterizing the therapeutic composition.

In one aspect, the first detection step comprises attempting to detect the undesired entity at a concentration of about less than or equal to 1×10⁻⁴ the concentration of the desired entity, and wherein the second detection step comprises attempting to detect the undesired entity at a concentration of about less than or equal to 1×10⁻⁴ the concentration of the desired entity. In another aspect, the first detection step comprises attempting to detect the undesired entity at a concentration of about less than or equal to 1×10⁻⁵ the concentration of the desired entity, and wherein the second detection step comprises attempting to detect the undesired entity at a concentration of about less than or equal to 1×10⁻⁵ the concentration of the desired entity.

In some aspects, the desired entity comprises a plurality of desired entities. In other aspects, the at least one desired entity comprises a bacteria. In one embodiment, the at least one undesired entity comprises a bacterium, yeast, virus or combination thereof.

In another embodiment, the first detection step and the second detection step are performed simultaneously. In some embodiments, the first detection step and the second detection step are performed sequentially. In another embodiment, the second detection step detects a product of the first detection step. In other embodiments, the undesired entity is not detectably present in the characterized therapeutic composition at a concentration of about greater than or equal to 1×10⁻⁷ the concentration of the desired entity. In yet another embodiment, the component of the undesired entity comprises a nucleic acid.

In other embodiments, a method is provided for characterizing a bacterial composition, comprising the steps of: (a) providing a composition comprising at least one desired bacterial species and optionally comprising at least one undesired entity; (b) subjecting the therapeutic composition to a first detection step and a second detection step, wherein the first detection step comprises attempting to detect the at least one undesired entity and the first detection step has a sensitivity for the undesired entity of at least 1×10⁻³, and wherein the second detection step comprises attempting to detect the at least one undesired entity and the second detection step has a sensitivity for the undesired entity of at least 1×10⁻³, wherein the first and second detection steps are not identical and have a combined sensitivity for the undesired entity of at least 1×10⁻⁶.

In some embodiments, the first detection step comprises attempting to detect the at least one undesired entity and the first detection step has a sensitivity for the undesired entity of at least 1×10⁻⁴, and wherein the second detection step comprises attempting to detect the at least one undesired entity and the second detection step has a sensitivity for the undesired entity of at least 1×10⁻⁴. In certain embodiments, the first detection step comprises attempting to detect the at least one undesired entity and the first detection step has a sensitivity for the undesired entity of at least 1×10⁻⁵, and wherein the second detection step comprises attempting to detect the at least one undesired entity and the second detection step has a sensitivity for the undesired entity of at least 1×10⁻⁵. In one embodiment, the at least one desired bacterial species comprises a plurality of desired bacterial species.

In certain aspects, the first detection step is performed prior to the second detection step. In one aspect, the first detection step and the second detection step are performed concurrently. In another aspect, the first detection step is carried out using a product of the second detection step. In yet another aspect, second detection step is carried out using a product of the first detection step.

In some embodiments, a method is provided for characterizing a spore population present in a composition comprising the steps of: (a) purifying the spore population present in a composition from a fecal donation; and (b) deriving the spore population present in a composition through culture methods. In one embodiment, the spore population present in a composition is purified via solvent, acid, detergent, or heat treatment, or a density gradient separation, filtration, or any combination of methods. In certain embodiments, the purifying increases the purity, potency, and/or concentration of spores in a sample. In certain embodiments, the spore population is derived starting from isolated spore former species or spore former OTUs or from a mixture of such species. In another embodiment, the spore population is in vegetative or spore form. In some embodiments, the spores can be purified from natural sources including but not limited to feces, soil, and water.

In some embodiments, the spore population is a non-limiting subset of a microbial composition. In one embodiment, the ethanol treated fecal suspensions are a non-limiting additional subset of a microbial composition enriched for spores and spore formers. In another embodiment, the spore population comprises spore forming species wherein residual non-spore forming species have been inactivated by chemical or physical treatments. In yet another embodiment, the chemical or physical treatments include ethanol, detergent, heat or sonication.

In one aspect, the non-spore forming species have been removed from the spore preparation by various separation steps. In another aspect, the separation steps include density gradients, centrifugation, filtration and chromatography. In yet another aspect, the inactivation and separation methods are combined to make the spore preparation. In some aspects, the spore preparation comprises spore forming species that are enriched over viable non-spore formers or vegetative forms of spore formers.

In another aspect, the spores are enriched by 2-fold, 5-fold, 10-fold, 50-fold, 100-fold, 1000-fold, 10,000 fold or greater than 10,000-fold compared to all vegetative forms of bacteria. In some aspects, the spores in the spore preparation undergo partial germination during processing and formulation such that the final composition comprises spores and vegetative bacteria derived from spore forming species.

### BRIEF DESCRIPTION OF THE DRAWINGS

FIG. 1 shows the hypervariable regions mapped onto a 16s sequence and the sequence regions corresponding to these sequences on a sequence map. FIG. 1 shows variable regions mapped onto the 16s sequence and annotated 16s sequence with bolded variable regions.

FIG. 2 shows the reference sequence used in FIG. 1.

FIG. 3 shows the linear range of DPA assay compared to CFU counts/ml.

FIG. 4 shows the detection of Tb-DPA complex fluorescence from a dilution series of a pure sample of dipicolonic acid.

FIG. 5 shows the detection of Tb-DPA complex fluorescence from a dilution series of a purified sporulated preparation of Clostridium bifermentans.

FIG. 6 shows different germinant treatments having variable effects on CFU counts from donor A (top) and donor B (bottom). The Y-Axes are spore CFU per ml.

FIG. 7 shows that germinants increase the diversity of cultured spore forming OTUs observed by plating.

FIG. 8 shows heat activation as a germination treatment with BHIS+oxgall.

FIG. 9 shows the effect of lysozyme and shows a lysozyme treatment enhances germination in a subset of conditions.

FIG. 10 shows the correlation between concentration of E. durans spiked into 20% ethanol treated feces and concentration calculated from colony counts on selective media (Enterococcosel Agar).

FIG. 11 shows the microbial diversity measured in the ethanol treated spore treatment sample and patient pre- and post-treatment samples. Total microbial diversity is defined using the Chao1 Alpha-Diversity Index and is measured at different genomic sampling depths to confirm adequate sequence coverage to assay the microbiome in the target samples. The patient pretreatment (purple) harbored a microbiome that was significantly reduced in total diversity as compared to the ethanol treated spore treatment (red) and patient post treatment at days 5 (blue), 14 (orange), and 25 (green).

FIG. 12 shows patient microbial ecology was shifted by treatment with an ethanol treated spore treatment from a dysbiotic state to a state of health.

FIG. 13 shows the augmentation of Bacteroides species in patients.

FIG. 14 shows species engrafting versus species augmenting in patients microbiomes after treatment with a bacterial composition such as but not limited to an ethanol-treated spore population.

FIG. 15 shows that heat and ethanol treatments reduce cell viability.

FIG. 16 shows reduction in non-spore forming vegetative cells by treatment at 60° C. for 5 min.

FIG. 17 shows time course demonstrates ethanol reduces both anaerobic and aerobic bacterial CFUs.

FIG. 18 shows donation spore concentrations from clinical donors.

FIG. 19 shows spores initially present in ethanol treated spore preparation as measured by DPA and CFU/ml grown on specified media.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Definitions

As used herein, the terms “detect,” “detection,” and related terms mean the act or method of identifying an entity, particularly a microbial pathogen or environmental contaminant, or the presence thereof (without by necessity knowing the specific entity) in a material.

“Microbiota” refers to the community of microorganisms that occur (sustainably or transiently) in and on an animal subject, typically a mammal such as a human, including single cell and multicellular eukaryotes such as protozoan, helminthic and fungal eukaryotes, archaea, bacteria, and viruses (including bacterial viruses, i.e., phage). As used herein, “detectably cultured” mean the state, e.g., of a bacteria, of being cultured as provided herein so that such culture can be detected using the means provided herein or otherwise known in the art.

The term “microorganism” as used herein refers to an organism of microscopic or ultramicroscopic size such as a prokaryotic or a eukaryotic microbial species or a virus. The term “prokaryotic” refers to a microbial species which contains no nucleus or other organelles in the cell, which includes but is not limited to bacteria and archaea. The term “eukaryotic” refers to a microbial species that contains a nucleus and other cell organelles in the cell, which includes but is not limited to eukarya such as yeast and filamentous fungi, protozoa, algae, or higher Protista.

The terms “manufacturing environment” and “manufacturing process” relate to the environments and processes under which the therapeutic compositions and isolated bacteria as provided herein are produced, including good manufacturing process (GMP) and non-GMP environments and processes.

“Microbiome” refers to the genetic content of the communities of microbes that live in and on the human body, both sustainably and transiently, including eukaryotes (including spores), archaea, bacteria (including spores), and viruses (including bacterial viruses (i.e., phage)), wherein “genetic content” includes genomic DNA, RNA such as ribosomal RNA, the epigenome, plasmids, and all other types of genetic information.

“Dysbiosis” refers to a state of the microbiome of the gut or other body area, including mucosal or skin surfaces in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological network that no longer provides an essential function to the host and therefore no longer promotes health. A dysbiosis may be induced by illness or treatment with antibiotics or other environmental factors.

An “enrichment” or an “enrichment step” means the state of having a higher level of a quality including concentration, amount, percentage weight or dry volume, or absence of contaminants as compared to a reference.

The term “subject” refers to any animal subject including but not limited to humans, laboratory animals (e.g., primates, rats, mice) including rodents and other animals useful as models for human disease states, livestock (e.g., cows, sheep, goats, pigs, turkeys, chickens, fish), and household pets (e.g., dogs, cats, rodents, reptiles, etc.). The subject may be suffering from a dysbiosis, including, but not limited to, an infection due to a gastrointestinal pathogen or may be at risk of developing or transmitting to others an infection due to a gastrointestinal pathogen.

The term “pathobiont” refer to specific bacterial species found in healthy hosts that may trigger immune-mediated pathology and/or disease in response to certain genetic or environmental factors. Chow et al., (2011) Curr. Op. Immunol. Pathobionts of the intestinal microbiota and inflammatory disease. 23: 473-80. Thus, a pathobiont is a pathogen that is mechanistically distinct from an acquired infectious organism. Thus, the term “pathogen” includes both acquired infectious organisms and pathobionts.

The terms “pathogen”, “pathobiont” and “pathogenic” in reference to a bacterium or any other organism or entity includes any such organism or entity that is capable of causing or affecting a disease, disorder or condition of a host organism containing the organism or entity.

“Phylogenetic tree” refers to a graphical representation of the evolutionary relationships of one genetic sequence to another that is generated using a defined set of phylogenetic reconstruction algorithms (e.g. parsimony, maximum likelihood, or Bayesian). Nodes in the tree represent distinct ancestral sequences and the confidence of any node is provided by a bootstrap or Bayesian posterior probability, which measures branch uncertainty.

“Operational taxonomic units,” “OTU” (or plural, “OTUs”) refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. In 16S embodiments, OTUs that share ≧97% average nucleotide identity across the entire 16S or some variable region of the 16S are considered the same OTU (see e.g. Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940.). In embodiments involving the complete genome, MLSTs, specific genes, or sets of genes OTUs that share ≧95% average nucleotide identity are considered the same OTU (see e.g. Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940.). OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence.

Table 1 below shows a List of Operational Taxonomic Units (OTU) with taxonomic assignments made to Genus, Species, and Phylogenetic Clade. Clade membership of bacterial OTUs is based on 16S sequence data. Clades are defined based on the topology of a phylogenetic tree that is constructed from full-length 16S sequences using maximum likelihood methods familiar to individuals with ordinary skill in the art of phylogenetics. Clades are constructed to ensure that all OTUs in a given clade are: (i) within a specified number of bootstrap supported nodes from one another, and (ii) within 5% genetic similarity. OTUs that are within the same clade can be distinguished as genetically and phylogenetically distinct from OTUs in a different clade based on 16S-V4 sequence data, while OTUs falling within the same clade are closely related. OTUs falling within the same clade are evolutionarily closely related and may or may not be distinguishable from one another using 16S-V4 sequence data. Members of the same clade, due to their evolutionary relatedness, play similar functional roles in a microbial ecology such as that found in the human gut. Compositions substituting one species with another from the same clade are likely to have conserved ecological function and therefore are useful in the present invention. All OTUs are denoted as to their putative capacity to form spores and whether they are a Pathogen or Pathobiont (see Definitions for description of “Pathobiont”). NIAID Priority Pathogens are denoted as ‘Category-A’, ‘Category-B’, or ‘Category-C’, and Opportunistic Pathogens are denoted as ‘OP’. OTUs that are not pathogenic or for which their ability to exist as a pathogen is unknown are denoted as ‘N’. The ‘SEQ ID Number’ denotes the identifier of the OTU in the Sequence Listing File and ‘Public DB Accession’ denotes the identifier of the OTU in a public sequence repository.

16s Sequencing, 16s, 16s-rRNA, 16s-NGS: In microbiology, “16S sequencing” or “165-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to a reference sequence and identifying the hypervariable regions based on similarity to the reference hypervariable regions, or alternatively, one can employ Whole Genome Shotgun (WGS) sequence characterization of microbes or a microbial community.

The term “phenotype” refers to a set of observable characteristics of an individual entity. As example an individual subject may have a phenotype of “health” or “disease”. Phenotypes describe the state of an entity and all entities within a phenotype share the same set of characteristics that describe the phenotype. The phenotype of an individual results in part, or in whole, from the interaction of the entities genome and/or microbiome with the environment.

A “spore population” refers to a plurality of spores and spore forming organisms present in a composition. Synonymous terms used herein include spore composition, spore preparation, ethanol treated spore fraction and spore ecology. A spore population may be purified from a fecal donation, e.g. via solvent, acid, detergent, or heat treatment, or a density gradient separation, centrifugation, chromatographic separation, filtration, or any combination of methods described herein to increase the purity, potency and/or concentration of spores in a sample. A spore population may be derived through culture methods starting from isolated spore former species or spore former OTUs or from a mixture of such species, either in vegetative or spore form. Spores can be purified from natural sources including but not limited to feces, soil, and water. Furthermore a spore population, or preparation is a non-limiting subset of a microbial composition. Additional, ethanol treated fecal suspensions are a non-limiting additional subset of a microbial composition enriched for spores and spore formers.

In one embodiment, the spore preparation comprises spore forming species wherein residual non-spore forming species have been inactivated by chemical or physical treatments including ethanol, detergent, heat, sonication, and the like; or wherein the non-spore forming species have been removed from the spore preparation by various separations steps including density gradients, centrifugation, filtration and/or chromatography; or wherein inactivation and separation methods are combined to make the spore preparation. In yet another embodiment, the spore preparation comprises spore forming species that are enriched over viable non-spore formers or vegetative forms of spore formers. In this embodiment, spores are enriched by 2-fold, 5-fold, 10-fold, 50-fold, 100-fold, 1000-fold, 10,000-fold or greater than 10,000-fold compared to all vegetative forms of bacteria. In yet another embodiment, the spores in the spore preparation undergo partial germination during processing and formulation such that the final composition comprises spores and vegetative bacteria derived from spore forming species.

The term “isolated” encompasses a bacterium or other entity or substance that has been (1) separated from at least some of the components with which it was associated when initially produced (whether in nature or in an experimental setting) and/or (2) produced, prepared, purified, and/or manufactured by the hand of man. Isolated bacteria include those bacteria that are cultured, even if such cultures are not monocultures. Isolated bacteria may be separated from at least about 10%, about 20%, about 30%, about 40%, about 50%, about 60%, about 70%, about 80%, about 90%, or more of undesired bacteria, or, alternatively, one or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. In some embodiments, the isolated bacteria are 99.5%, 99.6%, 99.7%, 99.8%, 99.9%, or at least 99.99%, or at least 99.999% pure. As used herein, a substance is “pure” if it is substantially free of other components. The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, or by passage through culture, and a purified bacterium or bacterial population may contain other materials (exclusive of water) up to about 10%, about 20%, about 30%, about 40%, about 50%, about 60%, about 70%, about 80%, about 90%, or about 99% or and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Microbial compositions, bacterial compositions, and the bacterial components thereof are generally purified from residual habitat products.

“Residual habitat products” refers to material derived from the habitat for microbiota within or on a human or animal. For example, microbiota live in feces in the gastrointestinal tract, on the skin itself, in saliva, mucus of the respiratory tract, or secretions of the genitourinary tract (i.e., biological matter associated with the microbial community). Substantially free of residual habitat products means that the bacterial composition no longer contains the biological matter associated with the microbial environment on or in the human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the bacterial composition contains no detectable cells from a human or animal and that only microbial cells are detectable. In one embodiment, substantially free of residual habitat products may also mean that the bacterial composition contains no detectable viral (including bacterial viruses (i.e., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸ of the viable cells in the bacterial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g. PCR, DNA sequencing), serology and antigen analysis, enzymatic and metabolic analysis, and methods using instrumentation such as flow cytometry with reagents that distinguish desired constituents from contaminants.

“Inhibition” of a pathogen encompasses the inhibition of any desired function or activity of the bacterial compositions of the present invention. Demonstrations of pathogen inhibition, such as decrease in the growth of a pathogenic bacterium or reduction in the level of colonization of a pathogenic bacterium are provided herein and otherwise recognized by one of ordinary skill in the art. Inhibition of a pathogenic bacterium's “growth” may include inhibiting the increase in size of the pathogenic bacterium and/or inhibiting the proliferation (or multiplication) of the pathogenic bacterium. Inhibition of colonization of a pathogenic bacterium may be demonstrated by measuring the amount or burden of a pathogen before and after a treatment. An “inhibition” or the act of “inhibiting” includes the total cessation and partial reduction of one or more activities of a pathogen, such as growth, proliferation, colonization, and function.

A “germinant” is a material or composition or physical-chemical process capable of inducing vegetative growth of a bacterium that is in a dormant spore form, or group of bacteria in the spore form, either directly or indirectly in a host organism and/or in vitro.

A “sporulation induction agent” is a material or physical-chemical process that is capable of inducing sporulation in a bacterium, either directly or indirectly, in a host organism and/or in vitro.

To “increase production of bacterial spores” includes an activity or a sporulation induction agent. “Production” includes conversion of vegetative bacterial cells into spores and augmentation of the rate of such conversion, as well as decreasing the germination of bacteria in spore form, decreasing the rate of spore decay in vivo, or ex vivo, or to increasing the total output of spores (e.g. via an increase in volumetric output of fecal material).

A “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

Compositions and Methods of the Invention

Materials and Compositions Suitable for Testing

Encompassed by the present invention are any materials in solid or liquid form suitable for testing using the methods and systems described herein. Non-limiting examples of such materials include solids or liquids from a biological environment, foods or beverages including medical foods or beverages, specimens, therapeutic compositions, nutraceuticals and probiotics, organ and tissue transplants, sterile products such as bandages and dressings, synthetic compounds, and any material in an environment requiring a determination of the presence, and optionally the concentration of microbial and other pathogens or a measurement of the potency, purity, identity or safety of said materials.

In some embodiments the invention provides validated therapeutic compositions, meaning compositions intended for administration to a mammalian subject to treat or prevent a disease, disorder or condition. Such therapeutic compositions include one or more bacteria, yeast, virus, (e.g., phage), or combinations thereof. In particular, provided are combinations of bacteria of the human gut microbiota with the capacity to meaningfully provide functions of a healthy microbiota or to catalyze the formation of a healthy microbiota when administered to mammalian hosts.

Microbial compositions may contain at least two types of bacteria, yeast, virus (e.g., phage) or combinations thereof. For instance, a bacterial composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or more than 20 types of bacteria, as defined by species or an operational taxonomic unit (OTU) encompassing such species.

Microbial compositions may consist essentially of no greater than a number of types of bacteria, yeast, virus (e.g., phage) or combinations thereof. For instance, a bacterial composition may comprise no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, or no more than 20 types of bacteria, as defined by species or an operational taxonomic unit (OTU) encompassing such species. In some embodiments, the number of OTUs can range from 5 to 150, in others from 5-15, and in still others 40-80 OTUs may be present in a bacterial composition. In preferred embodiments, the composition contains 5-10 organisms comprising at least 90% of the microbial composition.

Bacterial compositions may consist essentially of a range of numbers of species of these preferred bacteria, but the precise number of species in a given composition is not known. For instance, a bacterial composition may consist essentially of between 2 and 10, 3 and 10, 4 and 10, 5 and 10, 6 and 10, 7 and 10, 8 and 10, or 9 and 10; or 2 and 9, 3 and 9, 4 and 9, 5 and 9, 6 and 9, 7 and 8 or 8 and 9; or 2 and 8, 3 and 8, 4 and 8, 5 and 8, 6 and 8 or 7 and 8; or 2 and 7, 3 and 7, 4 and 7, 5 and 7, or 6 and 7; or 2 and 6, 3 and 6, 4 and 6 or 5 and 6; or 2 and 5, 3 and 5 or 4 and 5; or 2 and 4 or 3 and 4; or 2 and 3, as defined by species or operational taxonomic unit (OTU) encompassing such species. In some embodiments, the number of OTUs can range from 5 to 150, in others from 5-15, and in still others 40-80 OTUs may be present in a bacterial composition. In preferred embodiments, the composition contains 5-10 organisms comprising at least 90% of the viable material (e.g., bacterial cells) present in the microbial composition.

Microbial compositions containing a plurality of species may be provided such that the relative concentration of a given species in the composition to any other species in the composition is known or unknown. Such relative concentrations of any two species, or OTUs, may be expressed as a ratio, where the ratio of a first species or OTU to a second species or OTU is 1:1 or any ratio other than 1:1, such as 1:2, 1:3, 1:4, 1:5, 1:6, 1:7, 1:8, 1:9, 1:10, 1:15, 1:20, 1:25; 1:50; 1:75, 1:100, 1:200, 1:500; 1:1000, 1:10,000, 1:100,000 or greater than 1:100,000. The ratio of strains present in a microbial composition may be determined by the ratio of the strains in a reference mammalian subject or population, e.g., healthy humans not suffering from or at known risk of developing a dysbiosis.

Microbial compositions containing a plurality of bacteria, yeast and/or virus (e.g., phage) may be provided such that the amount of a given bacteria, yeast and/or virus (e.g., phage), or the aggregate of all such entities, is between 1×104 and 1×1015 viable microbes per gram of composition or per administered dose. For example the amount of a given bacteria, yeast and/or virus (e.g., phage), or the aggregate of all such entities, is e.g., 1×104, 1×105, 1×106, 1×107, 1×108, 1×109, 1×1010, 1×1011, 1×1012, 1×1013, 1×1014, 1×1015, or greater than 1×1015 viable microbes per gram of composition or per administered dose. Alternatively, the amount of a given bacteria, yeast and/or virus (e.g., phage), or the aggregate of all bacteria, yeast and/or virus (e.g., phage), is below a given concentration e.g., below 1×104, 1×105, 1×106, 1×107, 1×108, 1×109, 1×1010, 1×1011, 1×1012, 1×1013, 1×1014, or below 1×1015 viable microbes per gram of composition or per administered dose.

Without being limited to a specific mechanism, it is thought that the validated therapeutic compositions, when administered to a mammalian subject in need thereof, inhibit the growth of a pathogen such as C. difficile, Salmonella spp., enteropathogenic E. coli, Enterococcus spp., Vibrio spp., Yersinia spp., Streptococcus spp., Shigella spp., vancomycin-resistant Enterococcus spp., Klebsiella spp, carbapenem resistant Klebsiella and other carbapenem resistant Gram negative species or OTUs, Candida spp. so that a healthy, diverse and protective microbiota can be maintained or, in the case of pathogenic bacterial infections such as recurrent C. difficile infection, and either directly repopulate or cause the repopulation of other bacteria in the intestinal lumen to reestablish ecological control over potential pathogens. In one embodiment preferred OTUs include those found in Table 1 and OTUs with 16S sequences that are 97% similar to these OTUs and corresponding sequences. In other embodiments OTUs are from the same phylogenetic clade as present in Table 1.

In other embodiments preferred microbial species include but are not limited to: Eubacterium rectale, Alistipes putredinis, Coprococcus comes, Eubacterium ventriosum, Faecalibacterium prausnitzii, Odoribacter splanchnicus, Ruminococcus bromii, Bacteroides caccae, Bacteroides finegoldii, Coprococcus catus, Dorea longicatena, Ruminococcus torques, Subdoligranulum variabile, Alistipes shahii, Eubacterium eligens, Roseburia inulinivorans, Ruminococcus obeum, Eubacterium hallii, Roseburia intestinalis, Bacteroides dorei, Bacteroides ovatus, Collinsella aerofaciens, Dorea formicigenerans, Ruminococcus lactaris, Streptococcus thermophilus, Bacteroides stercoris, Bacteroides xylanisolvens, Ruminococcus gnavus, Gordonibacter pamelaeae, Veillonella parvula, Holdermania filiformis, Streptococcus mitis, Butyricicoccus pullicaecorum, Clostridiales bacterium, Lachnospiraceae bacterium 3 1 57FAA CT1, Oscillibacter valericigenes, Roseburia hominis, Eubacterium siraeum, Ruminococcaceae bacterium D16, Alistipes sp HGB5, Blautia stercoris, Clostridiales sp SM4/1, Clostridium symbiosum, Eubacterium hadrum, Bacteroides fragilis, Bacteroides galacturonicus, Blautia wexlerae, Faecalibacterium cf, Bacteroides sp 3 1 19, Blautia luti, Christensenella minuta, Eubacterium cellulosolvens, Bacteroides sp D20, Bacteroides vulgatus, Clostridium leptum, Anaerotruncus colihominis, Bacteroides thetaiotaomicron, Bacteroides sp 1 1 30, Clostridium clostridioforme, Burkholderiales bacterium, Parabacteroides distasonis, Blautia producta, Escherichia coli, Flavonifractor plautii, Bacteroides pectinophilus, Clostridium sp YIT 12069, Ruminococcus albus, Bacteroides sp 9 1 42FAA, Bacteroides sp WAL 11050, Clostridium botulinum, Clostridium sp L2 50, Clostridium sp NML 04A032, Coprococcus eutactus, Cronobacter turicensis, Desulfovibrio piger, Eubacterium brachy, Eubacterium ramulus, Lachnospiraceae 4, Oscillibacter sp G2, Roseburia faecalis, Alistipes indistinctus, Bacteroides eggerthii, Bacteroides sp 2 1 56FAA, Bacteroides sp 20 3, Bacteroides sp 3 1 23, Bifidobacterium longum, Blautia hydrogenotrophica, Butyricimonas virosa, Clostridiales sp SS3 4, Clostridium saccharolyticum, Clostridium sp D5, Bacteroides sp 4 3 47FAA, Bifidobacterium adolescentis, Clostridium hathewayi, Clostridium nexile, Ethanoligenens harbinense, Lachnospiraceae 5, Parabacteroides goldsteinii, Parabacteroides merdae, Acidaminococcus sp D21, Akkermansia muciniphila, Anaerostipes sp 3 2 56FAA, Bacteroides cellulosilyticus, Blautia hansenii, Campylobacter concisus, Clostridium asparagiforme, Clostridium bartlettii, Clostridium bolteae, Clostridium scindens, Clostridium sp YIT 12070, Lactobacillus johnsonii, Lactobacillus reuteri, Pantoea ananatis, Parasutterella excrementihominis, Bacteroides intestinalis, Bacteroides uniformis, Bilophila wadsworthia, Citrobacter koseri, Citrobacter youngae, Clostridiales 1, Desulfovibrio desulfuricans, Edwardsiella tarda, Enterobacter sp SCSS, Enterococcus faecalis, Enterococcus gallinarum, Enterococcus hirae, Fusobacterium sp CM1, Klebsiella sp SRC DSD6, Lachnospiraceae 6, Lactobacillus casei, Lactobacillus fermentum, Lactobacillus gasseri, Lactobacillus plantarum, Leminorella grimontii, Leuconostoc citreum, Morganella sp JB T16, Streptococcus salivarius, Bacteroides sp 3 2 5, Citrobacter amalonaticus, Citrobacter sp KMSI 3, Enterococcus durans, Enterococcus raffinosus, Fusobacterium sp 11 3 2, Klebsiella pneumoniae, Klebsiella sp Co9935, Lactobacillus salivarius, Megasphaera micronuciformis, Proteus penneri, Proteus vulgaris, Shigella flexneri, Streptococcus parasanguinis, Veillonella atypica, Klebsiella sp enrichment culture clone, Clostridium difficile, A. hydrogenalis, A. Pleuropneumonaie, A. stercorihominis, B. adolescentis, B. angulatum, B. animalis, B. bifidum, B. breve, B. capillosus, B. catenulatum, B. coprophilus, B. crossotus, B. dertium, B. fibrisolvens, B. gallicum, B. plebeius, B. pseudocatenulatum, Bacteroides sp 2 1 7, Bacteroides sp 2 2 4, Bacteroides sp D1, Bacteroides sp D4, Blautia cocccoides, C. aerofaciens, C. concisus, C. hylemonae, C. intestinalis, C. methylpentosum, C. perfringens, C. phytofermentans, C. ramosum, C. stercoris, C. Sulcia muelleri, Citrobacter so.30 2, Citrobacter sp., Clostridiales sp SS2 1, Clostridium indolis, Clostridium lavalense, Clostridium saccharogumia, Clostridium sp., Clostridium sp. MLG0555, Clostridium sp. 7 2 43FAA, Clostridium cocleatum, D. vulgaris, E. cancerogenus, E. dolichum, E. fergusonii, E. sakazakii, Enterobacter sp 638, Eubacterium contortum, Eubacterium desmolans, Eubacterium limosum, F. magna, H. influenzae, H. parasuis, L. helveticus, L. ultunensis, lachnospira bacterium DJF VP30, Lachnospira pectinoshiza, Lachnospiraceae bacterium DJF VP30, M. formatexigens, Mollicutes bacriumD7, P. gingivalis, P. mirabilis, P. multocida, P. pentosaceus, Routella sp, Ruminococcus sp. ID8, Ruminococcus sp srl 5, S. enterica, S. gordonii, S. infantarius, S. mutans, S. pneumoniae, S. pyogenes, S. sanguinis, S. suis.

In some embodiments, bacterial species and combinations thereof are selected from Acidaminococcus intestine, Adlercreutzia equolifaciens, Akkermansia muciniphila, Alistipes putredinis, Alistipes shahii, Alkaliphilus metalliredigenes, Alkaliphilus oremlandii, Anaerococcus hydrogenalis, Anaerofustis stercorihominis, Anaerostipes caccae, Anaerotruncus colihominis, Bacillus alcalophilus, Bacillus amyloliquefaciens, Bacillus cereus, Bacillus circulans, Bacillus coagulans, Bacillus licheniformis, Bacillus pumilis, Bacillus subtilis, Bacteroides caccae, Bacteroides cellulosilyticus, Bacteroides coprocola, Bacteroides coprophilus, Bacteroides dorei, Bacteroides eggerthii, Bacteroides finegoldii, Bacteroides fragilis, Bacteroides intestinalis, Bacteroides ovatus, Bacteroides pectinophilus, Bacteroides plebeius, Bacteroides stercoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium animalis, Bifidobacterium bifidum, Bifidobacterium breve, Bifidobacterium catenulatum, Bifidobacterium infantis, Bifidobacterium longum, Bifidobacterium pseudocatenulatum, Bifidobacterium thermophilum, Bilophila wadsworthia, Blautia hansenii, Blautia hydrogenotrophica, Blautia luti, Blautia producta, Blautia wexlerae, Bryantella formatexigens, Butyrivibrio crossotus, Butyrivibrio fibrisolvens, Campylobacter concisus, Campylobacter curvus, Catenibacterium mitsuokai, Clostridium asparagiforme, Clostridium bartlettii, Clostridium bifermentans, Clostridium bolteae, Clostridium butyricum, Clostridium celatum, Clostridium citroniae, Clostridium clostridioforme, Clostridium cocleatum, Clostridium hathewayi, Clostridium hiranonis, Clostridium hylemonae, Clostridium indolis, Clostridium innocuum, Clostridium lavalense, Clostridium leptum, Clostridium methylpentosum, Clostridium nexile, Clostridium orbiscindens, Clostridium perfringens, Clostridium ramosum, Clostridium saccharolyticum, Clostridium scindens, Clostridium sordellii, Clostridium spiroforme, Clostridium sporogenes, Clostridium sticklandii, Clostridium symbiosum, Clostridium tetani, Collinsella aerofaciens, Coprococcus catus, Coprococcus comes, Coprococcus eutactus, Desulfovibrio piger, Dorea formicigenerans, Dorea longicatena, Enterococcus durans, Enterococcus faecalis, Enterococcus faecium, Enterococcus hirae, Escherichia coli, Eubacterium biforme, Eubacterium cylindroides, Eubacterium desmolans, Eubacterium dolichum, Eubacterium eligens, Eubacterium hadrum, Eubacterium hallii, Eubacterium limosum, Eubacterium rectale, Eubacterium siraeum, Eubacterium ventriosum, Eubacterium yurii, Faecalibacterium prausnitzii, Filifactor alocis, Finegoldia magna, Flavonifractor plautii, Holdemania filiformis, Lachnospira pectinoshiza, Lactobacillus acidophilus, Lactobacillus amylolyticus, Lactobacillus brevis, Lactobacillus bulgaricus, Lactobacillus casei, Lactobacillus fermentum, Lactobacillus gasseri, Lactobacillus helveticus, Lactobacillus johnsonii, Lactobacillus paracasei, Lactobacillus plantarum, Lactobacillus reuteri, Lactobacillus rhamnosus, Lactobacillus salivarius, Lactococcus lactis, Odoribacter laneus, Odoribacter splanchnicus, Oxalobacter formigenes, Parabacteroides distasonis, Parabacteroides johnsonii, Parabacteroides merdae, Parasutterella excrementihominis, Parvimonas micra, Pediococcus acidilactici, Pediococcus pentosaceus, Peptostreptococcus anaerobius, Peptostreptococcus stomatis, Prevotella copri, Prevotella oralis, Prevotella salivae, Propionibacterium freudenreichii, Pseudoflavonifractor capillosus, Rhodopseudomonas palustris, Roseburia faecis, Roseburia intestinalis, Roseburia inulinivorans, Ruminococcus bromii, Ruminococcus gnavus, Ruminococcus lactaris, Ruminococcus obeum, Ruminococcus torques, Shigella flexneri, Staphylococcus aureus, Staphylococcus pasteuri, Staphylococcus warneri, Streptococcus anginosus, Streptococcus mitis, Streptococcus salivarius, Streptococcus thermophiles, Subdoligranulum variabile, Sutterella wadsworthensis, and Veillonella parvula.

In some embodiments, bacterial species and combinations thereof are provided in Hamilton M J, Weingarden A R, Unno T, Khoruts A, Sadowsky M J (2013) High-throughput DNA sequence analysis reveals stable engraftment of gut microbiota following transplantation of previously frozen fecal bacteria. Gut Microbes 4: 125-135; Nishio J, Atarashi K, Tanoue T, Baba M, Negishi H, et al. (2013) Impact of TCR repertoire on intestinal homeostasis. Keystone Symposium. The Gut Microbiome: The Effector/Regulatory Immune Network; Petrof E O, Gloor G B, Vanner S J, Weese S J, Carter D, et al. (2013) Stool substitute transplant therapy for the eradication of Clostridium difficile infection: “RePOOPulating” the gut. Microbiome 1: 3; Lozupone C, Faust K, Raes J, Faith J J, Frank D N, et al. (2012) Identifying genomic and metabolic features that can underlie early successional and opportunistic lifestyles of human gut symbionts. Genome Res. 22: 1974-1984; Lawley T D, Clare S, Walker A W, Stares M D, Connor T R, et al. (2012) Targeted Restoration of the Intestinal Microbiota with a Simple, Defined Bacteriotherapy Resolves Relapsing Clostridium difficile Disease in Mice. PLoS Pathog. 8: e1002995; Hell M, Bernhofer C, Stalzer P, Kern J M, and Claassen E. 2013. Probiotics in Clostridium difficile infection: reviewing the need for a multistrain probiotic. Benef Microbes 4: 39-51; Faust K, Sathirapongsasuti J F, Izard J, Segata N, Gevers D, et al. (2012) Microbial co-occurrence relationships in the human microbiome. PLoS Comput. Biol. 8: e1002606; Van Nood E, Vrieze A, Nieuwdorp M, Fuentes S, Zoetendal E G, et al. (2012) Duodenal Infusion of Donor Feces for Recurrent Clostridium difficile. New England Journal of Medicine @nejm.org/doi/full/10.1056/NEJMoa1205037 on 17 Jan. 2013; Shahinas D, Silverman M, Sittler T, Chiu C, Kim P, et al. (2012) Toward an Understanding of Changes in Diversity Associated with Fecal Microbiome Transplantation Based on 16S rRNA Gene Deep Sequencing. MBio 3:5; Khoruts A, Dicksved J, Jansson J K, Sadowsky M J (2010) Changes in the composition of the human fecal microbiome after bacteriotherapy for recurrent Clostridium difficile-associated diarrhea. J. Clin. Gastroenterol. 44: 354-360; Chang J Y, Antonopoulos D A, Kalra A, Tonelli A, Khalife W T, et al. (2008) Decreased diversity of the fecal Microbiome in recurrent Clostridium difficile-associated diarrhea. J. Infect. Dis. 197: 435-438; and Tvede M, Rask-Madsen J (1989) Bacteriotherapy for chronic relapsing Clostridium difficile diarrhoea in six patients. Lancet 1: 1156-1160. The contents of these references are incorporated by reference herein in their entireties.

In one embodiment, the microbial composition comprises at least one and preferably more than one of the following: Barnesiella intestinihominis; Lactobacillus reuteri; a species characterized as one of Enterococcus hirae, Enterococus faecium, or Enterococcus durans; a species characterized as one of Anaerostipes caccae or Clostridium indolis; a species characterized as one of Staphylococcus warneri or Staphylococcus pasteuri; and Adlercreutzia equolifaciens. In an alternative embodiment, at least one of the preceding species is not substantially present in the composition.

In one embodiment, the microbial composition comprises at least one and preferably more than one (e.g., 2, 3, 4, 5, 6, 7, 8, 9, 10, or more than 10) of the following: Clostridium absonum, Clostridium argentinense, Clostridium baratii, Clostridium bartlettii, Clostridium bifermentans, Clostridium botulinum, Clostridium butyricum, Clostridium cadaveris, Clostridium camis, Clostridium celatum, Clostridium chauvoei, Clostridium clostridioforme, Clostridium cochlearium, Clostridium difficile, Clostridium fallax, Clostridium felsineum, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium ramosum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an alternative embodiment, at least one of the preceding species is not substantially present in the bacterial composition.

In one embodiment, the microbial composition comprises at least one and preferably more than one of the following: Clostridium innocuum, Clostridum bifermentans, Clostridium butyricum, Bacteroides fragilis, Bacteroides thetaiotaomicron, Bacteroides uniformis, three strains of Escherichia coli, and Lactobacillus sp. In an alternative embodiment, at least one of the preceding species is not substantially present in the bacterial composition.

In one embodiment, the microbial composition comprises at least one and preferably more than one of the following: Clostridium bifermentans, Clostridium innocuum, Clostridium butyricum, three strains of Escherichia coli, three strains of Bacteroides, and Blautia producta. In an alternative embodiment, at least one of the preceding species is not substantially present in the composition.

In one embodiment, the microbial composition comprises at least one and preferably more than one of the following: Bacteroides sp., Escherichia coli, and non-pathogenic Clostridia, including Clostridium innocuum, Clostridium bifermentans and Clostridium ramosum. In an alternative embodiment, at least one of the preceding species is not substantially present in the bacterial composition.

In one embodiment, the microbial composition comprises at least one and preferably more than one of the following: Bacteroides species, Escherichia coli and non-pathogenic Clostridia, such as Clostridium butyricum, Clostridium bifermentans and Clostridium innocuum. In an alternative embodiment, at least one of the preceding species is not substantially present in the microbial composition.

In certain embodiments, provided are microbial compositions containing a plurality of Bacteroides species. In such exemplary embodiments, the microbial composition comprises at least one and preferably more than one of the following: Bacteroides caccae, Bacteroides capillosus, Bacteroides coagulans, Bacteroides distasonis, Bacteroides eggerthii, Bacteroides forsythus, Bacteroides fragilis, Bacteroides fragilis-ryhm, Bacteroides gracilis, Bacteroides levii, Bacteroides macacae, Bacteroides merdae, Bacteroides ovatus, Bacteroides pneumosintes, Bacteroides putredinis, Bacteroides pyogenes, Bacteroides splanchnicus, Bacteroides stercoris, Bacteroides tectum, Bacteroides thetaiotaomicron, Bacteroides uniformis, Bacteroides ureolyticus, and Bacteroides vulgatus. In an alternative embodiment, at least one of the preceding species is not substantially present in the composition.

In one embodiment, the microbial composition comprises at least one and preferably more than one of the following: Bacteroides, Eubacteria, Fusobacteria, Propionibacteria, Lactobacilli, anaerobic cocci, Ruminococcus, Escherichia coli, Gemmiger, Desulfomonas, and Peptostreptococcus. In an alternative embodiment, at least one of the preceding species is not substantially present in the microbial composition.

In one embodiment, the microbial composition comprises at least one and preferably more than one of the following: Bacteroides fragilis ss. Vulgatus, Eubacterium aerofaciens, Bacteroides fragilis ss. Thetaiotaomicron, Blautia producta (previously known as Peptostreptococcus productus II), Bacteroides fragilis ss. Distasonis, Fusobacterium prausnitzii, Coprococcus eutactus, Eubacterium aerofaciens III, Blautia producta (previously known as Peptostreptococcus productus I), Ruminococcus bronii, Bifidobacterium adolescentis, Gemmiger formicilis, Bifidobacterium longum, Eubacterium siraeum, Ruminococcus torques, Eubacterium rectale III-H, Eubacterium rectale IV, Eubacterium eligens, Bacteroides eggerthii, Clostridium leptum, Bacteroides fragilis ss. A, Eubacterium biforme, Bifidobacterium infantis, Eubacterium rectale III-F, Coprococcus comes, Bacteroides capillosus, Ruminococcus albus, Eubacterium formicigenerans, Eubacterium hallii, Eubacterium ventriosum I, Fusobacterium russii, Ruminococcus obeum, Eubacterium rectale II, Clostridium ramosum I, Lactobacillus leichmanii, Ruminococcus cailidus, Butyrivibrio crossotus, Acidaminococcus fermentans, Eubacterium ventriosum, Bacteroides fragilis ss. fragilis, Bacteroides AR, Coprococcus catus, Eubacterium hadrum, Eubacterium cylindroides, Eubacterium ruminantium, Eubacterium CH-1, Staphylococcus epidermidis, Peptostreptococcus BL, Eubacterium limosum, Bacteroides praeacutus, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Eubacterium AG, -AK, -AL, -AL-1, -AN; Bacteroides fragilis ss. ovatus, -ss. d, -ss. f; Bacteroides L-1, L-5; Fusobacterium nucleatum, Fusobacterium mortiferum, Escherichia coli, Streptococcus morbiliorum, Peptococcus magnus, Peptococcus G, AU-2; Streptococcus intermedius, Ruminococcus lactaris, Ruminococcus CO Gemmiger X, Coprococcus BH, -CC; Eubacterium tenue, Eubacterium ramulus, Eubacterium AE, -AG-H, -AG-M, -AJ, -BN-1; Bacteroides clostridiiformis ss. clostridliformis, Bacteroides coagulans, Bacteroides orails, Bacteroides ruminicola ss. brevis, -ss. ruminicola, Bacteroides splanchnicus, Desuifomonas pigra, Bacteroides L-4, -N-i; Fusobacterium H, Lactobacillus G, and Succinivibrio A. In an alternative embodiment, at least one of the preceding species is not substantially present in the composition.

Heterogeneous Bacterial Compositions

Also provided are compositions containing material obtained or derived from natural sources containing microbial materials, and such compositions are in some embodiments substantially heterogeneous in the microbial and non-microbial components contained therein. For example, such natural sources may be fecal material obtained from one or more healthy subjects, or one or more subjects having or at risk of developing a disease, disorder or condition associated with a dysbiosis. Other such natural or manipulated sources include environmental samples, e.g., ground water, open freshwater and sea water, soils, earth and rocks, plants, mosses, lichens and other natural microbial communities, non-human animals (other than animals included as “subjects” as defined herein, and their microbiota), raw foods, fermented foods, fermented beverages, animal feeds, or silage.

In one embodiment the microbial compositions are therapeutic compositions containing non-pathogenic, germination-competent bacterial spores, for the prevention, control, and treatment of gastrointestinal diseases, disorders and conditions and for general nutritional health. These compositions are advantageous in being suitable for safe administration to humans and other mammalian subjects and are efficacious in numerous gastrointestinal diseases, disorders and conditions and in general nutritional health. While spore-based compositions are known, these are generally prepared according to various techniques such as lyophilization or spray-drying of liquid bacterial cultures, resulting in poor efficacy, instability, substantial variability and lack of adequate safety and efficacy.

It has now been found that populations of bacterial spores can be obtained from biological materials obtained from mammalian subjects, including humans. These populations are formulated into compositions as provided herein, and administered to mammalian subjects using the methods as provided herein.

Provided herein are therapeutic compositions containing a purified population of bacterial spores. As used herein, the terms “purify”, “purified” and “purifying” refer to the state of a population (e.g., a plurality of known or unknown amount and/or concentration) of desired bacterial spores, that have undergone one or more processes of purification, e.g., a selection or an enrichment of the desired bacterial spore, or alternatively a removal or reduction of residual habitat products as described herein. In some embodiments, a purified population has no detectable undesired activity or, alternatively, the level or amount of the undesired activity is at or below an acceptable level or amount. In other embodiments, a purified population has an amount and/or concentration of desired bacterial spores at or above an acceptable amount and/or concentration. In other embodiments, the ratio of desired-to-undesired activity (e.g. spores compared to vegetative bacteria), has changed by 2-, 5-, 10-, 30-, 100-, 300-, 1×104, 1×105, 1×106, 1×107, 1×108, or greater than 1×108. In other embodiments, the purified population of bacterial spores is enriched as compared to the starting material (e.g., a fecal material) from which the population is obtained. This enrichment may be by 10%, 20%, 30%, 40%, 50%, 60%, 70%, 80%, 90%, 95%, 96%, 97%, 98%, 99%, 99.9%, 99.99%, 99.999%, 99.9999%, 99.9999%, or greater than 99.999999% as compared to the starting material.

In certain embodiments, the purified populations of bacterial spores have reduced or undetectable levels of one or more pathogenic activities, such as toxicity, an ability to cause infection of the mammalian recipient subject, an undesired immunomodulatory activity, an autoimmune response, a metabolic response, or an inflammatory response or a neurological response. Such a reduction in a pathogenic activity may be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In other embodiments, the purified populations of bacterial spores have reduced sensory components as compared to fecal material, such as reduced odor, taste, appearance, and umami.

Provided are purified populations of bacterial spores that are substantially free of residual habitat products. In certain embodiments, this means that the bacterial spore composition no longer contains a substantial amount of the biological matter associated with the microbial community while living on or in the human or animal subject, and the purified population of spores may be 100% free, 99% free, 98% free, 97% free, 96% free, or 95% free of any contamination of the biological matter associated with the microbial community. Substantially free of residual habitat products may also mean that the bacterial spore composition contains no detectable cells from a human or animal, and that only microbial cells are detectable, in particular, only desired microbial cells are detectable. In another embodiment, it means that fewer than 1×10-2%, 1×10-3%, 1×10-4%, 1×10-5%, 1×10-6%, 1×10-7%, 1×10-8% of the cells in the bacterial composition are human or animal, as compared to microbial cells. In another embodiment, the residual habitat product present in the purified population is reduced at least a certain level from the fecal material obtained from the mammalian donor subject, e.g., reduced by at least about 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999%.

In one embodiment, substantially free of residual habitat products or substantially free of a detectable level of a pathogenic material means that the bacterial composition contains no detectable viral (including bacterial viruses (i.e., phage)), fungal, or mycoplasmal or toxoplasmal contaminants, or a eukaryotic parasite such as a helminth. Alternatively, the purified spore populations are substantially free of an acellular material, e.g., DNA, viral coat material, or non-viable bacterial material. Alternatively, the purified spore population may processed by a method that kills, inactivates, or removes one or more specific undesirable viruses, such as an enteric virus, including norovirus, poliovirus or hepatitis A virus.

As described herein, purified spore populations can be demonstrated by genetic analysis (e.g., PCR, DNA sequencing), serology and antigen analysis, microscopic analysis, microbial analysis including germination and culturing, and methods using instrumentation such as flow cytometry with reagents that distinguish desired bacterial spores from non-desired, contaminating materials.

Exemplary biological materials include fecal materials such as feces or materials isolated from the various segments of the small and large intestines. Fecal materials are obtained from a mammalian donor subject, or can be obtained from more than one donor subject, e.g., 2, 3, 4, 5, 6, 7, 8, 9, 10, 15, 20, 25, 30, 35, 40, 45, 50, 75, 100, 200, 300, 400, 500, 750, 1000 or from greater than 1000 donors, where such materials are then pooled prior to purification of the desired bacterial spores. In another embodiment, fecal materials can be obtained from a single donor subject over multiple times and pooled from multiple samples e.g. 1, 2, 3, 4, 5, 6, 7, 8, 9, 10, 15, 20, 25, 30, 32, 35, 40, 45, 48, 50, 100 samples from a single donor.

In alternative embodiments, the desired bacterial spores are purified from a single fecal material sample obtained from a single donor, and after such purification are combined with purified spore populations from other purifications, either from the same donor at a different time, or from one or more different donors, or both.

Mammalian donor subjects are generally of good health and have microbiota consistent with such good health. Often, the donor subjects have not been administered antibiotic compounds within a certain period prior to the collection of the fecal material. In certain embodiments, the donor subjects are not obese or overweight, and may have body mass index (BMI) scores of below 25, such as between 18.5 and 24.9. In other embodiments, the donor subjects are not mentally ill or have no history or familial history of mental illness, such as anxiety disorder, depression, bipolar disorder, autism spectrum disorders, schizophrenia, panic disorders, attention deficit (hyperactivity) disorders, eating disorders or mood disorders. In other embodiments, the donor subjects do not have irritable bowel disease (e.g., crohn's disease, ulcerative colitis), irritable bowel syndrome, celiac disease, colorectal cancer or a family history of these diseases. In other embodiments, donors have been screened for blood borne pathogens and fecal transmissible pathogens using standard techniques known to one in the art (e.g. nucleic acid testing, serological testing, antigen testing, culturing techniques, enzymatic assays, assays of cell free fecal filtrates looking for toxins on susceptible cell culture substrates).

In some embodiments, donors are also selected for the presence of certain genera and/or species that provide increased efficacy of therapeutic compositions containing these genera or species. In other embodiments, donors are preferred that produce relatively higher concentrations of spores in fecal material than other donors. In further embodiments, donors are preferred that provide fecal material from which spores having increased efficacy are purified; this increased efficacy is measured using in vitro or in animal studies as described below. In some embodiments, the donor may be subjected to one or more pre-donation treatments in order to reduce undesired material in the fecal material, and/or increase desired spore populations.

It is advantageous to screen the health of the donor subject prior to and optionally, one or more times after, the collection of the fecal material. Such screening identifies donors carrying pathogenic materials such as viruses (HIV, hepatitis, polio) and pathogenic bacteria. Post-collection, donors are screened about one week, two weeks, three weeks, one month, two months, three months, six months, one year or more than one year, and the frequency of such screening may be daily, weekly, bi-weekly, monthly, bi-monthly, semi-yearly or yearly. Donors that are screened and do not test positive, either before or after donation or both, are considered “validated” donors.

Solvent Treatments

To purify the bacterial spores, the fecal material is subjected to one or more solvent treatments. A solvent treatment is a miscible solvent treatment (either partially miscible or fully miscible) or an immiscible solvent treatment. Miscibility is the ability of two liquids to mix with each to form a homogeneous solution. Water and ethanol, for example, are fully miscible such that a mixture containing water and ethanol in any ratio will show only one phase. Miscibility is provided as a wt/wt %, or weight of one solvent in 100 g of final solution. If two solvents are fully miscible in all proportions, their miscibility is 100%. Provided as fully miscible solutions with water are alcohols, e.g., methanol, ethanol, isopropanol, butanol, propanediol, butanediol, etc. The alcohols can be provided already combined with water; e.g., a solution containing 10%, 20%, 25%, 30%, 35%, 40%, 45%, 50%, 55%, 60%, 65%, 70%, 75%, 89%, 85%, 90%, 95% or greater than 95%. Other solvents are only partially miscible, meaning that only some portion will dissolve in water. Diethyl ether, for example, is partially miscible with water. Up to 7 grams of diethyl ether will dissolve in 93 g of water to give a 7% (wt/wt %) solution. If more diethyl ether is added, a two-phase solution will result with a distinct diethyl ether layer above the water. Other partially miscible materials include ethers, propanoate, butanoate, chloroform, dimethoxyethane, or tetrahydrofuran. In contrast, an oil such as an alkane and water are immiscible and form two phases. Further, immiscible treatments are optionally combined with a detergent, either an ionic detergent or a non-ionic detergent. Exemplary detergents include Triton X-100, Tween 20, Tween 80, Nonidet P40, a pluronic, or a polyol. The solvent treatment steps reduces the viability of non-spore forming bacterial species by 10%, 20%, 30%, 40%, 50%, 60%, 70%, 80%, 85%, 90%, 95%, 99%, 99.9%, 99.99%, 99.999%, or 99.9999%, and it may optionally reduce the viability of contaminating protists, parasites and/or viruses.

Chromatography treatments. To purify spore populations, the fecal materials are subjected to one or more chromatographic treatments, either sequentially or in parallel. In a chromatographic treatment, a solution containing the fecal material is contacted with a solid medium containing a hydrophobic interaction chromatographic (HIC) medium or an affinity chromatographic medium. In an alternative embodiment, a solid medium capable of absorbing a residual habitat product present in the fecal material is contacted with a solid medium that adsorbs a residual habitat product. In certain embodiments, the HIC medium contains sepharose or a derivatized sepharose such as butyl sepharose, octyl sepharose, phenyl sepharose, or butyl-s sepharose. In other embodiments, the affinity chromatographic medium contains material derivatized with mucin type I, II, III, IV, V, or VI, or oligosaccharides derived from or similar to those of mucins type I, II, III, IV, V, or VI. Alternatively, the affinity chromatographic medium contains material derivatized with antibodies that recognize spore-forming bacteria.

Mechanical Treatments

Provided herein is the physical disruption of the fecal material, particularly by one or more mechanical treatment such as blending, mixing, shaking, vortexing, impact pulverization, and sonication. As provided herein, the mechanical disrupting treatment substantially disrupts a non-spore material present in the fecal material and does not substantially disrupt a spore present in the fecal material, or it may disrupt the spore material less than the non-spore material, e.g. 2-fold less, 5-, 10-, 30-, 100-, 300-, 1000- or greater than 1000-fold less. Furthermore, mechanical treatment homogenizes the material for subsequent sampling, testing, and processing. Mechanical treatments optionally include filtration treatments, where the desired spore populations are retained on a filter while the undesirable (non-spore) fecal components to pass through, and the spore fraction is then recovered from the filter medium. Alternatively, undesirable particulates and eukaryotic cells may be retained on a filter while bacterial cells including spores pass through. In some embodiments the spore fraction retained on the filter medium is subjected to a diafiltration step, wherein the retained spores are contacted with a wash liquid, typically a sterile saline-containing solution or other diluent such as a water compatible polymer including a low-molecular polyethylene glycol (PEG) solution, in order to further reduce or remove the undesirable fecal components.

Thermal Treatments

Provided herein is the thermal disruption of the fecal material. Generally, the fecal material is mixed in a saline-containing solution such as phosphate-buffered saline (PBS) and subjected to a heated environment, such as a warm room, incubator, water-bath, or the like, such that efficient heat transfer occurs between the heated environment and the fecal material. Preferably the fecal material solution is mixed during the incubation to enhance thermal conductivity and disrupt particulate aggregates. Thermal treatments can be modulated by the temperature of the environment and/or the duration of the thermal treatment. For example, the fecal material or a liquid comprising the fecal material is subjected to a heated environment, e.g., a hot water bath of at least about 20, 25, 30, 35, 40, 45, 50, 55, 60, 65, 70, 75, 80, 85, 90, 95, 100 or greater than 100 degrees Celsius, for at least about 1, 5, 10, 15, 20, 30, 45 seconds, or 1, 2, 3, 4, 5, 6, 7, 8, 9, 10, 15, 20, 25, 30, 40, or 50 minutes, or 1, 2, 3, 4, 5, 6, 7, 8, 9, 10 or more than 10 hours. In certain embodiments the thermal treatment occurs at two different temperatures, such as 30 seconds in a 100 degree Celsius environment followed by 10 minutes in a 50 degree Celsius environment. In preferred embodiments the temperature and duration of the thermal treatment are sufficient to kill or remove pathogenic materials while not substantially damaging or reducing the germination-competency of the spores. In other preferred embodiments, the temperature and duration of the thermal treatment is short enough to reduce the germination of the spore population.

Irradiation Treatments

Provided are methods of treating the fecal material or separated contents of the fecal material with ionizing radiation, typically gamma irradiation, ultraviolet irradiation or electron beam irradiation provided at an energy level sufficient to kill pathogenic materials while not substantially damaging the desired spore populations. For example, ultraviolet radiation at 254 nm provided at an energy level below about 22,000 microwatt seconds per cm2 will not generally destroy desired spores.

Centrifugation and Density Separation Treatments

Provided are methods of separating desired spore populations from the other components of the fecal material by centrifugation. A solution containing the fecal material is subjected to one or more centrifugation treatments, e.g., at about 200×g, 1000×g, 2000×g, 3000×g, 4000×g, 5000×g, 6000×g, 7000×g, 8000×g or greater than 8000×g. Differential centrifugation separates desired spores from undesired non-spore material; at low forces the spores are retained in solution, while at higher forces the spores are pelleted while smaller impurities (e.g., virus particles, phage, microscopic fibers, biological macromolecules such as free protein, nucleic acids and lipids) are retained in solution. For example, a first low force centrifugation pellets fibrous materials; a second, higher force centrifugation pellets undesired eukaryotic cells, and a third, still higher force centrifugation pellets the desired spores while smaller contaminants remain in suspension. In some embodiments density or mobility gradients or cushions (e.g., step cushions), such as CsCl, Percoll, Ficoll, Nycodenz, Histodenz or sucrose gradients, are used to separate desired spore populations from other materials in the fecal material.

Also provided herein are methods of producing spore populations that combine two or more of the treatments described herein in order to synergistically purify the desired spores while killing or removing undesired materials and/or activities from the spore population. It is generally desirable to retain the spore populations under non-germinating and non-growth promoting conditions and media, in order to minimize the growth of pathogenic bacteria present in the spore populations and to minimize the germination of spores into vegetative bacterial cells.

Purified Spore Populations

As described herein, purified spore populations contain combinations of commensal bacteria of the human gut microbiota with the capacity to meaningfully provide functions of a healthy microbiota when administered to a mammalian subject. Without being limited to a specific mechanism, it is thought that such compositions inhibit the growth of a pathogen such as C. difficile, Salmonella spp., enteropathogenic E. coli, Fusobacterium spp., Klebsiella spp. and vancomycin-resistant Enterococcus spp., so that a healthy, diverse and protective microbiota can be maintained or, in the case of pathogenic bacterial infections such as C. difficile infection, repopulate the intestinal lumen to reestablish ecological control over potential pathogens. In one embodiment, the purified spore populations can engraft in the host and remain present for 1 day, 2 days, 3 days, 4 days, 5 days, 6 days, 7 days, 10 days, 14 days, 21 days, 25 days, 30 days, 60 days, 90 days, or longer than 90 days. Additionally, the purified spore populations can induce other healthy commensal bacteria found in a healthy gut to engraft in the host that are not present in the purified spore populations or present at lesser levels and therefore these species are considered to “augment” the delivered spore populations. In this manner, commensal species augmentation of the purified spore population in the recipient's gut leads to a more diverse population of gut microbiota then present initially.

Preferred bacterial genera include Acetanaerobacterium, Acetivibrio, Alicyclobacillus, Alkaliphilus, Anaerofustis, Anaerosporobacter, Anaerostipes, Anaerotruncus, Anoxybacillus, Bacillus, Bacteroides, Blautia, Brachyspira, Brevibacillus, Bryantella, Bulleidia, Butyricicoccus, Butyrivibrio, Catenibacterium, Chlamydiales, Clostridiaceae, Clostridiales, Clostridium, Collinsella, Coprobacillus, Coprococcus, Coxiella, Deferribacteres, Desulfitobacterium, Desulfotomaculum, Dorea, Eggerthella, Erysipelothrix, Erysipelotrichaceae, Ethanoligenens, Eubacterium, Faecalibacterium, Filifactor, Flavonifractor, Flexistipes, Fulvimonas, Fusobacterium, Gemmiger, Geobacillus, Gloeobacter, Holdemania, Hydrogenoanaerobacterium, Kocuria, Lachnobacterium, Lachnospira, Lachnospiraceae, Lactobacillus, Lactonifactor, Leptospira, Lutispora, Lysinibacillus, Mollicutes, Moorella, Nocardia, Oscillibacter, Oscillospira, Paenibacillus, Papillibacter, Pseudoflavonifractor, Robinsoniella, Roseburia, Ruminococcaceae, Ruminococcus, Saccharomonospora, Sarcina, Solobacterium, Sporobacter, Sporolactobacillus, Streptomyces, Subdoligranulum, Sutterella, Syntrophococcus, Thermoanaerobacter, Thermobifida, Turicibacter

Preferred bacterial species are provided at Table 1 and demarcated as spore formers. Where specific strains of a species are provided, one of skill in the art will recognize that other strains of the species can be substituted for the named strain.

In some embodiments, spore-forming bacteria are identified by the presence of nucleic acid sequences that modulate sporulation. In particular, signature sporulation genes are highly conserved across members of distantly related genera including Clostridium and Bacillus. Traditional approaches of forward genetics have identified many, if not all, genes that are essential for sporulation (spo). The developmental program of sporulation is governed in part by the successive action of four compartment-specific sigma factors (appearing in the order σF, σE, σG and σK), whose activities are confined to the forespore (σF and σG) or the mother cell (σE and σK). In other embodiments, spore-forming bacteria are identified by the biochemical activity of DPA producing enzymes or by analyzing DPA content of cultures. As part of the bacterial sporulation, large amounts of DPA are produced, and comprise 5-15% of the mass of a spore. Because not all viable spores germinate and grow under known media conditions, it is difficult to assess a total spore count in a population of bacteria. As such, a measurement of DPA content highly correlates with spore content and is an appropriate measure for characterizing total spore content in a bacterial population.

Provided are spore populations containing more than one type of bacterium. As used herein, a “type” or more than one “types” of bacteria may be differentiated at the genus level, the species, level, the sub-species level, the strain level or by any other taxonomic method, as described herein and otherwise known in the art.

In some embodiments all or essentially all of the bacterial spores present in a purified population are obtained from a fecal material treated as described herein or otherwise known in the art. In alternative embodiments, one or more than one bacterial spores or types of bacterial spores are generated in culture and combined to form a purified spore population. In other alternative embodiments, one or more of these culture-generated spore populations are combined with a fecal material-derived spore population to generate a hybrid spore population. Bacterial compositions may contain at least two types of these preferred bacteria, including strains of the same species. For instance, a bacterial composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or more than 20 types of bacteria, as defined by species or operational taxonomic unit (OTU) encompassing such species.

Thus, provided herein are methods for production of a composition containing a population of bacterial spores suitable for therapeutic administration to a mammalian subject in need thereof. And the composition is produced by generally following the steps of: (a) providing a fecal material obtained from a mammalian donor subject; and (b) subjecting the fecal material to at least one purification treatment or step under conditions such that a population of bacterial spores is produced from the fecal material. The composition is formulated such that a single oral dose contains at least about 1×104 colony forming units of the bacterial spores, and a single oral dose will typically contain about 1×104, 1×105, 1×106, 1×107, 1×108, 1×109, 1×1010, 1×1011, 1×1012, 1×1013, 1×1014, 1×1015, or greater than 1×1015 CFUs of the bacterial spores. The presence and/or concentration of a given type of bacterial spore may be known or unknown in a given purified spore population. If known, for example the concentration of spores of a given strain, or the aggregate of all strains, is e.g., 1×104, 1×105, 1×106, 1×107, 1×108, 1×109, 1×1010, 1×1011, 1×1012, 1×1013, 1×1014, 1×1015, or greater than 1×1015 viable bacterial spores per gram of composition or per administered dose.

In some formulations, the composition contains at least about 0.5%, 1%, 2%, 5%, 10%, 20%, 30%, 40%, 50%, 60%, 70%, 80%, 90% or greater than 90% spores on a mass basis. In some formulations, the administered dose does not exceed 200, 300, 400, 500, 600, 700, 800, 900 milligrams or 1, 1.1, 1.2, 1.3, 1.4, 1.5, 1.6, 1.7, 1.8, or 1.9 grams in mass.

The bacterial spore compositions are generally formulated for oral or gastric administration, typically to a mammalian subject. In particular embodiments, the composition is formulated for oral administration as a solid, semi-solid, gel, or liquid form, such as in the form of a pill, tablet, capsule, or lozenge. In some embodiments, such formulations contain or are coated by an enteric coating to protect the bacteria through the stomach and small intestine, although spores are generally resistant to the stomach and small intestines. In other embodiments, the bacterial spore compositions may be formulated with a germinant to enhance engraftment, or efficacy. In yet other embodiments, the bacterial spore compositions may be co-formulated or co-administered with prebiotic substances, to enhance engraftment or efficacy.

The bacterial spore compositions may be formulated to be effective in a given mammalian subject in a single administration or over multiple administrations. For example, a single administration is substantially effective to reduce Cl. difficile and/or Cl. difficile toxin content in a mammalian subject to whom the composition is administered.

Substantially effective means that Cl. difficile and/or Cl. difficile toxin content in the subject is reduced by at least 10%, 20%, 30%, 40%, 50%, 60%, 70%, 80%, 90%, 95%, 98%, 99% or greater than 99% following administration of the composition. Alternatively, efficacy may be measured by the absence of diarrheal symptoms or the absence of carriage of C. difficile or C. difficile toxin after 2 day, 4 days, 1 week, 2 weeks, 4 weeks, 8 weeks or longer than 8 weeks.

Microbial Compositions Described by Operational Taxonomic Unit (OTU)

A microbial composition may be prepared comprising at least two types of isolated bacteria, wherein a first type is a first OTU comprising a bacterial species herein, and the second type is a second OTU characterized by, i.e., at least 93%, 94%, 95%, 96%, 97%, 98%, 99%, 99.9%, 99.99% or including 100% sequence identity to the first OTU. Alternatively, the first and second type of OTU may share less than 93% sequence identity. In some embodiments, two types of bacteria are provided in a composition, and the first bacteria and the second bacteria are not the same OTU.

A microbial composition may be prepared comprising at least an isolated bacteria, wherein a first type is a first OTU comprising a bacterial species herein, and the second type is a second OTU characterized by, i.e., at least 93%, 94%, 95%, 96%, 97%, 98%, 99%, 99.9%, 99.99% or including 100% sequence identity to the first OTU. In some embodiments, two types of bacteria are provided in a composition, and the first bacteria and the second bacteria are not the same OTU.

Genetic similarity among OTUs is determined by comparison of one or more nucleic acid sequences representing a given OTU with nucleic acid sequences representing other OTUs. OTUs are defined and compared using both sequence similarity and position in phylogenetic tree. A phylogenetic tree refers to a graphical representation of the evolutionary relationships of one genetic sequence to another that is generated using a defined set of phylogenetic reconstruction algorithms (e.g. parsimony, maximum likelihood, or Bayesian). Nodes in the tree represent distinct ancestral sequences and the confidence of any node is provided by a bootstrap or Bayesian posterior probability, which is a measure of branch uncertainty. OTUs are terminal leaves in a phylogenetic tree (i.e. branch end points) and are defined by a specific genetic sequence and all sequences that share sequence identity to this sequence at the level of species. The specific genetic sequence may be the 16S sequence, portion of the 16S sequence, full genome sequence, or some portion of the full genome sequence. OTUs share at least 95%, 96%, 97%, 98%, or 99% sequence identity. OTUs are frequently defined by comparing sequences between organisms. Sequences with less than 95% sequence identity are not considered to form part of the same OTU. Further, genetic sequences representing a single OTU will form a monophyletic clade (i.e. set of sequences all originating from a single node in the tree).

Detection of Pathogens or Undesired Contaminants

A. Enrichment of Undesired Bacterial Strains and/or Pathogens in Bacterial Compositions

The methods of the invention provide mechanisms by which contaminating bacterial strains (herein “undesired bacteria” or “undesired bacterial strains”) or other pathogens or contaminating materials such as yeast, viruses including phage, or eukaryotic parasites, present at very low levels in a therapeutic bacterial composition or other bacteria-containing materials can be detected and, optionally, quantified. In embodiments of the invention, contaminating bacterial strains present at a ratio of about 10⁻⁵, 10⁻⁶, 10⁻⁷, 10⁻⁸, 10⁻⁹, 10⁻¹⁰, or below 10⁻¹⁰ compared to the non-contaminating strains. In some embodiments, the undesired bacteria are enriched from a bacterial composition prior to performing one or more detection steps on the composition, as provided herein. Multiple methods of enrichment and detection are provided, and one of skill in the art would recognize that one or more enrichment steps can be combined with one or more detection steps. Additionally, the methods of enrichment and/or detection may be repeated one or more times for the same undesired bacterial strain or to address multiple undesired bacterial strains (e.g., one configuration of enrichment steps and detection steps may be performed for the detection of anaerobic contaminants whereas another configuration may be performed for the detection of aerobic contaminants).

In a first method, an enrichment step may be carried out as follows: an antibody or other protein, lectin or other ligand (such as a DNA or RNA aptamer) specific for each of the desired bacterial strains (i.e., the strains intended to be present in the microbial composition) can be attached to a solid support and used to selectively bind to or remove the product strains. The selective removal process may be conducted in: a batch mode, whereby the bacterial composition is contacted with the solid support material to which the antibodies are bound. After an appropriate incubation period, the solid support is removed by filtration, centrifugation or any other method of separation to selectively remove the bound product strains and selectively enrich for the contaminants in the supernatant that is left behind; or a flow mode, whereby the bacterial composition is flowed over the solid support to which the antibody is bound, with the contaminants being selectively enriched in the eluate. In an alternative embodiment a spore fraction can be selectively enriched or removed from a microbial mixture by using a chromatographic separation based on hydrophobic interactions. This can be performed in batch mode or flow mode. In yet another alternative embodiment, the antibody may selectively bind to the suspected contaminant, with subsequent filtration, centrifugation or separation designed to enrich the solid support from which the contaminant can be detected by methods described below.

In a second method, an enrichment step may be carried out as follows: adding to the bacterial composition an antibody specific for each desired bacterial strain, followed by the addition of serum complement to selectively kill or inactivate the desired bacterial strains, thus enriching the undesired bacterial strains. In this method, it is important to select an antibody whose Fc region is capable of being recognized by complement when bound to its target. Thus, IgM would be particularly useful, as would any other IgG subtype that is capable of being recognized by activated complement, but an IgG4 subtype antibody would not generally be appropriate. The method provides for altering parameters of the method based on the number of bacteria in the bacterial composition, e.g., antibody concentration, ionic strength, serum complement concentration and temperature, in order to maximize the killing of the desired bacterial strains and the enrichment of viable contaminants.

In a third method, a conjugated antibody may be used in a homogenous format to bind to and inactivate the desired bacterial strains. In particular, the use of antibodies conjugated to toxins is a means of localizing the toxin activity in the region of the bacteria that one desires to deplete. Many forms of toxins can be envisioned. For instance, the antibody can be covalently paired with an enzyme that converts a non-toxic substrate into a toxin, which then acts locally. The conjugate may be toxic itself or it may be hydrolyzed from the antibody to yield a toxic product. The toxin may be a photoactivatable agent, such as a porphyrin derivative, that forms activated singlet oxygen species in the presence of an appropriate wavelength of light. The enzymatic and photosensitizer approaches have the advantage of temporal separation between the antibody binding event and the toxin activation event. Thus, excess free antibody or antibody that is non-specifically adsorbed to contaminants can be removed by washing before activating the toxin. In the case of using a photosensitizer, the wavelength of light is chosen such that the light by itself has no effect on bacterial viability.

In a fourth method, biological means are provided for selectively enriching for the contaminant (or a product of the contaminant). For example, bacterial viruses (or phage) can be identified that have exquisite sensitivity for replicating in bacteria of a specific genus, species or strain. Thus, phage may be selected that are specific to the product strains but do not replicate in the undesired bacterial strain(s). For instance, phage that replicate in and lyse Bacteroides vulgatus would not have the same effect on Salmonella contaminants. Thus, an appropriately selected population of bacteriophage could be used to selectively enrich the undesired bacterial strains by killing or lysing the desired bacterial strains. Another method employing phage is to selectively enrich the contaminants (or a product of the contaminants) by using phage that grow in an undesired bacterial species. Thus, a coliphage could be added to a mixed bacterial product (i.e., a product known or believed to contain one or more undesired bacterial strains) that is not itself intended to have a coliform bacterium. If the E. coli were present as a contaminant, the phage would bind to and replicate in these contaminating organisms. The phage itself is amplified through this procedure and the amplification product could be detected in a subsequent step.

In a fifth method, bacteriophage can be introduced into a population to induce growth of one or more specified host undesired bacteria. In specific embodiments, phage are engineered to target one or more than one undesired bacteria, and to control the rate of growth of the host bacteria.

In a sixth method, selective culture conditions can be employed to address mixed populations of aerobic and anaerobic bacteria. For example, the mixed population is selectively cultured under or exposed to aerobic conditions. Resulting from this, obligate anaerobes will be killed over a period of time dependent on their oxygen sensitivity. For example, if in a mixed population containing desired bacterial strains and undesired bacterial strains, 4 of 5 strains present are anaerobes, this aerobic cultivation step selectively eliminates the viable anaerobes. As a result, the remaining contaminant is detected as one would for a non-mixed bacterial product containing one desired bacterium and potential non-product contaminants. Optionally, aerobic exposure is followed by one or more selective growth conditions (e.g., selecting against the growth of the remaining aerobic organism) to selectively grow the undesired bacteria. It is then straightforward to define one or more selective media, and each of these are utilized separately to detect the presence of undesired bacterial strains. Examples of selective media are given in the United States Pharmacopeia (USP) Chapters 61, 62, 2021 and 2022 (herein USP <61>, <62>, <2021>, and <2022>), and in Wadsworth-KTL Anaerobic Bacteriology Manual (Star Publishing Company, 6th Edition), Manual of Clinical Microbiology (ASM Press, 10th Edition). By way of non-limiting example, undesired microbes include Pseudomonas aeruginosa, Salmonella spp., Candida albicans, Klebsiella pneumoniae, Aspergillus brasiliensis, Staphylococcus aureus, Clostridium sporogenes, Clostridium difficile, E. coli spp., and Bacillus subtilis, and combinations thereof. Such selective media and their combinations may be used to selectively detect contamination with undesired pathogens and microbes. Media may be validated to detect pathogenic bacteria by testing using model organisms that mimic undesired bacteria.

In a seventh method, mixed populations may be enriched by depletion of classes of microbes that are amenable to separations, or sensitive to treatments. As an example, bacteria of different sizes or morphologies may be sorted from others by flow cytometry using light scattering properties or sorting in a flow cytometer after binding of fluorescently labeled antibodies using distinct fluorophores, or imaged via microscopy and destroyed in situ (see e.g.—Cytometry Part A, 61A:153-161, 2004). Antibiotic treatments and their combinations can selectively deplete major populations, for example gram negative desired strains can be depleted by certain aminoglycoside antibiotics to enrich for gram positive contaminants. Contact with bacteriocins, may also be used for selective depletion of populations (e.g. colicins against E. coli).

In an eighth method, elements of the innate immune system such as pattern recognition receptors may be used to recognize and selectively trap and thus enrich contaminating populations, e.g. mannose binding lectin to bind yeast and other cells, L-ficolin to trap gram positive cells. Enzymatic treatment of the sample to enhance binding of the target population, e.g. treatment with sialidase to enhance binding to asialoglycoprotein receptor, may be performed to enhance binding and depletion/enrichment of populations. Recognition and depletion strategies may be combined with selective killing methods such as combination of mannose binding lectin with complement.

In a ninth method, nucleic acid sequences, e.g., sequences representative of undesired bacterial strains, are enriched, using methods known in the art. For example, nucleic acid probes may be utilized to selectively deplete the sequence of the desired bacterial strains, thus enriching the nucleic acid sequences of the undesired bacterial strains. As an example, hybrid selection using nucleic acid mixtures comprised of DNA, cDNA and/or RNA from a bacterial culture or clinical patient infected with the bacterial strain of interest can be used to selectively enrich, or deplete a target as appropriate. (See, e.g., Melnikov et al., 2011. Genome Biology, 12:R73). In another embodiment, depletion may target nucleic acids known to be in the sample at high concentrations. As a non-limiting example, tRNAs in a sample are derived from a mammalian subject could be viewed as contaminating nucleic acid sequences in a nucleic acid preparation searching for pathogenic species including but not limited to bacterial 16S sequences, antibiotic resistance genes, pathogenic island sequences, toxin genes or other pathogenetic nucleic acid signatures known to one skilled in the art (e.g. see Hacker et al Pathogenicity islands of virulent bacteria: structure, function and impact on microbial evolution. Mol Microbiology 23(6): 1089-1097. 1997). In order to obtain nucleic acid sequences of interest, all bacteria in a bacterial composition are lysed, e.g., through a combination of heat, detergent, enzymatic digestion and/or alkaline pH, followed by steps to purify the total DNA or RNA from other macromolecules. To obtain RNA, cDNA is amplified using methods known in the art, and the DNA and/or cDNA is then subjected to shearing or enzymatic digestion to fragments of appropriate size, in the range of 1000-10,000 base pairs on average. The DNA is denatured by transiently heating. To this denatured DNA mixture, a variety of DNA captures probes are added (alternatively the probes are added prior to heating). These capture probes are designed to bind to known sequences on both strands of the genes of the desired bacterial strains. Furthermore, the capture probes are tagged (e.g.—biotinylated), typically on a 5′ or 3′ end. After an appropriate incubation period to form duplexes between the capture probes and target sequences, the mixture is incubated with a solid matrix to which a tag-binding component (e.g. streptavidin or any other biotin-binding reagent) is attached. Multiple different incubation periods and annealing temperature profiles may be used during the annealing process to selectively capture nucleic acid fragments harboring specific characteristics. The tag-binding matrix selectively binds to the target DNA sequence and removes it from solution. The matrix is removed through a number of means including filtration or centrifugation. The remaining DNA sequences are significantly enriched in contaminant sequences. This procedure may be carried out multiple times in series to achieve successive enrichment of contaminant DNA. By way of non-limiting example, an enrichment using 16S rDNA sequences from the desired bacterial strains enriches for the 16S sequences of contaminating undesired bacterial strains. The resulting enriched mixture may then be evaluated by 16S rDNA deep sequencing to detect the contaminant 16S sequences. Similarly, one may select capture probes that selectively target any other region of the product strain genome. An additional example includes the use of CRISPRs (clustered regularly interspaced short palindromic repeats) to selectively enrich for specific bacterial targets or classes of bacteria.

In a tenth method, one can selectively amplify the nucleic acids in the sample, either as a stand-alone process or after using any of the enrichment methods described herein. Amplification may involve polymerase chain reaction (PCR) or related methods using degenerate primers for highly conserved genes, targeted primers for specific genes known to be harbored by contaminants of interest, or linker ligation strategies for non-specific amplification of all the (remaining) genomes in a sample. An example using degenerate primers would be the set of primers used for 16S rDNA sequencing of microbial specimens—using this method after one or more of the enrichment steps above will selectively amplify contaminant rDNA sequences. Nucleic acid sequences can be detected by sequencing, hybridization to targets, restriction fragment polymorphism or any method for identifying a nucleic acid molecule.

B. Detection in Microbial Compositions.

The methods described herein are useful for detecting one or more species, strains, or other related group of pathogenic or otherwise undesired (i.e., contaminating) microbes. Additionally multiple classes of undesired entities can be simultaneously detected in a material such as a therapeutic bacterial composition. For example, the presence of any two classes of pathogens including pathogenic bacteria, viruses, and fungi, or more than two classes, are simultaneously or sequentially determined in a composition.

Sensitivity of Detection

In some embodiments provided are methods that comprise one or more steps of detecting, or attempting to detect, an undesired entity in a material. In some embodiments, these detection steps individually have a sensitivity for the undesired entity of at least about 1×10², such as 1×10³, 1×10⁴, 1×10⁵, 1×10⁶, or greater than 1×10⁶. When more than one detection step is employed, the combination of two or more detection steps provides a combined sensitivity for the undesired entity of at least about 1×10³, such as 1×10⁴, 1×10⁵, 1×10⁶, 1×10, 1×10⁸, 1×10⁹, 1×10¹⁰, 1×10¹¹, or greater than 1×10¹¹.

In other embodiments, the detection steps individually have a sensitivity to detect the undesired entity at a concentration below that concentration required to detect the desired entity. For example, one detection step, or a combination of two or more detection steps, has the sensitivity to detect the undesired entity, if present in the material, at a concentration below about 1×10⁻² the concentration of the desired entity, such as below about 1×10⁻³, 1×10⁻⁴, 1×10⁻⁵, 1×10⁻⁶, 1×10⁻⁷, 1×10⁻⁸, or below about 1×10⁻⁸ the concentration of the desired entity.

Polymerase chain reaction (PCR), culture and colony counting methods, immunology-based methods and biosensor methods are useful detection steps for detection of pathogen or other undesired biological entities as described herein. Such detection steps can be performed individually, combinatorially, serially, or sequentially. Such detection steps require amplified DNA, RNA, cDNA analysis; counting of bacteria; antigen-antibody interactions; and detection of biological recognition elements (e.g., enzymes, antibodies and nucleic acids), respectively.

Polymerase chain reaction. PCR is a nucleic acid amplification technology based on the isolation, amplification and quantification of one or more DNA sequences including the undesired bacteria's genetic material. Examples of different PCR methods developed for bacterial detection are: (i) real-time PCR, (ii) multiplex PCR and (iii) reverse transcriptase PCR (RT-PCR). There are also methods coupling PCR to other techniques. Multiplex PCR is very useful as it allows the simultaneous detection of several undesired bacteria by introducing different primers to amplify DNA regions coding for specific genes of each undesired bacteria or bacterial strain. One of the limitations of PCR is that the user cannot discriminate between viable and non-viable undesired bacteria because DNA is generally present regardless of the viability of the undesired bacteria. Reverse transcriptase PCR (RT-PCR) was developed may be adapted in order to preferentially detect viable cells. PCR may also be augmented by additional technologies and techniques such as “the most probable number counting method” (MPN-PCR), surface plasmon resonance and PCR-acoustic wave sensors, LightCycler real-time PCR (LC-PCR) and PCR-enzyme-linked immunosorbent assay (PCR-ELISA), a sandwich hybridization assay (SHA) or FISH (fluorescence in situ hybridization) detection, and digital color-coded barcode technologies.

Culture and Colony Counting Methods

The culturing and plating method is generally cited as a standard detection method. Generally, selective and/or differential media are used to detect particular undesired bacteria species or strains. The selective media may contain inhibitors (in order to stop or delay the growth of strains other than undesired bacterial strains) or particular substrates that only the undesired bacteria can degrade or that confers a particular color to the growing colonies. The selective media may contain inhibitors (for example, antibiotics or bile salts) that to prevent or delay the growth of certain species, substrates that allow growth of only certain organisms (for example, cellibiose as the key carbon source such that only cellibiose-utilizing species can grow), and/or particular substrates that yield differential colony morphologies (for example, only the undesired bacteria can degrade a substrate which confers a particular color to the growing colonies). Detection is then carried out using optical methods, mainly by ocular inspection or the use of automated colony counters, sometimes in combination with image analysis, e.g., to identify particular colony morphologies, and color-coded barcode technologies.

Immunology-Based Methods

The field of immunology-based methods for undesired bacteria detection provides analytical tools for a wide range of targets. For example, immunomagnetic separation (IMS) can be used to capture and extract the undesired bacterial strain from the therapeutic composition by introducing antibody coated magnetic beads. IMS is useful in combination with almost any detection method, e.g., optical, magnetic force microscopy, magnetoresistance and Hall effect. Other detection methods are based on immunological techniques, e.g., the enzyme-linked immunosorbent assay (ELISA).

Biosensor-Based Methods in Pathogenic Bacteria or Other Contaminating Material Detection

Biosensors are analytical devices incorporating a biological material, a biologically derived material, or a biomimic associated with or integrated within a physicochemical transducer or transducing microsystem, such as an optical, electrochemical, thermometric, piezoelectric, magnetic or micromechanical systems. There are four main classes of biological recognition elements that are used in biosensor applications: (i) enzymes, (ii) antibodies, (iii) nucleic acids, and (iv) phage.

See, e.g., the following, which are incorporated by reference in their entireties. Abdel-Hamid, 1999. Biosens. Bioelectron. 14,309-316; Blais, 2004. Lett. Appl. Microbiol.; Daly, 2004. J. Appl. Microbiol. 96,419-429; Fu, 2005. Int. J. Food Microbiol. 99, 47-57; Higgins, 2003. Biosens. Bioelectron. 18, 1115-1123; Tims, 2003. J. Microbiol. Methods 55, 141-147; Radke, 2005. Biosens. Bioelectron. 20,1662-1667.

Other embodiments will be apparent to those skilled in the art from consideration of the specification and practice of the embodiments.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Species Identification

The identity of the bacterial species which grew up from a complex fraction can be determined in multiple ways. First, individual colonies can be picked into liquid media in a 96 well format, grown up and saved as 15% glycerol stocks at −80° C. Aliquots of the cultures can be placed into cell lysis buffer and colony PCR methods can be used to amplify and sequence the 16S rDNA gene (Example 3). Alternatively, colonies may be streaked to purity in several passages on solid media. Well separated colonies are streaked onto the fresh plates of the same kind and incubated for 48-72 hours at 37° C. The process is repeated multiple times in order to ensure purity. Pure cultures can be analyzed by phenotypic- or sequence-based methods, including 16S rDNA amplification and sequencing as described in Examples 3 & 4. Sequence characterization of pure isolates or mixed communities e.g. plate scrapes and spore fractions can also include whole genome shotgun sequencing. The latter is valuable to determine the presence of genes associated with sporulation, antibiotic resistance, pathogenicity, and virulence. Colonies can also be scraped from plates en masse and sequenced using a massively parallel sequencing method as described in Examples 3 & 4 such that individual 16S signatures can be identified in a complex mixture. Optionally, the sample can be sequenced prior to germination (if appropriate DNA isolation procedures are used to lsye and release the DNA from spores) in order to compare the diversity of germinable species with the total number of species in a spore sample. As an alternative or complementary approach to 16S analysis, MALDI-TOF-mass spec can also be used for species identification (as reviewed in Anaerobe 22:123).

### Example 2

### Microbiological Strain Identification Approaches

Pure bacterial isolates can be identified using microbiological methods as described in Wadsworth-KTL Anaerobic Microbiology Manual (Jousimies-Somer, et al 2002) and The Manual of Clinical Microbiology (ASM Press, 10th Edition). These methods rely on phenotypes of strains and include Gram-staining to confirm Gram positive or negative staining behavior of the cell envelope, observance of colony morphologies on solid media, motility, cell morphology observed microscopically at 60× or 100× magnification including the presence of bacterial endospores and flagella. Biochemical tests that discriminate between genera and species are performed using appropriate selective and differential agars and/or commercially available kits for identification of Gram negative and Gram positive bacteria and yeast, for example, RapID tests (Remel) or API tests (bioMerieux). Similar identification tests can also be performed using instrumentation such as the Vitek 2 system (bioMerieux). Phenotypic tests that discriminate between genera and species and strains (for example the ability to use various carbon and nitrogen sources) can also be performed using growth and metabolic activity detection methods, for example the Biolog Microbial identification microplates. The profile of short chain fatty acid production during fermentation of particular carbon sources are used as a way to discriminate between species (Wadsworth-KTL Anaerobic Microbiology Manual, Jousimies-Somer, et al 2002). MALDI-TOF-mass spectrometry can also be used for species identification (as reviewed in Anaerobe 22:123).

### Example 3

### Sequence-Based Genomic Characterization of Operational Taxonomic Units (OTU) and Functional Genes

### Method for Determining 16S rDNA Gene Sequence

OTUs are defined either by full 16S sequencing of the rRNA gene, by sequencing of a specific hypervariable region of this gene (i.e. V1, V2, V3, V4, V5, V6, V7, V8, or V9), or by sequencing of any combination of hypervariable regions from this gene (e.g. V1-3 or V3-5). The bacterial 16S rRNA gene is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most microbes. rRNA gene sequencing methods are applicable to both the analysis of non-enriched samples, but also for identification of microbes after enrichment steps that either enrich the microbes of interest from the microbial composition and/or the nucleic acids that harbor the appropriate rDNA gene sequences as described below. For example, enrichment treatments prior to 16S rDNA gene characterization will increase the sensitivity of 16S as well as other molecular-based characterization nucleic acid purified from the microbes.

Using well known techniques to determine the full 16S sequence or the sequence of any hypervariable region of the 16S rRNA sequence, genomic DNA was extracted from a bacterial sample, the 16S rDNA (full region or specific hypervariable regions) amplified using polymerase chain reaction (PCR), the PCR products cleaned, and nucleotide sequences delineated to determine the genetic composition of 16S gene or subdomain of the gene. If full 16S sequencing is performed, the sequencing method used may be, but is not limited to, Sanger sequencing. If one or more hypervariable regions are used, such as the V4 region, the sequencing may be, but is not limited to being, performed using the Sanger method or using a next-generation sequencing method, such as an Illumina (sequencing by synthesis) method using barcoded primers allowing for multiplex reactions.

Method for Determining 18S rDNA and ITS Gene Sequence

Methods to assign and identify fungal OTUs by genetic means are accomplished by analyzing 18S sequences and the internal transcribed spacer (ITS). The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

Using well known techniques, in order to determine the full 18S and ITS sequences or a smaller hypervariable section of these sequences, genomic DNA is extracted from a microbial sample, the rDNA amplified using polymerase chain reaction (PCR), the PCR products cleaned, and nucleotide sequences delineated to determine the genetic composition rDNA gene or subdomain of the gene. The sequencing method used may be, but is not limited to, Sanger sequencing or using a next-generation sequencing method, such as an Illumina (sequencing by synthesis) method using barcoded primers allowing for multiplex reactions.

Method for Determining Other Marker Gene Sequences

In addition to the 16S and 18S rRNA gene, one may define an OTU by sequencing a selected set of genes that are known to be marker genes for a given species or taxonomic group of OTUs. These genes may alternatively be assayed using a PCR-based screening strategy. As example, various strains of pathogenic Escherichia coli can be distinguished using DNAs from the genes that encode heat-labile (LTI, LTIIa, and LTIIb) and heat-stable (STI and STII) toxins, verotoxin types 1, 2, and 2e (VT1, VT2, and VT2e, respectively), cytotoxic necrotizing factors (CNF1 and CNF2), attaching and effacing mechanisms (eaeA), enteroaggregative mechanisms (Eagg), and enteroinvasive mechanisms (Einv). The optimal genes to utilize for taxonomic assignment of OTUs by use of marker genes are familiar to one with ordinary skill of the art of sequence based taxonomic identification.

Genomic DNA Extraction

Genomic DNA is extracted from pure microbial cultures using a hot alkaline lysis method. 1 μl of microbial culture is added to 9 μl of Lysis Buffer (25 mM NaOH, 0.2 mM EDTA) and the mixture is incubated at 95° C. for 30 minutes. Subsequently, the samples are cooled to 4° C. and neutralized by the addition of 10 μl of Neutralization Buffer (40 mM Tris-HCl) and then diluted 10-fold in Elution Buffer (10 mM Tris-HCl). Alternatively, genomic DNA is extracted from pure microbial cultures using commercially available kits such as the Mo Bio Ultraclean® Microbial DNA Isolation Kit (Mo Bio Laboratories, Carlsbad, Calif.) or by standard methods known to those skilled in the art. For fungal samples, DNA extraction can be performed by methods described previously (US20120135127) for producing lysates from fungal fruiting bodies by mechanical grinding methods.

Amplification of 16S Sequences for Downstream Sanger Sequencing

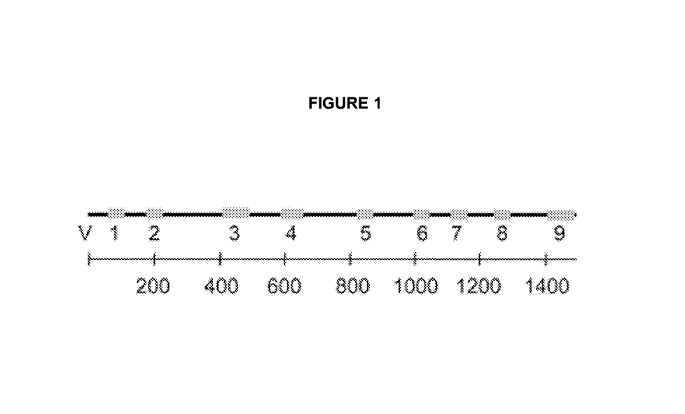

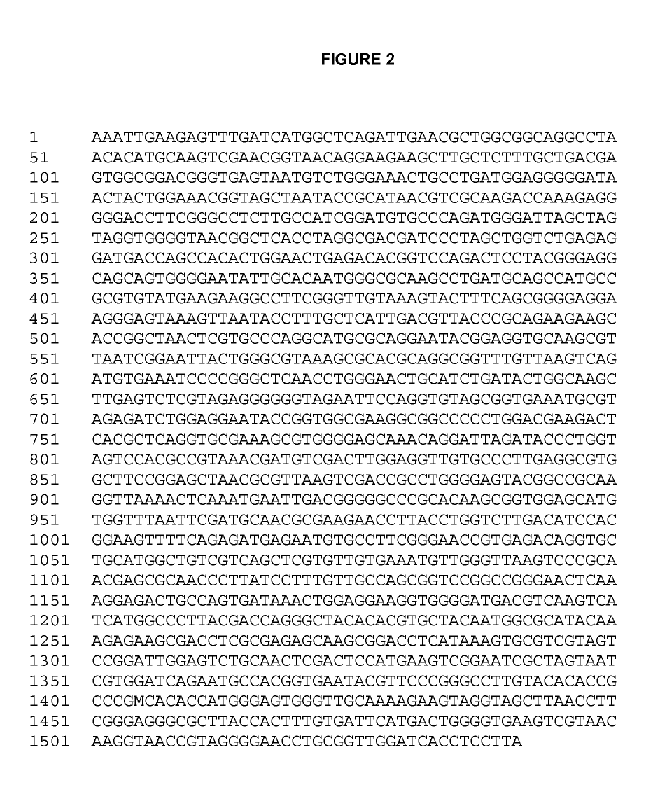

To amplify bacterial 16S rDNA (FIG. 1A), 2 μl of extracted gDNA is added to a 20 μl final volume PCR reaction. For full-length 16 sequencing the PCR reaction also contains 1× HotMasterMix (SPRIME, Gaithersburg, Md.), 250 nM of 27f (AGRGTTTGATCMTGGCTCAG, IDT, Coralville, Iowa), and 250 nM of 1492r (TACGGYTACCTTGTTAYGACTT, IDT, Coralville, Iowa), with PCR Water (Mo Bio Laboratories, Carlsbad, Calif.) for the balance of the volume. Alternatively, other universal bacterial primers or thermostable polymerases known to those skilled in the art are used. For example primers are available to those skilled in the art for the sequencing of the the “V1-V9 regions” of the 16S rRNA (FIG. 1A). These regions refer to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA (in FIG. 1) by comparing the candidate sequence in question to the reference sequence (FIG. 2) and identifying the hypervariable regions based on similarity to the reference hypervariable regions.

FIG. 1 shows the hypervariable regions mapped onto a 16s sequence and the sequence regions corresponding to these sequences on a sequence map.

The PCR is performed on commercially available thermocyclers such as a BioRad MyCycler™ Thermal Cycler (BioRad, Hercules, Calif.). The reactions are run at 94° C. for 2 minutes followed by 30 cycles of 94° C. for 30 seconds, 51° C. for 30 seconds, and 68° C. for 1 minute 30 seconds, followed by a 7 minute extension at 72° C. and an indefinite hold at 4° C. Following PCR, gel electrophoresis of a portion of the reaction products is used to confirm successful amplification of a ˜1.5 kb product.

To remove nucleotides and oligonucleotides from the PCR products, 2 μl of HT ExoSap-IT (Affymetrix, Santa Clara, Calif.) is added to 5 μl of PCR product followed by a 15 minute incubation at 37° C. and then a 15 minute inactivation at 80° C.

Amplification of 16S Sequences for Downstream Characterization by Massively Parallel Sequencing Technologies

Amplification performed for downstream sequencing by short read technologies such as Illumina require amplification using primers known to those skilled in the art that additionally include a sequence-based barcoded tag. As example, to amplify the 16s hypervariable region V4 region of bacterial 16S rDNA, 2 μl of extracted gDNA is added to a 20 μl final volume PCR reaction. The PCR reaction also contains 1× HotMasterMix (5PRIME, Gaithersburg, Md.), 200 nM of V4_{—}515_f adapt (AATGATACGGCGACCACCGAGATCTACACTATGGTAATTGTGTGCCAGCMGCCG CGGTAA, IDT, Coralville, Iowa), and 200 nM of barcoded 806rbc (CAAGCAGAAGACGGCATACGAGAT_{—}12bpGolayBarcode_AGTCAGTCAGCCGGACT ACHVGGGTWTCTAAT, IDT, Coralville, Iowa), with PCR Water (Mo Bio Laboratories, Carlsbad, Calif.) for the balance of the volume. These primers incorporate barcoded adapters for Illumina sequencing by synthesis. Optionally, identical replicate, triplicate, or quadruplicate reactions may be performed. Alternatively other universal bacterial primers or thermostable polymerases known to those skilled in the art are used to obtain different amplification and sequencing error rates as well as results on alternative sequencing technologies.

The PCR amplification is performed on commercially available thermocyclers such as a BioRad MyCycler™ Thermal Cycler (BioRad, Hercules, Calif.). The reactions are run at 94° C. for 3 minutes followed by 25 cycles of 94° C. for 45 seconds, 50° C. for 1 minute, and 72° C. for 1 minute 30 seconds, followed by a 10 minute extension at 72° C. and a indefinite hold at 4° C. Following PCR, gel electrophoresis of a portion of the reaction products is used to confirm successful amplification of a ˜1.5 kb product. PCR cleanup is performed as specified in the previous example.

Sanger Sequencing of Target Amplicons from Pure Homogeneous Samples

To detect nucleic acids for each sample, two sequencing reactions are performed to generate a forward and reverse sequencing read. For full-length 16s sequencing primers 27f and 1492r are used. 40 ng of ExoSap-IT-cleaned PCR products are mixed with 25 pmol of sequencing primer and Mo Bio Molecular Biology Grade Water (Mo Bio Laboratories, Carlsbad, Calif.) to 15 μl total volume. This reaction is submitted to a commercial sequencing organization such as Genewiz (South Plainfield, N.J.) for Sanger sequencing.

Amplication of 18S and ITS Regions for Downstream Sequencing

To amplify the 18S or ITS regions, 2 μL, fungal DNA were amplified in a final volume of 30 μL, with 15 μL, AmpliTaq Gold 360 Mastermix, PCR primers, and water. The forward and reverse primers for PCR of the ITS region are 5′-TCCTCCGCTTATTGATATGC-3′ and 5′-GGAAGTAAAAGTCGTAACAAGG-3′ and are added at 0.2 uM concentration each. The forward and reverse primers for the 18s region are 5′-GTAGTCATATGCTTGTCTC-3′ and 5′-CTTCCGTCAATTCCTTTAAG-3′ and are added at 0.4 uM concentration each. PCR is performed with the following protocol: 95 C for 10 min, 35 cycles of 95 C for 15 seconds, 52 C for 30 seconds, 72 C for 1.5s; and finally 72 C for 7 minutes followed by storage at 4 C. All forward primers contained the M13F-20 sequencing primer, and reverse primers included the M13R-27 sequencing primer. PCR products (3 μL) were enzymatically cleaned before cycle sequencing with 1 μL, ExoSap-IT and 1 μL, Tris EDTA and incubated at 37° C. for 20 min followed by 80° C. for 15 min. Cycle sequencing reactions contained 5 μL, cleaned PCR product, 2 μL, BigDye Terminator v3.1 Ready Reaction Mix, 1 μL, 5× Sequencing Buffer, 1.6 pmol of appropriate sequencing primers designed by one skilled in the art, and water in a final volume of 10 μL. The standard cycle sequencing protocol is 27 cycles of 10 s at 96° C., 5 s at 50° C., 4 min at 60° C., and hold at 4° C. Sequencing cleaning is performed with the BigDye XTerminator Purification Kit as recommended by the manufacturer for 10-μL volumes. The genetic sequence of the resulting 18S and ITS sequences is performed using methods familiar to one with ordinary skill in the art using either Sanger sequencing technology or next-generation sequencing technologies such as but not limited to Illumina.

Preparation of Extracted Nucleic Acids for Metagenomic Characterization by Massively Parallel Sequencing Technologies

Extracted nucleic acids (DNA or RNA) are purified and prepared by downstream sequencing using standard methods familiar to one with ordinary skill in the art and as described by the sequencing technology's manufactures instructions for library preparation. In short, RNA or DNA are purified using standard purification kits such as but not limited to Qiagen's RNeasy Kit or Promega's Genomic DNA purification kit. For RNA, the RNA is converted to cDNA prior to sequence library construction. Following purification of nucleic acids, RNA is converted to cDNA using reverse transcription technology such as but not limited to Nugen Ovation RNA-Seq System or Illumina Truseq as per the manufacturer's instructions. Extracted DNA or transcribed cDNA are sheared using physical (e.g. Hydroshear), acoustic (e.g. Covaris), or molecular (e.g. Nextera) technologies and then size selected as per the sequencing technologies manufacturer's recommendations. Following size selection, nucleic acids are prepared for sequencing as per the manufacturer's instructions for sample indexing and sequencing adapter ligation using methods familiar to one with ordinary skill in the art of genomic sequencing.

Massively Parallel Sequencing of Target Amplicons from Heterogeneous Samples

DNA Quantification & Library Construction

The cleaned PCR amplification products are quantified using the Quant-iT™ PicoGreen® dsDNA Assay Kit (Life Technologies, Grand Island, N.Y.) according to the manufacturer's instructions. Following quantification, the barcoded cleaned PCR products are combined such that each distinct PCR product is at an equimolar ratio to create a prepared Illumina library.

Nucleic Acid Detection

The prepared library is sequenced on Illumina HiSeq or MiSeq sequencers (Illumina, San Diego, Calif.) with cluster generation, template hybridization, isothermal amplification, linearization, blocking and denaturation and hybridization of the sequencing primers performed according to the manufacturer's instructions. 16SV4SeqFw (TATGGTAATTGTGTGCCAGCMGCCGCGGTAA), 16SV4SeqRev (AGTCAGTCAGCCGGACTACHVGGGTWTCTAAT), and 16SV4Index (ATTAGAWACCCBDGTAGTCCGGCTGACTGACT) (IDT, Coralville, Iowa) are used for sequencing. Other sequencing technologies can be used such as but not limited to 454, Pacific Biosciences, Helicos, Ion Torrent, and Nanopore using protocols that are standard to someone skilled in the art of genomic sequencing.

### Example 4

### Sequence Read Annotation

### Primary Read Annotation

Nucleic acid sequences are analyzed and annotated to define taxonomic assignments using sequence similarity and phylogenetic placement methods or a combination of the two strategies. A similar approach can be used to annotate protein names, protein function, transcription factor names, and any other classification schema for nucleic acid sequences. Sequence similarity based methods include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. Common databases include, but are not limited to the Human Microbiome Project, NCBI non-redundant database, Greengenes, RDP, and Silva for taxonomic assignments. For functional assignments reads are mapped to various functional databases such as but not limited to COG, KEGG, BioCyc, and MetaCyc. Further functional annotations can be derived from 16S taxonomic annotations using programs such as PICRUST (M. Langille, et al 2013. Nature Biotechnology 31,814-821). Phylogenetic methods can be used in combination with sequence similarity methods to improve the calling accuracy of an annotation or taxonomic assignment. Here tree topologies and nodal structure are used to refine the resolution of the analysis. In this approach we analyze nucleic acid sequences using one of numerous sequence similarity approaches and leverage phylogenetic methods that are well known to those skilled in the art, including but not limited to maximum likelihood phylogenetic reconstruction (see e.g. Liu K, Linder C R, and Warnow T. 2011. RAxML and FastTree: Comparing Two Methods for Large-Scale Maximum Likelihood Phylogeny Estimation. PLoS ONE 6: e27731. McGuire G, Denham M C, and Balding D J. 2001. Models of sequence evolution for DNA sequences containing gaps. Mol. Biol. Evol 18: 481-490. Wrobel B. 2008. Statistical measures of uncertainty for branches in phylogenetic trees inferred from molecular sequences by using model-based methods. J. Appl. Genet. 49: 49-67.) Sequence reads (e.g. 16S, 18S, or ITS) are placed into a reference phylogeny comprised of appropriate reference sequences. Annotations are made based on the placement of the read in the phylogenetic tree. The certainty or significance of the OTU annotation is defined based on the OTU's sequence similarity to a reference nucleic acid sequence and the proximity of the OTU sequence relative to one or more reference sequences in the phylogeny. As an example, the specificity of a taxonomic assignment is defined with confidence at the the level of Family, Genus, Species, or Strain with the confidence determined based on the position of bootstrap supported branches in the reference phylogenetic tree relative to the placement of the OTU sequence being interrogated. Nucleic acid sequences can be assigned functional annotations using the methods described above.

Clade Assignments

The ability of 16S-V4 OTU identification to assign an OTU as a specific species depends in part on the resolving power of the 16S-V4 region of the 16S gene for a particular species or group of species. Both the density of available reference 16S sequences for different regions of the tree as well as the inherent variability in the 16S gene between different species will determine the definitiveness of a taxonomic annotation. Given the topological nature of a phylogenetic tree and the fact that tree represents hierarchical relationships of OTUs to one another based on their sequence similarity and an underlying evolutionary model, taxonomic annotations of a read can be rolled up to a higher level using a clade-based assignment procedure. Using this approach, clades are defined based on the topology of a phylogenetic tree that is constructed from full-length 16S sequences using maximum likelihood or other phylogenetic models familiar to individuals with ordinary skill in the art of phylogenetics. Clades are constructed to ensure that all OTUs in a given clade are: (i) within a specified number of bootstrap supported nodes from one another (generally, 1-5 bootstraps), and (ii) within a 5% genetic similarity. OTUs that are within the same clade can be distinguished as genetically and phylogenetically distinct from OTUs in a different clade based on 16S-V4 sequence data. OTUs falling within the same clade are evolutionarily closely related and may or may not be distinguishable from one another using 16S-V4 sequence data. The power of clade based analysis is that members of the same clade, due to their evolutionary relatedness, are likely to play similar functional roles in a microbial ecology such as that found in the human gut. Compositions substituting one species with another from the same clade are likely to have conserved ecological function and therefore are useful in the present invention. Notably in addition to 16S-V4 sequences, clade-based analysis can be used to analyze 18S, ITS, and other genetic sequences.

Notably, 16S sequences of isolates of a given OTU are phylogenetically placed within their respective clades, sometimes in conflict with the microbiological-based assignment of species and genus that may have preceded 16S-based assignment. Discrepancies between taxonomic assignment based on microbiological characteristics versus genetic sequencing are known to exist from the literature.

Metaenomic Read Annotation

Metagenomic or whole genome shotgun sequence data is annotated as described above, with the additional step that sequences are either clustered or assembled prior to annotation. Following sequence characterization as described above, sequence reads are demultiplexed using the indexing (i.e. barcodes). Following demultiplexing sequence reads are either: (i) clustered using a rapid clustering algorithm such as but not limited to UCLUST (http://drive5.com/usearch/manual/uclust_algo.html) or hash methods such VICUNA (Xiao Yang, Patrick Charlebois, Sante Gnerre, Matthew G Coole, Niall J. Lennon, Joshua Z. Levin, James Qu, Elizabeth M. Ryan, Michael C. Zody, and Matthew R. Henn (2012) De novo assembly of highly diverse viral populations. BMC Genomics 13:475). Following clustering a representative read for each cluster is identified based and analyzed as described above in “Primary Read Annotation”. The result of the primary annotation is then applied to all reads in a given cluster. (ii) A second strategy for metagenomic sequence analysis is genome assembly followed by annotation of genomic assemblies using a platform such as but not limited to MetAMOS (T J. Treangen et al. 2013 Geneome Biology 14:R2) and other methods familiar to one with ordinary skill in the art.

### Example 5

### qPCR Detection of a Microbial Contaminant in a Microbial Composition

qPCR primers are specifically designed to a the genome of a pathogen of interest and thus detect the pathogen in a microbial composition by presence of its nucleic acid after an appropriate preparation. Standard techniques are followed to generate a standard curve for the pathogen of interest from a known concentration of DNA from that pathogen for comparison. Genomic DNA is extracted from samples using commercially-available kits, such as the Mo Bio Powersoil®-htp 96 Well Soil DNA Isolation Kit (Mo Bio Laboratories, Carlsbad, Calif.), the Mo Bio Powersoil® DNA Isolation Kit (Mo Bio Laboratories, Carlsbad, Calif.), or the QIAamp DNA Stool Mini Kit (QIAGEN, Valencia, Calif.) according to the manufacturer's instructions. The qPCR is conducted using HotMasterMix (SPRIME, Gaithersburg, Md.) and primers specific for the pathogen of interest, and is conducted on a MicroAmp® Fast Optical 96-well Reaction Plate with Barcode (0.1 mL) (Life Technologies, Grand Island, N.Y.) and performed on a BioRad C1000™ Thermal Cycler equipped with a CFX96™ Real-Time System (BioRad, Hercules, Calif.), with fluorescent readings of the FAM and ROX channels. The Cq value for each well on the FAM channel is determined by the CFX Manager™ software version 2.1. The log 10 (cfu/ml) of each experimental sample is calculated by inputting a given sample's Cq value into linear regression model generated from the standard curve comparing the Cq values of the standard curve wells to the known log 10 (cfu/ml) of those samples. The skilled artisan may employ alternative qPCR modes. This technique is employed as an optional alternative detection technique with optional nucleic acid enrichment steps before qPCR or optional microbial enrichment steps before cell lysis.

### Example 6

### Germinating Spores

Microbial compositions comprising bacteria can include species that are in spore form and to culture and enrich these a germination procedure can increase the diversity and counts of bacteria cultivated for detection purposes. Germinating a spore fraction increases the number of viable bacteria that will grow on various media types. To germinate a population of spores, the sample is moved to the anaerobic chamber, resuspended in prereduced PBS, mixed and incubated for 1 hour at 37 C to allow for germination. Germinants can include amino-acids (e.g., alanine, glycine), sugars (e.g., fructose), nucleosides (e.g., inosine), bile salts (e.g., cholate and taurocholate), metal cations (e.g., Mg2+, Ca2+), fatty acids, and long-chain alkyl amines (e.g., dodecylamine, Germination of bacterial spores with alkyl primary amines” J. Bacteriology, 1961.). Mixtures of these or more complex natural mixtures, such as rumen fluid or Oxgall, can be used to induce germination. Oxgall is dehydrated bovine bile composed of fatty acids, bile acids, inorganic salts, sulfates, bile pigments, cholesterol, mucin, lecithin, glycuronic acids, porphyrins, and urea. The germination can also be performed in a growth medium like prereduced BHIS/oxgall germination medium, in which BHIS (Brain heart infusion powder (37 g/L), yeast extract (5 g/L), L-cysteine HCl (1 g/L)) provides peptides, amino acids, inorganic ions and sugars in the complex BHI and yeast extract mixtures and Oxgall provides additional bile acid germinants.

In addition, pressure may be used to germinate spores (Gould and Sale (1970) J. Gen. Microbiol. 60: 335). The selection of germinants can vary with the microbe being sought. Different species require different germinants and different isolates of the same species can require different germinants for optimal germination. Finally, it is important to dilute the mixture prior to plating because some germinants are inhibitory to growth of the vegetative-state microorganisms. For instance, it has been shown that alkyl amines must be neutralized with anionic lipophiles in order to promote optimal growth. Bile acids can also inhibit growth of some organisms despite promoting their germination, and must be diluted away prior to plating for viable cells.

For example, BHIS/oxgall solution is used as a germinant and contains 0.5×BHIS medium with 0.25% oxgall (dehydrated bovine bile) where 1×BHIS medium contains the following per L of solution: 6 g Brain Heart Infusion from solids, 7 g peptic digest of animal tissue, 14.5 g of pancreatic digest of casein, 5 g of yeast extract, 5 g sodium chloride, 2 g glucose, 2.5 g disodium phosphate, and 1 g cysteine. Additionally, Ca-DPA is a germinant and contains 40 mM CaCl2, and 40 mM dipicolinic acid (DPA). Rumen fluid (Bar Diamond, Inc.) is also a germinant. Simulated gastric fluid (Ricca Chemical) is a germinant and is 0.2% (w/v) Sodium Chloride in 0.7% (v/v) Hydrochloric Acid. Mucin medium is a germinant and prepared by adding the following items to 1 L of distilled sterile water: 0.4 g KH2PO4, 0.53 g Na2HPO4, 0.3 g NH4C1, 0.3 g NaCl, 0.1 g MgCl2×6H2O, 0.11 g CaCl2, 1 ml alkaline trace element solution, 1 ml acid trace element solution, 1 ml vitamin solution, 0.5 mg resazurin, 4 g NaHCO3, 0.25 g Na2S×9 H2O. The trace element and vitamin solutions prepared as described previously (Stams et al., 1993). All compounds were autoclaved, except the vitamins, which were filter-sterilized. The basal medium was supplemented with 0.7% (v/v) clarified, sterile rumen fluid and 0.25% (v/v) commercial hog gastric mucin (Type III; Sigma), purified by ethanol precipitation as described previously (Miller & Hoskins, 1981). This medium is referred herein as mucin medium.

Fetal Bovine Serum (Gibco) can be used as a germinant and contains 5% FBS heat inactivated, in Phosphate Buffered Saline (PBS, Fisher Scientific) containing 0.137M Sodium Chloride, 0.0027M Potassium Chloride, 0.0119M Phosphate Buffer. Thioglycollate is a germinant as described previously (Kamiya et al Journal of Medical Microbiology 1989) and contains 0.25M (pH10) sodium thioglycollate. Dodecylamine solution containing 1 mM dodecylamine in PBS is a germinant. A sugar solution can be used as a germinant and contains 0.2% fructose, 0.2% glucose, and 0.2% mannitol. Amino acid solution can also be used as a germinant and contains 5 mM alanine, 1 mM arginine, 1 mM histidine, 1 mM lysine, 1 mM proline, 1 mM asparagine, 1 mM aspartic acid, 1 mM phenylalanine A germinant mixture referred to herein as Germix 3 can be a germinant and contains 5 mM alanine, 1 mM arginine, 1 mM histidine, 1 mM lysine, 1 mM proline, 1 mM asparagine, 1 mM aspartic acid, 1 mM phenylalanine, 0.2% taurocholate, 0.2% fructose, 0.2% mannitol, 0.2% glucose, 1 mM inosine, 2.5 mM Ca-DPA, and 5 mM KCl. BHIS medium+DPA is a germinant mixture and contains BHIS medium and 2 mM Ca-DPA. Escherichia coli spent medium supernatant referred to herein as EcSN is a germinant and is prepared by growing E. coli MG1655 in SweetB/Fos inulin medium anaerobically for 48 hr, spinning down cells at 20,000 rcf for 20 minutes, collecting the supernatant and heating to 60 C for 40 min. Finally, the solution is filter sterilized and used as a germinant solution.

### Example 7

### Selection of Media for Growth

It is important to select appropriate media to support growth, including preferred carbon sources. For example, some organisms prefer complex sugars such as cellobiose over simple sugars. Examples of media used in the isolation of sporulating organisms include EYA, BHI, BHIS, and GAM (see below for complete names and references). Multiple dilutions were plated out to ensure that some plates had well isolated colonies on them for analysis, or alternatively plates with dense colonies were scraped and suspended in PBS to generate a mixed diverse community. Various medias will enrich for certain organisms and thus culturing itself is a method of selection and enrichment.

Plates were incubated anaerobically or aerobically at 37 C for 48-72 or more hours, targeting anaerobic or aerobic spore formers, respectively.

Solid plate media include Gifu Anaerobic Medium (GAM, Nissui) without dextrose supplemented with fructooligosaccharides/inulin (0.4%), mannitol (0.4%), inulin (0.4%), or fructose (0.4%), or a combination thereof, Sweet GAM [Gifu Anaerobic Medium (GAM, Nissui)] modified, supplemented with glucose, cellobiose, maltose, L-arabinose, fructose, fructooligosaccharides/inulin, mannitol and sodium lactate), Brucella Blood Agar (BBA, Atlas, Handbook of Microbiological Media, 4th ed, ASM Press, 2010), PEA sheep blood (Anaerobe Systems; 5% Sheep Blood Agar with Phenylethyl Alcohol),

Egg Yolk Agar (EYA) (Atlas, Handbook of Microbiological Media, 4th ed, ASM Press, 2010), Sulfite polymyxin milk agar (Mevissen-Verhage et al., J. Clin. Microbiol. 25:285-289 (1987)), Mucin agar (Derrien et al., IJSEM 54: 1469-1476 (2004)),

Polygalacturonate agar (Jensen & Canale-Parola, Appl. Environ. Microbiol. 52:880-997 (1986)),

M2GSC (Atlas, Handbook of Microbiological Media, 4th ed, ASM Press, 2010),

M2 agar (Atlas, Handbook of Microbiological Media, 4th ed, ASM Press, 2010), supplemented with starch (1%), mannitol (0.4%), lactate (1.5 g/L) or lactose (0.4%),

Sweet B—Brain Heart Infusion agar (Atlas, Handbook of Microbiological Media, 4th ed, ASM Press, 2010) supplemented with yeast extract (0.5%), hemin, cysteine (0.1%), maltose (0.1%), cellobiose (0.1%), soluble starch (sigma, 1%), MOPS (50 mM, pH 7),

PY-salicin (peptone-yeast extract agar supplemented with salicin) (Atlas, Handbook of Microbiological Media, 4th ed, ASM Press, 2010)., Modified Brain Heart Infusion (M-BHI) [[sweet and sour]] contains the following per L: 37.5 g Brain Heart Infusion powder (Remel), 5 g yeast extract, 2.2 g meat extract, 1.2 g liver extract, 1 g cystein HCl, 0.3 g sodium thioglycolate, 10 mg hemin, 2 g soluble starch, 2 g FOS/Inulin, 1 g cellobiose, 1 g L-arabinose, 1 g mannitol, 1 Na-lactate, 1 mL Tween 80, 0.6 g MgSO4×7H2O, 0.6 g CaCl2, 6 g (NH4)2SO4, 3 g KH2PO4, 0.5 g K2HPO4, 33 mM Acetic acid, 9 mM propionic acid, 1 mM Isobutyric acid, 1 mM isovaleric acid, 15 g agar, and after autoclaving add 50 mL of 8% NaHCO3 solution and 50 mL 1M MOPS-KOH (pH 7).

Noack-Blaut Eubacterium agar (See Noack et al. J. Nutr. (1998) 128:1385-1391),

BHIS azl/ge2-BHIS az/ge agar (Reeves et. al. Infect. Immun. 80:3786-3794 (2012)) [Brain Heart Infusion agar (Atlas, Handbook of Microbiological Media, 4th ed, ASM Press, 2010) supplemented with yeast extract 0.5%, cysteine 0.1%, 0.1% cellobiose, 0.1% inulin, 0.1% maltose, aztreonam 1 mg/L, gentamycin 2 mg/L],

BHIS CInM azl/ge2-BHIS CInM [Brain Heart Infusion agar (Atlas, Handbook of Microbiological Media, 4th ed, ASM Press, 2010) supplemented with yeast extract 0.5%, cysteine 0.1%, 0.1% cellobiose, 0.1% inulin, 0.1% maltose, aztreonam 1 mg/L, gentamycin 2 mg/L].

### Example 8

### Qualification of Fecal Donor as Healthy

To determine that a donor of fecal material is a healthy, normal individual, testing is performed to determine their general health and the state of the individuals microbiome. Briefly, the individual is questioned on risk factors for dsybiosis and exposure to pathogens ensuring no oral antibiotic use in the past 3 to 6 months, no recent bouts of diarrhea, no travel outside of the united states, canada, or to locations at risk for malaria exposure, and other questions contained on the AABB questionnaire as previously described (e.g. see http://www.aabb.org/resources/donation/questionnaires/Pages/dhqaabb.aspx). A medical history will be assessed with a focus on gastrointestinal history including a history of IBD, colitis, colorectal cancer, C. difficile infection, diarrhea. A rectal exam is also performed to assess colorectal health. Optionally, donors will also be assessed for drug use including smoking, alcohol use, and other common illicit drugs known to one skilled in the art. A fecal sample will be assessed for spore content using methods described herein (e.g. see examples 14 and 15). Additionally fecal based pathogens will be tested for using standard culture and moleculer tests that are commercially available and performed in clinical microbiological labs (e.g. see Versalovic et al 2011 Manual of Clinical Microbiology. American Society for Microbiology, 10th edition or http://www.questdiagnostics.com/testcenter/TestCenterHome.action). Tests performed on feces are obtained and are tested for infectious agents including but not limited to C. difficile, E. coli 0157, camplyobacter, yersinia, salmonella, shigella, cryptosporidium, cyclospora, isospora, rotavirus, norovirus, ova and parasite testing on a fecal smear with acid fast staining, giardia, vibrio cholera. Health donors may also be qualified by having regular bowel movements with stool appearance typically Type 2, 3, 4, 5 or 6 on the Bristol Stool Scale, and having a BMI ≧18 kg/m2 and <25 kg/m2. Blood may optionally be drawn and tested for the presence of infectious agents including but not limited to treponema pallidum, HAV, HBV, HCV, HIV 1/2 HTLV I/II, westnile virus by methods known to one skilled in the art (e.g. see http://www.questdiagnostics.com/testcenter/TestCenterHome.action and http://www.fda.gov/BiologicsBloodVaccines/BloodBloodProducts/ApprovedProducts/Licens edProductsBLAs/BloodDonorScreening/InfectiousDisease/ucm080466.htm). Finally normal blood biochemistry can also be assessed to demonstrate a donor is healthy by evaluating the biochemical and chemical blood metabolite markers including but not limited to complete blood count with platelets, sodium, potassium, chloride, albumin, total protein, glucos, blood urea nitrogen (BUN), creatinine, uric acid, aspartate aminotrasferase (AST), Alanine aminotransferase (ALT), gamma-glutamyltranspeptidase (GGT), creatine kinase (CK), alkaline phosphatase, total bilirubin, direct bilirubin, lactate dehrogenase, calcium, cholesterol, triglycerides by methods known to one skilled in the art and commercially available (e.g. see http://www.questdiagnostics.com/testcenter/TestCenterHome.action). A complete urinalysis can also be performed to assess health. Additionally one or more specific OTUs or Clades desired in the microbial composition can be detected by methods described herein using genetic e.g. PCR, qPCR, 16S, etc., biochemical e.g. serological testing with antibodies, enzymatic activity, etc., microbiological techniques e.g. culturing, etc. or a combination thereof described herein.

Other exclusion criteria generally included significant chronic or acute medical conditions including renal, hepatic, pulmonary, gastrointestinal, cardiovascular, genitourinary, endocrine, immunologic, metabolic, neurologic or hematological disease, a family history of, inflammatory bowel disease including Crohn's disease and ulcerative colitis, Irritable bowel syndrome, colon, stomach or other gastrointestinal malignancies, or gastrointestinal polyposis syndromes, or recent use of yogurt or commercial probiotic materials in which an organism(s) is a primary component.

### Example 9

### Purification and Isolation of a Spore Forming Fraction From Feces

To enrich a spore fraction or generate an ethanol treated fecal suspension from a greater microbial composition e.g. stool or other composition, for further testing the following non-limiting example presents a protocol for isolating a spore forming fraction from a microbial composition e.g. feces. To purify and selectively isolate efficacious spores from fecal material a stool donation was first blended with saline using a homogenization device (e.g., laboratory blender) to produce a 20% slurry (w/v). 100% ethanol was added for an inactivation treatment that lasts 10 seconds to 1 hour. The final alcohol concentration ranged from 30-90%, preferably 50-70%. High speed centrifugation (3200 rcf for 10 min) was performed to remove solvent and the pellet was retained and washed.

Once the washed pellet was resuspended, a low speed centrifugation step (200 rcf for 4 min) was performed to remove large particulate vegetative matter and the supernatant containing the spores was retained. Low-speed centrifugation selectively removes large particles, and therefore removes up to 7-61% of fibrous material, with a recovery of spores of between 50 and 85%. Alternatively, the resuspended pellet can be filtered through 600 um, 300 um, 200 um, 150 um, 100 um, 75 um, 60 um, 50 um, 20 um pore-size filters. This similarly selectively removes large particles, allowing spores to pass through the filters, removing 15-80% of solids while retaining 80-99% of spores, as measured by DPA.

High speed centrifugation (3200 rcf for 10 min) was performed on the supernatant to concentrate the spore material. The pellet was then washed and resuspended to generate a 20% slurry. This was the ethanol treated fecal suspension. The concentrated slurry was then separated with a density based gradient e.g. a CsCl gradient, sucrose gradient or combination of the two generating a ethanol treated, gradient-purified spore preparation. For example, a CsCl gradient was performed by loading a 20% volume of spore suspension on top a 80% volume of a stepwise CsCl gradient (w/v) containing the steps of 64%, 50%, 40% CsCl (w/v) and centrifuging for 20 min at 3200 rcf. The spore fraction was then run on a sucrose step gradient with steps of 67%, 50%, 40%, and 30% (w/v). When centrifuged in a swinging bucket rotor for 10 min at 3200 rcf. The spores ran roughly in the 30% and 40% sucrose fractions. The lower spore fraction was then removed and washed to produce a concentrated ethanol treated, gradient-purified spore preparation. Taking advantage of the refractive properties of spores observed by phase contrast microscopy (spores are bright and refractive while germinated spores and vegetative cells are dark) one could see an enrichment of the spore fraction from a fecal bacterial cell suspension compared to an ethanol treated, CsCl gradient purified, spore preparation, and to an ethanol treated, CsCl gradient purified, sucrose gradient purified, spore preparation.

Furthermore, growth of spores after treatment with a germinant was used to quantify a viable spore population. Samples were incubated with a germinant (Oxgall, 0.25% for up to 1 hour), diluted and plated anaerobically on BBA (Brucella Blood Agar) or similar media as described herein. Individual colonies were picked and DNA isolated for full-length 16S sequencing to identify the species composition. This microbial composition e.g. ethanol treated spore preparation or any preparation combination of steps described above served as test material for subsequent enrichment and detection of microbes of interest.

Fibrous material in a stool suspension can be quantified, most easily by taking dry weight measurements. A stool suspension was divided into two equal 3-5 mL samples. One was centrifuged at 3200 rcf for ten minutes, and the supernatant was retained. Three to five mL of the homogenous stool suspension was loaded onto a moisture analyzer and baked until the mass levels off, and the moisture analyzer automatically calculated the percent solids in the sample. The supernatant of the pelleted stool suspension was run as a control to measure dissolved solids. Quantifying undissolved solids was accomplished by subtracting dissolved solids from total solids. This gave an estimate of fibrous contaminants in a stool suspension, as the non-spore, non-bacterial solids make up the bulk of a stool suspension. Quantifying bacterial spores is most easily done by measuring the DPA contents of a sample, and comparing this DPA content to a sample of known spore content (see example above). Expressing DPA content per unit dry material in a suspension gives a measure of the purity of the spore suspension. Eliminating dry material that doesn't contain spores (i.e. fibre) will increase this metric.

### Example 10

### Enrichment and Purification of Bacteria

To purify individual bacterial strains for subsequent detection and identification, dilution plates are selected in which the density enables distinct separation of single colonies. Colonies are picked with a sterile implement (either a sterile loop or toothpick) and re-streaked to BBA or other solid media. Plates are incubated at 37° C. for 3-7 days. One or more well-isolated single colonies of the major morphology type are re-streaked. This process is repeated at least three times until a single, stable colony morphology is observed. The isolated microbe is then cultured anaerobically in liquid media for 24 hours or longer to obtain a pure culture of 106-1010 cfu/ml. Liquid growth medium might include Brain Heart Infusion-based medium (Atlas, Handbook of Microbiological Media, 4th ed, ASM Press, 2010) supplemented with yeast extract, hemin, cysteine, and carbohydrates (for example, maltose, cellobiose, soluble starch) or other media described previously (e.g. see example 7). The culture is centrifuged at 10,000×g for 5 min to pellet the bacteria, the spent culture media is removed, and the bacteria were resuspended in sterile PBS. Sterile 75% glycerol is added to a final concentration of 20%. An aliquot of glycerol stock is titered by serial dilution and plating. The remainder of the stock is frozen on dry ice for 10-15 min and then placed at −80 C for long term storage.

### Example 11

### Titer Determination

The number of viable cells per ml were determined on the freshly harvested, washed and concentrated culture by plating serial dilutions of the RCB to Brucella blood agar or other solid media, and varied from 106 to 1010 cfu/ml. The impact of freezing on viability was determined by titering the banks after one or two freeze-thaw cycles on dry ice or at −80° C., followed by thawing in an anaerobic chamber at room temperature. Some strains displayed a 1-3 log drop in viable cfu/ml after the 1st and/or 2nd freeze thaw, while the viability of others were unaffected.

### Example 12

### Treatment of Fecal Suspensions with Ethanol or Heat Reduces Vegetative Cell Numbers and Results in an Enrichment of Spore Forming Species

Treatment of a sample, preferably a human fecal sample, in a manner to inactivate or kill substantially all of the vegetative forms of bacteria present in the sample results in selection and enrichment of the spore fraction. Methods for inactivation can include heating, sonication, detergent lysis, enzymatic digestion (such as lysozyme and/or proteinase K), ethanol or acid treatment, exposure to solvents (Tetrahydrofuran, 1-butanol, 2-butanol, 1,2 propanediol, 1,3 propanediol, butanoate, propanoate, chloroform, dimethyl ether and a detergent like triton X-100, diethyl ether), or a combination of these methods. To demonstrate the efficacy of ethanol induced inactivation of vegetative cells, a 10% fecal suspension was mixed with absolute ethanol in a 1:1 ratio and vortexed to mix for 1 min. The suspension was incubated at room temperature for 30 min, 1 h, 4 h or 24 h. After incubation the suspension was centrifuged at 13,000 rpm for 5 min to pellet spores. The supernatant was discarded and the pellet was resuspended in equal volume of PBS. Viable cells were measured as described below.

To demonstrate the efficacy of heat treatment on vegetative cell inactivation a 10-20% fecal suspension was incubated at 70 C, 80 C, 90 C or 100 C for 10 min or 1 h.

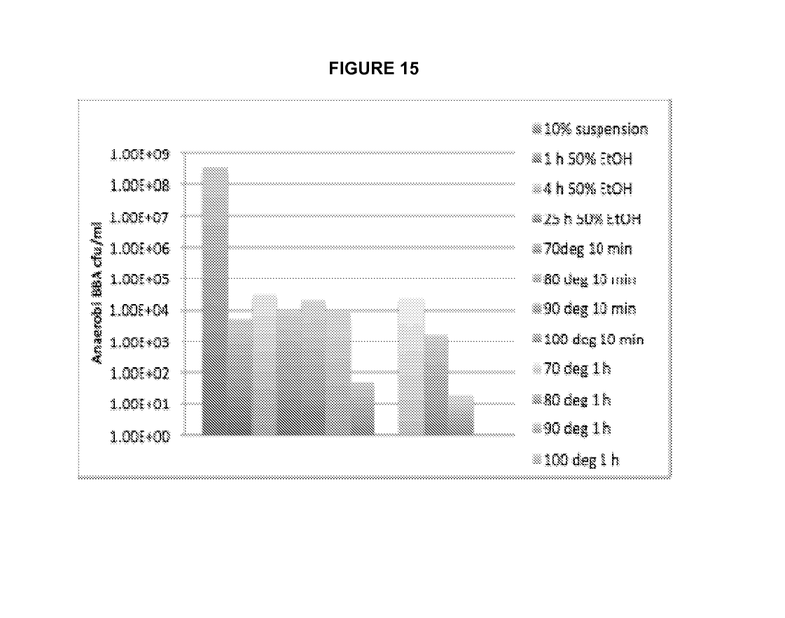

After ethanol or heat treatment, remaining viable cells were measured after 24 h incubation on plates by determining the bacterial titer on Brucella blood agar (BBA) as a function of treatment and time (See FIG. 15). Ethanol treatment for 1 h and 25 h have similar effects, reducing the number of viable cells by approximately 4 logs, while increasing temperature and time at high temperature leads to higher losses in viable cell number, with no colonies detectable at 100° C. at either 10 min or 1 h. No germinants were used. After several days of additional growth on plates, a number of colonies were picked from these treated samples and identified by 16S rDNA analysis (e.g. see Examples 3 and 4). These included known spore forming Clostridium spp. as well as species not previously reported to be spore formers including Ruminococcus bromii, and Anaerotruncus colihominis (Lawson, et al 2004), and a Eubacterium sp. (Table 16). See FIG. 15: Heat and ethanol treatments reduce cell viability

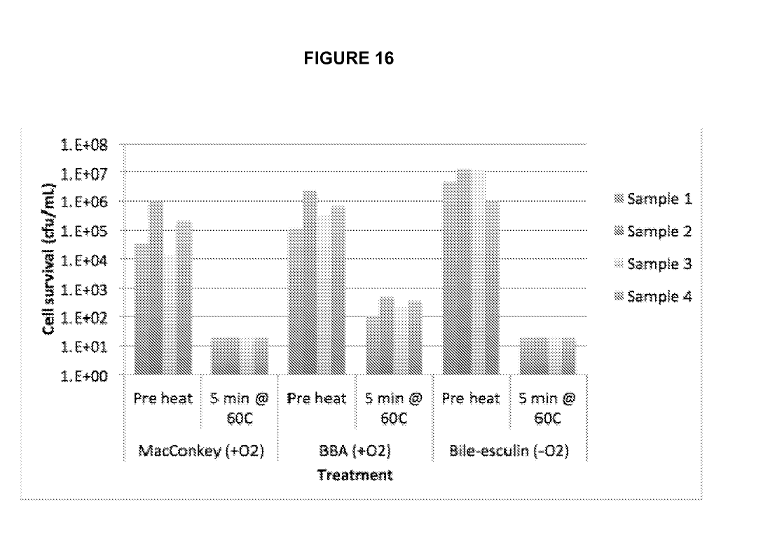

To demonstrate that vegetative cells are reduced by ethanol treatment, known non-spore forming bacteria were ethanol treated as described previously (e.g. see Example 9) and viability was determined by plating on BBA in anaerobic conditions (e.g. see Example 7). Fecal material from four independent donors was exposed to 60 C for 5 min and subsequently plated on three types of selective media under either aerobic (+O2) or anaerobic conditions (—O2) (BBA+aerobic, MacConkey lactose+aerobic, Bacteroides Bile esculin+anaerobic) to identify known nonsporeforming Enterobacteria (survivors on MacConkey agar) and Bacteroides fragilis group species (survivors on Bacteroides Bile Esculin plates). The detectable limit for these assays was roughly 20 cfu/mL. Germinants were not used in this experiment (FIG. 16). Both ethanol and heat inactivation reduces the cell viability from fecal material to the limit of detection under using MacConkey lactose agar and BBE agar. The remaining cells identified on BBA media grown in anaerobic conditions comprise the non-germinant dependent spore forming species. See FIG. 16: Reduction in non-spore forming vegetative cells by treatment at 60° C. for 5 min

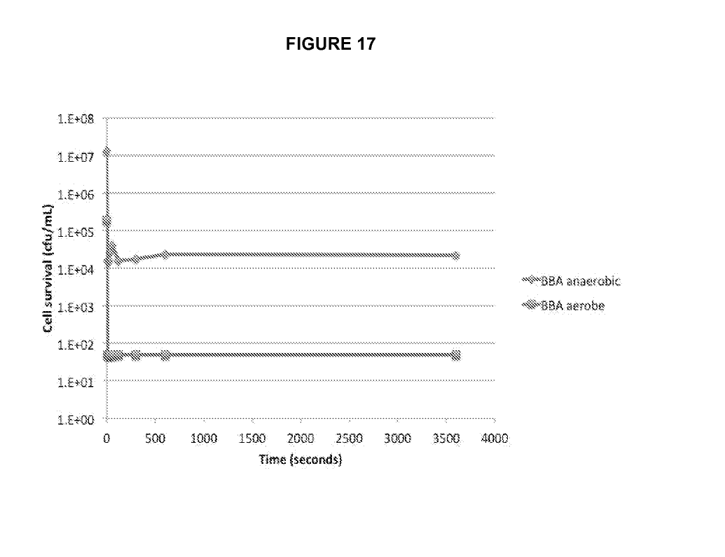

The ethanol treatment was shown to rapidly kill both aerobic and non-spore forming anaerobic colony forming units in 10% fecal suspensions as determined by plating on rich (BBA) media. The reduction of plated CFUs decreases four orders of magnitude in seconds as shown in FIG. 17.

See FIG. 17: Time course demonstrates ethanol reduces both anaerobic and aerobic bacterial CFUs

### Example 13

### Species Identified and Isolated as Spore Formers by Ethanol Treatment

To demonstrate that spore-forming species were enriched by heat or ethanol treatment methods, a comparison of >7000 colony isolates was performed to identify species in a repeatable fashion (e.g., identified independently in multiple preparations, see examples 1, 3, and 4) only isolated from fecal suspensions treated with 50% ethanol or heat treatment and not from untreated fecal suspensions (Table 17). These data demonstrate the ability to select for spore forming species from fecal material, and identify organisms as spore formers not previously described as such in the literature. In each case, organisms were picked as an isolated colony, grown anaerobically, and then subjected to full-length 16S sequencing in order to assign species identity.

To further identify spore formers, ethanol treated fecal samples from donors A, B, C, D, E and F were plated to a variety of solid media types, single colonies were picked and grown up in broth in a 96 well format (Tables 18-23). The 16S rRNA gene was then amplified by PCR and direct cycle sequencing was performed (See examples 3 and 4). The ID is based on the forward read from direct cycle sequencing of the 16S rRNA gene.

There is surprising heterogeneity in the microbiome from one individual to another (Clemente et al., 2012) and this has consequences for determining the potential efficacy of various donors to generate useful spore compositions. The method described below is useful for screening donors when, for instance, a particular quantity or diversity of spore forming organisms is useful or desired for repopulating the microbiome following antibiotic treatment or treating a particular disease or condition. Further, such screening is useful when there is a need to screen donors for the purpose of isolating microorganisms capable of spore formation, or when a purified preparation of spore forming organisms is desired from a particular donor.

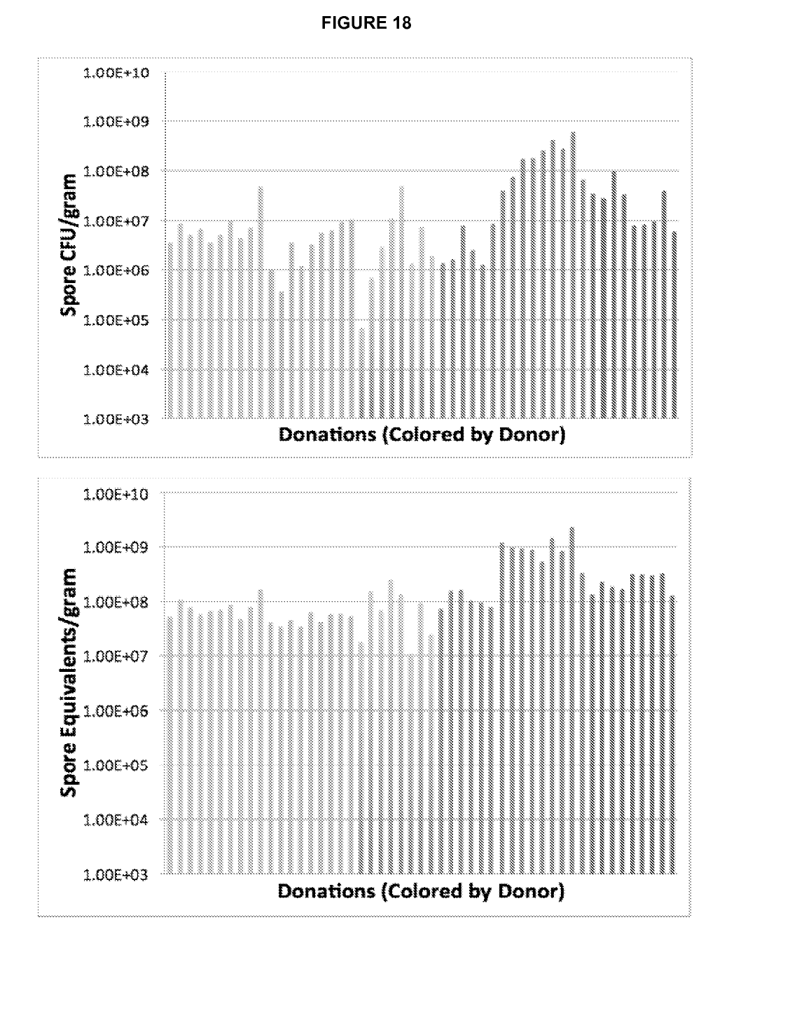

Total spore count is also a measure of potency of a particular donation or purified spore preparation and is vital to determine the quantity of material required to achieve a desired dose level. To understand the variability in total spore counts, donor samples were collected and processed as described in prior examples. Donor spore counts in CFU/g were then determined by growth on media plates at various titrations to determine the spore content of the donation. Furthermore, DPA assays were used to assess spore content (expressed as spore equivalents) as described in Example 14. As seen in FIG. 18, there is as much as two logs difference in an individual donor over time and can be up to three logs difference between donors. The difference in spore content measures is that nonviable spores and non-germinable spores will not be observed by plating but will have measurable DPA content. The variability between species of DPA content in spores making some complex mixtures containing high DPA spores while other mixtures contain low DPA content spores. Selecting donors with high spore counts is important in determining productivity of isolating spores from fecal donations by identifying preferred donors.

A fresh fecal sample from donor F was treated as described in Example 15 to generate an ethanol treated spore fraction, germinated with BHIS/Oxgall for 1 h as a described (e.g. see Example 6), then plated to a variety of media (e.g. See example 7). Colonies were picked with a focus on picking several of each type of morphologically distinct colony on each plate to capture as much diversity as possible. Colonies were counted on a plate of each media type with well isolated colonies such that the number of colony forming units per ml can be calculated (Table 24). Colonies were picked into one of several liquid media and the 16S rDNA sequences (e.g. see Examples 3 and 4) were determined and analyzed as described above. The number of unique OTUs for each media type is shown below with the media with the most unique OTUs at the top (Table 24). Combinations of 3 to 5 of the top 5 media types capture diversity, and some other can be chosen to target specific species of interest. Colony forming units were calculated for a given species using the 16S data, and were used to determine whether a sufficient level of a given organism is present. The spore complement from Donor F includes these 52 species as determined by 16S sequencing (Table 24).

To screen human donors for the presence of a diversity of spore forming bacteria and/or for specific spore-forming bacteria, fecal samples were prepared using germinants and selective plating conditions and colonies were picked (e.g. see Examples 6 and 7) and analyzed for 16S diversity as described previously (see Examples 3 and 4). An assessment of donor diversity included the cfu/ml of ethanol resistant cells on a given media type, or cfu/ml of a given species using the 16S analysis of colonies picked from that media to determine the level of spores of a given species of interest. This culture-based analysis was complemented by culture-independent methods such as qPCR with probes specific to species or genera of interest or metagenomic sequencing of spore preparations, or 16S profiling of spore preparations using Illumina 16S variable region sequencing approaches (e.g. see Examples 3 and 4).

### Example 14

### Quantification of Spore Concentrations in a Microbial Composition Using DPA Assay

Methods to assess spore concentration in microbial compositions typically require the separation and selection of spores and subsequent growth of individual species to determine the colony forming units. The art does not teach how to quantitatively germinate all the spores in such a microbial composition as there are many species for which appropriate germinants have not been identified. Furthermore, sporulation is thought to be a stochastic process as a result of evolutionary selection, meaning that not all spores from a single species germinate with same response to germinant concentration, time and other environmental conditions. Alternatively, a key metabolite of bacterial spores, dipicolinic acid (DPA) has been developed to quantify spores particles in a sample and avoid interference from fecal contaminants. This method can also be used to determine the presence of spores in other products including but not limited to liquid cultures, liquid beverages, resuspended powders and other products not designed to contain spore forming microbes. Thus, the DPA assay described provides a sensitive way of detecting contaminating spores in a complex product in addition to the utility described herein. The assay utilizes the fact that DPA chelates Terbium 3+ to form a luminescent complex (Fichtel et al, FEMS Microbiology Ecology, 2007; Kort et al, Applied and Environmental Microbiology, 2005; Shafaat and Ponce, Applied and Environmental Microbiology, 2006; Yang and Ponce, International Journal of Food Microbiology, 2009; Hindle and Hall, Analyst, 1999). A time-resolved fluorescence assay detects terbium luminescence in the presence of DPA giving a quantitative measurement of DPA concentration in a solution.

The assay was performed by taking 1 mL of the spore standard to be measured and transferring it to a 2 mL microcentrifuge tube. The samples were centrifuged at 13000 RCF for 10 min and the samples were washed in 1 mL sterile deionized H2O. The samples were washed an additional time by repeating the centrifugation. The 1 mL solutions were transferred to hungate tubes and samples were autoclaved on a steam cycle for 30 min at 250 C. 100 uL of 30 uM TbCl3 solution (400 mM sodium acetate, pH 5.0, 30 μM TbCl3) was added to each sample. Serial dilutions of the autoclaved material were made and the fluorescence of each sample was measured by exciting with 275 nm light and measuring the emission wavelength of 543 nm for an integration time of 1.25 ms and a 0.1 ms delay.

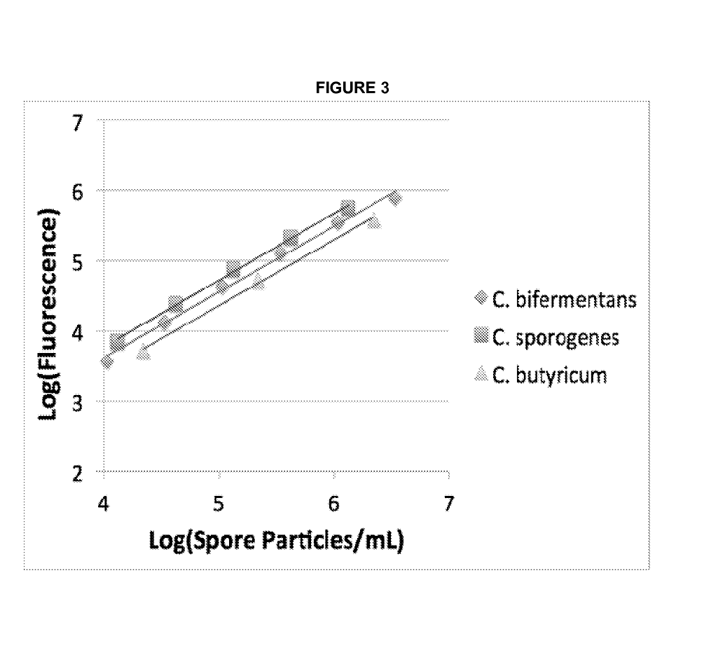

Purified spores were produced as described previously (e.g. see http://www.epa.gov/pesticides/methods/MB-28-00.pdf). Serial dilutions of purified spores from C. bifermentans, C. sporogenes, and C. butyricum cultures were prepared and measured by plating on BBA media and incubating overnight at 37 C to determine CFU/ml. FIG. 3 shows the linear correspondence across different spore producing bacteria across several logs demonstrating the DPA assay as means to assess spore content.

FIG. 3 shows the linear range of DPA assay compared to CFU counts/ml. Purified spores of C. bifermentans, C. sporogenes, and C. butyricum were titered by assessing spore CFU through a germination procedure and by the DPA assay and compared.

The discrepancy for complex spore populations between spore counts measured by germinable spore CFU and by DPA has important implications for determining the potency of an ethanol treated spore preparation for clinical use. Table 2 shows spore content data from 3 different ethanol treated spore preparations used to successfully treat 3 patients suffering from recurrent C. difficile infection. The spore content of each spore preparation is characterized using the two described methods.

Table 2 shows spore content data from 3 different ethanol treated spore preparations used to successfully treat 3 patients suffering from recurrent C. difficile infection. The spore content of each spore preparation is characterized using the two described methods.

Spore content varies per 30 capsules. As measured by germinable SCFU, spore content varies by greater than 10,000-fold. As measured by DPA, spore content varies by greater than 100-fold. In the absence of the DPA assay, it would be difficult to set a minimum dose for administration to a patient. For instance, without data from the DPA assay, one would conclude that a minimum effective dose of spores is 4×105 or less using the SCFU assay (e.g. Preparation 1, Table 2). If that SCFU dose was used to normalize dosing in a clinical setting, however, then the actual spore doses given to patients would be much lower for other ethanol treated spore preparations as measured as by the DPA assay (Table 3).

Table 3 shows the DPA doses in Table 2 normalized to 4×105 sCFU per dose.

It becomes clear from the variability of SCFU and DPA counts across various donations that using SCFU as the measure of potency would lead to significant underdosing or overdosing in certain cases. For instance, setting a dose specification of 4×105 SCFU (the apparent effective dose from donor Preparation 1) for product Preparation 3 would lead to a potential underdosing of more than 100-fold. This can be rectified only by setting potency specifications based on the DPA assay, which better reflects total spore counts in an ethanol treated spore preparation. The unexpected finding of this work is that the DPA assay is uniquely suited to set potency and determine dosing for an ethanol treated spore preparation and potentially other microbial compositions.

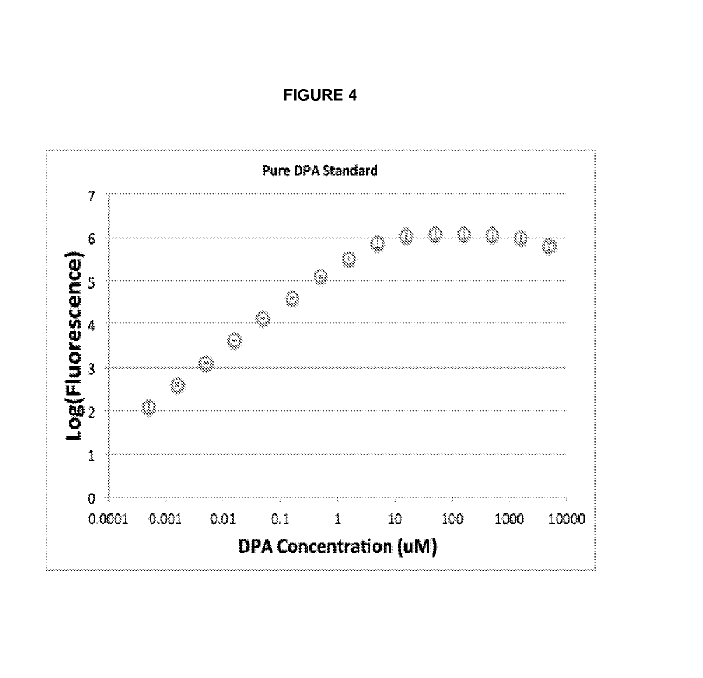

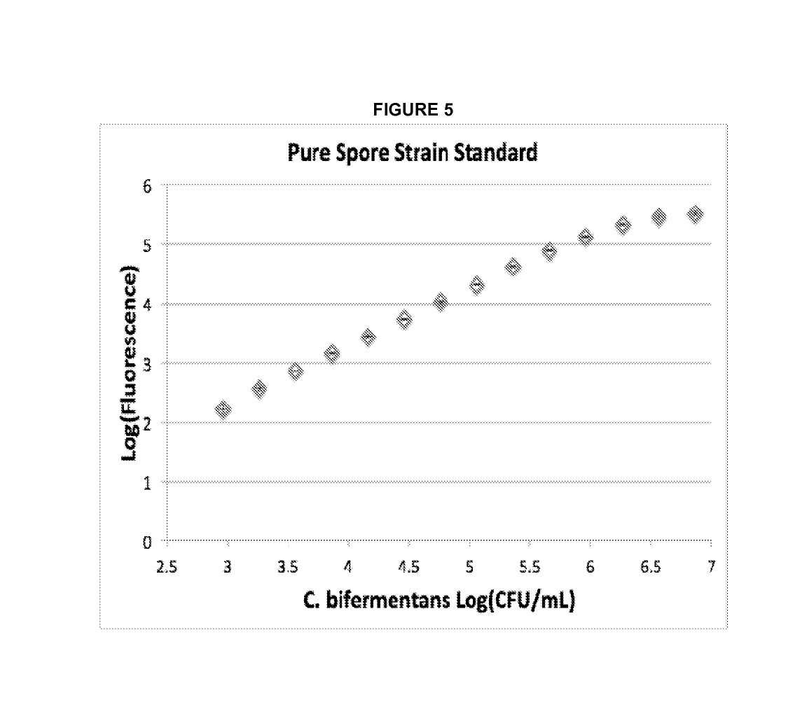

Because DPA is a constituent only of bacterial spores and not of vegetative cells, detection of DPA using terbium chloride can be used to determine if a composition or sample contains contaminating bacterial spores. Once free DPA was washed from the sample and the sample was heated to release DPA from any spores present, it was shown that a given sample that has a DPA content that is above the limit of detection (LOD) is an indication that bacterial spores are present. FIG. 4 shows a dilution series of a pure sample of DPA and indicates that the LOD for DPA is approximately 0.5 nM. FIG. 5 shows a dilution series of a purified, sporulated strain Clostridium bifermentans and indicates a LOD for bacterial spores ofapproximately 1*10⁴ spores/mL.

### Example 15

### Demonstration of Enhanced Growth with a Germinant

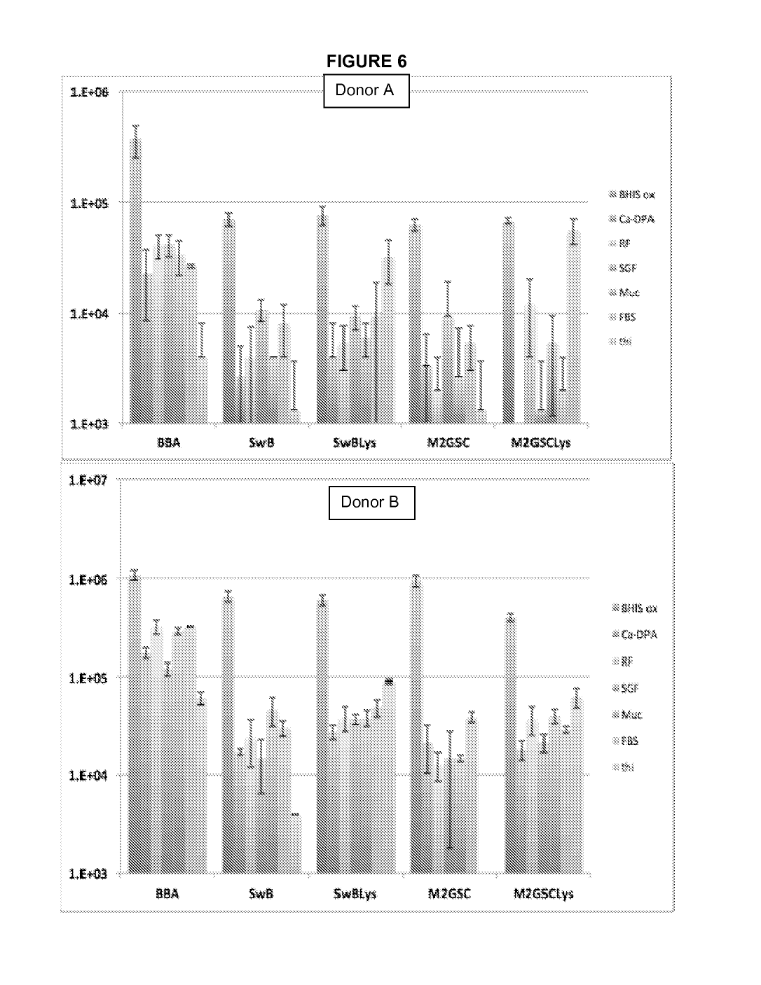

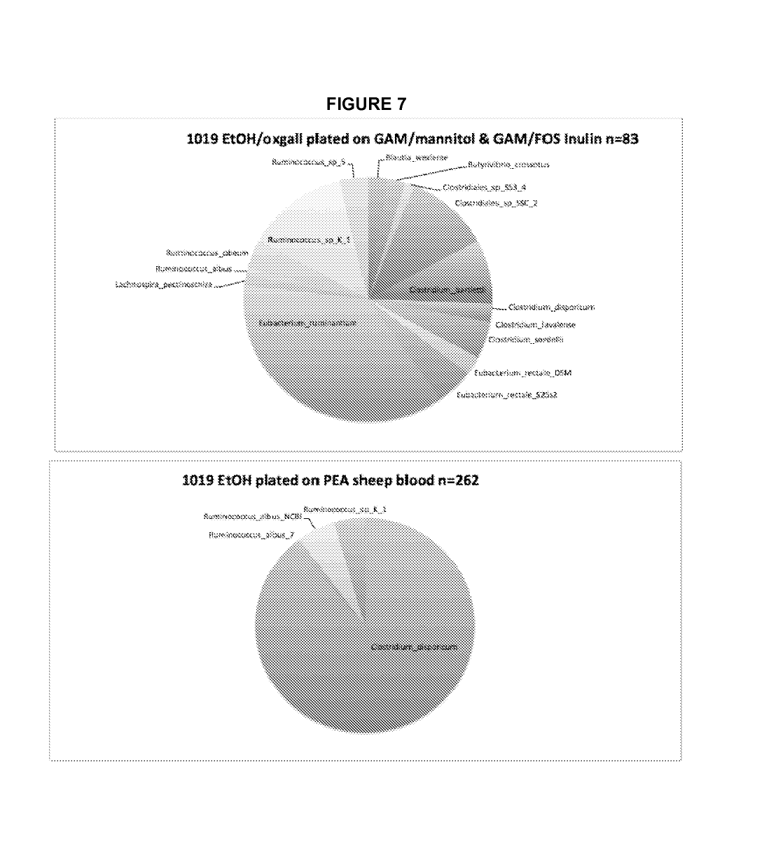

To enhance the detection of spore forming microbes in a microbial composition, adding a germination step to the culturing increases the enrichment of this method. As a non-limiting example, a microbial composition of ethanol treated spores is enriched by various germination strategies. To demonstrate the ethanol treated spore germination capability and spore viability, spores from three different donors were germinated by various treatments and plated on various media. Germination with BHIS/oxgall (BHIS ox), Ca-DPA, rumen fluid (RF), simulated gastric fluid (SGF), mucin medium (Muc), fetal bovine serum (FBS), or thioglycollate (Thi) for 1 hour at 37 C in anaerobic conditions was performed as described previously (e.g. see Examples 6 and 7) with samples derived from two independent donors (FIG. 6). The spore-germinant mixture was serially diluted and plated on different plate media including BBA, Sweet B, Sweet B+lysozyme (tug/ml), M2GSC and M2GSC+lysozyme (tug/ml) as previously described (e.g. see Examples 6 and 7) to determine spore germination. Colony forming units were tallied and titers were determined using standard techniques by one skilled in the art. As FIG. 6 shows, maximum colony forming units were derived from BHI-oxgall treatment. This germination treatment also increases the diversity as measured by the number of OTUs identified when samples were submitted for 16S sequencing (e.g. see Examples 3 and 4) compared to plating without a germination step (FIG. 7).

FIG. 6 depicts different germinant treatments having variable effects on CFU counts from donor A (top) and donor B (bottom). The Y-Axes are spore CFU per ml.

FIG. 7 depicts germinates increase the diversity of cultured spore forming OTUs observed by plating.

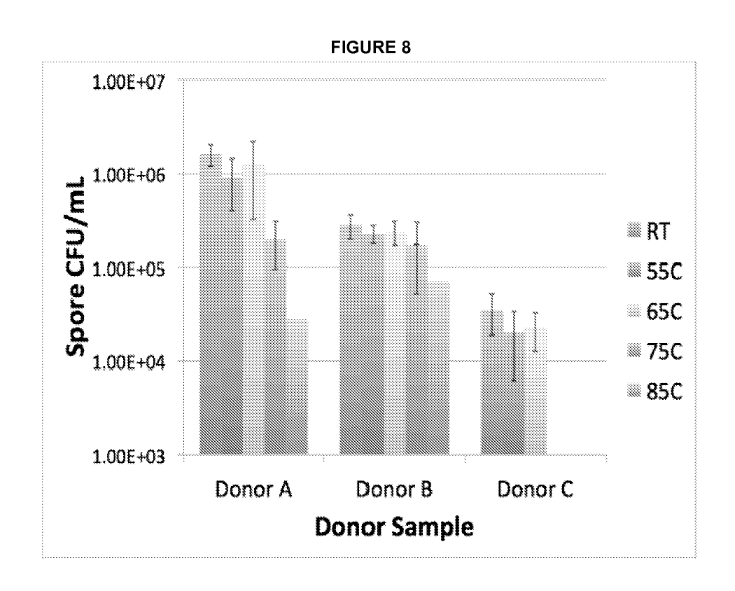

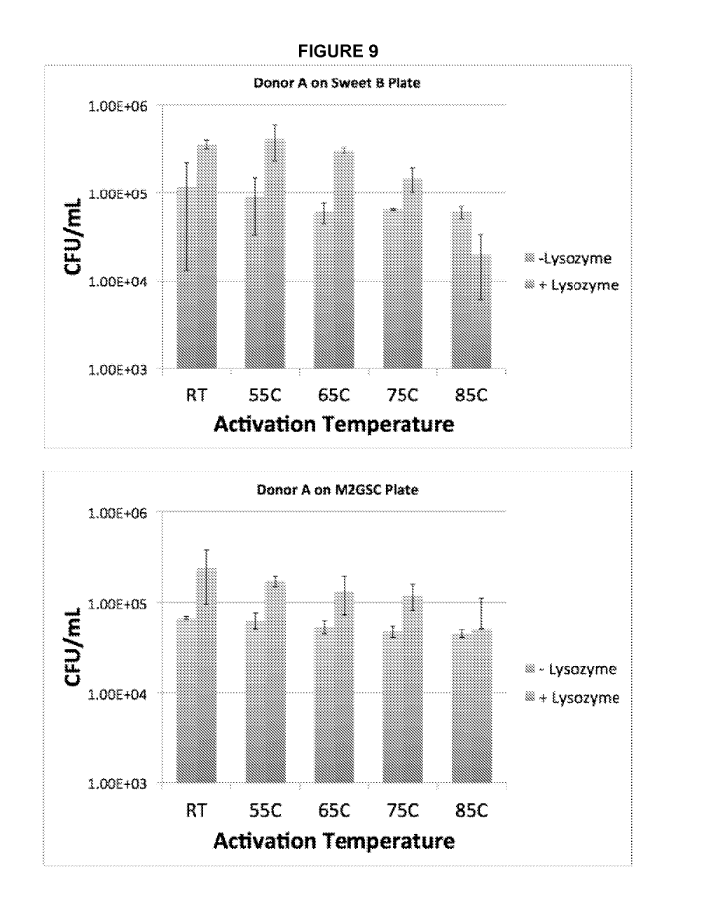

To test the effect of heat activation to promote germination, ethanol treated fecal samples were treated for 15 min at room temperature, 55 C, 65 C, 75 C or 85 C from three different donors and germinated subsequently with BHIS+Oxgall for 1 hr at 37 C then plated on BBA media (FIG. 8) as previously described (e.g. see Examples 6 and 7). Pretreatment at room temperature produced equal if not more spores than the elevated temperatures in all three donors. The temperature of germinating was also examined by incubating samples at room temperature or 37 C for 1 hr in anaerobic conditions before plating on BBA. No difference in the number of CFUs was observed between the two conditions. Lysozyme addition to the plates (2 ug/ml) was also tested on a single donor sample by the testing of various activation temperature followed by an incubation in the presence or absence of lysozyme. The addition of lysozyme had a small effect when plated on Sweet B or M2GSC media but less so than treatment with BHIS oxgall without lysozyme for 1 hr (FIG. 9).

FIG. 8 depicts heat activation as a germination treatment with BHIS+oxgall.

See FIG. 9 depicts the effect of lysozyme and shows a lysozyme treatment enhances germination in a subset of conditions.

Germination time was also tested by treating a 10% suspension of a single donor ethanol treated feces (e.g. see Example 9) incubated in either BHIS, taurocholate, oxgall, or germix for 0, 15, 30, or 60 minutes and subsequently plated on BHIS, EYA, or BBA media (e.g. see Examples 6 and 7). 60 minutes resulted in the most CFU units across all various combinations germinates and plate media tested.

### Example 16

### Demonstrating Efficacy of Terminable and Sporulatable Fractions of Ethanol Treated Spores

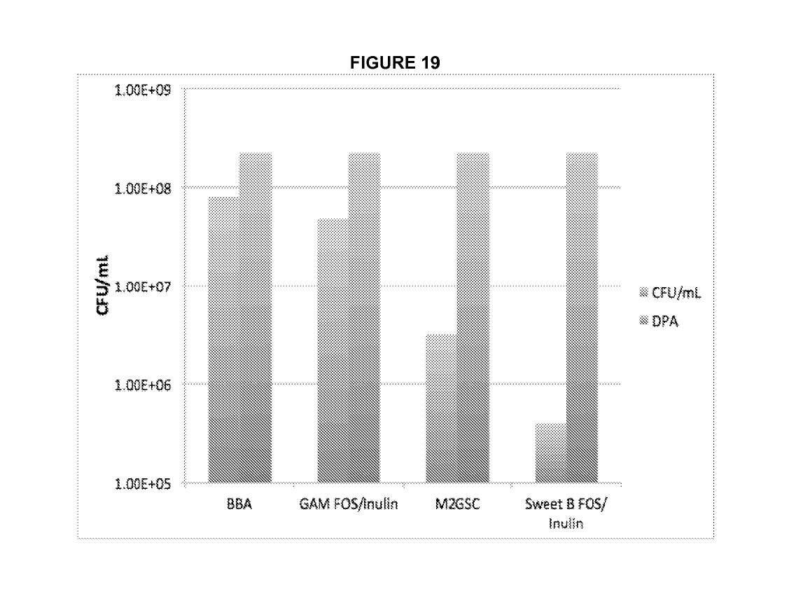

To define methods for characterization and purification, and to improve (e.g., such as by modulating the diversity of the compositions) the active spore forming ecology derived from fecal donations, the ethanol treated spore population (as described in Example 9) was further fractionated. A “germinable fraction” was derived by treating the ethanol-treated spore preparation with oxgall, plating to various solid media, and then, after 2 days or 7 days of growth, scraping all the bacterial growth from the plates into 5 mL of PBS per plate to generate a bacterial suspension. A “sporulatable fraction” was derived as above except that the cells were allowed to grow on solid media for 2 days or 7 days (the time was extended to allow sporulation, as is typical in sporulation protocols), and the resulting bacterial suspension was treated with 50% ethanol to derive a population of “sporulatable” spores, or species that were capable of forming spores. In preparing these fractions, fecal material from donor A was used to generate an ethanol treated spore preparation as previously described herein; then spore content was determined by DPA assay and CFU/ml grown on various media (FIG. 19) as previously described (see Example 14 and 15). See FIG. 19: Spores initially present in ethanol treated spore preparation as measured by DPA and CFU/ml grown on specified media.

To characterize the fraction that is sporulatable, the 2 day and 7 day “germinable” fractions were assessed for CFU and DPA content before and after ethanol treatment to generate a spore fraction. Bacterial suspensions were treated with ethanol, germinated with Oxgall, and plated on the same types of media that the “germinable” fraction was grown on. DPA data showed that growth on plates for 2 and 7 days produced the same amount of total spores. Colonies on the several types of media were picked for 16S sequence analysis to identify the spore forming bacteria present (Table 7).

A 2 day “germinable” fraction and a 7 day “sporulatable” fraction were used as a treatment in the mouse prophylaxis assay as follows. As a control, a 10% fecal suspension prepared from a donor (Donor B) was also administered to mice to model fecal microbiota transplant (FMT) (e.g. see example 17). Weight loss and mortality of the various test and control arms of the study are plotted in Figure S17 and summarized in Table 8 which also contains the dosing information. Clinical score is based on a combined phenotypic assessment of the mouse's health on a scale of 0-4 in several areas including appearance (0-2 pts based on normal, hunched, piloerection, or lethargic), and clinical signs (0-2 points based on normal, wet tail, cold-to-the-touch, or isolation from other animals). The data show both the “germinable” and “sporulatable” fractions are efficacious in providing protection against C. difficile challenge in a prophylaxis mouse model (e.g. see Example 17). The efficacy of these fractions further demonstrates that the species present are responsible for the efficacy of the spore fraction, as the fractionation further dilutes any potential contaminant from the original spore preparation.

See FIG. S16: Titer of “germinable” fraction after 2 days (left) and Sporulatable fraction (right) by DPA and CFU/ml. The “sporulatable” fraction made following 7 days of growth was measured as previously described using germination and growth assays or DPA content as previously described (see Example 14).

The species present in the “germinable” and “sporulatable” fractions were determined by full length 16S sequencing of colony picks and by 16S NGS sequencing of the fractions themselves. The colony pick data indicate Clostridium species are very abundant in both fractions, while the NGS data reveal other spore forming organisms that are typically found in ethanol treated spore preparations are present.

Results are shown in the following: See Table 7. Species identified as “germinable” and “sporulatable” by colony picking approach. See Table 5. Species identified as “germinable” using 16S-V4 NGS approach. See Table 6. Species identified as “sporulatable” using 16s-V4 NGS approach. See Figure S17: Mouse prophylaxis model demonstrates “germinable” and “sporulatable” preparations are protective against C. difficile challenge. Each plot tracks the change in the individual mouse's weight relative to day −1 over the course of the experiment. The number of deaths over the course of the experiment is indicated at the top of the chart and demonstrated by a line termination prior to day 6. The top panels (from left to right) are the vehicle control arm, the fecal suspension arm, and the untreated naive control arm, while the bottom panels are the ethanol treated, gradient purified spore preparation; the ethanol treated, gradient purified, “germinable” preparation, and ethanol treated, gradient purified, “sporulatable” preparation. See Table 8: Results of the prophylaxis mouse model and dosing information

### Example 17

### Bacterial Compositions Prevent C. difficile Infection in a Mouse Model

To test the therapeutic potential of the bacterial compositions a prophylactic mouse model of C. difficile infection (model based on Chen, et al., A mouse model of Clostridium difficile associated disease, Gastroenterology 135(6):1984-1992) was used. Two cages of five mice each were tested for each arm of the experiment. All mice received an antibiotic cocktail consisting of 10% glucose, kanamycin (0.5 mg/ml), gentamicin (0.044 mg/ml), colistin (1062.5 U/ml), metronidazole (0.269 mg/ml), ciprofloxacin (0.156 mg/ml), ampicillin (0.1 mg/ml) and Vancomycin (0.056 mg/ml) in their drinking water on days −14 through −5 and a dose of 10 mg/kg Clindamycin by oral gavage on day −3. On day −1, they received either the test article or vehicle control via oral gavage. On day 0 they were challenged by administration of approximately 4.5 log 10 cfu of C. difficile (ATCC 43255) via oral gavage. Optionally a positive control group received vancomycin from day −1 through day 3 in addition to the antibiotic protocol and C. difficile challenge specified above. Feces were collected from the cages for analysis of bacterial carriage, mortality was assessed every day from day 0 to day 6 and the weight and subsequent weight change of the animal was assessed with weight loss being associated with C. difficile infection. Mortality and reduced weight loss of the test article compared to the vehicle were used to assess the success of the test article. Additionally, a C. difficile symptom scoring was performed each day from day −1 through day 6. Clinical Score was based on a 0-4 scale by combining scores for Appearance (0-2 pts based on normal, hunched, piloerection, or lethargic), and Clinical Signs (0-2 points based on normal, wet tail, cold-to-the-touch, or isolation from other animals).

In a naive control arm, animals were challenged with C. difficile. In the vancomycin positive control arm animals were dosed with C. difficile and treated with vancomycin from day −1 through day 3. The negative control was gavaged with PBS alone and no bacteria. The test arms of the experiment tested 1×, 0.1×, 0.01× dilutions derived from a single donor preparation of ethanol treated spores (e.g. see example 6) or the heat treated feces prepared by treating a 20% slurry for 30 min at 80 C. Dosing for CFU counts was determined from the final ethanol treated spores and dilutions of total spores were administered at 1×, 0.1×, 0.01× of the spore mixture for the ethanol treated fraction and a 1× dose for the heat treated fraction.

Weight loss and mortality were assessed on day 3. The negative control, treated with C. difficile only, exhibits 20% mortality and weight loss on Day 3, while the positive control of 10% human fecal suspension displays no mortality or weight loss on Day 3 (Table 15). EtOH-treated feces prevents mortality and weight loss at three dilutions, while the heat-treated fraction was protective at the only dose tested. These data indicate that the spore fraction is efficacious in preventing C. difficile infection in the mouse.

### Example 18

### Assay for Environmental Contaminants During Processing of Microbial Compositions

The presence of contaminating organisms from the processing environment can be assessed following the guidelines of USP <62>, Microbial examination of nonsterile products: Tests for specified organisms, and USP <61>, Microbial examination of nonsterile products: Microbial Enumeration Tests, although these guidelines are directed towards products that do not include viable organisms. Detecting contaminants in a complex background of product species means that USP <61> and <62> cannot be directly applied. Potential environmental contaminants of regulatory interest that might be introduced during the manufacture of microbial compositions include, without limitation, the following organisms: Bile-Tolerant Gram negative organisms, Escherichia coli, Salmonella, Pseudomonas aeruginosa, Staphylococcus aureus, and Candida albicans. In other settings (i.e. non-spore comprising complex microbial mixtures), clostridia are a class of organisms of interest as well. As known to one skilled in the art, there is no such entity as a perfect medium, so species other than those targeted by the selective conditions may be encountered that can grow on a given medium; the nature of the specimens and the physiologic state of the organisms can influence recovery of desired species, as well as modify the effects of inhibitory characteristics of this medium.

For Bile-Tolerant Gram negative organisms, their presence can be determined in two modes. The first mode is a “test for absence” in which the sensitivity for detection is enhanced via an enrichment growth step that allows small numbers of organisms to expand into a larger detectable population. The second mode is a “quantitative test” in which organisms in the product are directly cultured and their levels can be quantitatively determined. To “Test for Absence” of Bile-Tolerant Gram negative organisms, 1 g of the test material was inoculated into Soybean-casein broth and incubated at 20-25° C. for at least two hours to resuscitate the bacteria (but less than 5 h, to avoid bacterial growth), after which it was it was either used to inoculate the enrichment broth Enterobacteria Enrichment Broth Mossel and incubated at 30-35° C. for 24-48 h, and then plated to Violet Red Bile agar and incubated at 30-35° C. for 18-24 h to detect colonies. The absence of colonies indicates the absence of Bile-Tolerant Gram negative organisms in the product. In a “Quantitative Test” for Bile-Tolerant Gram negative organisms, 1 g of the test material (ethanol treated suspension or final product material) was inoculated into Soybean-casein broth and incubated at 20-25° C. for at least two hours to resuscitate the bacteria (but less than 5 h, to avoid bacterial growth) after which it is diluted into Enterobacteria Enrichment Broth Mossel to the equivalent of 0.1 g, 0.01 g and 0.001 g of material (or 0.1 mL, 0.01 mL and 0.001 mL) and incubated at 30-35 C for 24-48 h, after which 100 ul is plated to Violet Red Bile Glucose Agar, and incubated at 30-35 C for 18-24 h. Growth of colonies for any of the 3 dilutions plated indicates the presence of a presumptive contaminant. A table from USP <62> was then used to determine a probable number of Bacteria per g or mL or product as below (Table 4 from USP <62>). Colonies may be picked and their identities are determined by either 16S rDNA sequencing or by microbiological analysis

The above methods for Bile-Tolerant Gram negative organisms were performed with different broths and selective agars to detect Salmonella (broth, Rappaport Vassiliadis Salmonalla Enrichment Broth; selective agar, Xylose Lysine Deoxycholate Agar), Pseudomonas (broth, Soybean-Casein Digest Broth; selective agar, Cetrimide Agar), and Staphylococcus aureus (broth, Soybean-Casein Digest Broth; selective agar, Mannitol Salt Agar). Colonies that appear on these media are picked and their identities are determined by either 16S rDNA sequencing or by microbiological analysis.

### Example 19

### Residual Assay for Bile-Tolerant Gram Negative Aerobic Organisms

As a non-limiting example of a microbial composition, an ethanol treated fecal suspension is used to test the bile acid tolerance of gram negative aerobic organisms. An ethanol treated fecal suspension was assayed for the presence of residual bile-tolerant Gram-negative species by plating to Violet Red Bile Glucose Agar aerobically, which is recommended for the detection and enumeration of Enterobacteriaceae (including in USP <62>, Microbial examination of nonsterile products: Tests for specified organisms, and USP <61>, Microbial examination of nonsterile products: Microbial Enumeration Tests). Organisms that grow on this selective medium include Escherichia spp, Salmonella spp, Pseudomonas spp, while Gram positive organisms such as Streptococcus and Enterococcus spp do not. Bile salts and crystal violet inhibit gram-positive bacteria, and neutral red is a pH indicator that allows glucose fermenters to produce red colonies with red-purple halos of precipitated bile. Aerobic incubation prevents the growth of bile-tolerant anaerobes. A 20% suspension of feces treated with 50% Ethanol for 1 hr was assayed by creating 10 fold serial dilutions and plating (100 uL) to Violet Red Bile Glucose Agar (BD #218661). A pre-ethanol treatment sample was plated in parallel. Plates are incubated aerobically at 37° C. for 48 hr, at which time colonies are counted to determine cfu/g pre and post-ethanol treatment. Inactivation of presumptive bile-tolerant Gram-negative aerobes is indicated by reduced cfu/ml. Colonies from the ethanol treated sample are considered presumptive bile-tolerant Gram-negative aerobe, but as known to one skilled in the art, there is no such entity as a perfect medium, so species other than those targeted by the selective conditions may be encountered that can grow on a given medium; the nature of the specimens and the physiologic state of the organisms can influence recovery of desired species, as well as modify the effects of inhibitory characteristics of this medium. Colonies are picked and their identities are determined by either 16S rDNA sequencing or by microbiological analysis.

### Example 20

### Residual Assay for the Presence of the Gram Negative Organism Pseudomonas aeruginosa

As a non-limiting example of a microbial composition, an ethanol treated fecal suspension is used. An ethanol treated fecal suspension was assayed for the presence of residual bile-tolerant Gram-negative species by plating to Cetrimide Agar aerobically, which is recommended for the detection and enumeration of Pseudomonas aeruginosa (including in USP <62>, Microbial examination of nonsterile products: Tests for specified organisms, and USP <61>, Microbial examination of nonsterile products: Microbial Enumeration Tests). Cetrimide is a quaternary ammonium compound with bactericidal activity against a broad range of Gram-positive organisms and some Gram-negative organisms. Aerobic incubation prevents the growth of anaerobes. Presumptive Pseudomonas colonies are yellow-green or yellow brown in colour and fluoresce under UV light. A 20% suspension of feces treated with 50% Ethanol for 1 hr was assayed by creating 10-fold serial dilutions and plating (100 uL) to Cetrimide Agar (BD #285420). A pre-ethanol treatment sample was plated in parallel. Plates are incubated aerobically at 37° C. for 48 hr, at which time colonies are counted to determine cfu/g pre and post-ethanol treatment. Inactivation of presumptive Pseudomonas is indicated by reduced cfu/ml. As known to one skilled in the art, there is no such entity as a perfect medium, so species other than those targeted by the selective conditions may be encountered that can grow on a given medium; the nature of the specimens and the physiologic state of the organisms can influence recovery of desired species, as well as modify the effects of inhibitory characteristics of this medium. Presumptive Pseudomonas colonies are picked and their identities are determined by either 16S rDNA sequencing or by microbiological analysis.

### Example 21

### Residual Assay for the Presence of the Gram Positive Staphylococci

As a non-limiting example of a microbial composition, an ethanol treated fecal suspension is used. An ethanol treated fecal suspension was assayed for the presence of residual Gram positive Staphylococcus species by plating to Mannitol Salt Agar aerobically, which is recommended for the detection and enumeration of Staphylococcus species including Staphylococcus aureus and Staphylococcus epidermidis (including in USP <62>, Microbial examination of nonsterile products: Tests for specified organisms, and USP <61>, Microbial examination of nonsterile products: Microbial Enumeration Tests). Mannitol Salt Agar is a nutritive medium due to its content of peptones and beef extract, which supply essential growth factors, such as nitrogen, carbon, sulfur and trace nutrients. The 7.5% concentration of sodium chloride results in the partial or complete inhibition of bacterial organisms other than staphylococci. Mannitol fermentation, as indicated by a change in the phenol red indicator, aids in the differentiation of staphylococcal species. Presumptive Staphylococcus aureus and Staphylococcus epidermidis colonies have yellow zones and red/purple zones, respectively. A 20% suspension of feces treated with 50% Ethanol for 1 hr was assayed by creating 10 fold serial dilutions and plating (100 uL) to Mannitol Salt Agar (BD #221173). A pre-ethanol treatment sample was plated in parallel. Plates are incubated aerobically at 37° C. for 48 hr, at which time colonies are counted to determine cfu/g pre and post ethanol treatment. Inactivation of presumptive Staphylococci is indicated by reduced cfu/ml. As known to one skilled in the art, there is no such entity as a perfect medium, so species other than those targeted by the selective conditions may be encountered that can grow on a given medium; the nature of the specimens and the physiologic state of the organisms can influence recovery of desired species, as well as modify the effects of inhibitory characteristics of this medium. Presumptive Staphylococci colonies are picked and their identities are determined by either 16S rDNA sequencing or by microbiological analysis.

### Example 22

### Residual Assay for the Presence of Fungi Including Candida spp

As a non-limiting example of a microbial composition, an ethanol treated fecal suspension is used. An ethanol treated fecal suspension was assayed for the presence of residual Candida spp by plating to Sabouraud Dextrose Agar which is used for the enumeration of pathogenic and nonpathogenic fungi, particularly dermatophytes (including in USP <62>, Microbial examination of nonsterile products: Tests for specified organisms, and USP <61>, Microbial examination of nonsterile products: Microbial Enumeration Tests). The high glucose concentration in Sabouraud Dextrose Agar provides an advantage for the growth of the (osmotically stable) fungi while most bacteria do not tolerate the high sugar concentration. In addition, the low pH is optimal for fungi, but not for many bacteria. Other medium used in isolation of fungi include Potato Dextrose agar, Czapeck dox agar (Sigma-Aldrich) supplemented with chloramphenicol (0.05 g/l) and gentamycin (0.1 g/l), Dixon agar supplemented with chloramphenicol (0.05 mg/mL) and cycloheximide (0.2 mg/mL). Candida spp that may be isolated from human feces include Candida albicans, Candida tropicalis, Candida krusei, Candida glabrata, and Candida guilleirmondii. A 15% suspension of feces treated with 50% Ethanol for 1 hr was assayed by creating 10-fold serial dilutions and plating (100 uL) to Sabouraud Dextrose Agar (BD #211584). A pre-ethanol treatment sample was plated in parallel. Plates are incubated aerobically at 20-25° C. for up 5 days, at which time colonies are counted to determine cfu/g pre and post ethanol treatment. Inactivation of presumptive fungi Candida is indicated by reduced cfu/ml. As known to one skilled in the art, there is no such entity as a perfect medium, so species other than those targeted by the selective conditions may be encountered that can grow on a given medium; the nature of the specimens and the physiologic state of the organisms can influence recovery of desired species, as well as modify the effects of inhibitory characteristics of this medium. Presumptive fungal colonies are picked and their identities are determined by either 18S rDNA or internal transcribed spacer region (ITS) sequencing or by microbiological analysis.

### Example 23

### Residual Assay for the Presence of the Gram Negative Organisms Escherichia, Salmonella Spp, Shigella Spp, Enterobacter Spp, Klebsiella spp and Pseudomonas spp

As a non-limiting example of a microbial composition, an ethanol treated fecal suspension is used. An ethanol treated fecal suspension was assayed for the presence of residual Gram-negative species including Escherichia, Salmonella, Shigella, Enterobacter, Klebsiella and Pseudomonas by plating to Xylose-Lysine-Desoxycholate (XLD) Agar aerobically, which is the agar recommended for the detection and enumeration of Salmonella spp (including in USP <62>, Microbial examination of nonsterile products: Tests for specified organisms, and USP <61>, Microbial examination of nonsterile products: Microbial Enumeration Tests), and allows the growth of other Gram negative species as well. XLD Agar is both a selective and differential medium. It contains yeast extract as a source of nutrients and vitamins. It utilizes sodium desoxycholate as the selective agent and, therefore, is inhibitory to gram-positive microorganisms. Xylose is incorporated into the medium since it is fermented by practically all enterics except for the shigellae and this property enables the differentiation of Shigella species. Lysine is included to enable the Salmonella group to be differentiated from the non pathogens since without lysine, salmonellae rapidly would ferment the xylose and be indistinguishable from nonpathogenic species. After the salmonellae exhaust the supply of xylose, the lysine is attacked via the enzyme lysine decarboxylase, with reversion to an alkaline pH which mimics the Shigella reaction. To prevent similar reversion by lysine decarboxylase-positive coliforms, lactose and sucrose are added to produce acid in excess. To add to the differentiating ability of the formulation, an H2S indicator system, consisting of sodium thiosulfate and ferric ammonium citrate, is included for the visualization of the hydrogen sulfide produced, resulting in the formation of colonies with black centers. The non pathogenic H2S-producers do not decarboxylate lysine; therefore, the acid reaction produced by them prevents the blackening of the colonies which takes place only at neutral or alkaline pH. Aerobic incubation prevents the growth of anaerobes. Differential colony morphologies are as follows: E. coli, large, yellow, flat; Enterobacter/Klebsiella, mucoid, yellow; Proteus, Red to yellow. Most strains have black centers; Salmonella, H2S-positive, Red-yellow with black centers, Red-yellow with black centers, Red; Pseudomonas, Red.

A 20% suspension of feces treated with 50% Ethanol for 1 hr was assayed by creating 10 fold serial dilutions and plating (100 uL) to XLD Agar (BD #254055). A pre-ethanol treatment sample was plated in parallel. Plates were incubated aerobically at 37° C. for 48 hr, at which time colonies were counted to determine cfu/g pre and post ethanol treatment. Inactivation of presumptive Gram negative spp was indicated by reduced cfu/ml. As known to one skilled in the art, there is no such entity as a perfect medium, so species other than those targeted by the selective conditions may be encountered that can grow on a given medium; the nature of the specimens and the physiologic state of the organisms can influence recovery of desired species, as well as modify the effects of inhibitory characteristics of this medium. Presumptive colonies of different species were picked based on their morphologies and their identities are determined by either 16S rDNA sequencing or by microbiological analysis.

### Example 24

### Detection of Undesired Gram-Negative Organisms Via LPS

Gram-negative organisms contain lipopolysaccharide (LPS) in their outer membranes. LPS is expressed on the cell surface and is also referred to as endotoxin, as it elicits a variety of inflammatory responses, and is toxic to animals, causing fever and disease when in the bloodstream. LPS can be used as the basis of an assay to detect the presence of undesired Gram-negative organisms in a mixed bacterial community that consists of only Gram positive organisms.

Endotoxin can be detected via a limulous amoebocyte lysate test (LAL test). This assay is based in the biology of the horseshoe crab (Limulous), which produces LAL enzymes in blood cells (amoebocytes) to bind and inactivate endotoxin from invading bacteria. A gel clot based assay is performed as follows: equal volumes of LAL reagents are mixed with undiluted or diluted test article and observed for clot formation. The dilutions are selected to cover the potential range of endotoxin in the sample and to reduce interference by the test material making the gel clot LAL test semi-quantitative. The sensitivity of this assay is 0.06 EU/ml. The USP chromogenic method of the LAL test is based on the activation of a serine protease (coagulase) by the endotoxin, which is the rate-limiting step of the clotting cascade. The assay measures the activation of the serine protease as opposed to the end result of this activation, which is clotting. The natural substrate, coagulogen, is replaced by a chromogenic substrate. On cleavage of this substrate a chromophore is released from the chromogenic peptide and is measured by spectrophotometry. The USP chromogenic method is quantitative and can provide a greater sensitivity over a wider range. The sensitivity of this assay is 0.10 EU/ml. This assay could be performed on the mixed community in its product form, or to increase sensitivity, it could be performed after a sample of the product has been grown in enrichment culture to expand the population of any contaminant Gram negative organism that might be present.

### Example 25

### Detection of Undesired Gram-Positive Organisms

The cell walls of Gram positive organisms consist of peptidoglycan and teichoic acids. Teichoic acids are polymers with glycerol or ribitol joined together through phosphodiester linkages. Many of these polymers have glucosyl or D-alanyl residues and are located exclusively in the walls, capsules or membranes of gram-positive bacteria. The teichoic acids may be divided into two groups by their cellular localization—the membrane teichoic acids or lipoteichoic acids linked covalently to lipids, and the wall teichoic acids linked covalently to the peptidoglycan. Wall teichoic acids may be composed of glycerol phosphate, ribitol phosphate and sugar-1-phosphate residues. Most of the ribitol containing teichoic acids also contain D-alanine residues.

As teichoic acids are a discriminating feature of Gram-positive cells, and are not found in Gram negative organisms they can thus be used as an indicator of the presence of undesired Gram positive organisms in a mixed bacterial community that consists of only Gram negative organisms, such as a community solely comprising Gram negative commensal Bacteroides spp.

Teichoic acids can be detected in the supernatant of a mixed bacterial community using an antiteichoic acid ELISA. Antiteichoic antibodies may also be used to detect Gram positive organisms via flow cytometry (e.g. see, Jung et al J Immunology, 2012).

Anti-teichoic acid antibodies with varying specificities may be used to detect different Gram positive organisms, including environmental contaminants such as Staphylococcus epidermidis or Bacillus spp.

### Example 26

### Rapid Detection of Spore Forming Organisms

Degenerate qPCR primers for the spo0A gene (primers described in Bueche et al, AEM, 2013), which encodes the master regulator of sporulation in spore forming organisms, may be used to detect the presence of sporeforming organisms in a mixed community, or to determine whether an organism which forms a colony in a microbiological colony forming unit QC assay is a spore former or not.

### Example 27

### Rapid Determination of Gram Positive or Gram Negative Status of Individual Cultures or Mixed Communities

Gram negative and gram positive cells respond differentially to treatment with detergent under alkaline conditions, with Gram negative organisms typically displaying rapid lysis, while Gram positives are more resistant. This is well known, and alkaline lysis of gram negatives is standard in DNA preparations, as is the need for additional treatments to achieve efficient lysis and DNA recovery from Gram positives. Differential lysis can be used to determine whether a community of only Gram negative organisms contains an undesired Gram positive component, or to determine whether a colony in a microbiological colony forming units assay is Gram positive or negative. In one version of this assay, the mixed community culture or a single colony derived from said community is resuspended in 1 mL of buffer and analyzed on an automated urine particle analyzer UF-1000i (Sysmex Corporation). The UF-1000i has a dedicated analytical flow channel named “BACT channel”, which employs specialized reagents and algorithm for bacteria detection and counting. These aspects of UF-1000i realize precise counting of bacteria in urine specimen or other samples in a short time (Wada et al PLoS One 2012). This is a rapid assay in which a 5 minute treatment with alkaline SDS followed by flow cytometry yields cell counts indicating lysis of Gram negative cells relative to untreated control samples, or resistance to lysis indicating the presence of Gram positive cells. For colony identification, this could be combined with subsequent microbiological identification strategies targeted at either Gram positives or Gram negatives.

### Example 28

### Residual Assay for Gram-Positive Aerobic Organisms (Enterococcus spp)

As a specific non-limiting example, a microbial composition e.g. an ethanol treated fecal suspension can be assayed for the presence of residual Enterococcus species by plating to selective media. Two 20% suspensions of feces (Sample1 and Sample2) were treated with 50% ethanol for 1 hr and assayed by creating 10 fold serial dilutions and plated (100 uL) to Enterococcosel Agar (BD #212205). A pre-ethanol treatment sample was also plated in parallel. Similar media selective for Enterococcus species such as m-Enterococcus Agar (BD #274610) can also be used. Enterococcosel Agar is suitable for the growth of Enterococcus faecalis and Enterococcus faecium and other Enterococcus spp. The selective and differential properties of this media are as follows. Enterococci hydrolyze the glycoside, esculin, to esculetin and dextrose. Esculetin reacts with an iron salt, ferric ammonium citrate, to form a dark brown or black complex. Oxgall is used to inhibit gram-positive bacteria other than enterococci. Sodium azide is inhibitory for gram-negative microorganisms. Other organisms that may grow on these plates include Listeria monocytogenes, Streptococcus bovis Group, pediococci and staphylococci. Plates were incubated aerobically at 37° C. for 48 hr. After incubation colonies were counted and used to back calculate the concentration of residual viable cells of Enterococcus. Any colonies with a black or brown precipitate are considered presumptive Enterococcus species until confirmed by identification by 16S rDNA amplification and sequencing. No colonies were detected on the ethanol treated Enterococcosel plates (limit of detection 10 CFU/mL). Selective media does not always counter select all other species that might be present in the sample being plated. Any colonies that grow need to be identified by amplification and sequencing of the 16S rDNA gene. For Sample1, colonies were counted on plates from the pre-ethanol 20% suspension and used to back-calculate a concentration of 4.75 Log CFU/mL of presumptive Enterococcus (3.75 Log reduction in titer to limit of detection) (Table 11). Four presumptive Enterococcus colonies from the pre-ethanol 20% suspension were picked for 16S rDNA amplification and sequencing and identified as Streptococcous bovis and Streptococcus pasteurianus (Table 9). For Sample2, colonies were counted on plates from the pre-ethanol 20% suspension and used to back-calculate a concentration of 5.14 Log CFU/mL of presumptive Enterococcus (4.14 Log reduction in titer to limit of detection) (Table 12). Four presumptive Enterococcus colonies from the pre-ethanol 20% suspension were picked for 16S rDNA amplification and sequencing and identified as Enterococcus faecium (Table 10).

### Example 29

### Residual Assay for Gram-Positive Aerobic Organisms (Streptococcus spp Assay)

As a specific non-limiting example, a microbial composition e.g. an ethanol treated fecal suspension can be assayed for the presence of residual Streptococcus species by plating to selective media. A 20% suspension of feces treated with 50% ethanol for 1 hr was assayed by creating 10 fold serial dilutions and plated to Mitis Salivarius Agar (BD #229810). Enzymatic Digest of Casein and Enzymatic Digest of Animal Tissue provide carbon, nitrogen, and amino acids used for general growth requirements in Mitis Salivarius Agar. Sucrose and Dextrose are carbohydrate sources. Dipotassium Phosphate is the buffering agent. Trypan Blue is absorbed by the colonies, producing a blue color. Crystal Violet and Potassium Tellurite inhibit most Gram-negative bacilli and Gram-positive bacteria except streptococci. Agar is the solidifying agent. A pre-ethanol treatment sample was also plated in parallel. Plates were incubated aerobically at 37° C. for 48 hr. After incubation colonies were counted and used to back calculate the concentration of residual viable cells of Streptococcus. Based on colony counts for Sample1 from the appropriate dilution plate a concentration of presumptive Streptococcus was determined to be 4.92 Log CFU/mL for the pre-ethanol sample and 1 Log CFU/mL for the ethanol treated sample (3.92 Log reduction in titer) (Table 11). Based on colony counts for Sample2 from the appropriate dilution plate a concentration of presumptive Streptococcus was determined to be 5.25 Log CFU/mL for the pre-ethanol sample and 1.90 Log CFU/mL for the ethanol treated sample (3.34 Log reduction in titer) (Table 12). Any colonies which appear are considered presumptive Streptococcus species until confirmed by identification by 16S rDNA amplification and sequencing. Colonies were picked from pre-ethanol plates and from ethanol treated and identified by 16S rDNA amplification and sequencing for each sample (Tables 9 and 10). Selective media does not always counter select all other species that might be present in the sample being plated. Any colonies that grow need to be identified by amplification and sequencing of the 16S rDNA gene.

### Example 30

### Residual Assay for Gram-Positive Anaerobic Organisms (Bifidobacterium spp Assay)

As a specific non-limiting example, a microbial composition e.g. an ethanol treated fecal suspension can be assayed for the presence of residual Bifidobacterium species by plating to selective media. A 20% suspension of feces treated with 50% ethanol for 1 hr was assayed by creating 10 fold serial dilutions and plated to Bifidobacterium Selective Agar (BIFIDO) (Anaerobe Systems #AS-6423) and Raffinose-Bifidobacterium Agar (Hartemink, et. al., Journal of Microbiological Methods, 1996). Bifidobacterium Selective Agar (BIFIDO) is a selective medium for the isolation and enumeration of Bifidobacterium species. BIFIDO contains Reinforced Clostridial Agar as the basal medium and Polymixin, Kanamycin, and Nalidixic acid as selective agents. The differential compounds of iodoacetate and 2, 3, 5-triphenyltetrazolium chloride are also added. Raffinose-Bifidobacterium Agar medium owes its selectivity to the presence of propionate (15 g/L) and lithium chloride (3 g/L) as inhibitory agents, and raffinose (7.5 g/L) as a selective carbon source. In addition, casein (5 g/L) is used as a protein source, which results in a zone of precipitation around the colonies of bifidobacteria. Plates were incubated anaerobically at 37° C. for 48 hr. After incubation colonies were counted and used to back calculate the concentration of residual viable cells of Bifidobacterium. Any colonies which appear are considered presumptive Bifidobacterium species until confirmed by identification by 16S rDNA amplification and sequencing. Colonies appearing on ethanol treated 20% fecal suspension were identified by 16S rDNA amplification and sequencing (Tables 9 and 10). Selective media does not always counter select all other species that might be present in the sample being plated. Any colonies that grow need to be identified by amplification and sequencing of the 16S rDNA gene.

### Example 31

### Residual Assay for Determining the Limit of Detection of Gram-Positive Anaerobic Organisms in Ethanol Treated Feces (Enterococcus Assay)

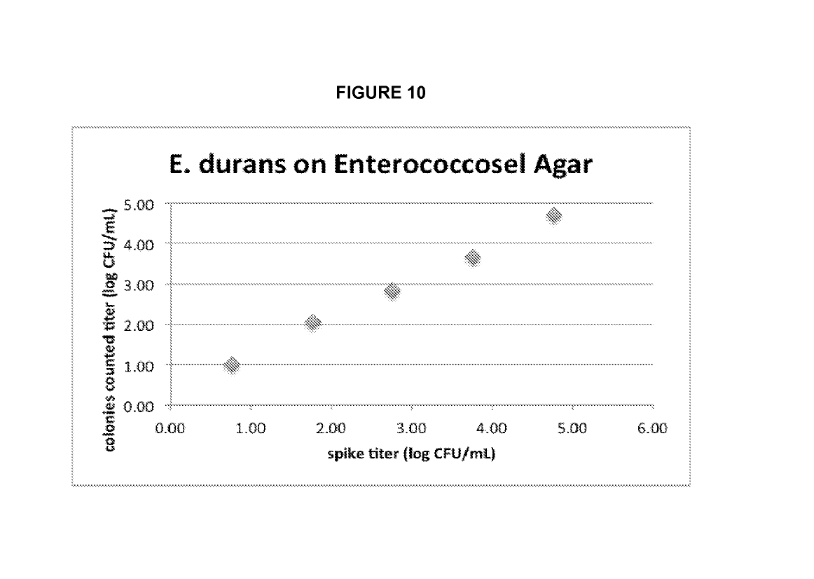

A spiking experiment was performed to determine the limit of detection of a representative Enterococcus isolate (Enterococcus durans) added to a microbial composition e.g. ethanol treated 20% fecal suspension. A 20% suspension of feces was treated with ethanol for 1 hr, split into multiple aliquots and then spiked with 0.77, 1.77, 2.77, 3.77 and 4.77 Log CFU/mL of Enterococcus durans. Each sample was serially diluted and 100 uL of each dilution was plated to Enterococcosel Agar and then incubated aerobically for 48 hr. Based on colony counts a limit of detection of 58 CFU/mL was determined for the assay in current format. The limit of detection could be reduced by plating additional volume of sample to multiple plates and checking for colonies. The concentration of spiked E. durans was plotted against the value calculated for colony counts on selective media (FIG. 10). Selective media does not always prevent growth of all other species that might be present in the sample being plated. Any colonies that grow need to be identified by amplification and sequencing of the 16S rDNA gene.

### Example 32

### Selective Enrichment Through Addition of a Live Bacterial Culture

The selective enrichment of a bacterial species or clade can be achieved by first pre-treating a bacterial mixture with a pure culture of a particular bacterial or fungal species before plating to general or selective agar plates. The bacterial suspension is mixed with a pure culture of a species which can produce an antibiotic, bacteriocin, short chain fatty acid, vitamin, acidic end product, sugar or other compounds which alter the media in a way to enrich for the bacterial species of interest. The sample is then plated to a general nutrient or selective media and incubated at 37 C for 1-5 days to grow colonies. Plates are incubated either aerobically or anaerobically depending on the growth requirements of the species being selected (See Tables 9-12 and FIG. 10.

Table 9 depicts the 16S rDNA identification of colony picks from plating a 20% fecal suspension (Sample1) or from plating a ethanol treated suspension to selective media (number of colony picks matching each species in parentheses).

Table 10 depicts the 16S rDNA identification of colony picks from plating a 20% fecal suspension (Sample2) or from plating a ethanol treated suspension to selective media (number of colony picks matching each species in parentheses).

Table 11 depicts the estimated concentration of a 20% fecal suspension and the ethanol treated spore composition Colonies were counted from plating a 20% feces suspension (Sample1) or ethanol treated suspension to selective media and used to back-calculate the concentration of presumptive cells in each sample (Log CFU/mL).

Table 12 depicts the estimated concentration of a 20% fecal suspension and the ethanol treated spore composition Colonies were counted from plating a 20% feces suspension (Sample2) or ethanol treated suspension to selective media and used to back-calculate the concentration of presumptive cells in each sample (Log CFU/mL).

FIG. 10 depicts the correlation between concentration of E. durans spiked into 20% ethanol treated feces and concentration calculated from colony counts on selective media (Enterococcosel Agar).

### Example 33

### Screening of Ethanol-Treated Fecal Samples for the Presence of Ethanol-Sensitive Gram-Negative Aerobic and Anaerobic Bacteria

As a specific non-limiting example, a microbial composition e.g. spore fraction derived from fecal material as previously described was used. Briefly, the suspensions of fecal material were treated with 200-proof ethanol at a 50% v/v concentration for 1 hour. To characterize killing of vegetative cells via ethanol treatment, after multiple steps of washing to remove residual ethanol, samples were collected for plating on Bacteroides Bile Esculin (BBE) agar and MacConkey II lactose agar. BBE agar is selective for the B. fragilis group of Gram-negative bacteria. MacConkey agar is selective for Enterobacteriaceae and a variety of other Gram negative bacteria. 100 uL of sample were plated on each plate type and spread with a sterile spreader. The BBE agar plates were incubated anaerobically at 37° C. for 48 hours. The MacConkey plates were incubated aerobically at 37° C. for 48 hours. After 48 hours, plates were inspected for the presence of colonies. The results are shown in this table:

Table 13 depicts the results of plating an ethanol treated fecal suspension on BBE and MacConkey II lactose agar showing no residual colonies observed. The limit of detection of this method is ten colonies per ml of sample.

Table 14 depicts the results from Sabouraud Dextrose agar plating of fecal suspensions before and after treatment with 50% Ethanol.

15% suspension samples from 4 different donors were treated with 50% ethanol for 1 hour. Samples were serial diluted in 1×PBS and spot plated on Sabouraud Dextrose Agar both before and after ethanol treatment. Ethanol was washed out of each sample by centrifuging the sample at 13000 rpm, removing the supernatant fluid, and resuspending the pellet in fresh 1×PBS. Plates were incubated at 30° C. aerobically for 48 hours before analyzing colony counts. Colonies were counted to determine the reduction in cfu/mL due to treatment with ethanol.

The sensitivity of this method can be increased by plating additional volume of sample for enumeration. Alternatively, an enrichment step can be added in which the sample is inoculated into growth medium and incubated for 24-48 h, followed by plating to BBE or MacConkey lactose agar. Detection of any colony forming units would indicate the presence of organisms.

### Example 34

### Enrichment of a Spore Fraction by Chromatographic Separation from a Microbial Compositions

A microbial composition sample is pelleted by centrifugation at 15,000×g for 15 minutes at 4° C. and is resuspended phosphate buffered saline supplemented with NaCl to a final concentration of 4M total salt and contacted with octyl Sepharose 4 Fast Flow to bind the hydrophobic spore fraction. The resin is washed with 4M NaCl to remove less hydrophobic components, and the spores are eluted with distilled water, and the desired enriched spore fraction is collected via UV absorbance. Bacterial identification in the spore fraction can then proceed by the genomic and microbiological methods described herein.

### Example 35

### Spore Purification by Chromatographic Separation of Fecal Material

A spore-enriched population such as obtained from Examples 1-5 above, is mixed with NaCl to a final concentration of 4M total salt and contacted with octyl Sepharose 4 Fast Flow to bind the hydrophobic spore fraction. The resin is washed with 4M NaCl to remove less hydrophobic components, and the spores are eluted with distilled water, and the desired enriched spore fraction is collected via UV absorbance.

### Example 36

### Spore Purification by Filtration of Fecal Material

To reduce residual habitat products from a microbial composition filtration protocol is used. As a specific non-limiting example the ethanol treated fecal suspension is used as the microbial composition. The ethanol treated fecal suspension (e.g. see example 9) above is diluted 1:10 with PBS, and placed in the reservoir vessel of a tangential flow microfiltration system. A 0.2 um pore size mixed cellulose ester hydrophilic tangential flow filter is connected to the reservoir such as by a tubing loop. The diluted spore preparation is recirculated through the loop by pumping, and the pressure gradient across the walls of the microfilter forces the supernatant liquid through the filter pores. By appropriate selection of the filter pore size the desired bacterial spores are retained, while smaller contaminants such as cellular debris, and other contaminants in feces such as bacteriophage pass through the filter. Fresh PBS buffer is added to the reservoir periodically to enhance the washout of the contaminants. At the end of the diafiltration, the spores are concentrated approximately ten-fold to the original concentration. The purified spores are collected from the reservoir and stored as provided herein.

### Example 37

### Enrichment of Microbes by Affinity Chromatography

Microbes including but not limited to bacteria, fungus, virus, and phage contain immunogenic proteins, lipids, and other chemical moieties on their surfaces that can be used to specifically identify and serve as means to purify these components from a composition. With an appropriate affinity reagent including e.g. antibody, receptor, etc, specific microbes are selectively enriched from a microbial mixture as previously described (Accoceberry et al One Step Purification of Enterocytozoon bieneusi Spores from Human Stools by immunoaffinity expanded bed adsorption (EBA). J. of Clinical Microbiology, 39(5). 2001). As a specific, non-limiting example of the method, Enterocytozoon bieneusi spores can be enriched by from a microbial composition e.g. stool. Briefly a 1 kg scale, and a ‘stomacher’ BagMixer (Interscience, cat #023 230) is placed in the hood to allow all work to be done within containment. A 125 g stool sample is transferred to a filter bag. 475 mL of suspension solution (0.9% saline, 18.75% glycerol) is added to the bag. The bag is clamped in place in an Interscience BagMixer for 30 seconds to produce a slurry. The microbial sample is then removed from the filtered side of the bag for further enrichment. The microbial sample is centrifuged at 500×g for 6 min to eliminate large particles, and the sieved spores in the supernatant are pelleted by centrifugation at 2,500×g for 20 min. The pellet was resuspended in PBS (⅓ [vol/vol]) to produce a 25% slurry. Penicillin (5 IU/ml) and streptomycin (100 mg/ml) are added to the final slurry. For a microbial composition one can simply resuspend the the material in buffer to generate an appropriate suspension for further enrichment.

Production of Monoclonal Antibodies (MAbs) to a Microbial Contaminant or Pathogen

Two species-specific MAbs of pathogen specific surface marker e.g. E. bieneusi spore walls are produced as described previously (e.g. see Harlow and Lane, Antibodies: a laboratory Manual, 1988 or Accoceberry, I., M. Thellier, I. Desportes-Livage, A. Achbarou, S. Biligui, M. Danis, and A. Datry. 1999. Production of monoclonal antibodies directed against the microsporidium Enterocytozoon bieneusi. J. Clin. Microbiol. 37: 4107-4112). Briefly, 6E52D9, isotyped as IgG2a, is directed against the exospore, and 3B82H2, isotyped as IgM, is directed against the endospore. The MAbs are purified from hybridoma culture supernatants by affinity protein A chromatography for the 6E52D9 MAb and with Dynabead M-450 rat anti-mouse IgM (Dynal, Compiegne, France), according to the manufacturer's instructions, for the 3B82H2 MAb. The purified supernatants are stored at −20° C. until their use. The 6E52D9 IgG2a can be used as ligand in the immunoaffinity process. A total of 2×106 cells from the hybridoma line are injected via the intraperitoneal route into pristane-primed female BALB/c mice (Charles River Laboratories, Saint-Aubain-les-Elbeuf, France) to produce ascitic fluid that is collected 10 to 15 days later. The ascitic fluids generated are incubated 1 h at 37° C. and overnight at 4° C. and then centrifuged at 3,000×g for 10 min. The supernatants are collected and screened by an immunofluorescence antibody test (see below) using purified E. bieneusi spores or the antigen to which the antibodies are raised against, as previously described (e.g. see Harlow and Lane, Antibodies: a laboratory Manual, 1988 or Accoceberry, I., M. Thellier, I. Desportes-Livage, A. Achbarou, S. Biligui, M. Danis, and A. Datry. 1999. Production of monoclonal antibodies directed against the microsporidium Enterocytozoon bieneusi. J. Clin. Microbiol. 37: 4107-4112). Ascitic fluids yielding high titers are pooled, precipitated by adding an equal volume of saturated ammonium sulfate, and incubated at 4° C. for 4 h. The purified mouse immunoglobulin is recovered by centrifugation at 10,000×g at 4° C. for 20 min. The pellet is dissolved in a small volume of 0.05 M Tris-HCl (pH 9) and injected into a desalting Sephadex G-25 column (Amersham Pharmacia Biotech, Saclay, France) equilibrated with 1 M NaCl-0.05 M Tris-HCl (pH 9) to remove the residual ammonium sulfate and condition the MAb in the binding buffer. Alternatively, if recombinant antigen is used to generate the antibody, an affinity matrix of the antigen can be used to purify antibodies from the supernatant of the hybridomas. Immunoglobulin content can be determined by absorbance at 280 nm using a UV spectrophotometer or by Bradford assay.

Immunofluorescence Antibody Test

Briefly, the antigen is applied to 18-well slides (2 ml per 5-mm well) and incubated sequentially with purified supernatants, diluted at 1:64 in 0.1% bovine serum albumin in PBS, and fluorescein isothiocyanate-labeled goat antimouse IgG-IgM-IgA (1:200 dilution; Sigma Laboratories). Slides are washed, mounted with buffered glycerol mounting fluid, and examined with epifluorescence microscope using standard techniques. Alternatively a western blot or ELISA assay is used to determine the antibody production of a hybridoma supernatant using the antigen e.g. recombinant protein from the surface of the pathogen, purified protein from surface of the pathogen, whole pathogen (ELISA only).

Chromatographic System and EBA Method

The chromatographies are performed with fast-protein liquid chromatography and Biopilot workstations (Amersham Pharmacia Biotech). The Streamline rProtein A matrix (Amersham Pharmacia Biotech) is used for EBA of immunoglobulins. rProtein A is a recombinant protein. The base matrix is a 4% agarose derivative with an inert metal alloy core that provides the density required to use the adsorbent in expanded-bed mode. These porous beads have a size distribution of 80 to 165 mm and a particle density of 1.3 g/ml. The matrix is poured into a Streamline 25 column (Amersham Pharmacia Biotech). This is a glass column with an inner diameter of 25 mm, with a specially designed liquid distributor at the base of the column and a top mobile adapter. The bed is expanded by upward liquid flow. Adsorbent particles are suspended in equilibrium due to the balance between particle sedimentation velocity and upward flow. The sample is applied to the expanded bed with an upward flow. Target molecules and cells are captured on the adsorbent while cell debris, cells, particulates, and contaminants pass through unhindered. Flow is then reversed. The adsorbent particles settle quickly and target molecules are desorbed by an elution buffer, as in conventional packed-bed chromatography. The column is prepared by flowing the purified antibody specific to the microbe to be purified and enriched e.g. an antibody specific to Enterocytozoons bieneusi and allowing it to bind to the rProtein A matrix. It is then crosslinked and quenched. The column is then washed with PBS buffer to remove excess antibody and cross linker as previously described (Reeves, H. C., R. Heeren, and P. Malloy. 1981. Enzyme purification using antibody crosslinked to protein A agarose: application to Escherichia coli NADP-isocitrate dehydrogenase. Anal. Biochem. 115:194-196)

Purifying Bacterial Spores from a Microbial Suspension

A microbial suspension (75 ml) is injected into the prepared column and incubated with the gel at room temperature overnight. The gel is then expanded and washed, to remove all large particles and unbound spores, at an upward flow velocity of 32 ml/min, until the UV baseline is reached. PBS buffer (pH 7.2) is used during expansion and washing. The workstation pump is then turned off to allow the bed to settle. The column adapter was moved down toward the sedimented bed surface. After a wash with PBS, the run is performed at a downward flow rate of 15 ml/min. The elution buffer is run at the same flow rate. Several potential elution buffers are tested to determine the proper conditions empirically. The conditions that can be tested include the following: glycine at 50 mM (pH 2.49), ethylene glycol at 25%, 4 M guanidine HCl, and 6 M guanidine HCl. The elution fractions are then collected into 50-ml Falcon centrifuge tubes, sedimented at 2,500×g for 20 min, and washed four times by centrifugation in PBS to remove residual elution buffer. The pellets are pooled, resuspended in 5 ml of PBS, and stored at 4° C. Resulting spores or bacteria can be further analyzed by genetic or serological methods.

### Example 38

### Serological Identification and Enrichment by Flow Cytometry

Single cells and microbes including but not limited to bacteria and fungi are isolated, enriched, and identified by flow cytometry from a microbial composition using fluorescently labeled tags. These methods have been described previously (Nebe-von-Caron, G., Stephens, P. J. & Hewitt, C. J. Analysis of bacterial function by multi-colour fluorescence flow cytometry and single cell sorting. Journal of Microbiological Methods, 2000). Briefly, a specific affinity reagent e.g. antibody or receptor to a surface marker can be generated (e.g. see example 37) and fluorescently labeled by a variety of methods known to one skilled in the art via biochemical conjugation techniques previously described (e.g. see Hermanson. Bioconjugation, 2008) to commercially available fluorescent dyes, quantum dots, and fluorescent proteins. The process can be multiplexed to identify and enrich multiple different specific bacteria in the same microbial composition by labeling different specific antibody reagents with different color dyes. The single or multiple fluorescent antibody mix is incubated with a microbial composition for 16 hours at 4° C. to allow the fluorescent labeled antibodies to bind the specific bacteria of interest. Multiple wash steps are performed by pelleting the cells at 16,000×g for 5 minutes, resuspending with PBS, and repeating the process 5 times. The microbial composition can then be sorted on a flow cytometer enriching the population of fluorescently labeled microbes. Unlabeled cells can serve as controls to establish appropriate gates to identify fluorescent signal from background. Additionally, recombinant cells expressing ectopic surface antigens can be used as positive controls in a mixture of labeled cells and known ratios of antigen positive cells and antigen negative cells can be mixed to establish and validate the technique. The sorted cells can then be cultured or directly assessed via genetic techniques e.g. 16S sequencing to confirm the serological identity of the enriched cells. Furthermore, a nonspecific dye or light scattering properties can be used to assess total microbial cell counts in a separate sample of cells from the microbial suspension.

As an alternative method, a microbial suspension sample can be fixed, permeabilized, labeled by fluorescent in situ hybridization (FISH) with specific fluorescently labeled oligonucleotide probes to specific 16S rRNA hypervariable sequences and submitted for flow cytometry as previously described (e.g. see Zoetendal, E. G. et al. Quantification of Uncultured Ruminococcus obeum-Like Bacteria in Human Fecal Samples by Fluorescent In Situ Hybridization and Flow Cytometry Using 16S rRNA-Targeted Probes. Applied Environmental Microbiology (2002)). Nonspecific dyes like propidium iodide can be used to count total cell number in one sample and unlabeled cells can be used as negative controls to establish gates for Fluorescence Assisted Cell Sorting (FACS).

Once sorted these enriched cells can be submitted for 16S sequence analysis to further validate and confirm the cell identity.

### Example 39

### Use of Phage to Destroy Abundant Microbes Resulting in Enrichment of Resistant Microbes of Interest from a Microbial Composition

A major issue in detecting low levels of a contaminant of interest is the relatively high levels of other microbes in a microbial composition. One method of enriching a pathogen for further isolation and identification involves using a bacteriophage to lyse the abundant microbes in the composition leaving only phage resistant microbes including the contaminants of interest. As a specific example, phage phi-CD27 isolated previously (e.g. see Mayer, M. J., Narbad, A. & Gasson, M. J. Molecular Characterization of a Clostridium difficile Bacteriophage and Its Cloned Biologically Active Endolysin. Journal of Bacteriology 190, 6734-6740, 2008) is used to clear out clostridium species from a mixed microbial composition. Additionally, phage identified from various sources known to infect Bacteroides species (e.g. Payan, A. et al. Method for Isolation of Bacteroides Bacteriophage Host Strains Suitable for Tracking Sources of Fecal Pollution in Water. Applied and Environmental Microbiology 71, 5659-5662, 2005) is isolated and used to clear abundant bacteria in a microbial composition leaving behind viable, enriched contaminant microbes resistant to the exogenously added phage. The procedure involves mixing high titer of known phage to a microbial sample, incubating for a period of time for infection and lysis to occur. Afterward, the remaining microbes can be pelleted and washed of extraneous cell debris repeatedly leaving only viable microbes of interest behind. Alternatively washes are performed by using a 1 um filter trapping larger microbes of interest while allowing phage and small lysed particulate to be washed away. Subsequent microbes can be further cultured, enriched or identified and detected by other methods described herein.

### Example 40

### Use of Phage to Detect Pathogens of Interest from Microbial Composition

As a specific non-limiting example of the use of a phage for detection and biosensor, recombinant phage expressing reporter genes are used to detect a pathogen of interest at low levels in a microbial composition as previously described (e.g. see Loessner, M. J., Rudolf, M. & Scherer, S. Evaluation of luciferase reporter bacteriophage A511::luxAB for detection of Listeria monocytogenes in contaminated foods. Applied and environmental Microbiology, 1997). Briefly, the bacteriophage A511::luxAB detects listeria by transducing the bioluminescence protein bacterial luciferase (luxAB) generating a luminescence when decanal or other substrate is added to the sample. To test a microbial composition for the presence of listeria, test samples of the microbial composition are added to Brain heart infusion (BHI) medium (Oxoid) and incubated for 2 days at 30° C. as an initial enrichment step. Samples of 1 mL are removed from the enrichment cultures and are transferred to 4 mL of 0.5×BHI broth, and incubated at 30° C. for 2 h. Duplicate 1-mL portions of each sample are mixed with 30 uL of phage suspension (3×108 A511::luxAB Plaque forming units (PFU), which are pre-dispensed into clear polystyrene tubes (75 by 12 mm; Sarstedt) suitable for the luminometer. For expression of phage-encoded luciferase, samples are incubated at 20° C. for 140 min, before bioluminescence is measured in a photon-counting, single-tube luminometer (Lumat LB 9501/16; Berthold). Following injection of 50 ul of 0.25% nonanal (Aldrich) in 70% ethanol, light emission was determined with a 0.5-s delay and the output was integrated over a 10-s period. Results are expressed in relative light units (RLU), as a mean value from the duplicate tubes. Negative controls are samples without the lux phage added and vehicle with lux phage only. A sample is considered positive for Listeria when the phage-infected tube yields RLU at least 100 above the background level indicated by the negative control.

Recombinant methods for building such a phage starting with a wild-type strain are known to one skilled in the art and have been previously described (e.g. see Loessner, M. J., Rees, C. E., Stewart, G. S. & Scherer, S. Construction of luciferase reporter bacteriophage A511::luxAB for rapid and sensitive detection of viable Listeria cells. Applied and Environmental Microbiology 62, 1133-1140, 1996). These methods are used to build other phage to detect other microbes permissive to othorthogal phage infection.

### Example 41

### Selective Removal of Microbes by Targeting a Toxic Substrate to Undesired Cells

Abundant and unwanted species of microbes contained in a microbial composition can be selectively inactivated by targeting a toxin or toxigenic substances to these bacteria via an affinity reagent. Specifically, a Nile blue EtNBS compound, 5-ethylamino-9-diethylaminobenzo [a] phenthiazinium chloride described previously (see Vecchio, D. et al. Structure-function relationships of Nile blue (EtNBS) derivatives as antimicrobial photosensitizers. European Journal of Medicinal Chemistry (2014). doi:10.1016/j.ejmech.2014.01.064) is conjugated to an affinity reagent e.g. antibody selective for a particular microbe as described (see e.g. example 37 and Hermanson, Bioconjugation. Pierce, 2008). This reagent is added to a sample of the microbial composition and incubated for 16 hours at 4° C. in the dark. The microbial composition can then be pelleted by centrifugation at 10,000×g for 10 min and washed by repeating this procedure five times to remove excess antibody conjugate. Resuspending the microbial composition in PBS and exposing the sample to 635 nm light at 50 mW/cm2 for 1 minute to 1 hour will result in the production of radical oxygen species that can damage cellular components. The high local concentration of the photosensitizer results in damage preferentially occurring to the unwanted cells bound by the antibody conjugate. The microbial composition can then be washed of inactivated cells or further enriched and analyzed by techniques presented herein.

### Example 42

### Selective Killing of Microbes Recognized by Antibodies when Serum is Added

To enrich the pathogenic or contaminant microbes to be detected in a microbial composition, serum based inactivation is used to eliminate the microbial composition that would interfere with downstream assays. As a non-limiting, specific example, Pseudomonas aeruginosa is removed from a mixture containing Salmonella as previously described (Xiao et al New role of antibody in bacterial isolation J of AOAC Int. 95: 1. 2012). Briefly, a rabbit polyclonal antibody against P. aeruginosa is prepared by inoculating four New Zealand rabbits with the pathogen P. aeruginosa. The antiserum is purified using saturated ammonium sulfate and added into Rappaport-Vassiliadis medium with soya (RVS) broth and Muller-Kauffmann tetrathionate novobiocin broth (MKTTn broth) to evaluate whether it could inhibit the growth of P. aeruginosa. Alternatively, methods previously described for producing monoclonal antibodies could be used (e.g. see example 37) and added to the medias above to observe inhibition. Observations by scanning electron microscopy are used to demonstrate that P. aeruginosa is attacked and destroyed by the antibody when incubated for 10 min at 37° C. The activity of the antibody is also tested against other strains of P. aeruginosa. Twenty-six strains of Salmonella are mixed with P. aeruginosa in RVS and MKTTn broth at 37 C for 12 h, respectively, and the cultures are plated on Salmonella chromogenic medium (SCM; Oxoid, Basingstoke, UK) to validate the effectiveness of the antibody in a defined microbial composition. The experiment is then repeated in other microbial compositions as a mechanism for enriching Salmonella. It is expected that only Salmonella will grew on SCM; five colonies are randomly selected for identification by VITEK 2 (bioMerieux, Lyon, France) or other previously defined methods (e.g. see examples 1, 3, 4). Additionally, this method can be multiplexed for multiple pathogens of interest by adding a cocktail of antibodies to the microbial composition to inactivate other non-pathogens. Other methods previously described herein are used to identify and further enrich pathogens for detection purposes.

### Example 43

### Purification of DNA Sequences on a Bead Matrix

The limit of detection for determining the presence of a particular nucleic acid sequence can be problematic if the abundance of a sequence of interest is so low that it is not present in 1-2 ug for PCR amplification. Using techniques described above, DNA is purified from a microbial sample. To enrich sequences of interest, an amount of greater than used for PCR is enriched for sequences of interest by contacting the sample with a solid phase comprising bound DNA oligonucleotides that selectively bind to sequences of interest via hybridization and thus enrich them. Suitable solid phase materials include, by way of example and without limitation, polystyrene or magnetic beads, silicon chip surfaces, silica beads, or other suitable systems known to one skilled in the art. As a specific non-limiting example, short oligonucleotides (20-60 bp) are synthesized with biotin at the 5′ or 3′ ends and are bound to magnetic streptavidin beads (Life Sciences). Alternatively, longer probes are developed by using the biotinylated oligonucleotides as PCR primers to amplify sequences of interest, purifying these longer probes, attaching them to the bead matrix and washing away the complementary strand not labeled with biotin under conditions that denature DNA but not the biotin streptavidin linkage (Holmberg et al. The biotin streptavidin interaction can be reversibly broken using water at elevated temperatures, Electrophoresis 26:501-510, 2005). Alternative methods for attaching probes to beads are also possible and have been previously described (e.g. see U.S. Pat. No. 6,288,220 B1, Biophysical Journal 71, 1079-1086 (1996), and Analytical Biochemistry 247, 96-101 (1997)).

With the probe-bead complex generated, one can contact nucleic acid derived from the sample with the beads and incubate the mixture at a suitable temperature to allow the probes to capture the nucleic acid sequences of interest. The undesired, non-hybridizing nucleic acid can then be washed away. The captured DNA can be separated from the substrate using conditions that denature the hybrid including heat or alkaline pH, known to one skilled in the art, or by detaching the probe from the bead by treating the sample with conditions that break the biotin streptavidin interaction (Holmberg et al. The biotin streptavidin interaction can be reversibly broken using water at elevated temperatures, Electrophoresis 26:501-510, 2005).

The enriched DNA sequences can then be sequenced by techniques described (see e.g. examples 3 and 4) or detected by qPCR based techniques to quantify the amount of a particular DNA sequence present.

### Example 44

### Removal of Contaminating DNA Sequences Using the CRISPr System

The CRISPr system can specifically cleave undesired nucleic acid sequences and thus reduce their contaminating effects on downstream DNA detection methods. Systems like those described previously (e.g. see Jinek et al A programmable Dual-RNA-Guided DNA endonuclease in adaptive bacterial immunity. Science. 2012) are used to perform this cleavage of contaminating DNA in vitro. Briefly, the CRISPr protein complex is purified, synthetic RNAs designed to guide the system to cleave target sequences are loaded onto the system, and the complex is incubated with the DNA sample of interest to allow cleavage to ensue. Alternatively, there are several commercial sources for the generation of specific custom CRISPr systems to perform cleavage and these are amenable to in vitro cleavage techniques (e.g. see Sigma and Blue Heron).

Purification of the Cas9 System

The Cas9-CRISPR is commercially available and reagents are purchased from Sigma and all reagents can be designed according to the manufacturer's instructions. (http://www.sigmaaldrich.com/catalog/product/sigma/crispr?lang=en&region=US). Alternatively, the following protocol contains the protocol to produce a custom system based on the work previously published. Briefly, the sequence encoding Cas9 (residues 1-1368) on a custom pET-based expression vector using ligation-independent cloning (LIC) is used for this protocol as previously described (Jinek et al A programmable Dual-RNA-Guided DNA endonuclease in adaptive bacterial immunity. Science. 2012.) The resulting fusion construct contained an N-terminal hexahistidine-maltose binding protein (His6-MBP) tag, followed by a peptide sequence containing a tobacco etch virus (TEV) protease cleavage site is expressed in in E. coli strain BL21 Rosetta 2 (DE3) (EMD Biosciences), grown in 2×TY medium at 18° C. for 16 h following induction with 0.2 mM IPTG. The protein was purified by a combination of affinity, ion exchange and size exclusion chromatographic steps. Briefly, cells are lysed in 20 mM Tris pH 8.0, 500 mM NaCl, 1 mM TCEP (supplemented with protease inhibitor cocktail (Roche)) in a homogenizer (Avestin). Clarified lysate is bound in batch to Ni-NTA agarose (Qiagen). The resin is washed extensively with 20 mM Tris pH 8.0, 500 mM NaCl and the bound protein is eluted in 20 mM Tris pH 8.0, 250 mM NaCl, 10% glycerol. The His6-MBP affinity tag is removed by cleavage with TEV protease, while the protein is dialyzed overnight against 20 mM HEPES pH 7.5, 150 mM KCl, 1 mM TCEP, 10% glycerol. The cleaved Cas9 protein is separated from the fusion tag by purification on a 5 ml SP Sepharose HiTrap column (GE Life Sciences), eluting with a linear gradient of 100 mM-1 M KCl. The protein is further purified by size exclusion chromatography on a Superdex 200 16/60 column in 20 mM HEPES pH 7.5, 150 mM KCl and 1 mM TCEP. Eluted protein is concentrated to −8 mg·ml-1, flash-frozen in liquid nitrogen and stored at −80° C. Optionally, all four Cas9 proteins are purified by an additional heparin sepharose step prior to gel filtration, eluting the bound protein with a linear gradient of 100 mM-2 M KCl. All proteins are concentrated to 1-8 mg·ml-1 in 20 mM HEPES pH 7.5, 150 mM KCl and 1 mM TCEP, flash-frozen in liquid N2 and stored at −80° C.

Template RNA Generation

Templates for cleaving undesired sequences are cloned onto an appropriate plasmid based vector containing a T7 flash transcription site by standard molecular biological techniques known to one skilled in the art (Sambrook and Russell, Molecular Cloning, a laboratory manual, third edition, 2001). As a non-limiting specific example, short 16S sequences from bacteria found in the microbial composition can be cloned and subsequently generate RNA based templates to remove dominant 16S sequences leaving behind 16S sequences that are derived from pathogenic species. These sequences are designed as follows: ˜21 nucleotides of complementarity to the 16S region to be cleaved with an extra GG sequence at the followed by the tracrRNA sequence described previously (see Sigma, http://www.sigmaaldrich.comitechnical-documents/articles/biology/crispr-cas9-genome-editing.html). The short 16S regions will be cloned into the CRISPr gene in the spacer regions with the appropriate RNA based motifs in the repeat regions required for proper Cas9 processing. Importantly the protospacer adjacent motif (PAM) must be considered when designing where the template will cut and must be present in the DNA sequence that will be cut. Various cas9 systems have different PAM sequences to further expand the utility of this method. RNA templates are in vitro transcribed using T7 Flash in vitro Transcription Kit (Epicentre, Illumina company) and PCR-generated DNA templates carrying a T7 promoter sequence. RNAs are gel-purified and quality-checked prior to use.

Cleavage of Undesired Sequences

Synthetic or in vitro-transcribed RNAs are pre-annealed prior to the reaction by heating to 95° C. and slowly cooling down to room temperature. The DNA sample is incubated for 60 min at 37° C. with purified Cas9 protein mixture (50-500 nM) and RNA duplex (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl2. Higher concentrations of Cas9 and guide RNA can be added to scale the process up or longer incubation times can allow for more complete cleavage of undesired DNA sequences. The reactions are stopped with 5× loading buffer containing 50 mM Tris PH 8.0 and 250 mM EDTA with 50% glycerol, and are resolved by 0.8 or 1% agarose gel electrophoresis and visualized by ethidium bromide staining by standard techniques known to one skilled in the art. Alternatively the DNA can be gel purified by phenol chloroform extraction, ethanol extraction or other comparable methods described herein or known to one skilled in the art. DNA can then be further enriched, PCR amplified or sequenced by methods described herein.

### Example 45

### Rapid Detection of Microorganisms by Fluorescence Methods

To enable the rapid detection of microorganisms diluted to countable colonies a rapid detection test based on the EZ-fluo rapid detection system is described. The technique is a test for viable microorganisms and is not intrinsically specific to any particular organism. One skilled in the art will recognize many embodiments where a combination of previous examples generating specific enrichment of microorganisms as previous steps to this subsequent detection step will produce specificity for detection of various organisms. To ensure appropriate quantification of microorganisms using this method, the volume of liquid or resuspended sample used for this technique should be chosen to ensure less than 300 cfu are present. To ensure this concentration in an unknown sample, multiple dilutions of the test suspension should be performed and tested to determine the appropriate dilution factor and back calculate the concentration of microorganisms. For example if 10 ml of sample is to be applied to the filter then less than 30 cfu/ml should be present in the solution. As a nonlimiting example a culture of A. brasiliensis and C. albicans is prepared and tested with the EZ-Fluo™ Rapid Detection System (EMD Millipore, Billerica, Mass.) as previously described (e.g. see http://www.foodsafetymagazine.com/signature-series/rapid-detection-of-microorganisms-in-food-and-beverage-by-fluorescence/). Briefly, C. albicans and A. brasiliensis are spiked are spiked into sterile liquid media at 50-70 cfu/mL. 2 and 3 ml of solution is used on culture or optionally 2 and 3 ml are diluted to 10 ml in sterile culture and applied to the membrane.

The following steps are performed in accordance with the EZ-Fluo rapid detection method. The sample is filtered over the appropriate membrane according to the manufacturing instructions with a vacuum manifold device as previously describe (e.g. see Microfil® & S-Pak® Membrane Filters/Microfil® & EZ-Pak® Systems User Guide and EZ-Stream™ Pump User Guide, EMD Millipore). The membrane is then transferred into a Petri-Pad Petri dish containing EZ-Fluo reagent for 30 minutes at 30-35° C. Fluorescent micro-colonies are counted using the EZ-fluo reader and camera reading assistance (optionally) to facilitate counting. As a confirmatory test the membrane can be incubated on a petri dish with various media to transfer colonies and these colonies can be grown as previously described in aforementioned examples for subsequent analysis and detection by genomic or microbiological mechanisms described herein.

### Example 46

### Identifying Pathogenicity Islands and Molecular Detection of Components in a Microbial Composition

To validate the microbial composition is substantially free of pathogens, virulence factors and mechanisms of pathogenic horizontal gene transfer including but not limited to pathogenicity island identification, plasmid identification, and transposon elements can be examined by genetic techniques. As a non-limiting specific example, pathogenicity islands are identified in E. faecalis, validated by genetic manipulation of the genome and tested in animal toxicology models, and finally developed into a screenable test using PCR or other similar molecular tests.

In the literature, a handful of E. faecalis genes have been characterized as virulence factors. They include the genes in the cytolysin operon that encode a cytolytic toxin (Coburn et al., 2003), the esp gene encoding a surface protein that contributes to urinary tract colonization and biofilm formation (Shankar et al., Infection derived Enterococcus faecalis strains are enriched in esp, a gene encoding a novel surface protein, Infect Immun. 67(1) 1999 and Tendolkar et al., Enterococcal surface protein, Esp, enhances biofilm formation by Enterococcus faecalis. Infect Immun. 72(10). 2004), and the agg gene encoding a surface protein necessary for conjugative gene transfer that also seems to enhance adherence to and internalization into eukaryotic cells (Rakita et al., 1999; Vanek et al. 1999; Kreft et al., 1992; Olmsted et al., 1994; Waters et al., 2004). These traits are enriched in clinical isolates as compared to isolates from healthy individuals (Lempiainen et al., 2005), but the correlation between infection and characterized virulence traits is not absolute. Similarly, genetic loci that confer resistance to antibiotics such as gentamicin and vancomycin (Zervos et al., 1987; Boyce et al., 1992) are enriched in clinical isolates, but are not essential for infection.

Using esp gene to identify a possible larger cassette conferring virulence, further elucidation of the pathogenicity island is determined by using one of the E. faecalis virulence factors, esp, and sequencing 1000 random clones derived from the genome of a Madison hospital outbreak strain MMH594. Closer examination of the esp locus in MMH594 and related strains that turned up in a St. Louis hospital outbreak revealed the presence of a pathogenicity island. With a size of approximately 150 kb, a G+C content of 32% (as compared to 38% for the rest of the genome), and terminally repeated 10 by flanking sequences, this element possesses all of the hallmarks of a typical pathogenicity island (Shankar et al., 2002). The PAI codes for 129 open reading frames (ORFs), and includes a number of genes of unknown function in addition to the known virulence traits cytolysin, Esp, and aggregation substance. Importantly, the island encodes additional, previously unstudied genes with putative functions that could have important roles in adaptation and survival in hostile environments. The lack of these genes in most non-infection-derived E. faecalis isolates suggests a class of potential new targets associated with disease, that are not essential for the commensal behavior of the organism. As such, this genetic marker can serve as a molecular marker of pathogenicity in a microbial composition.

The roles of genes and gene products, including toxins, in pathogenicity can be validated by deleting or disrupting these genes by standard genetic techniques and testing these strains in appropriate toxicology animal models. A given gene may be deleted via recombination with a DNA molecule carrying a deletion of that gene (a molecule in which the coding region of the gene has been deleted and flanking sequences have been joined to create a novel junction). The gene deletion sequence is created in vitro using standard molecular methods ((e.g. see Sambrook and Russell, Molecular cloning: a laboratory manual) and introduced into E. faecalis using conjugation or transformation (e.g., see Kristich, et al. 2005).

Once a specific gene or genomic loci is identified and validated as important for conferring pathogenicity or as being associated with a clinical isolates, or as a marker of a horizontal gene transfer element that carries pathogenicity factors, a molecular test is developed to detect the gene directly through qPCR techniques. Probes and appropriate primers are designed by one skilled in the art (e.g. see example 3 and 5). The protocol described herein for qPCR is then be performed on a microbial composition to identify the presence or absence of the pathogenic elements.

Alternatively, if the specific gene is a toxin or other protein product e.g. esp that is highly expressed in the pathogen or present on the surface, a recombinant version of the whole gene or a smaller antigenic piece (e.g. the external facing region of the gene of esp) of the gene is affinity tagged by a 6×His tag, MBP, or other common tags of the protein is expressed in a common expression system e.g. E. coli, S. cerevisiae, S2 insect cells, or baculovirus infected SF9 expression systems and purified by standard biochemical techniques using affinity chromatography. The protein is then used to produce two orthogonal antibodies by methods described herein (e.g. see Example 37 or Harlow and Lane, Antibodies: a laboratory Manual, 1988 or Accoceberry, I., M. Thellier, I. Desportes-Livage, A. Achbarou, S. Biligui, M. Danis, and A. Datry. 1999. Production of monoclonal antibodies directed against the microsporidium Enterocytozoon bieneusi. J. Clin. Microbiol. 37: 4107-4112). The two antibodies are derived from two different organisms e.g. mouse and rabbit, or rabbit and rat and must be able to simultaneously bind to the toxin or protein product in order to construct a sandwich ELISA assay. Monoclonal antibodies can also be used but should be derived from different animals and have unique, non-overlapping binding sites. Polyclonal antibodies derived from two different species from the a large antigenic fragment will have likely have this property. Optionally, the antibody reagents are generated from two different recombinant subunits of the same protein to ensure they can both bind and recognize non overlapping antigenic sites.

Kits are commercially available to generate an ELISA assay (Pierce Protein Biology Products, http://www.piercenet.com/cat/western-blotting-elisa-cell-imaging). Briefly, to perform an ELISA a first antibody or polyclonal antibody preparation is immobilized to the surface of a 96 well plate by chemical conjugation or physical adsorption techniques known to one skilled in the art, and excess is washed away (e.g. see Hermanson. Bioconjugation, 2008). Various dilutions of the test article, PBS buffer (negative control), or buffer containing various concentrations of the recombinant protein or toxin (positive control), are then incubated in separate wells of the plate for 16 hours at 4° C. with gentle rocking The wells are then washed to remove unbound material and the second orthogonal antibody is added, incubated for 1 hour, then washed five times. Finally the detection antibody (e.g. rabbit anti-mouse) or probe (e.g. streptavidin with a label if the second antibody is biotinylated) is added containing either the fluorescent, chemiluminescent, enzyme or other detection probe for 1 hour and subsequently washed per the manufacturer's instructions. Detection probe is used to determine the quantitative amount of toxin present and standard curves based on the positive control dilution are used to estimate the amount of protein or toxin present in a test solution. Test solutions derived from microbial compositions include but are not limited to the lysate of such microbial compositions, the spent media of a liquid culture from a microbial composition, and other embodiments are easily recognizable by one skilled in the art. One skilled in the art will also recognize several embodiments of the antigen based detection techniques or the genetic based techniques that are provided herein.

### Example 47

### Detection of C. difficile Toxin

To detect pathogenicity, toxins and other genes products unique to pathogens are used to detect the presence of a pathogen in a microbial composition. As a non-limiting example the following protocol demonstrates this methodology for detecting C. difficile toxin in a microbial composition as previously described (see e.g. Russman et al Evaluation of three rapid assays for detections of clostridium difficile toxin A and toxin B in stool specimens. Eur J Clin Microbiol Infect Dis. 26: 115-119, 2007). The commercially available kits are the rapid enzyme immunoassay Ridascreen Clostridium difficile Toxin A/B (R-Biopharm, Darmstadt, Germany) test, the C. difficile Tox A/B II Assay (TechLab, Blacksburg, Va., USA) and the ProSpecT C. difficile Toxin A/B Microplate Assay (Remel, Lenexa, Kans., USA). Similar assays can be adapted for other toxin products and will be recognized as other embodiments of this protocol by one skilled in the art. All three enzyme immuno assays (EIA) used are qualitative 96-well microplate assays to detect toxin A and toxin B of C. difficile. Assays are carried out and interpreted according to the manufacturers' instructions. All three tests are performed from the same portion of stool homogenized with a wooden applicator stick on the same day, after a single thaw at room temperature of the stored specimen or alternatively by methods previously described herein. Optionally, other microbial compositions are produced by alternative methods described herein to generate a suspension for testing. Washing of microplates between steps is done manually. Microplates for all assays are read spectrophotometrically. The C. difficile strain VPI 10463 (ATCC 43255) is used as an internal positive control.

In the RIDASCREEN® Clostridium difficile Toxin A/B test, monoclonal antibodies are used in a sandwich-type method. Monoclonal antibodies against toxins A and B of Clostridium difficile are bound to the surface of the microwells of the microtiter plate. A suspension of the stool sample to be examined and the controls, together with biotinylated monoclonal anti-toxin A and B antibodies (Conjugate 1), are pipetted into the well in the microwell plate at ambient temperature (20-25° C.) for incubation. After a wash step, polystreptavidin peroxidase conjugate (Conjugate 2) is added and the microwell plate incubated again at ambient temperature (20-25° C.). If toxin A and B are present in the stool sample, a sandwich complex is formed made up of the immobilised antibodies, the toxins and the antibodies conjugated with the biotine streptavidin peroxidase complex. Unbound enzyme-labelled antibodies are removed in another washing step. After adding substrate, the bound enzyme with positive samples transforms the colourless solution in the microwells in a blue solution. By addition of stop reagent a colour RIDASCREEN® Clostridium difficile Toxin A/B 12-05-24 3 change from blue to yellow occurs. The measured absorbance of the colour is proportional to the concentration of the existing Toxins A and B in the sample. The following protocol is from the manufacturer instructions (e.g. see http://www.r-biopharm.com/wp-content/uploads/items/ridascreen-clostridium-difficile-toxin-ab-3865/C0801-Clostridium-difficileToxin-AB_{—}12-05-24_GB.pdf) and all references to buffers are commercially available to allow the procedure to be performed)

All reagents and the microwell plate Plate must be brought to room temperature (20-25° C.) before use. The microwell strips must not be removed from the aluminium bag until they have reached room temperature. The reagents must be thoroughly mixed immediately before use. After use, the microwell strips (in sealed bags) and the reagents must be stored at 2-8° C. Once used, the microwell strips must not be used again. The reagents and microwell strips must not be used if the packaging is damaged or the vials are leaking. In order to prevent cross contamination, the samples must be prevented from coming into direct contact with the kit components. The test must not be carried out in direct sunlight. We recommend that the microwell plate be covered or sealed with film in order to prevent evaporation losses. Mix 1 part wash buffer concentrate with 9 parts distilled water. Any crystals present in the concentrate must be dissolved beforehand by warming in a water bath at 37° C. Place 1 ml RIDASCREEN® sample dilution buffer Diluent-1 in a labelled test tube. Suck up liquid stool in a disposable pipette (Article no Z0001) until it passes the second thickening (approx. 100 μl) and suspend it in the sample dilution buffer. With solid stools, take an equivalent amount (100 mg) with a spatula or a disposable inoculation loop and suspend it in solution. Homogenise the stool suspension by suction and ejection from a disposable pipette or, alternatively, by mixing in a vortex mixer. After leaving for a short time for the coarse stool particles to settle, the clarified supernatant of the stool suspension can be used directly in the test. If the test procedure is carried out in an automated ELISA system, the supernatant must be particle-free. In this case, it is advisable to centrifuge the sample at 2500 G for 5 minutes. In order to test colonies after culturing them on solid media (CCF agar or Schaedler agar), remove them from the agar plate with an inoculation loop and suspend them in 1 ml sample dilution buffer Diluent-1 and mix well. After this, centrifuge the suspension (5 minutes at 2500 g). The clear supernatant can be used in the test directly. To test liquid cultures, suspend 100 μl of this in 1 ml sample dilution buffer Diluent |1 and mix well. After this, centrifuge the suspension (5 minutes at 2500 g). The clear supernatant can be used in the test directly. After selecting a sufficient number of wells in the frame, pipette 2 drops (or 100 μl) of positive control Control+, the sample dilution buffer Diluent 1 (=negative control) or the stool suspension in the wells. Then add 2 drops (100 μl) of the biotin-conjugated antibody Conjugate 1 and, after mixing thoroughly (by lightly tapping on the edge of the plate), incubate at room temperature (20-25° C.) for 60 minutes. Careful washing is important in order to achieve the correct results and should therefore take place strictly according to the instructions. The incubated substance in the wells must be emptied into a waste container containing hypochlorite for disinfection. After this, knock out the plate onto absorbent paper in order to remove the residual moisture. Then wash the plate 5 times using 300 μl wash buffer each time.

Make sure that the wells are emptied completely by knocking them out after each wash on a part of the absorbent paper which is still dry and unused. Add 2 drops (100 μl) of the polystreptavidin peroxidase conjugate Conjugate 2 to the wells and incubate at room temperature (20-25° C.) for 30 minutes. Repeat washing step then proceed. Add 2 drops (100 μl) of substrate Substrate to each well. Then incubate the plate at room temperature (20-25° C.) for 15 minutes in the dark. After this, stop the reaction by adding 1 drop (50 μl) of stop reagent Stop to each well. After carefully mixing (slight tipping on the plate frame) the absorbance is measured at 450 nm (optional: reference wave length ≧600 nm). Then calibrate the zero against air, that means without microtiter plate. In order to establish the cut-off, 0.15 extinction units are added to the measured extinction for the negative control. Cut-off=Extinction for the negative control+0.15. The quantitative change in color of the reagent can be measured with a standard plate reader and positives are evaluated by standard techniques known to one skilled in the art e.g. 3 standard deviations above the negative control or significantly different after multiple replicates are performed.

The CBA (C. difficile TOX-B Test; TechLab) is performed either with supernatants from stool suspensions. The cytotoxin assay is carried out in 96-well plates according to the manufacturer's instructions using Vero cells (ATCC CCL-81). Briefly, Vero cells are incubated with the respective supernatants for 48 h. Cells are checked for cytotoxic effects after 24 and 48 h.

### Example 47

### Identification of Keystone OTUs and Functions

The human body is an ecosystem in which the microbiota, and the microbiome, play a significant role in the basic healthy function of human systems (e.g. metabolic, immunological, and neurological). The microbiota and resulting microbiome comprise an ecology of microorganisms that co-exist within single subjects interacting with one another and their host (i.e., the mammalian subject) to form a dynamic unit with inherent biodiversity and functional characteristics. Within these networks of interacting microbes (i.e. ecologies), particular members can contribute more significantly than others; as such these members are also found in many different ecologies, and the loss of these microbes from the ecology can have a significant impact on the functional capabilities of the specific ecology. Robert Paine coined the concept “Keystone Species” in 1969 (see Paine R T. 1969. A note on trophic complexity and community stability. The American Naturalist 103: 91-93.) to describe the existence of such lynchpin species that are integral to a given ecosystem regardless of their abundance in the ecological community. Paine originally describe the role of the starfish Pisaster ochraceus in marine systems and since the concept has been experimentally validated in numerous ecosystems.

Keystone OTUs and/or Functions are computationally-derived by analysis of network ecologies elucidated from a defined set of samples that share a specific phenotype. Keystone OTUs and/or Functions are defined as all Nodes within a defined set of networks that meet two or more of the following criteria. Using Criterion 1, the node is frequently observed in networks, and the networks in which the node is observed are found in a large number of individual subjects; the frequency of occurrence of these Nodes in networks and the pervasiveness of the networks in individuals indicates these Nodes perform an important biological function in many individuals. Using Criterion 2, the node is frequently observed in networks, and each the networks in which the node is observed contain a large number of Nodes—these Nodes are thus “super-connectors”, meaning that they form a nucleus of a majority of networks and as such have high biological significance with respect to their functional contributions to a given ecology. Using Criterion 3, the node is found in networks containing a large number of Nodes (i.e. they are large networks), and the networks in which the node is found occur in a large number of subjects; these networks are potentially of high interest as it is unlikely that large networks occurring in many individuals would occur by chance alone strongly suggesting biological relevance. Optionally, the required thresholds for the frequency at which a node is observed in network ecologies, the frequency at which a given network is observed across subject samples, and the size of a given network to be considered a Keystone node are defined by the 50th, 70th, 80th, or 90th percentiles of the distribution of these variables. Optionally, the required thresholds are defined by the value for a given variable that is significantly different from the mean or median value for a given variable using standard parametric or non-parametric measures of statistical significance. In another embodiment a Keystone node is defined as one that occurs in a sample phenotype of interest such as but not limited to “health” and simultaneously does not occur in a sample phenotype that is not of interest such as but not limited to “disease.” Optionally, a Keystone Node is defined as one that is shown to be significantly different from what is observed using permuted test datasets to measure significance.

### Example 48

### Microbial Population (Engraftment and Augmentation) and Reduction of Pathogen Carriage in Patients Treated with Spore Compositions

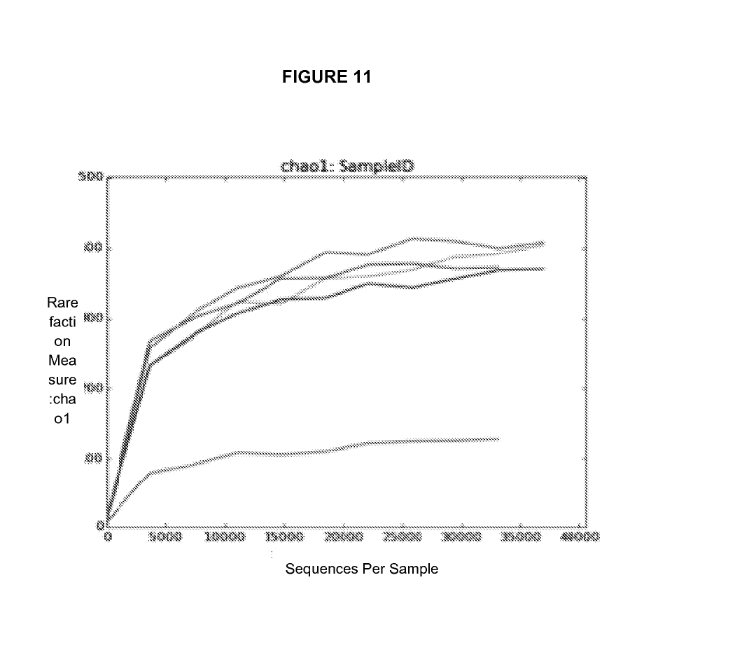

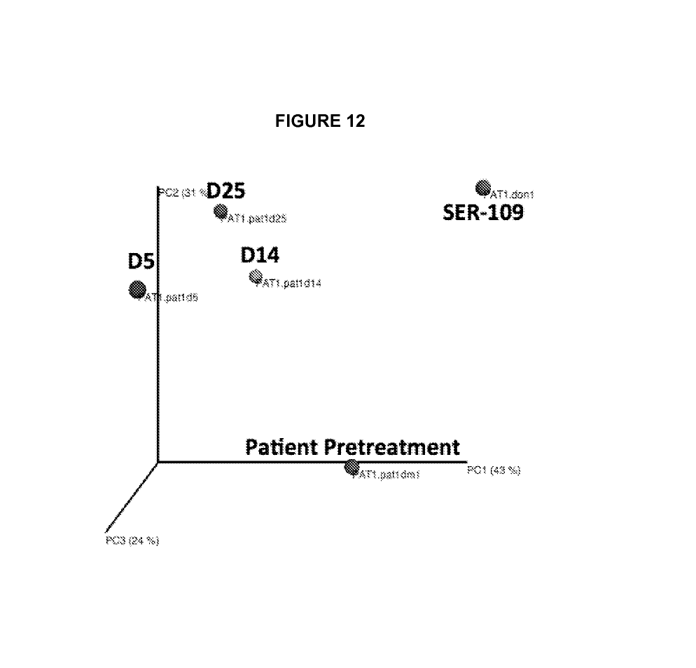

The following example is a non-limiting example of how one could determine what is present in the microbial composition using genomic techniques. Complementary genomic and microbiological methods were used to characterize the composition of the microbiota from Patient 1, 2, 3, 4, and 5, 6, 7, 8, 9, and 10 at pretreatment (pretreatment) and on up to 4 weeks post-treatment. To determine the OTUs that engraft from treatment with an ethanol treated spore preparation in the patients and how their microbiome changed in response, the microbiome was characterized by 16S-V4 sequencing prior to treatment (pretreatment) with an ethanol treated spore preparation and up to 25 days after receiving treatment. Alternatively, one might use a bacterial composition in the vegetative state, or a mixture of vegetative bacteria and bacterial spores. For example, the treatment of patient 1 with an ethanol treated spore preparation led to microbial population via the engraftment of OTUs from the spore treatment and augmentation in the microbiome of the patient (FIG. 11 and FIG. 12). By day 25 following treatment, the total microbial carriage was dominated by species of the following taxonomic groups: Bacteroides, Sutterella, Ruminococcus, Blautia, Eubacterium, Gemmiger/Faecalibacterium, and the non-sporeforming Lactobacillus (see Table 26 for specific OTUs). The first two genera represent OTUs that do not form spores while the latter taxonomic groups represent OTUs that are believed to form spores.

Table 26 shows bacterial OTUs associated with engraftment and ecological augmentation and establishment of a more diverse microbial ecology in patients treated with an ethanol treated spore preparation. OTUs that comprise an augmented ecology are not present in the patient prior to treatment and/or exist at extremely low frequencies such that they do not comprise a significant fraction of the total microbial carriage and are not detectable by genomic and/or microbiological assay methods. OTUs that are members of the engrafting and augmented ecologies were identified by characterizing the OTUs that increase in their relative abundance post treatment and that respectively are: (i) present in the ethanol treated spore preparation and absent in the patient pretreatment (engrafting OTUs), or (ii) absent in the ethanol treated spore preparation, but increase in their relative abundance through time post treatment with the preparation due to the formation of favorable growth conditions by the treatment (augmenting OTUs). Notably, the latter OTUs can grow from low frequency reservoirs in the patient, or be introduced from exogenous sources such as diet. OTUs that comprise a “core” augmented or engrafted ecology can be defined by the percentage of total patients in which they are observed to engraft and/or augment; the greater this percentage the more likely they are to be part of a core ecology responsible for catalyzing a shift away from a dysbiotic ecology. The dominant OTUs in an ecology can be identified using several methods including but not limited to defining the OTUs that have the greatest relative abundance in either the augmented or engrafted ecologies and defining a total relative abundance threshold. As example, the dominant OTUs in the augmented ecology of Patient-1 were identified by defining the OTUs with the greatest relative abundance, which together comprise 60% of the microbial carriage in this patient's augmented ecology.

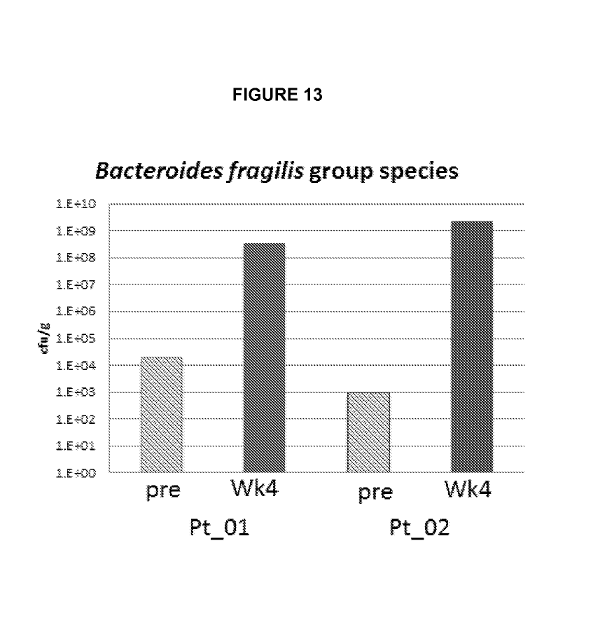

Patient treatment with the ethanol treated spore preparation led to the population of a microbial ecology that has greater diversity than prior to treatment (FIG. 11). Genomic-based microbiome characterization confirmed engraftment of a range of OTUs that were absent in the patient pretreatment (Table 26). These OTUs comprised both bacterial species that were capable and not capable of forming spores, and OTUs that represent multiple phylogenetic clades. Organisms absent in Patient 1 pre-treatment either engraft directly from the ethanol treated spore fraction or are augmented by the creation of a gut environment favoring a healthy, diverse microbiota. Furthermore, Bacteroides fragilis group species were increased by 4 and 6 logs in patients 1 and 2 (FIG. 13).

FIG. 12 shows patient microbial ecology is shifted by treatment with an ethanol treated spore treatment from a dysbiotic state to a state of health. Principle Coordinates Analysis based on the total diversity and structure of the microbiome (Bray-Curtis Beta-Diversity) of the patient pre- and post-treatment delineates that the engraftment of OTUs from the spore treatment and the augmentation of the patient microbial ecology leads to a microbial ecology that is distinct from both the pretreatment microbiome and the ecology of the ethanol treated spore treatment (Table 26).

FIG. 13 shows the augmentation of Bacteroides species in patients. Comparing the number of Bacteroides fragilis groups species per cfu/g of feces pre-treatment and in week 4 post treatment reveals an increase of 4 logs or greater. The ability of 16S-V4 OTU identification to assign an OTU as a specific species depends in part on the resolution of the 16S-V4 region of the 16S gene for a particular species or group of species. Both the density of available reference 16S sequences for different regions of the tree as well as the inherent variability in the 16S gene between different species will determine the definitiveness of a taxonomic annotation to a given sequence read. Given the topological nature of a phylogenetic tree and that the tree represents hierarchical relationships of OTUs to one another based on their sequence similarity and an underlying evolutionary model, taxonomic annotations of a read can be rolled up to a higher level using a clade-based assignment procedure (Table 1). Using this approach, clades are defined based on the topology of a phylogenetic tree that is constructed from full-length 16S sequences using maximum likelihood or other phylogenetic models familiar to individuals with ordinary skill in the art of phylogenetics. Clades are constructed to ensure that all OTUs in a given clade are: (i) within a specified number of bootstrap supported nodes from one another (generally, 1-5 bootstraps), and (ii) within a 5% genetic similarity. OTUs that are within the same clade can be distinguished as genetically and phylogenetically distinct from OTUs in a different clade based on 16S-V4 sequence data. OTUs falling within the same clade are evolutionarily closely related and may or may not be distinguishable from one another using 16S-V4 sequence data. The power of clade based analysis is that members of the same clade, due to their evolutionary relatedness, play similar functional roles in a microbial ecology such as that found in the human gut. Compositions substituting one species with another from the same clade are likely to have conserved ecological function and therefore are useful in the present invention.

Stool samples were aliquoted and resuspended 10×vol/wt in either 100% ethanol (for genomic characterization) or PBS containing 15% glycerol (for isolation of microbes) and then stored at −80° C. until needed for use. For genomic 16S sequence analysis colonies picked from plate isolates had their full-length 16S sequence characterized as described in Examples 2 and 3, and primary stool samples were prepared targeting the 16S-V4 region using the method for heterogeneous samples described herein.

Notably, 16S sequences of isolates of a given OTU are phylogenetically placed within their respective clades despite that the actual taxonomic assignment of species and genus may suggest they are taxonomically distinct from other members of the clades in which they fall. Discrepancies between taxonomic names given to an OTU is based on microbiological characteristics versus genetic sequencing are known to exist from the literature. The OTUs footnoted in this table are known to be discrepant between the different methods for assigning a taxonomic name.

Engraftment of OTUs from the ethanol treated spore preparation treatment into the patient as well as the resulting augmentation of the resident microbiome led to a significant decrease in and elimination of the carriage of pathogenic species other than C. difficile in the patient. 16S-V4 sequencing of primary stool samples demonstrated that at pretreatment, 20% of reads were from the genus Klebsiella and an additional 19% were assigned to the genus Fusobacterium. These data are evidence of a profoundly dysbiotic microbiota associated with recurrent C. difficile infection and chronic antibiotic use. In healthy individuals, Klebsiella is a resident of the human microbiome in only about 2% of subjects based on an analysis of HMP database (www.hmpdacc.org), and the mean relative abundance of Klebsiella is only about 0.09% in the stool of these people. The 20% relative abundance in Patient 1 before treatment is an indicator of a proinflammatory gut environment enabling a “pathobiont” to overgrow and outcompete the commensal organisms normally found in the gut. Similarly, the dramatic overgrowth of Fusobacterium indicates a profoundly dysbiotic gut microbiota. One species of Fusobacterium, F. nucleatum (an OTU phylogenetically indistinguishable from Fusobacterium sp. 3_{—}1_{—}33 based on 16S-V4), has been termed “an emerging gut pathogen” based on its association with IBD, Crohn's disease, and colorectal cancer in humans and its demonstrated causative role in the development of colorectal cancer in animal models [Allen-Vercoe, Gut Microbes (2011) 2:294-8]. Importantly, neither Klebsiella nor Fusobacterium was detected in the 16S-V4 reads by Day 25 (Table 27).

To further characterize the colonization of the gut by Klebsiella and other Enterobacteriaceae and to speciate these organisms, pretreatment and Day 25 fecal samples stored at −80 C as PBS-glycerol suspensions were plated on a variety of selective media including MacConkey lactose media (selective for gram negative enterobacteria) and Simmons Citrate Inositol media (selective for Klebsiella spp) [Van Cregten et al, J. Clin. Microbiol. (1984) 20: 936-41]. Enterobacteria identified in the patient samples included K. pneumoniae, Klebsiella sp. Co_{—}9935 and E. coli. Strikingly, each Klebsiella species was reduced by 2-4 logs whereas E. coli, a normal commensal organism present in a healthy microbiota, was reduced by less than 1 log (Table 28 below). This decrease in Klebsiella spp. carriage is consistent across multiple patients. Four separate patients were evaluated for the presence of Klebsiella spp. pre treatment and 4 weeks post treatment. Klebsiella spp. were detected by growth on selective Simmons Citrate Inositol media as previously described. Serial dilution and plating, followed by determining cfu/mL titers of morphologically distinct species and 16S full length sequence identification of representatives of those distinct morphological classes, allowed calculation of titers of specific species.

The genus Bacteroides is an important member of the gastrointestinal microbiota; 100% of stool samples from the Human Microbiome Project contain at least one species of Bacteroides with total relative abundance in these samples ranging from 0.96% to 93.92% with a median relative abundance of 52.67% (www.hmpdacc.org reference data set HMSMCP). Bacteroides in the gut has been associated with amino acid fermentation and degradation of complex polysaccharides. Its presence in the gut is enhanced by diets rich in animal-derived products as found in the typical western diet [David, L. A. et al, Nature (2013) doi:10.1038/nature12820]. Prior to treatment, fewer than 0.008% of the 16S-V4 reads from Patient 1 mapped to the genus Bacteroides strongly suggesting that Bacteroides species were absent or that viable Bacteroides were reduced to an extremely minor component of the patient's gut microbiome. Post treatment, >42% of the 16S-V4 reads were assigned to the genus Bacteroides within 5 days of treatment and by Day 25 post treatment 59.48% of the patients gut microbiome was comprised of Bacteroides. These results were confirmed microbiologically by the absence of detectable Bacteroides in the pretreatment sample plated on two different Bacteroides selective media: Bacteroides Bile Esculin (BBE) agar which is selective for Bacteroides fragilis group species [Livingston, S. J. et al J. Clin. Microbiol (1978). 7: 448-453] and Polyamine Free Arabinose (PFA) agar [Noack et al. J. Nutr. (1998) 128: 1385-1391; modified by replacing glucose with arabinose]. The highly selective BBE agar had a limit of detection of <2×103 cfu/g, while the limit of detection for Bacteroides on PFA agar was approximately 2×107 cfu/g due to the growth of multiple non-Bacteroides species in the pretreatment sample on that medium. Colony counts of Bacteroides species on Day 25 were up to 2×1010 cfu/g, consistent with the 16S-V4 sequencing, demonstrating a profound reconstitution of the gut microbiota in Patient 1 (Table 29 below).

The significant abundance of Bacteroides in Patient 1 on Day 25 (and as early as Day 5 as shown by 16S-V4 sequencing) is remarkable. Viable Bacteroides fragilis group species were not present in the ethanol treated spore population based on microbiological plating (limit of detection of 10 cfu/ml). Thus, administration of the ethanol treated spore population to Patient 1 resulted in microbial population of the patient's GI tract, not only due to the engraftment of bacterial species such as but not limited to spore forming species, but also the restoration of high levels of non-spore forming species commonly found in healthy individuals through the creation of a niche that allowed for the repopulation of Bacteroides species. These organisms were most likely either present at extremely low abundance in the GI tract of Patient 1, or present in a reservoir in the GI tract from which they could rebound to high titer. Those species may also be reinoculated via oral uptake from food following treatment. We term this healthy repopulation of the gut with OTUs that are not present in the bacterial composition such as but not limited to a spore population or ethanol treated spore population, “Augmentation.” Augmentation is an important phenomenon in that it shows the ability to use an ethanol treated spore ecology or other bacterial composition to restore a healthy microbiota by seeding a diverse array or commensal organisms beyond the actual component organisms in the bacterial composition such as but not limited to an ethanol treated spore population itself; specifically the spore composition treatment itself and the engraftment of OTUs from the spore composition create a niche that enables the outgrowth of OTUs required to shift a dysbiotic microbiome to a microbial ecology that is associated with health. The diversity of Bacteroides species and their approximate relative abundance in the gut of Patient 1 is shown in Table 30, comprising at least 8 different species.

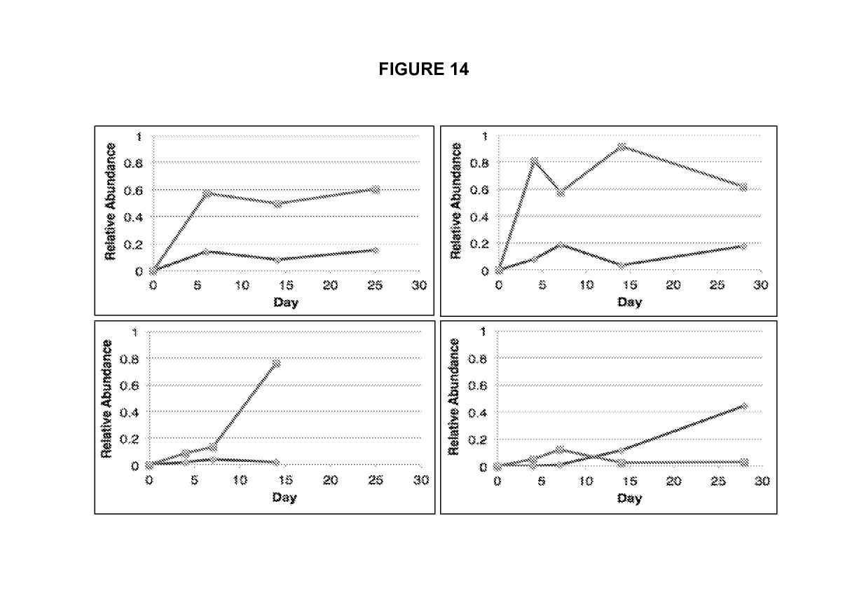

FIG. 14 shows species engrafting versus species augmenting in patients microbiomes after treatment with a bacterial composition such as but not limited to an ethanol-treated spore population. Relative abundance of species that engrafted or augmented as described were determined based on the number of 16S sequence reads. Each plot is from a different patient treated with the bacterial composition such as but not limited to an ethanol-treated spore population for recurrent C. difficile.

The impact of the bacterial composition such as but not limited to an ethanol treated spore population treatment on carriage of imipenem resistant Enterobacteriaceae was assessed by plating pretreatment and Day 28 clinical samples from Patients 2, 4 and 5 on MacConkey lactose plus 1 ug/mL of imipenem. Resistant organisms were scored by morphology, enumerated and DNA was submitted for full length 16S rDNA sequencing as described above. Isolates were identified as Morganella morganii, Providencia rettgeri and Proteus pennerii. Each of these are gut commensal organisms; overgrowth can lead to bacteremia and/or urinary tract infections requiring aggressive antibiotic treatment and, in some cases, hospitalization [Kim, B-N, et al Scan J. Inf Dis (2003) 35: 98-103; Lee, I-K and Liu, J-W J. Microbiol Immunol Infect (2006) 39: 328-334; O'Hara et al, Clin Microbiol Rev (2000) 13: 534]. The titer of organisms at pretreatment and Day 28 by patient is shown in Table 31. Importantly, administration of the bacterial composition such as but not limited to an ethanol treated spore preparation resulted in greater than 100-fold reduction in 4 of 5 cases of Enterobacteriaceae carriage with multiple imipenem resistant organisms (See Table 31 which shows titers (in cfu/g) of imipenem-resistant M. morganii, P. rettgeri and P. pennerii from Patients 2, 4 & 5).

In addition to speciation and enumeration, multiple isolates of each organism from Patient 4 were grown overnight in 96-well trays containing a 2-fold dilution series of imipenem in order to quantitatively determine the minimum inhibitory concentration (MIC) of antibiotic. Growth of organisms was detected by light scattering at 600 nm on a SpectraMax M5e plate reader. In the clinical setting, these species are considered resistant to imipenem if they have an MIC of 1 ug/mL or greater. M. morganii isolates from pretreatment samples from Patient D had MICs of 2-4 ug/mL and P. pennerii isolates had MICs of 4-8 ug/mL. Thus, the bacterial composition, such as but not limited to, an ethanol treated spores administered to Patient 4 caused the clearance of 2 imipenem resistant organisms (Table 26). While this example specifically uses a spore preparation, the methods herein describe how one skilled in the art would use a more general bacterial composition to achieve the same effects. The specific example should not be viewed as a limitation of the scope of this disclosure.

### Example 49

### Identifying the Core Ecology from the Bacterial Combination

To identify the composition of microbes in a complex microbial composition, genomic methods were employed. Ten different bacterial compositions were made by the ethanol treated spore preparation methods from 6 different donors (as described in Example 9). The spore preparations were used to treat 10 patients, each suffering from recurrent C. difficile infection. Patients were identified using the inclusion/exclusion criteria described in herein, and donors were identified using the criteria described in AAAJ. None of the patients experienced a relapse of C. difficile in the 4 weeks of follow up after treatment, whereas the literature would predict that 70-80% of subjects would experience a relapse following cessation of antibiotic [Van Nood, et al, NEJM (2013)]. Thus, the ethanol treated spore preparations derived from multiple different donors and donations showed remarkable clinical efficacy. These ethanol treated spore preparations are a subset of the bacterial compositions described herein and the results should not be viewed as a limitation on the scope of the broader set of bacterial compositions.

To define the Core Ecology underlying the remarkable clinical efficacy of the bacterial compositions e.g. ethanol treated spore preparations, the following analysis was carried out. The OTU composition of the spore preparation was determined by 16S-V4 rDNA sequencing and computational assignment of OTUs per Example 3. A requirement to detect at least ten sequence reads in the ethanol treated spore preparation was set as a conservative threshold to define only OTUs that were highly unlikely to arise from errors during amplification or sequencing. Methods routinely employed by those familiar to the art of genomic-based microbiome characterization use a read relative abundance threshold of 0.005% (see e.g. Bokulich, A. et al. 2013. Quality-filtering vastly improves diversity estimates from Illumina amplicon sequencing. Nature Methods 10: 57-59), which would equate to ≧2 reads given the sequencing depth obtained for the samples analyzed in this example, as cut-off which is substantially lower than the ≧10 reads used in this analysis. All taxonomic and clade assignments were made for each OTU as described in Example 4. The resulting list of OTUs, clade assignments, and frequency of detection in the spore preparations are shown in Table 32. Table 32 shows OTUs detected by a minimum of ten 16S-V4 sequence reads in at least a one ethanol treated spore preparation (pan-microbiome). OTUs that engraft in a treated patients and the percentage of patients in which they engraft are denoted, as are the clades, spore forming status, and Keystone OTU status. Starred OTUs occur in ≧80% of the ethanol preps and engraft in ≧50% of the treated patients.

Next, it was reasoned that for an OTU to be considered a member of the Core Ecology of the bacterial composition, that OTU was shown to engraft in a patient. Engraftment is important for two reasons. First, engraftment is a sine qua non of the mechanism to reshape the microbiome and eliminate C. difficile colonization. OTUs that engraft with higher frequency are highly likely to be a component of the Core Ecology of the spore preparation or broadly speaking a set bacterial composition. Second, OTUs detected by sequencing a bacterial composition (as in Table 32 may include non-viable cells or other contaminant DNA molecules not associated with the composition. The requirement that an OTU was shown to engraft in the patient eliminates OTUs that represent non-viable cells or contaminating sequences. Table 32 also identifies all OTUs detected in the bacterial composition that also were shown to engraft in at least one patient post-treatment. OTUs that are present in a large percentage of the bacterial composition e.g. ethanol spore preparations analyzed and that engraft in a large number of patients represent a subset of the Core Ecology that are highly likely to catalyze the shift from a dysbiotic disease ecology to a healthy microbiome.

A third lens was applied to further refine insights into the Core Ecology of the bacterial composition e.g. spore preparation. Computational-based, network analysis has enabled the description of microbial ecologies that are present in the microbiota of a broad population of healthy individuals. These network ecologies are comprised of multiple OTUs, some of which are defined as Keystone OTUs. Keystone OTUs form a foundation to the microbially ecologies in that they are found and as such are central to the function of network ecologies in healthy subjects. Keystone OTUs associated with microbial ecologies associated with healthy subjects are often are missing or exist at reduced levels in subjects with disease. Keystone OTUs may exist in low, moderate, or high abundance in subjects. Table 32 further notes which of the OTUs in the bacterial composition e.g. spore preparation are Keystone OTUs exclusively associated with individuals that are healthy and do not harbor disease.

A relatively small number of species, 16 in total, are detected in all of the spore preparations from 6 donors and 10 donations. The HMP database (www.hmpdacc.org) describes the enormous variability of commensal species across healthy individuals. The presence of a small number of consistent OTUs lends support to the concept of a Core Ecology. The engraftment data further supports this conclusion. A regression analysis shows a significant correlation between frequency of detection in a spore preparation and frequency of engraftment in a donor: R=0.43 (p<0.001). There is no a priori requirement that an OTU detected frequently in the bacterial composition e.g. spore preparation will or should engraft. For instance, Lutispora thermophila, a spore former found in all ten spore preparations, did not engraft in any of the patients. Bilophila wadsworthia, a gram negative anaerobe, is present in 9 of 10 donations, yet it does not engraft in any patient, indicating that it is likely a non-viable contaminant in the ethanol treated spore preparation. Finally, it is worth noting the high preponderance of previously defined Keystone OTUs among the most frequent OTUs in the spore preparations.

These three factors—prevalence in the bacterial composition such as but not limited to a spore preparation, frequency of engraftment, and designation as a Keystone OTUs—enabled the creation of a “Core Ecology Score” (CES) to rank individual OTUs. CES was defined as follows:
40% weighting for presence of OTU in spore preparation
   multiplier of 1 for presence in 1-3 spore preparations
   multiplier of 2.5 for presence in 4-8 spore preparations
   multiplier of 5 for presences in ≧9 spore preparations
40% weighting for engraftment in a patient
   multiplier of 1 for engraftment in 1-4 patients
   multiplier of 2.5 for engraftment in 5-6 patients
   multiplier of 5 for engraftment in ≧7 patients
20% weighting to Keystone OTUs
   multiplier of 1 for a Keystone OTU
   multiplier of 0 for a non-Keystone OTU

Using this guide, the CES has a maximum possible score of 5 and a minimum possible score of 0.8. As an example, an OTU found in 8 of the 10 bacterial composition such as but not limited to a spore preparations that engrafted in 3 patients and was a Keystone OTU would be assigned the follow CES:

CES=(0.4×2.5)+(0.4×1)+(0.2×1)=1.6

Table 33 ranks the top 20 OTUs by CES with the further requirement that an OTU was shown to engraft to be a considered an element of a core ecology.

### Example 50

### Defining Efficacious Subsets of the Core Ecology

The number of organisms in the human gastrointestinal tract, as well as the diversity between healthy individuals, is indicative of the functional redundancy of a healthy gut microbiome ecology (see The Human Microbiome Consortia. 2012. Structure, function and diversity of the healthy human microbiome. Nature 486: 207-214). This redundancy makes it highly likely that subsets of the Core Ecology describe therapeutically beneficial components of the bacterial composition such as but not limited to an ethanol treated spore preparation and that such subsets may themselves be useful compositions for populating the GI tract and for the treatment of C. difficile infection given the ecologies functional characteristics. Using the CES, individual OTUs can be prioritized for evaluation as an efficacious subset of the Core Ecology.

Another aspect of functional redundancy is that evolutionarily related organisms (i.e. those close to one another on the phylogenetic tree, e.g. those grouped into a single clade) will also be effective substitutes in the Core Ecology or a subset thereof for treating C. difficile.

To one skilled in the art, the selection of appropriate OTU subsets for testing in vitro (e.g. see Example 51 below) or in vivo is straightforward. Subsets may be selected by picking any 2, 3, 4, 5, 6, 7, 8, 9, 10, or more than 10 OTUs from Table 32, with a particular emphasis on those with higher CES, such as the OTUs described Table 33. In addition, using the clade relationships defined in Example 3 and Table 1 above, related OTUs can be selected as substitutes for OTUs with acceptable CES values. These organisms can be cultured anaerobically in vitro using the appropriate media (selected from those described in Example 5 above), and then combined in a desired ratio. A typical experiment in the mouse C. difficile model utilizes at least 104 and preferably at least 105, 106, 107, 108, 109 or more than 109 colony forming units of a each microbe in the composition. Variations in the culture yields may sometimes mean that organisms are combined in unequal ratios, e.g. 1:10, 1:100, 1:1,000, 1:10,000, 1:100,000, or greater than 1:100,000. What is important in these compositions is that each strain be provided in a minimum amount so that the strain's contribution to the efficacy of the Core Ecology subset can be measured. Using the principles and instructions described here, it is straightforward for one of skill in the art to make clade-based substitutions to test the efficacy of subsets of the Core Ecology. Table 32 describes the clades for each OTU detected in a spore preparation and Table 1 describes the OTUs that can be used for substitutions based on clade relationships.

### Example 51

### Testing Subsets of the Core Ecology in the Mouse Model

Several subsets of the Core Ecology were tested in the C. difficile mouse model. The negative control was phosphate buffered saline and the positive control was a 10% human fecal suspension. The subsets are described in Table 34 (Subsets of the Core Ecology tested in the C. difficile mouse model).

Two cages of five mice each were tested for each arm of the experiment. All mice received an antibiotic cocktail consisting of 10% glucose, kanamycin (0.5 mg/ml), gentamicin (0.044 mg/ml), colistin (1062.5 U/ml), metronidazole (0.269 mg/ml), ciprofloxacin (0.156 mg/ml), ampicillin (0.1 mg/ml) and Vancomycin (0.056 mg/ml) in their drinking water on days −14 through −5 and a dose of 10 mg/kg Clindamycin by oral gavage on day −3. On day −1, they received either the test articles or control articles via oral gavage. On day 0, they were challenged by administration of approximately 4.5 log 10 cfu of C. difficile (ATCC 43255) via oral gavage. Mortality was assessed every day from day 0 to day 6 and the weight and subsequent weight change of the animal was assessed with weight loss being associated with C. difficile infection. Mortality and reduced weight loss of the test article compared to the empty vehicle was used to assess the success of the test article. Additionally, a C. difficile symptom scoring was performed each day from day −1 through day 6. Symptom scoring was based on Appearance (0-2 pts based on normal, hunched, piloerection, or lethargic), Respiration (0-2 pts based on normal, rapid or shallow, with abdominal breathing), Clinical Signs (0-2 points based on normal, wet tail, cold-to-the-touch, or isolation from other animals).

In addition to compiling the cumulative mortality for each arm, the average minimum relative weight is calculated as the mean of each mouse's minimum weight relative to Day −1 and the average maximum clinical score is calculated as the mean of each mouse's maximum combined clinical score with a score of 4 assigned in the case of death. The results are reported in Table 35 below (Results of bacterial compositions tested in a C. difficile mouse model).

### Example 52

### Defining Subsets of the Core Ecology in the In Vitro C. difficile Inhibition Assay

Vials of −80° C. glycerol stock banks were thawed and diluted to le8 CFU/mL. Selected strains and their clade assignment are given in Table 36. Each strain was then diluted 10× (to a final concentration of le7 CFU/mL of each strain) into 200 uL of PBS+15% glycerol in the wells of a 96-well plate. Plates were then frozen at −80° C. When needed for the assay, plates were removed from −80° C. and thawed at room temperature under anaerobic conditions when testing in a in vitro C. difficile inhibition assay (CivSim).

An overnight culture of Clostridium difficile was grown under anaerobic conditions in SweetB-FosIn or other suitable media for the growth of C. difficile. SweetB-FosIn is a complex media composed of brain heart infusion, yeast extract, cysteine, cellobiose, maltose, soluble starch, and fructooligosaccharides/inulin, and hemin, and is buffered with MOPs. After 24 hr of growth the culture was diluted 100,000 fold into a complex media such as SweetB-FosIn which is suitable for the growth of a wide variety of anaerobic bacterial species. The diluted C. difficile mixture was then aliquoted to wells of a 96-well plate (180 uL to each well). 20 uL of a subset Core Ecology is then added to each well at a final concentration of le6 CFU/mL of each species. Alternatively the assay can be tested each species at different initial concentrations (1e9 CFU/mL, le8 CFU/mL, le7 CFU/mL, le5 CFU/mL, le4 CFU/mL, le3 CFU/mL, le2 CFU/mL). Control wells only inoculated with C. difficile were included for a comparison to the growth of C. difficile without inhibition. Additional wells were used for controls that either inhibit or do not inhibit the growth of C. difficile. One example of a positive control that inhibits growth was a combination of Blautia producta, Clostridium bifermentans and Escherichia coli. One example of a control that shows reduced inhibition of C. difficile growth was a combination of Bacteroides thetaiotaomicron, Bacteroides ovatus and Bacteroides vulgatus. Plates were wrapped with parafilm and incubated for 24 hr at 37° C. under anaerobic conditions. After 24 hr the wells containing C. difficile alone were serially diluted and plated to determine titer. The 96-well plate was then frozen at −80 C before quantifying C. difficile by qPCR assay.

A standard curve was generated from a well on each assay plate containing only pathogenic C. difficile grown in SweetB+FosIn media and quantified by selective spot plating. Serial dilutions of the culture were performed in sterile phosphate-buffered saline. Genomic DNA was extracted from the standard curve samples along with the other wells.

Genomic DNA was extracted from 5 μl of each sample using a dilution, freeze/thaw, and heat lysis protocol. 5 μL of thawed samples is added to 45 μL of UltraPure water (Life Technologies, Carlsbad, Calif.) and mixed by pipetting. The plates with diluted samples were frozen at −20° C. until use for qPCR which includes a heated lysis step prior to amplification. Alternatively the genomic DNA was isolated using the Mo Bio Powersoil®-htp 96 Well Soil DNA Isolation Kit (Mo Bio Laboratories, Carlsbad, Calif.), Mo Bio Powersoil® DNA Isolation Kit (Mo Bio Laboratories, Carlsbad, Calif.), or the QIAamp DNA Stool Mini Kit (QIAGEN, Valencia, Calif.) according to the manufacturer's instructions.

The qPCR reaction mixture contains 1× SsoAdvanced Universal Probes Supermix, 900 nM of Wr-tcdB-F primer (AGCAGTTGAATATAGTGGTTTAGTTAGAGTTG, IDT, Coralville, Iowa), 900 nM of Wr-tcdB-R primer (CATGCTTTTTTAGTTTCTGGATTGAA, IDT, Coralville, Iowa), 250 nM of Wr-tcdB-P probe (6FAM-CATCCAGTCTCAATTGTATATGTTTCTCCA-MGB, Life Technologies, Grand Island, N.Y.), and Molecular Biology Grade Water (Mo Bio Laboratories, Carlsbad, Calif.) to 18 μl (Primers adapted from: Wroblewski, D. et al., Rapid Molecular Characterization of Clostridium difficile and Assessment of Populations of C. difficile in Stool Specimens, Journal of Clinical Microbiology 47:2142-2148 (2009)). This reaction mixture was aliquoted to wells of a Hard-shell Low-Profile Thin Wall 96-well Skirted PCR Plate (BioRad, Hercules, Calif.). To this reaction mixture, 2 μl of diluted, frozen, and thawed samples are added and the plate sealed with a Microseal ‘B’ Adhesive Seal (BioRad, Hercules, Calif.). The qPCR is performed on a BioRad C1000™ Thermal Cycler equipped with a CFX96™ Real-Time System (BioRad, Hercules, Calif.). The thermocycling conditions were 95° C. for 15 minutes followed by 45 cycles of 95° C. for 5 seconds, 60° C. for 30 seconds, and fluorescent readings of the FAM channel. Alternatively, the qPCR was performed with other standard methods known to those skilled in the art.

The Cq value for each well on the FAM channel was determined by the CFX Manager™ 3.0 software. The log 10 (cfu/mL) of C. difficile each experimental sample was calculated by inputting a given sample's Cq value into a linear regression model generated from the standard curve comparing the Cq values of the standard curve wells to the known log 10 (cfu/mL) of those samples. The log inhibition was calculated for each sample by subtracting the log 10 (cfu/mL) of C. difficile in the sample from the log 10 (cfu/mL) of C. difficile in the sample on each assay plate used for the generation of the standard curve that has no additional bacteria added. The mean log inhibition was calculated for all replicates for each composition.

A histogram of the range and standard deviation of each composition was plotted. Ranges or standard deviations of the log inhibitions that are distinct from the overall distribution are examined as possible outliers. If the removal of a single log inhibition datum from one of the binary pairs that is identified in the histograms would bring the range or standard deviation in line with those from the majority of the samples, that datum is removed as an outlier, and the mean log inhibition is recalculated.

The pooled variance of all samples evaluated in the assay is estimated as the average of the sample variances weighted by the sample's degrees of freedom. The pooled standard error is then calculated as the square root of the pooled variance divided by the square root of the number of samples. Confidence intervals for the null hypothesis are determined by multiplying the pooled standard error to the z score corresponding to a given percentage threshold. Mean log inhibitions outside the confidence interval are considered to be inhibitory if positive or stimulatory if negative with the percent confidence corresponding to the interval used. Ternary combinations with mean log inhibition greater than 0.312 are reported as ++++(≧99% confidence interval (C.I.) of the null hypothesis), those with mean log inhibition between 0.221 and 0.312 as +++(95%<C.I.<99%), those with mean log inhibition between 0.171 and 0.221 as ++(90%<C.I.<95%), those with mean log inhibition between 0.113 and 0.171 as +(80%<C.I.<90%), those with mean log inhibition between −0.113 and −0.171 as −(80%<C.I.<90%), those with mean log inhibition between −0.171 and −0.221 as −−(90%<C.I.<95%), those with mean log inhibition between −0.221 and −0.312 as −−−(95%<C.I.<99%), and those with mean log inhibition less than −0.312 as −−−−(99%<C.I.).

Table 36 below shows OTUs and their clade assignments tested in ternary combinations with results in the in vitro inhibition assay The CivSim shows that many ternary combinations inhibit C. difficile. 39 of 56 combinations show inhibition with a confidence interval >80%; 36 of 56 with a C.I.>90%; 36 of 56 with a C.I.>95%; 29 of 56 with a C.I. of >99%. Non-limiting but exemplary ternary combinations include those with mean log reduction greater than 0.171, e.g. any combination shown in Table 36 with a score of ++++, such as Colinsella aerofaciens, Coprococcus comes, and Blautia producta. Equally important, the CivSim assay describes ternary combinations that do not effectively inhibit C. difficile. 5 of 56 combinations promote growth with >80% confidence; 2 of 56 promote growth with >90% confidence; 1 of 56, Coprococcus comes, Clostridium symbiosum and Eubacterium rectale, promote growth with >95% confidence. 12 of 56 combinations are neutral in the assay, meaning they neither promote nor inhibit C. difficile growth to the limit of measurement.

It is straightforward for one of skill in the art to use the in vitro competition assay described below to determine efficacious subsets of the Core Ecology derived from the bacterial composition shown to be efficacious in treating C. difficile in humans.

### Example 53

### Use of In Vitro Competition Assay to Test Potential Bacterial Competitor Consortia for Functionality

An in vitro assay is performed to test the ability of a chosen species or combination of species to inhibit the growth of a pathogen such as Clostridium difficile in media that is otherwise suitable for growth of the pathogen. A liquid media suitable for growth of the pathogen is chosen, such as Brain Heart Infusion Broth (BHI) for C. difficile (see Example 7). The potential competitor species or a combination of competitor species were inoculated into 3 mL of the media and incubated anaerobically for 24 hr at 37° C. After incubation the cells were pelleted in a centrifuge at 10,000 rcf for 5 min. Supernatant was removed and filtered through a 0.22 μm filter to remove all cells. C. difficile or another pathogen of interest was then inoculated into the filtered spent supernatant and grown anaerobically at 37° C. for 24 hr. A control culture in fresh media was incubated in parallel. After incubation, the titer of C. difficile was determined by serially diluting and plating to Brucella Blood Agar (BBA) plates and incubated anaerobically for 24 hr at 37° C. Colonies were counted to determine the final titer of the pathogen after incubation in competitor conditioned media and control media. The percent reduction in final titer was calculated and considered inhibitory if a statistically significant reduction in growth was measured. Alternatively, the inhibition of pathogen growth was monitored by OD600 measurement of the test and control cultures.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### Tables

TABLE 1

| | SEQ ID | Public DB | | Spore | Pathogen |
|---|---|---|---|---|---|
| OTU | Number | Accession | Clade | Former | Status |
| Eubacterium saburreum | 858 | AB525414 | clade_178 | Y | N |
| Eubacterium sp. oral clone IR009 | 866 | AY349376 | clade_178 | Y | N |
| Lachnospiraceae bacterium ICM62 | 1061 | HQ616401 | clade_178 | Y | N |
| Lachnospiraceae bacterium MSX33 | 1062 | HQ616384 | clade_178 | Y | N |
| Lachnospiraceae bacterium oral taxon 107 | 1063 | ADDS01000069 | clade_178 | Y | N |
| Alicyclobacillus acidocaldarius | 122 | NR_074721 | clade_179 | Y | N |
| Clostridium baratii | 555 | NR_029229 | clade_223 | Y | N |
| Clostridium colicanis | 576 | FJ957863 | clade_223 | Y | N |
| Clostridium paraputrificum | 611 | AB536771 | clade_223 | Y | N |
| Clostridium sardiniense | 621 | NR_041006 | clade_223 | Y | N |
| Eubacterium budayi | 837 | NR_024682 | clade_223 | Y | N |
| Eubacterium moniliforme | 851 | HF558373 | clade_223 | Y | N |
| Eubacterium multiforme | 852 | NR_024683 | clade_223 | Y | N |
| Eubacterium nitritogenes | 853 | NR_024684 | clade_223 | Y | N |
| Anoxybacillus flavithermus | 173 | NR_074667 | clade_238 | Y | N |
| Bacillus aerophilus | 196 | NR_042339 | clade_238 | Y | N |
| Bacillus aestuarii | 197 | GQ980243 | clade_238 | Y | N |
| Bacillus amyloliquefaciens | 199 | NR_075005 | clade_238 | Y | N |
| Bacillus anthracis | 200 | AAEN01000020 | clade_238 | Y | Category-A |
| Bacillus atrophaeus | 201 | NR_075016 | clade_238 | Y | OP |
| Bacillus badius | 202 | NR_036893 | clade_238 | Y | OP |
| Bacillus cereus | 203 | ABDJ01000015 | clade_238 | Y | OP |
| Bacillus circulans | 204 | AB271747 | clade_238 | Y | OP |
| Bacillus firmus | 207 | NR_025842 | clade_238 | Y | OP |
| Bacillus flexus | 208 | NR_024691 | clade_238 | Y | OP |
| Bacillus fordii | 209 | NR_025786 | clade_238 | Y | OP |
| Bacillus halmapalus | 211 | NR_026144 | clade_238 | Y | OP |
| Bacillus herbersteinensis | 213 | NR_042286 | clade_238 | Y | OP |
| Bacillus idriensis | 215 | NR_043268 | clade_238 | Y | OP |
| Bacillus lentus | 216 | NR_040792 | clade_238 | Y | OP |
| Bacillus licheniformis | 217 | NC_006270 | clade_238 | Y | OP |
| Bacillus megaterium | 218 | GU252124 | clade_238 | Y | OP |
| Bacillus nealsonii | 219 | NR_044546 | clade_238 | Y | OP |
| Bacillus niabensis | 220 | NR_043334 | clade_238 | Y | OP |
| Bacillus niacini | 221 | NR_024695 | clade_238 | Y | OP |
| Bacillus pocheonensis | 222 | NR_041377 | clade_238 | Y | OP |
| Bacillus pumilus | 223 | NR_074977 | clade_238 | Y | OP |
| Bacillus safensis | 224 | JQ624766 | clade_238 | Y | OP |
| Bacillus simplex | 225 | NR_042136 | clade_238 | Y | OP |
| Bacillus sonorensis | 226 | NR_025130 | clade_238 | Y | OP |
| Bacillus sp. 10403023 MM10403188 | 227 | CAET01000089 | clade_238 | Y | OP |
| Bacillus sp. 2_A_57_CT2 | 230 | ACWD01000095 | clade_238 | Y | OP |
| Bacillus sp. 2008724126 | 228 | GU252108 | clade_238 | Y | OP |
| Bacillus sp. 2008724139 | 229 | GU252111 | clade_238 | Y | OP |
| Bacillus sp. 7_16AIA | 231 | FN397518 | clade_238 | Y | OP |
| Bacillus sp. AP8 | 233 | JX101689 | clade_238 | Y | OP |
| Bacillus sp. B27(2008) | 234 | EU362173 | clade_238 | Y | OP |
| Bacillus sp. BT1B_CT2 | 235 | ACWC01000034 | clade_238 | Y | OP |
| Bacillus sp. GB1.1 | 236 | FJ897765 | clade_238 | Y | OP |
| Bacillus sp. GB9 | 237 | FJ897766 | clade_238 | Y | OP |
| Bacillus sp. HU19.1 | 238 | FJ897769 | clade_238 | Y | OP |
| Bacillus sp. HU29 | 239 | FJ897771 | clade_238 | Y | OP |
| Bacillus sp. HU33.1 | 240 | FJ897772 | clade_238 | Y | OP |
| Bacillus sp. JC6 | 241 | JF824800 | clade_238 | Y | OP |
| Bacillus sp. oral taxon F79 | 248 | HM099654 | clade_238 | Y | OP |
| Bacillus sp. SRC_DSF1 | 243 | GU797283 | clade_238 | Y | OP |
| Bacillus sp. SRC_DSF10 | 242 | GU797292 | clade_238 | Y | OP |
| Bacillus sp. SRC_DSF2 | 244 | GU797284 | clade_238 | Y | OP |
| Bacillus sp. SRC_DSF6 | 245 | GU797288 | clade_238 | Y | OP |
| Bacillus sp. tc09 | 249 | HQ844242 | clade_238 | Y | OP |
| Bacillus sp. zh168 | 250 | FJ851424 | clade_238 | Y | OP |
| Bacillus sphaericus | 251 | DQ286318 | clade_238 | Y | OP |
| Bacillus sporothermodurans | 252 | NR_026010 | clade_238 | Y | OP |
| Bacillus subtilis | 253 | EU627588 | clade_238 | Y | OP |
| Bacillus thermoamylovorans | 254 | NR_029151 | clade_238 | Y | OP |
| Bacillus thuringiensis | 255 | NC_008600 | clade_238 | Y | OP |
| Bacillus weihenstephanensis | 256 | NR_074926 | clade_238 | Y | OP |
| Geobacillus kaustophilus | 933 | NR_074989 | clade_238 | Y | N |
| Geobacillus stearothermophilus | 936 | NR_040794 | clade_238 | Y | N |
| Geobacillus thermodenitrificans | 938 | NR_074976 | clade_238 | Y | N |
| Geobacillus thermoglucosidasius | 939 | NR_043022 | clade_238 | Y | N |
| Lysinibacillus sphaericus | 1193 | NR_074883 | clade_238 | Y | N |
| Clostridiales sp. SS3_4 | 543 | AY305316 | clade_246 | Y | N |
| Clostridium beijerinckii | 557 | NR_074434 | clade_252 | Y | N |
| Clostridium botulinum | 560 | NC_010723 | clade_252 | Y | Category-A |
| Clostridium butyricum | 561 | ABDT01000017 | clade_252 | Y | N |
| Clostridium chauvoei | 568 | EU106372 | clade_252 | Y | N |
| Clostridium favososporum | 582 | X76749 | clade_252 | Y | N |
| Clostridium histolyticum | 592 | HF558362 | clade_252 | Y | N |
| Clostridium isatidis | 597 | NR_026347 | clade_252 | Y | N |
| Clostridium limosum | 602 | FR870444 | clade_252 | Y | N |
| Clostridium sartagoforme | 622 | NR_026490 | clade_252 | Y | N |
| Clostridium septicum | 624 | NR_026020 | clade_252 | Y | N |
| Clostridium sp. 7_2_43FAA | 626 | ACDK01000101 | clade_252 | Y | N |
| Clostridium sporogenes | 645 | ABKW02000003 | clade_252 | Y | N |
| Clostridium tertium | 653 | Y18174 | clade_252 | Y | N |
| Clostridium carnis | 564 | NR_044716 | clade_253 | Y | N |
| Clostridium celatum | 565 | X77844 | clade_253 | Y | N |
| Clostridium disporicum | 579 | NR_026491 | clade_253 | Y | N |
| Clostridium gasigenes | 585 | NR_024945 | clade_253 | Y | N |
| Clostridium quinii | 616 | NR_026149 | clade_253 | Y | N |
| Clostridium hylemonae | 593 | AB023973 | clade_260 | Y | N |
| Clostridium scindens | 623 | AF262238 | clade_260 | Y | N |
| Lachnospiraceae bacterium 5_1_57FAA | 1054 | ACTR01000020 | clade_260 | Y | N |
| Clostridium glycyrrhizinilyticum | 588 | AB233029 | clade_262 | Y | N |
| Clostridium nexile | 607 | X73443 | clade_262 | Y | N |
| Coprococcus comes | 674 | ABVR01000038 | clade_262 | Y | N |
| Lachnospiraceae bacterium 1_1_57FAA | 1048 | ACTM01000065 | clade_262 | Y | N |
| Lachnospiraceae bacterium 1_4_56FAA | 1049 | ACTN01000028 | clade_262 | Y | N |
| Lachnospiraceae bacterium 8_1_57FAA | 1057 | ACWQ01000079 | clade_262 | Y | N |
| Ruminococcus lactaris | 1663 | ABOU02000049 | clade_262 | Y | N |
| Ruminococcus torques | 1670 | AAVP02000002 | clade_262 | Y | N |
| Paenibacillus lautus | 1397 | NR_040882 | clade_270 | Y | N |
| Paenibacillus polymyxa | 1399 | NR_037006 | clade_270 | Y | N |
| Paenibacillus sp. HGF5 | 1402 | AEXS01000095 | clade_270 | Y | N |
| Paenibacillus sp. HGF7 | 1403 | AFDH01000147 | clade_270 | Y | N |
| Eubacterium sp. oral clone JI012 | 868 | AY349379 | clade_298 | Y | N |
| Alicyclobacillus contaminans | 124 | NR_041475 | clade_301 | Y | N |
| Alicyclobacillus herbarius | 126 | NR_024753 | clade_301 | Y | N |
| Alicyclobacillus pomorum | 127 | NR_024801 | clade_301 | Y | N |
| Blautia coccoides | 373 | AB571656 | clade_309 | Y | N |
| Blautia glucerasea | 374 | AB588023 | clade_309 | Y | N |
| Blautia glucerasei | 375 | AB439724 | clade_309 | Y | N |
| Blautia hansenii | 376 | ABYU02000037 | clade_309 | Y | N |
| Blautia luti | 378 | AB691576 | clade_309 | Y | N |
| Blautia producta | 379 | AB600998 | clade_309 | Y | N |
| Blautia schinkii | 380 | NR_026312 | clade_309 | Y | N |
| Blautia sp. M25 | 381 | HM626178 | clade_309 | Y | N |
| Blautia stercoris | 382 | HM626177 | clade_309 | Y | N |
| Blautia wexlerae | 383 | EF036467 | clade_309 | Y | N |
| Bryantella formatexigens | 439 | ACCL02000018 | clade_309 | Y | N |
| Clostridium coccoides | 573 | EF025906 | clade_309 | Y | N |
| Eubacterium cellulosolvens | 839 | AY178842 | clade_309 | Y | N |
| Lachnospiraceae bacterium 6_1_63FAA | 1056 | ACTV01000014 | clade_309 | Y | N |
| Ruminococcus hansenii | 1662 | M59114 | clade_309 | Y | N |
| Ruminococcus obeum | 1664 | AY169419 | clade_309 | Y | N |
| Ruminococcus sp. 5_1_39BFAA | 1666 | ACII01000172 | clade_309 | Y | N |
| Ruminococcus sp. K_1 | 1669 | AB222208 | clade_309 | Y | N |
| Syntrophococcus sucromutans | 1911 | NR_036869 | clade_309 | Y | N |
| Bacillus alcalophilus | 198 | X76436 | clade_327 | Y | N |
| Bacillus clausii | 205 | FN397477 | clade_327 | Y | OP |
| Bacillus gelatini | 210 | NR_025595 | clade_327 | Y | OP |
| Bacillus halodurans | 212 | AY144582 | clade_327 | Y | OP |
| Bacillus sp. oral taxon F26 | 246 | HM099642 | clade_327 | Y | OP |
| Clostridium innocuum | 595 | M23732 | clade_351 | Y | N |
| Clostridium sp. HGF2 | 628 | AENW01000022 | clade_351 | Y | N |
| Clostridium perfringens | 612 | ABDW01000023 | clade_353 | Y | Category-B |
| Sarcina ventriculi | 1687 | NR_026146 | clade_353 | Y | N |
| Clostridium bartlettii | 556 | ABEZ02000012 | clade_354 | Y | N |
| Clostridium bifermentans | 558 | X73437 | clade_354 | Y | N |
| Clostridium ghonii | 586 | AB542933 | clade_354 | Y | N |
| Clostridium glycolicum | 587 | FJ384385 | clade_354 | Y | N |
| Clostridium mayombei | 605 | FR733682 | clade_354 | Y | N |
| Clostridium sordellii | 625 | AB448946 | clade_354 | Y | N |
| Clostridium sp. MT4 E | 635 | FJ159523 | clade_354 | Y | N |
| Eubacterium tenue | 872 | M59118 | clade_354 | Y | N |
| Clostridium argentinense | 553 | NR_029232 | clade_355 | Y | N |
| Clostridium sp. JC122 | 630 | CAEV01000127 | clade_355 | Y | N |
| Clostridium sp. NMBHI_1 | 636 | JN093130 | clade_355 | Y | N |
| Clostridium subterminale | 650 | NR_041795 | clade_355 | Y | N |
| Clostridium sulfidigenes | 651 | NR_044161 | clade_355 | Y | N |
| Dorea formicigenerans | 773 | AAXA02000006 | clade_360 | Y | N |
| Dorea longicatena | 774 | AJ132842 | clade_360 | Y | N |
| Lachnospiraceae bacterium 2_1_46FAA | 1050 | ADLB01000035 | clade_360 | Y | N |
| Lachnospiraceae bacterium 2_1_58FAA | 1051 | ACTO01000052 | clade_360 | Y | N |
| Lachnospiraceae bacterium 4_1_37FAA | 1053 | ADCR01000030 | clade_360 | Y | N |
| Lachnospiraceae bacterium 9_1_43BFAA | 1058 | ACTX01000023 | clade_360 | Y | N |
| Ruminococcus gnavus | 1661 | X94967 | clade_360 | Y | N |
| Ruminococcus sp. ID8 | 1668 | AY960564 | clade_360 | Y | N |
| Blautia hydrogenotrophica | 377 | ACBZ01000217 | clade_368 | Y | N |
| Lactonifactor longoviformis | 1147 | DQ100449 | clade_368 | Y | N |
| Robinsoniella peoriensis | 1633 | AF445258 | clade_368 | Y | N |
| Eubacterium infirmum | 849 | U13039 | clade_384 | Y | N |
| Eubacterium sp. WAL 14571 | 864 | FJ687606 | clade_384 | Y | N |
| Erysipelotrichaceae bacterium 5_2_54FAA | 823 | ACZW01000054 | clade_385 | Y | N |
| Eubacterium biforme | 835 | ABYT01000002 | clade_385 | Y | N |
| Eubacterium cylindroides | 842 | FP929041 | clade_385 | Y | N |
| Eubacterium dolichum | 844 | L34682 | clade_385 | Y | N |
| Eubacterium sp. 3_1_31 | 861 | ACTL01000045 | clade_385 | Y | N |
| Eubacterium tortuosum | 873 | NR_044648 | clade_385 | Y | N |
| Bulleidia extructa | 441 | ADFR01000011 | clade_388 | Y | N |
| Solobacterium moorei | 1739 | AECQ01000039 | clade_388 | Y | N |
| Coprococcus catus | 673 | EU266552 | clade_393 | Y | N |
| Lachnospiraceae bacterium oral taxon F15 | 1064 | HM099641 | clade_393 | Y | N |
| Clostridium cochlearium | 574 | NR_044717 | clade_395 | Y | N |
| Clostridium malenominatum | 604 | FR749893 | clade_395 | Y | N |
| Clostridium tetani | 654 | NC_004557 | clade_395 | Y | N |
| Acetivibrio ethanolgignens | 6 | FR749897 | clade_396 | Y | N |
| Anaerosporobacter mobilis | 161 | NR_042953 | clade_396 | Y | N |
| Bacteroides pectinophilus | 288 | ABVQ01000036 | clade_396 | Y | N |
| Clostridium aminovalericum | 551 | NR_029245 | clade_396 | Y | N |
| Clostridium phytofermentans | 613 | NR_074652 | clade_396 | Y | N |
| Eubacterium hallii | 848 | L34621 | clade_396 | Y | N |
| Eubacterium xylanophilum | 875 | L34628 | clade_396 | Y | N |
| Ruminococcus callidus | 1658 | NR_029160 | clade_406 | Y | N |
| Ruminococcus champanellensis | 1659 | FP929052 | clade_406 | Y | N |
| Ruminococcus sp. 18P13 | 1665 | AJ515913 | clade_406 | Y | N |
| Ruminococcus sp. 9SE51 | 1667 | FM954974 | clade_406 | Y | N |
| Anaerostipes caccae | 162 | ABAX03000023 | clade_408 | Y | N |
| Anaerostipes sp. 3_2_56FAA | 163 | ACWB01000002 | clade_408 | Y | N |
| Clostridiales bacterium 1_7_47FAA | 541 | ABQR01000074 | clade_408 | Y | N |
| Clostridiales sp. SM4_1 | 542 | FP929060 | clade_408 | Y | N |
| Clostridiales sp. SSC_2 | 544 | FP929061 | clade_408 | Y | N |
| Clostridium aerotolerans | 546 | X76163 | clade_408 | Y | N |
| Clostridium aldenense | 547 | NR_043680 | clade_408 | Y | N |
| Clostridium algidixylanolyticum | 550 | NR_028726 | clade_408 | Y | N |
| Clostridium amygdalinum | 552 | AY353957 | clade_408 | Y | N |
| Clostridium asparagiforme | 554 | ACCJ01000522 | clade_408 | Y | N |
| Clostridium bolteae | 559 | ABCC02000039 | clade_408 | Y | N |
| Clostridium celerecrescens | 566 | JQ246092 | clade_408 | Y | N |
| Clostridium citroniae | 569 | ADLJ01000059 | clade_408 | Y | N |
| Clostridium clostridiiformes | 571 | M59089 | clade_408 | Y | N |
| Clostridium clostridioforme | 572 | NR_044715 | clade_408 | Y | N |
| Clostridium hathewayi | 590 | AY552788 | clade_408 | Y | N |
| Clostridium indolis | 594 | AF028351 | clade_408 | Y | N |
| Clostridium lavalense | 600 | EF564277 | clade_408 | Y | N |
| Clostridium saccharolyticum | 620 | CP002109 | clade_408 | Y | N |
| Clostridium sp. M62_1 | 633 | ACFX02000046 | clade_408 | Y | N |
| Clostridium sp. SS2_1 | 638 | ABGC03000041 | clade_408 | Y | N |
| Clostridium sphenoides | 643 | X73449 | clade_408 | Y | N |
| Clostridium symbiosum | 652 | ADLQ01000114 | clade_408 | Y | N |
| Clostridium xylanolyticum | 658 | NR_037068 | clade_408 | Y | N |
| Eubacterium hadrum | 847 | FR749933 | clade_408 | Y | N |
| Lachnospiraceae bacterium 3_1_57FAA_CT1 | 1052 | ACTP01000124 | clade_408 | Y | N |
| Lachnospiraceae bacterium 5_1_63FAA | 1055 | ACTS01000081 | clade_408 | Y | N |
| Lachnospiraceae bacterium A4 | 1059 | DQ789118 | clade_408 | Y | N |
| Lachnospiraceae bacterium DJF VP30 | 1060 | EU728771 | clade_408 | Y | N |
| Lachnospiraceae genomosp. C1 | 1065 | AY278618 | clade_408 | Y | N |
| Clostridium difficile | 578 | NC_013315 | clade_409 | Y | OP |
| Eubacterium sp. AS15b | 862 | HQ616364 | clade_428 | Y | N |
| Eubacterium sp. OBRC9 | 863 | HQ616354 | clade_428 | Y | N |
| Eubacterium sp. oral clone OH3A | 871 | AY947497 | clade_428 | Y | N |
| Eubacterium yurii | 876 | AEES01000073 | clade_428 | Y | N |
| Clostridium acetobutylicum | 545 | NR_074511 | clade_430 | Y | N |
| Clostridium algidicarnis | 549 | NR_041746 | clade_430 | Y | N |
| Clostridium cadaveris | 562 | AB542932 | clade_430 | Y | N |
| Clostridium carboxidivorans | 563 | FR733710 | clade_430 | Y | N |
| Clostridium estertheticum | 580 | NR_042153 | clade_430 | Y | N |
| Clostridium fallax | 581 | NR_044714 | clade_430 | Y | N |
| Clostridium felsineum | 583 | AF270502 | clade_430 | Y | N |
| Clostridium frigidicarnis | 584 | NR_024919 | clade_430 | Y | N |
| Clostridium kluyveri | 598 | NR_074165 | clade_430 | Y | N |
| Clostridium magnum | 603 | X77835 | clade_430 | Y | N |
| Clostridium putrefaciens | 615 | NR_024995 | clade_430 | Y | N |
| Clostridium sp. HPB_46 | 629 | AY862516 | clade_430 | Y | N |
| Clostridium tyrobutyricum | 656 | NR_044718 | clade_430 | Y | N |
| Sutterella parvirubra | 1899 | AB300989 | clade_432 | Y | N |
| Acetanaerobacterium elongatum | 4 | NR_042930 | clade_439 | Y | N |
| Clostridium cellulosi | 567 | NR_044624 | clade_439 | Y | N |
| Ethanoligenens harbinense | 832 | AY675965 | clade_439 | Y | N |
| Eubacterium rectale | 856 | FP929042 | clade_444 | Y | N |
| Eubacterium sp. oral clone GI038 | 865 | AY349374 | clade_444 | Y | N |
| Lachnobacterium bovis | 1045 | GU324407 | clade_444 | Y | N |
| Roseburia cecicola | 1634 | GU233441 | clade_444 | Y | N |
| Roseburia faecalis | 1635 | AY804149 | clade_444 | Y | N |
| Roseburia faecis | 1636 | AY305310 | clade_444 | Y | N |
| Roseburia hominis | 1637 | AJ270482 | clade_444 | Y | N |
| Roseburia intestinalis | 1638 | FP929050 | clade_444 | Y | N |
| Roseburia inulinivorans | 1639 | AJ270473 | clade_444 | Y | N |
| Brevibacillus brevis | 410 | NR_041524 | clade_448 | Y | N |
| Brevibacillus laterosporus | 414 | NR_037005 | clade_448 | Y | N |
| Bacillus coagulans | 206 | DQ297928 | clade_451 | Y | OP |
| Sporolactobacillus inulinus | 1752 | NR_040962 | clade_451 | Y | N |
| Kocuria palustris | 1041 | EU333884 | clade_453 | Y | N |
| Nocardia farcinica | 1353 | NC_006361 | clade_455 | Y | N |
| Bacillus sp. oral taxon F28 | 247 | HM099650 | clade_456 | Y | OP |
| Catenibacterium mitsuokai | 495 | AB030224 | clade_469 | Y | N |
| Clostridium sp. TM_40 | 640 | AB249652 | clade_469 | Y | N |
| Coprobacillus cateniformis | 670 | AB030218 | clade_469 | Y | N |
| Coprobacillus sp. 29_1 | 671 | ADKX01000057 | clade_469 | Y | N |
| Clostridium rectum | 618 | NR_029271 | clade_470 | Y | N |
| Eubacterium nodatum | 854 | U13041 | clade_476 | Y | N |
| Eubacterium saphenum | 859 | NR_026031 | clade_476 | Y | N |
| Eubacterium sp. oral clone JH012 | 867 | AY349373 | clade_476 | Y | N |
| Eubacterium sp. oral clone JS001 | 870 | AY349378 | clade_476 | Y | N |
| Faecalibacterium prausnitzii | 880 | ACOP02000011 | clade_478 | Y | N |
| Gemmiger formicilis | 932 | GU562446 | clade_478 | Y | N |
| Subdoligranulum variabile | 1896 | AJ518869 | clade_478 | Y | N |
| Clostridiaceae bacterium JC13 | 532 | JF824807 | clade_479 | Y | N |
| Clostridium sp. MLG055 | 634 | AF304435 | clade_479 | Y | N |
| Erysipelotrichaceae bacterium 3_1_53 | 822 | ACTJ01000113 | clade_479 | Y | N |
| Clostridium cocleatum | 575 | NR_026495 | clade_481 | Y | N |
| Clostridium ramosum | 617 | M23731 | clade_481 | Y | N |
| Clostridium saccharogumia | 619 | DQ100445 | clade_481 | Y | N |
| Clostridium spiroforme | 644 | X73441 | clade_481 | Y | N |
| Coprobacillus sp. D7 | 672 | ACDT01000199 | clade_481 | Y | N |
| Clostridiales bacterium SY8519 | 535 | AB477431 | clade_482 | Y | N |
| Clostridium sp. SY8519 | 639 | AP012212 | clade_482 | Y | N |
| Eubacterium ramulus | 855 | AJ011522 | clade_482 | Y | N |
| Erysipelothrix inopinata | 819 | NR_025594 | clade_485 | Y | N |
| Erysipelothrix rhusiopathiae | 820 | ACLK01000021 | clade_485 | Y | N |
| Erysipelothrix tonsillarum | 821 | NR_040871 | clade_485 | Y | N |
| Holdemania filiformis | 1004 | Y11466 | clade_485 | Y | N |
| Mollicutes bacterium pACH93 | 1258 | AY297808 | clade_485 | Y | N |
| Coxiella burnetii | 736 | CP000890 | clade_486 | Y | Category-B |
| Clostridium hiranonis | 591 | AB023970 | clade_487 | Y | N |
| Clostridium irregulare | 596 | NR_029249 | clade_487 | Y | N |
| Clostridium orbiscindens | 609 | Y18187 | clade_494 | Y | N |
| Clostridium sp. NML 04A032 | 637 | EU815224 | clade_494 | Y | N |
| Flavonifractor plautii | 886 | AY724678 | clade_494 | Y | N |
| Pseudoflavonifractor capillosus | 1591 | AY136666 | clade_494 | Y | N |
| Ruminococcaceae bacterium D16 | 1655 | ADDX01000083 | clade_494 | Y | N |
| Acetivibrio cellulolyticus | 5 | NR_025917 | clade_495 | Y | N |
| Clostridium aldrichii | 548 | NR_026099 | clade_495 | Y | N |
| Clostridium clariflavum | 570 | NR_041235 | clade_495 | Y | N |
| Clostridium stercorarium | 647 | NR_025100 | clade_495 | Y | N |
| Clostridium straminisolvens | 649 | NR_024829 | clade_495 | Y | N |
| Clostridium thermocellum | 655 | NR_074629 | clade_495 | Y | N |
| Fusobacterium nucleatum | 901 | ADVK01000034 | clade_497 | Y | N |
| Eubacterium barkeri | 834 | NR_044661 | clade_512 | Y | N |
| Eubacterium callanderi | 838 | NR_026330 | clade_512 | Y | N |
| Eubacterium limosum | 850 | CP002273 | clade_512 | Y | N |
| Anaerotruncus colihominis | 164 | ABGD02000021 | clade_516 | Y | N |
| Clostridium methylpentosum | 606 | ACEC01000059 | clade_516 | Y | N |
| Clostridium sp. YIT 12070 | 642 | AB491208 | clade_516 | Y | N |
| Hydrogenoanaerobacterium saccharovorans | 1005 | NR_044425 | clade_516 | Y | N |
| Ruminococcus albus | 1656 | AY445600 | clade_516 | Y | N |
| Ruminococcus flavefaciens | 1660 | NR_025931 | clade_516 | Y | N |
| Clostridium haemolyticum | 589 | NR_024749 | clade_517 | Y | N |
| Clostridium novyi | 608 | NR_074343 | clade_517 | Y | N |
| Clostridium sp. LMG 16094 | 632 | X95274 | clade_517 | Y | N |
| Eubacterium ventriosum | 874 | L34421 | clade_519 | Y | N |
| Bacteroides galacturonicus | 280 | DQ497994 | clade_522 | Y | N |
| Eubacterium eligens | 845 | CP001104 | clade_522 | Y | N |
| Lachnospira multipara | 1046 | FR733699 | clade_522 | Y | N |
| Lachnospira pectinoschiza | 1047 | L14675 | clade_522 | Y | N |
| Lactobacillus rogosae | 1114 | GU269544 | clade_522 | Y | N |
| Bacillus horti | 214 | NR_036860 | clade_527 | Y | OP |
| Bacillus sp. 9_3AIA | 232 | FN397519 | clade_527 | Y | OP |
| Eubacterium brachy | 836 | U13038 | clade_533 | Y | N |
| Filifactor alocis | 881 | CP002390 | clade_533 | Y | N |
| Filifactor villosus | 882 | NR_041928 | clade_533 | Y | N |
| Clostridium leptum | 601 | AJ305238 | clade_537 | Y | N |
| Clostridium sp. YIT 12069 | 641 | AB491207 | clade_537 | Y | N |
| Clostridium sporosphaeroides | 646 | NR_044835 | clade_537 | Y | N |
| Eubacterium coprostanoligenes | 841 | HM037995 | clade_537 | Y | N |
| Ruminococcus bromii | 1657 | EU266549 | clade_537 | Y | N |
| Eubacterium siraeum | 860 | ABCA03000054 | clade_538 | Y | N |
| Clostridium viride | 657 | NR_026204 | clade_540 | Y | N |
| Oscillibacter sp. G2 | 1386 | HM626173 | clade_540 | Y | N |
| Oscillibacter valericigenes | 1387 | NR_074793 | clade_540 | Y | N |
| Oscillospira guilliermondii | 1388 | AB040495 | clade_540 | Y | N |
| Butyrivibrio crossotus | 455 | ABWN01000012 | clade_543 | Y | N |
| Clostridium sp. L2_50 | 631 | AAYW02000018 | clade_543 | Y | N |
| Coprococcus eutactus | 675 | EF031543 | clade_543 | Y | N |
| Coprococcus sp. ART55_1 | 676 | AY350746 | clade_543 | Y | N |
| Eubacterium ruminantium | 857 | NR_024661 | clade_543 | Y | N |
| Collinsella aerofaciens | 659 | AAVN02000007 | clade_553 | Y | N |
| Alkaliphilus metalliredigenes | 137 | AY137848 | clade_554 | Y | N |
| Alkaliphilus oremlandii | 138 | NR_043674 | clade_554 | Y | N |
| Clostridium sticklandii | 648 | L04167 | clade_554 | Y | N |
| Turicibacter sanguinis | 1965 | AF349724 | clade_555 | Y | N |
| Fulvimonas sp. NML 060897 | 892 | EF589680 | clade_557 | Y | N |
| Desulfitobacterium frappieri | 753 | AJ276701 | clade_560 | Y | N |
| Desulfitobacterium hafniense | 754 | NR_074996 | clade_560 | Y | N |
| Desulfotomaculum nigrificans | 756 | NR_044832 | clade_560 | Y | N |
| Lutispora thermophila | 1191 | NR_041236 | clade_564 | Y | N |
| Brachyspira pilosicoli | 405 | NR_075069 | clade_565 | Y | N |
| Eggerthella lenta | 778 | AF292375 | clade_566 | Y | N |
| Streptomyces albus | 1888 | AJ697941 | clade_566 | Y | N |
| Chlamydiales bacterium NS11 | 505 | JN606074 | clade_567 | Y | N |
| Anaerofustis stercorihominis | 159 | ABIL02000005 | clade_570 | Y | N |
| Butyricicoccus pullicaecorum | 453 | HH793440 | clade_572 | Y | N |
| Eubacterium desmolans | 843 | NR_044644 | clade_572 | Y | N |
| Papillibacter cinnamivorans | 1415 | NR_025025 | clade_572 | Y | N |
| Sporobacter termitidis | 1751 | NR_044972 | clade_572 | Y | N |
| Deferribacteres sp. oral clone JV006 | 744 | AY349371 | clade_575 | Y | N |
| Clostridium colinum | 577 | NR_026151 | clade_576 | Y | N |
| Clostridium lactatifermentans | 599 | NR_025651 | clade_576 | Y | N |
| Clostridium piliforme | 614 | D14639 | clade_576 | Y | N |
| Saccharomonospora viridis | 1671 | X54286 | clade_579 | Y | N |
| Thermobifida fusca | 1921 | NC_007333 | clade_579 | Y | N |
| Leptospira licerasiae | 1164 | EF612284 | clade_585 | Y | OP |
| Moorella thermoacetica | 1259 | NR_075001 | clade_590 | Y | N |
| Thermoanaerobacter pseudethanolicus | 1920 | CP000924 | clade_590 | Y | N |
| Flexistipes sinusarabici | 888 | NR_074881 | clade_591 | Y | N |
| Gloeobacter violaceus | 942 | NR_074282 | clade_596 | Y | N |
| Eubacterium sp. oral clone JN088 | 869 | AY349377 | clade_90 | Y | N |
| Clostridium oroticum | 610 | FR749922 | clade_96 | Y | N |
| Clostridium sp. D5 | 627 | ADBG01000142 | clade_96 | Y | N |
| Eubacterium contortum | 840 | FR749946 | clade_96 | Y | N |
| Eubacterium fissicatena | 846 | FR749935 | clade_96 | Y | N |
| Corynebacterium coyleae | 692 | X96497 | clade_100 | N | N |
| Corynebacterium mucifaciens | 711 | NR_026396 | clade_100 | N | N |
| Corynebacterium ureicelerivorans | 733 | AM397636 | clade_100 | N | N |
| Corynebacterium appendicis | 684 | NR_028951 | clade_102 | N | N |
| Corynebacterium genitalium | 698 | ACLJ01000031 | clade_102 | N | N |
| Corynebacterium glaucum | 699 | NR_028971 | clade_102 | N | N |
| Corynebacterium imitans | 703 | AF537597 | clade_102 | N | N |
| Corynebacterium riegelii | 719 | EU848548 | clade_102 | N | N |
| Corynebacterium sp. L_2012475 | 723 | HE575405 | clade_102 | N | N |
| Corynebacterium sp. NML 93_0481 | 724 | GU238409 | clade_102 | N | N |
| Corynebacterium sundsvallense | 728 | Y09655 | clade_102 | N | N |
| Corynebacterium tuscaniae | 730 | AY677186 | clade_102 | N | N |
| Prevotella maculosa | 1504 | AGEK01000035 | clade_104 | N | N |
| Prevotella oris | 1513 | ADDV01000091 | clade_104 | N | N |
| Prevotella salivae | 1517 | AB108826 | clade_104 | N | N |
| Prevotella sp. ICM55 | 1521 | HQ616399 | clade_104 | N | N |
| Prevotella sp. oral clone AA020 | 1528 | AY005057 | clade_104 | N | N |
| Prevotella sp. oral clone GI032 | 1538 | AY349396 | clade_104 | N | N |
| Prevotella sp. oral taxon G70 | 1558 | GU432179 | clade_104 | N | N |
| Prevotella corporis | 1491 | L16465 | clade_105 | N | N |
| Bacteroides sp. 4_1_36 | 312 | ACTC01000133 | clade_110 | N | N |
| Bacteroides sp. AR20 | 315 | AF139524 | clade_110 | N | N |
| Bacteroides sp. D20 | 319 | ACPT01000052 | clade_110 | N | N |
| Bacteroides sp. F_4 | 322 | AB470322 | clade_110 | N | N |
| Bacteroides uniformis | 329 | AB050110 | clade_110 | N | N |
| Prevotella nanceiensis | 1510 | JN867228 | clade_127 | N | N |
| Prevotella sp. oral taxon 299 | 1548 | ACWZ01000026 | clade_127 | N | N |
| Prevotella bergensis | 1485 | ACKS01000100 | clade_128 | N | N |
| Prevotella buccalis | 1489 | JN867261 | clade_129 | N | N |
| Prevotella timonensis | 1564 | ADEF01000012 | clade_129 | N | N |
| Prevotella oralis | 1512 | AEPE01000021 | clade_130 | N | N |
| Prevotella sp. SEQ072 | 1525 | JN867238 | clade_130 | N | N |
| Leuconostoc carnosum | 1177 | NR_040811 | clade_135 | N | N |
| Leuconostoc gasicomitatum | 1179 | FN822744 | clade_135 | N | N |
| Leuconostoc inhae | 1180 | NR_025204 | clade_135 | N | N |
| Leuconostoc kimchii | 1181 | NR_075014 | clade_135 | N | N |
| Edwardsiella tarda | 777 | CP002154 | clade_139 | N | N |
| Photorhabdus asymbiotica | 1466 | Z76752 | clade_139 | N | N |
| Psychrobacter arcticus | 1607 | CP000082 | clade_141 | N | N |
| Psychrobacter cibarius | 1608 | HQ698586 | clade_141 | N | N |
| Psychrobacter cryohalolentis | 1609 | CP000323 | clade_141 | N | N |
| Psychrobacter faecalis | 1610 | HQ698566 | clade_141 | N | N |
| Psychrobacter nivimaris | 1611 | HQ698587 | clade_141 | N | N |
| Psychrobacter pulmonis | 1612 | HQ698582 | clade_141 | N | N |
| Pseudomonas aeruginosa | 1592 | AABQ07000001 | clade_154 | N | N |
| Pseudomonas sp. 2_1_26 | 1600 | ACWU01000257 | clade_154 | N | N |
| Corynebacterium confusum | 691 | Y15886 | clade_158 | N | N |
| Corynebacterium propinquum | 712 | NR_037038 | clade_158 | N | N |
| Corynebacterium pseudodiphtheriticum | 713 | X84258 | clade_158 | N | N |
| Bartonella bacilliformis | 338 | NC_008783 | clade_159 | N | N |
| Bartonella grahamii | 339 | CP001562 | clade_159 | N | N |
| Bartonella henselae | 340 | NC_005956 | clade_159 | N | N |
| Bartonella quintana | 341 | BX897700 | clade_159 | N | N |
| Bartonella tamiae | 342 | EF672728 | clade_159 | N | N |
| Bartonella washoensis | 343 | FJ719017 | clade_159 | N | N |
| Brucella abortus | 430 | ACBJ01000075 | clade_159 | N | Category-B |
| Brucella canis | 431 | NR_044652 | clade_159 | N | Category-B |
| Brucella ceti | 432 | ACJD01000006 | clade_159 | N | Category-B |
| Brucella melitensis | 433 | AE009462 | clade_159 | N | Category-B |
| Brucella microti | 434 | NR_042549 | clade_159 | N | Category-B |
| Brucella ovis | 435 | NC_009504 | clade_159 | N | Category-B |
| Brucella sp. 83_13 | 436 | ACBQ01000040 | clade_159 | N | Category-B |
| Brucella sp. BO1 | 437 | EU053207 | clade_159 | N | Category-B |
| Brucella suis | 438 | ACBK01000034 | clade_159 | N | Category-B |
| Ochrobactrum anthropi | 1360 | NC_009667 | clade_159 | N | N |
| Ochrobactrum intermedium | 1361 | ACQA01000001 | clade_159 | N | N |
| Ochrobactrum pseudintermedium | 1362 | DQ365921 | clade_159 | N | N |
| Prevotella genomosp. C2 | 1496 | AY278625 | clade_164 | N | N |
| Prevotella multisaccharivorax | 1509 | AFJE01000016 | clade_164 | N | N |
| Prevotella sp. oral clone IDR_CEC_0055 | 1543 | AY550997 | clade_164 | N | N |
| Prevotella sp. oral taxon 292 | 1547 | GQ422735 | clade_164 | N | N |
| Prevotella sp. oral taxon 300 | 1549 | GU409549 | clade_164 | N | N |
| Prevotella marshii | 1505 | AEEI01000070 | clade_166 | N | N |
| Prevotella sp. oral clone IK053 | 1544 | AY349401 | clade_166 | N | N |
| Prevotella sp. oral taxon 781 | 1554 | GQ422744 | clade_166 | N | N |
| Prevotella stercorea | 1562 | AB244774 | clade_166 | N | N |
| Prevotella brevis | 1487 | NR_041954 | clade_167 | N | N |
| Prevotella ruminicola | 1516 | CP002006 | clade_167 | N | N |
| Prevotella sp. sp24 | 1560 | AB003384 | clade_167 | N | N |
| Prevotella sp. sp34 | 1561 | AB003385 | clade_167 | N | N |
| Prevotella albensis | 1483 | NR_025300 | clade_168 | N | N |
| Prevotella copri | 1490 | ACBX02000014 | clade_168 | N | N |
| Prevotella oulorum | 1514 | L16472 | clade_168 | N | N |
| Prevotella sp. BI_42 | 1518 | AJ581354 | clade_168 | N | N |
| Prevotella sp. oral clone P4PB_83 P2 | 1546 | AY207050 | clade_168 | N | N |
| Prevotella sp. oral taxon G60 | 1557 | GU432133 | clade_168 | N | N |
| Prevotella amnii | 1484 | AB547670 | clade_169 | N | N |
| Bacteroides caccae | 268 | EU136686 | clade_170 | N | N |
| Bacteroides finegoldii | 277 | AB222699 | clade_170 | N | N |
| Bacteroides intestinalis | 283 | ABJL02000006 | clade_171 | N | N |
| Bacteroides sp. XB44A | 326 | AM230649 | clade_171 | N | N |
| Bifidobacteriaceae genomosp. C1 | 345 | AY278612 | clade_172 | N | N |
| Bifidobacterium adolescentis | 346 | AAXD02000018 | clade_172 | N | N |
| Bifidobacterium angulatum | 347 | ABYS02000004 | clade_172 | N | N |
| Bifidobacterium animalis | 348 | CP001606 | clade_172 | N | N |
| Bifidobacterium breve | 350 | CP002743 | clade_172 | N | N |
| Bifidobacterium catenulatum | 351 | ABXY01000019 | clade_172 | N | N |
| Bifidobacterium dentium | 352 | CP001750 | clade_172 | N | OP |
| Bifidobacterium gallicum | 353 | ABXB03000004 | clade_172 | N | N |
| Bifidobacterium infantis | 354 | AY151398 | clade_172 | N | N |
| Bifidobacterium kashiwanohense | 355 | AB491757 | clade_172 | N | N |
| Bifidobacterium longum | 356 | ABQQ01000041 | clade_172 | N | N |
| Bifidobacterium pseudocatenulatum | 357 | ABXX02000002 | clade_172 | N | N |
| Bifidobacterium pseudolongum | 358 | NR_043442 | clade_172 | N | N |
| Bifidobacterium scardovii | 359 | AJ307005 | clade_172 | N | N |
| Bifidobacterium sp. HM2 | 360 | AB425276 | clade_172 | N | N |
| Bifidobacterium sp. HMLN12 | 361 | JF519685 | clade_172 | N | N |
| Bifidobacterium sp. M45 | 362 | HM626176 | clade_172 | N | N |
| Bifidobacterium sp. MSX5B | 363 | HQ616382 | clade_172 | N | N |
| Bifidobacterium sp. TM_7 | 364 | AB218972 | clade_172 | N | N |
| Bifidobacterium thermophilum | 365 | DQ340557 | clade_172 | N | N |
| Leuconostoc citreum | 1178 | AM157444 | clade_175 | N | N |
| Leuconostoc lactis | 1182 | NR_040823 | clade_175 | N | N |
| Alicyclobacillus acidoterrestris | 123 | NR_040844 | clade_179 | N | N |
| Alicyclobacillus cycloheptanicus | 125 | NR_024754 | clade_179 | N | N |
| Acinetobacter baumannii | 27 | ACYQ01000014 | clade_181 | N | N |
| Acinetobacter calcoaceticus | 28 | AM157426 | clade_181 | N | N |
| Acinetobacter genomosp. C1 | 29 | AY278636 | clade_181 | N | N |
| Acinetobacter haemolyticus | 30 | ADMT01000017 | clade_181 | N | N |
| Acinetobacter johnsonii | 31 | ACPL01000162 | clade_181 | N | N |
| Acinetobacter junii | 32 | ACPM01000135 | clade_181 | N | N |
| Acinetobacter lwoffii | 33 | ACPN01000204 | clade_181 | N | N |
| Acinetobacter parvus | 34 | AIEB01000124 | clade_181 | N | N |
| Acinetobacter schindleri | 36 | NR_025412 | clade_181 | N | N |
| Acinetobacter sp. 56A1 | 37 | GQ178049 | clade_181 | N | N |
| Acinetobacter sp. CIP 101934 | 38 | JQ638573 | clade_181 | N | N |
| Acinetobacter sp. CIP 102143 | 39 | JQ638578 | clade_181 | N | N |
| Acinetobacter sp. M16_22 | 41 | HM366447 | clade_181 | N | N |
| Acinetobacter sp. RUH2624 | 42 | ACQF01000094 | clade_181 | N | N |
| Acinetobacter sp. SH024 | 43 | ADCH01000068 | clade_181 | N | N |
| Lactobacillus jensenii | 1092 | ACQD01000066 | clade_182 | N | N |
| Alcaligenes faecalis | 119 | AB680368 | clade_183 | N | N |
| Alcaligenes sp. CO14 | 120 | DQ643040 | clade_183 | N | N |
| Alcaligenes sp. S3 | 121 | HQ262549 | clade_183 | N | N |
| Oligella ureolytica | 1366 | NR_041998 | clade_183 | N | N |
| Oligella urethralis | 1367 | NR_041753 | clade_183 | N | N |
| Eikenella corrodens | 784 | ACEA01000028 | clade_185 | N | N |
| Kingella denitrificans | 1019 | AEWV01000047 | clade_185 | N | N |
| Kingella genomosp. P1 oral cone MB2_C20 | 1020 | DQ003616 | clade_185 | N | N |
| Kingella kingae | 1021 | AFHS01000073 | clade_185 | N | N |
| Kingella oralis | 1022 | ACJW02000005 | clade_185 | N | N |
| Kingella sp. oral clone ID059 | 1023 | AY349381 | clade_185 | N | N |
| Neisseria elongata | 1330 | ADBF01000003 | clade_185 | N | N |
| Neisseria genomosp. P2 oral clone MB5_P15 | 1332 | DQ003630 | clade_185 | N | N |
| Neisseria sp. oral clone JC012 | 1345 | AY349388 | clade_185 | N | N |
| Neisseria sp. SMC_A9199 | 1342 | FJ763637 | clade_185 | N | N |
| Simonsiella muelleri | 1731 | ADCY01000105 | clade_185 | N | N |
| Corynebacterium glucuronolyticum | 700 | ABYP01000081 | clade_193 | N | N |
| Corynebacterium pyruviciproducens | 716 | FJ185225 | clade_193 | N | N |
| Rothia aeria | 1649 | DQ673320 | clade_194 | N | N |
| Rothia dentocariosa | 1650 | ADDW01000024 | clade_194 | N | N |
| Rothia sp. oral taxon 188 | 1653 | GU470892 | clade_194 | N | N |
| Corynebacterium accolens | 681 | ACGD01000048 | clade_195 | N | N |
| Corynebacterium macginleyi | 707 | AB359393 | clade_195 | N | N |
| Corynebacterium pseudogenitalium | 714 | ABYQ01000237 | clade_195 | N | N |
| Corynebacterium tuberculostearicum | 729 | ACVP01000009 | clade_195 | N | N |
| Lactobacillus casei | 1074 | CP000423 | clade_198 | N | N |
| Lactobacillus paracasei | 1106 | ABQV01000067 | clade_198 | N | N |
| Lactobacillus zeae | 1143 | NR_037122 | clade_198 | N | N |
| Prevotella dentalis | 1492 | AB547678 | clade_205 | N | N |
| Prevotella sp. oral clone ASCG10 | 1529 | AY923148 | clade_206 | N | N |
| Prevotella sp. oral clone HF050 | 1541 | AY349399 | clade_206 | N | N |
| Prevotella sp. oral clone ID019 | 1542 | AY349400 | clade_206 | N | N |
| Prevotella sp. oral clone IK062 | 1545 | AY349402 | clade_206 | N | N |
| Prevotella genomosp. P9 oral clone MB7_G16 | 1499 | DQ003633 | clade_207 | N | N |
| Prevotella sp. oral clone AU069 | 1531 | AY005062 | clade_207 | N | N |
| Prevotella sp. oral clone CY006 | 1532 | AY005063 | clade_207 | N | N |
| Prevotella sp. oral clone FL019 | 1534 | AY349392 | clade_207 | N | N |
| Actinomyces genomosp. C1 | 56 | AY278610 | clade_212 | N | N |
| Actinomyces genomosp. C2 | 57 | AY278611 | clade_212 | N | N |
| Actinomyces genomosp. P1 oral clone MB6_C03 | 58 | DQ003632 | clade_212 | N | N |
| Actinomyces georgiae | 59 | GU561319 | clade_212 | N | N |
| Actinomyces israelii | 60 | AF479270 | clade_212 | N | N |
| Actinomyces massiliensis | 61 | AB545934 | clade_212 | N | N |
| Actinomyces meyeri | 62 | GU561321 | clade_212 | N | N |
| Actinomyces odontolyticus | 66 | ACYT01000123 | clade_212 | N | N |
| Actinomyces orihominis | 68 | AJ575186 | clade_212 | N | N |
| Actinomyces sp. CCUG 37290 | 71 | AJ234058 | clade_212 | N | N |
| Actinomyces sp. ICM34 | 75 | HQ616391 | clade_212 | N | N |
| Actinomyces sp. ICM41 | 76 | HQ616392 | clade_212 | N | N |
| Actinomyces sp. ICM47 | 77 | HQ616395 | clade_212 | N | N |
| Actinomyces sp. ICM54 | 78 | HQ616398 | clade_212 | N | N |
| Actinomyces sp. oral clone IP081 | 87 | AY349366 | clade_212 | N | N |
| Actinomyces sp. oral taxon 178 | 91 | AEUH01000060 | clade_212 | N | N |
| Actinomyces sp. oral taxon 180 | 92 | AEPP01000041 | clade_212 | N | N |
| Actinomyces sp. TeJ5 | 80 | GU561315 | clade_212 | N | N |
| Haematobacter sp. BC14248 | 968 | GU396991 | clade_213 | N | N |
| Paracoccus denitrificans | 1424 | CP000490 | clade_213 | N | N |
| Paracoccus marcusii | 1425 | NR_044922 | clade_213 | N | N |
| Grimontia hollisae | 967 | ADAQ01000013 | clade_216 | N | N |
| Shewanella putrefaciens | 1723 | CP002457 | clade_216 | N | N |
| Afipia genomosp. 4 | 111 | EU117385 | clade_217 | N | N |
| Rhodopseudomonas palustris | 1626 | CP000301 | clade_217 | N | N |
| Methylobacterium extorquens | 1223 | NC_010172 | clade_218 | N | N |
| Methylobacterium podarium | 1224 | AY468363 | clade_218 | N | N |
| Methylobacterium radiotolerans | 1225 | GU294320 | clade_218 | N | N |
| Methylobacterium sp. 1sub | 1226 | AY468371 | clade_218 | N | N |
| Methylobacterium sp. MM4 | 1227 | AY468370 | clade_218 | N | N |
| Achromobacter denitrificans | 18 | NR_042021 | clade_224 | N | N |
| Achromobacter piechaudii | 19 | ADMS01000149 | clade_224 | N | N |
| Achromobacter xylosoxidans | 20 | ACRC01000072 | clade_224 | N | N |
| Bordetella bronchiseptica | 384 | NR_025949 | clade_224 | N | OP |
| Bordetella holmesii | 385 | AB683187 | clade_224 | N | OP |
| Bordetella parapertussis | 386 | NR_025950 | clade_224 | N | OP |
| Bordetella pertussis | 387 | BX640418 | clade_224 | N | OP |
| Microbacterium chocolatum | 1230 | NR_037045 | clade_225 | N | N |
| Microbacterium flavescens | 1231 | EU714363 | clade_225 | N | N |
| Microbacterium lacticum | 1233 | EU714351 | clade_225 | N | N |
| Microbacterium oleivorans | 1234 | EU714381 | clade_225 | N | N |
| Microbacterium oxydans | 1235 | EU714348 | clade_225 | N | N |
| Microbacterium paraoxydans | 1236 | AJ491806 | clade_225 | N | N |
| Microbacterium phyllosphaerae | 1237 | EU714359 | clade_225 | N | N |
| Microbacterium schleiferi | 1238 | NR_044936 | clade_225 | N | N |
| Microbacterium sp. 768 | 1239 | EU714378 | clade_225 | N | N |
| Microbacterium sp. oral strain C24KA | 1240 | AF287752 | clade_225 | N | N |
| Microbacterium testaceum | 1241 | EU714365 | clade_225 | N | N |
| Corynebacterium atypicum | 686 | NR_025540 | clade_229 | N | N |
| Corynebacterium mastitidis | 708 | AB359395 | clade_229 | N | N |
| Corynebacterium sp. NML 97_0186 | 725 | GU238411 | clade_229 | N | N |
| Mycobacterium elephantis | 1275 | AF385898 | clade_237 | N | OP |
| Mycobacterium paraterrae | 1288 | EU919229 | clade_237 | N | OP |
| Mycobacterium phlei | 1289 | GU142920 | clade_237 | N | OP |
| Mycobacterium sp. 1776 | 1293 | EU703152 | clade_237 | N | N |
| Mycobacterium sp. 1781 | 1294 | EU703147 | clade_237 | N | N |
| Mycobacterium sp. AQ1GA4 | 1297 | HM210417 | clade_237 | N | N |
| Mycobacterium sp. GN_10546 | 1299 | FJ497243 | clade_237 | N | N |
| Mycobacterium sp. GN_10827 | 1300 | FJ497247 | clade_237 | N | N |
| Mycobacterium sp. GN_11124 | 1301 | FJ652846 | clade_237 | N | N |
| Mycobacterium sp. GN_9188 | 1302 | FJ497240 | clade_237 | N | N |
| Mycobacterium sp. GR_2007_210 | 1303 | FJ555538 | clade_237 | N | N |
| Anoxybacillus contaminans | 172 | NR_029006 | clade_238 | N | N |
| Bacillus aeolius | 195 | NR_025557 | clade_238 | N | N |
| Brevibacterium frigoritolerans | 422 | NR_042639 | clade_238 | N | N |
| Geobacillus sp. E263 | 934 | DQ647387 | clade_238 | N | N |
| Geobacillus sp. WCH70 | 935 | CP001638 | clade_238 | N | N |
| Geobacillus thermocatenulatus | 937 | NR_043020 | clade_238 | N | N |
| Geobacillus thermoleovorans | 940 | NR_074931 | clade_238 | N | N |
| Lysinibacillus fusiformis | 1192 | FN397522 | clade_238 | N | N |
| Planomicrobium koreense | 1468 | NR_025011 | clade_238 | N | N |
| Sporosarcina newyorkensis | 1754 | AFPZ01000142 | clade_238 | N | N |
| Sporosarcina sp. 2681 | 1755 | GU994081 | clade_238 | N | N |
| Ureibacillus composti | 1968 | NR_043746 | clade_238 | N | N |
| Ureibacillus suwonensis | 1969 | NR_043232 | clade_238 | N | N |
| Ureibacillus terrenus | 1970 | NR_025394 | clade_238 | N | N |
| Ureibacillus thermophilus | 1971 | NR_043747 | clade_238 | N | N |
| Ureibacillus thermosphaericus | 1972 | NR_040961 | clade_238 | N | N |
| Prevotella micans | 1507 | AGWK01000061 | clade_239 | N | N |
| Prevotella sp. oral clone DA058 | 1533 | AY005065 | clade_239 | N | N |
| Prevotella sp. SEQ053 | 1523 | JN867222 | clade_239 | N | N |
| Treponema socranskii | 1937 | NR_024868 | clade_240 | N | OP |
| Treponema sp. 6:H:D15A_4 | 1938 | AY005083 | clade_240 | N | N |
| Treponema sp. oral taxon 265 | 1953 | GU408850 | clade_240 | N | N |
| Treponema sp. oral taxon G85 | 1958 | GU432215 | clade_240 | N | N |
| Porphyromonas endodontalis | 1472 | ACNN01000021 | clade_241 | N | N |
| Porphyromonas sp. oral clone BB134 | 1478 | AY005068 | clade_241 | N | N |
| Porphyromonas sp. oral clone F016 | 1479 | AY005069 | clade_241 | N | N |
| Porphyromonas sp. oral clone P2PB_52 P1 | 1480 | AY207054 | clade_241 | N | N |
| Porphyromonas sp. oral clone P4GB_100 P2 | 1481 | AY207057 | clade_241 | N | N |
| Acidovorax sp. 98_63833 | 26 | AY258065 | clade_245 | N | N |
| Comamonadaceae bacterium NML000135 | 663 | JN585335 | clade_245 | N | N |
| Comamonadaceae bacterium NML790751 | 664 | JN585331 | clade_245 | N | N |
| Comamonadaceae bacterium NML910035 | 665 | JN585332 | clade_245 | N | N |
| Comamonadaceae bacterium NML910036 | 666 | JN585333 | clade_245 | N | N |
| Comamonas sp. NSP5 | 668 | AB076850 | clade_245 | N | N |
| Delftia acidovorans | 748 | CP000884 | clade_245 | N | N |
| Xenophilus aerolatus | 2018 | JN585329 | clade_245 | N | N |
| Oribacterium sp. oral taxon 078 | 1380 | ACIQ02000009 | clade_246 | N | N |
| Oribacterium sp. oral taxon 102 | 1381 | GQ422713 | clade_246 | N | N |
| Weissella cibaria | 2007 | NR_036924 | clade_247 | N | N |
| Weissella confusa | 2008 | NR_040816 | clade_247 | N | N |
| Weissella hellenica | 2009 | AB680902 | clade_247 | N | N |
| Weissella kandleri | 2010 | NR_044659 | clade_247 | N | N |
| Weissella koreensis | 2011 | NR_075058 | clade_247 | N | N |
| Weissella paramesenteroides | 2012 | ACKU01000017 | clade_247 | N | N |
| Weissella sp. KLDS 7.0701 | 2013 | EU600924 | clade_247 | N | N |
| Mobiluncus curtisii | 1251 | AEPZ01000013 | clade_249 | N | N |
| Enhydrobacter aerosaccus | 785 | ACYI01000081 | clade_256 | N | N |
| Moraxella osloensis | 1262 | JN175341 | clade_256 | N | N |
| Moraxella sp. GM2 | 1264 | JF837191 | clade_256 | N | N |
| Brevibacterium casei | 420 | JF951998 | clade_257 | N | N |
| Brevibacterium epidermidis | 421 | NR_029262 | clade_257 | N | N |
| Brevibacterium sanguinis | 426 | NR_028016 | clade_257 | N | N |
| Brevibacterium sp. H15 | 427 | AB177640 | clade_257 | N | N |
| Acinetobacter radioresistens | 35 | ACVR01000010 | clade_261 | N | N |
| Lactobacillus alimentarius | 1068 | NR_044701 | clade_263 | N | N |
| Lactobacillus farciminis | 1082 | NR_044707 | clade_263 | N | N |
| Lactobacillus kimchii | 1097 | NR_025045 | clade_263 | N | N |
| Lactobacillus nodensis | 1101 | NR_041629 | clade_263 | N | N |
| Lactobacillus tucceti | 1138 | NR_042194 | clade_263 | N | N |
| Pseudomonas mendocina | 1595 | AAUL01000021 | clade_265 | N | N |
| Pseudomonas pseudoalcaligenes | 1598 | NR_037000 | clade_265 | N | N |
| Pseudomonas sp. NP522b | 1602 | EU723211 | clade_265 | N | N |
| Pseudomonas stutzeri | 1603 | AM905854 | clade_265 | N | N |
| Paenibacillus barcinonensis | 1390 | NR_042272 | clade_270 | N | N |
| Paenibacillus barengoltzii | 1391 | NR_042756 | clade_270 | N | N |
| Paenibacillus chibensis | 1392 | NR_040885 | clade_270 | N | N |
| Paenibacillus cookii | 1393 | NR_025372 | clade_270 | N | N |
| Paenibacillus durus | 1394 | NR_037017 | clade_270 | N | N |
| Paenibacillus glucanolyticus | 1395 | D78470 | clade_270 | N | N |
| Paenibacillus lactis | 1396 | NR_025739 | clade_270 | N | N |
| Paenibacillus pabuli | 1398 | NR_040853 | clade_270 | N | N |
| Paenibacillus popilliae | 1400 | NR_040888 | clade_270 | N | N |
| Paenibacillus sp. CIP 101062 | 1401 | HM212646 | clade_270 | N | N |
| Paenibacillus sp. JC66 | 1404 | JF824808 | clade_270 | N | N |
| Paenibacillus sp. R_27413 | 1405 | HE586333 | clade_270 | N | N |
| Paenibacillus sp. R_27422 | 1406 | HE586338 | clade_270 | N | N |
| Paenibacillus timonensis | 1408 | NR_042844 | clade_270 | N | N |
| Rothia mucilaginosa | 1651 | ACVO01000020 | clade_271 | N | N |
| Rothia nasimurium | 1652 | NR_025310 | clade_271 | N | N |
| Prevotella sp. oral taxon 302 | 1550 | ACZK01000043 | clade_280 | N | N |
| Prevotella sp. oral taxon F68 | 1556 | HM099652 | clade_280 | N | N |
| Prevotella tannerae | 1563 | ACIJ02000018 | clade_280 | N | N |
| Prevotellaceae bacterium P4P_62 P1 | 1566 | AY207061 | clade_280 | N | N |
| Porphyromonas asaccharolytica | 1471 | AENO01000048 | clade_281 | N | N |
| Porphyromonas gingivalis | 1473 | AE015924 | clade_281 | N | N |
| Porphyromonas macacae | 1475 | NR_025908 | clade_281 | N | N |
| Porphyromonas sp. UQD 301 | 1477 | EU012301 | clade_281 | N | N |
| Porphyromonas uenonis | 1482 | ACLR01000152 | clade_281 | N | N |
| Leptotrichia buccalis | 1165 | CP001685 | clade_282 | N | N |
| Leptotrichia hofstadii | 1168 | ACVB02000032 | clade_282 | N | N |
| Leptotrichia sp. oral clone HE012 | 1173 | AY349386 | clade_282 | N | N |
| Leptotrichia sp. oral taxon 223 | 1176 | GU408547 | clade_282 | N | N |
| Bacteroides fluxus | 278 | AFBN01000029 | clade_285 | N | N |
| Bacteroides helcogenes | 281 | CP002352 | clade_285 | N | N |
| Parabacteroides johnsonii | 1419 | ABYH01000014 | clade_286 | N | N |
| Parabacteroides merdae | 1420 | EU136685 | clade_286 | N | N |
| Treponema denticola | 1926 | ADEC01000002 | clade_288 | N | OP |
| Treponema genomosp. P5 oral clone MB3_P23 | 1929 | DQ003624 | clade_288 | N | N |
| Treponema putidum | 1935 | AJ543428 | clade_288 | N | OP |
| Treponema sp. oral clone P2PB_53 P3 | 1942 | AY207055 | clade_288 | N | N |
| Treponema sp. oral taxon 247 | 1949 | GU408748 | clade_288 | N | N |
| Treponema sp. oral taxon 250 | 1950 | GU408776 | clade_288 | N | N |
| Treponema sp. oral taxon 251 | 1951 | GU408781 | clade_288 | N | N |
| Anaerococcus hydrogenalis | 144 | ABXA01000039 | clade_289 | N | N |
| Anaerococcus sp. 8404299 | 148 | HM587318 | clade_289 | N | N |
| Anaerococcus sp. gpac215 | 156 | AM176540 | clade_289 | N | N |
| Anaerococcus vaginalis | 158 | ACXU01000016 | clade_289 | N | N |
| Propionibacterium acidipropionici | 1569 | NC_019395 | clade_290 | N | N |
| Propionibacterium avidum | 1571 | AJ003055 | clade_290 | N | N |
| Propionibacterium granulosum | 1573 | FJ785716 | clade_290 | N | N |
| Propionibacterium jensenii | 1574 | NR_042269 | clade_290 | N | N |
| Propionibacterium propionicum | 1575 | NR_025277 | clade_290 | N | N |
| Propionibacterium sp. H456 | 1577 | AB177643 | clade_290 | N | N |
| Propionibacterium thoenii | 1581 | NR_042270 | clade_290 | N | N |
| Bifidobacterium bifidum | 349 | ABQP01000027 | clade_293 | N | N |
| Leuconostoc mesenteroides | 1183 | ACKV01000113 | clade_295 | N | N |
| Leuconostoc pseudomesenteroides | 1184 | NR_040814 | clade_295 | N | N |
| Johnsonella ignava | 1016 | X87152 | clade_298 | N | N |
| Propionibacterium acnes | 1570 | ADJM01000010 | clade_299 | N | N |
| Propionibacterium sp. 434_HC2 | 1576 | AFIL01000035 | clade_299 | N | N |
| Propionibacterium sp. LG | 1578 | AY354921 | clade_299 | N | N |
| Propionibacterium sp. S555a | 1579 | AB264622 | clade_299 | N | N |
| Alicyclobacillus sp. CCUG 53762 | 128 | HE613268 | clade_301 | N | N |
| Actinomyces cardiffensis | 53 | GU470888 | clade_303 | N | N |
| Actinomyces funkei | 55 | HQ906497 | clade_303 | N | N |
| Actinomyces sp. HKU31 | 74 | HQ335393 | clade_303 | N | N |
| Actinomyces sp. oral taxon C55 | 94 | HM099646 | clade_303 | N | N |
| Kerstersia gyiorum | 1018 | NR_025669 | clade_307 | N | N |
| Pigmentiphaga daeguensis | 1467 | JN585327 | clade_307 | N | N |
| Aeromonas allosaccharophila | 104 | S39232 | clade_308 | N | N |
| Aeromonas enteropelogenes | 105 | X71121 | clade_308 | N | N |
| Aeromonas hydrophila | 106 | NC_008570 | clade_308 | N | N |
| Aeromonas jandaei | 107 | X60413 | clade_308 | N | N |
| Aeromonas salmonicida | 108 | NC_009348 | clade_308 | N | N |
| Aeromonas trota | 109 | X60415 | clade_308 | N | N |
| Aeromonas veronii | 110 | NR_044845 | clade_308 | N | N |
| Marvinbryantia formatexigens | 1196 | AJ505973 | clade_309 | N | N |
| Rhodobacter sp. oral taxon C30 | 1620 | HM099648 | clade_310 | N | N |
| Rhodobacter sphaeroides | 1621 | CP000144 | clade_310 | N | N |
| Lactobacillus antri | 1071 | ACLL01000037 | clade_313 | N | N |
| Lactobacillus coleohominis | 1076 | ACOH01000030 | clade_313 | N | N |
| Lactobacillus fermentum | 1083 | CP002033 | clade_313 | N | N |
| Lactobacillus gastricus | 1085 | AICN01000060 | clade_313 | N | N |
| Lactobacillus mucosae | 1099 | FR693800 | clade_313 | N | N |
| Lactobacillus oris | 1103 | AEKL01000077 | clade_313 | N | N |
| Lactobacillus pontis | 1111 | HM218420 | clade_313 | N | N |
| Lactobacillus reuteri | 1112 | ACGW02000012 | clade_313 | N | N |
| Lactobacillus sp. KLDS 1.0707 | 1127 | EU600911 | clade_313 | N | N |
| Lactobacillus sp. KLDS 1.0709 | 1128 | EU600913 | clade_313 | N | N |
| Lactobacillus sp. KLDS 1.0711 | 1129 | EU600915 | clade_313 | N | N |
| Lactobacillus sp. KLDS 1.0713 | 1131 | EU600917 | clade_313 | N | N |
| Lactobacillus sp. KLDS 1.0716 | 1132 | EU600921 | clade_313 | N | N |
| Lactobacillus sp. KLDS 1.0718 | 1133 | EU600922 | clade_313 | N | N |
| Lactobacillus sp. oral taxon 052 | 1137 | GQ422710 | clade_313 | N | N |
| Lactobacillus vaginalis | 1140 | ACGV01000168 | clade_313 | N | N |
| Brevibacterium aurantiacum | 419 | NR_044854 | clade_314 | N | N |
| Brevibacterium linens | 423 | AJ315491 | clade_314 | N | N |
| Lactobacillus pentosus | 1108 | JN813103 | clade_315 | N | N |
| Lactobacillus plantarum | 1110 | ACGZ02000033 | clade_315 | N | N |
| Lactobacillus sp. KLDS 1.0702 | 1123 | EU600906 | clade_315 | N | N |
| Lactobacillus sp. KLDS 1.0703 | 1124 | EU600907 | clade_315 | N | N |
| Lactobacillus sp. KLDS 1.0704 | 1125 | EU600908 | clade_315 | N | N |
| Lactobacillus sp. KLDS 1.0705 | 1126 | EU600909 | clade_315 | N | N |
| Agrobacterium radiobacter | 115 | CP000628 | clade_316 | N | N |
| Agrobacterium tumefaciens | 116 | AJ389893 | clade_316 | N | N |
| Corynebacterium argentoratense | 685 | EF463055 | clade_317 | N | N |
| Corynebacterium diphtheriae | 693 | NC_002935 | clade_317 | N | OP |
| Corynebacterium pseudotuberculosis | 715 | NR_037070 | clade_317 | N | N |
| Corynebacterium renale | 717 | NR_037069 | clade_317 | N | N |
| Corynebacterium ulcerans | 731 | NR_074467 | clade_317 | N | N |
| Aurantimonas coralicida | 191 | AY065627 | clade_318 | N | N |
| Aureimonas altamirensis | 192 | FN658986 | clade_318 | N | N |
| Lactobacillus acidipiscis | 1066 | NR_024718 | clade_320 | N | N |
| Lactobacillus salivarius | 1117 | AEBA01000145 | clade_320 | N | N |
| Lactobacillus sp. KLDS 1.0719 | 1134 | EU600923 | clade_320 | N | N |
| Lactobacillus buchneri | 1073 | ACGH01000101 | clade_321 | N | N |
| Lactobacillus genomosp. C1 | 1086 | AY278619 | clade_321 | N | N |
| Lactobacillus genomosp. C2 | 1087 | AY278620 | clade_321 | N | N |
| Lactobacillus hilgardii | 1089 | ACGP01000200 | clade_321 | N | N |
| Lactobacillus kefiri | 1096 | NR_042230 | clade_321 | N | N |
| Lactobacillus parabuchneri | 1105 | NR_041294 | clade_321 | N | N |
| Lactobacillus parakefiri | 1107 | NR_029039 | clade_321 | N | N |
| Lactobacillus curvatus | 1079 | NR_042437 | clade_322 | N | N |
| Lactobacillus sakei | 1116 | DQ989236 | clade_322 | N | N |
| Aneurinibacillus aneurinilyticus | 167 | AB101592 | clade_323 | N | N |
| Aneurinibacillus danicus | 168 | NR_028657 | clade_323 | N | N |
| Aneurinibacillus migulanus | 169 | NR_036799 | clade_323 | N | N |
| Aneurinibacillus terranovensis | 170 | NR_042271 | clade_323 | N | N |
| Staphylococcus aureus | 1757 | CP002643 | clade_325 | N | Category-B |
| Staphylococcus auricularis | 1758 | JQ624774 | clade_325 | N | N |
| Staphylococcus capitis | 1759 | ACFR01000029 | clade_325 | N | N |
| Staphylococcus caprae | 1760 | ACRH01000033 | clade_325 | N | N |
| Staphylococcus carnosus | 1761 | NR_075003 | clade_325 | N | N |
| Staphylococcus cohnii | 1762 | JN175375 | clade_325 | N | N |
| Staphylococcus condimenti | 1763 | NR_029345 | clade_325 | N | N |
| Staphylococcus epidermidis | 1764 | ACHE01000056 | clade_325 | N | N |
| Staphylococcus equorum | 1765 | NR_027520 | clade_325 | N | N |
| Staphylococcus haemolyticus | 1767 | NC_007168 | clade_325 | N | N |
| Staphylococcus hominis | 1768 | AM157418 | clade_325 | N | N |
| Staphylococcus lugdunensis | 1769 | AEQA01000024 | clade_325 | N | N |
| Staphylococcus pasteuri | 1770 | FJ189773 | clade_325 | N | N |
| Staphylococcus pseudintermedius | 1771 | CP002439 | clade_325 | N | N |
| Staphylococcus saccharolyticus | 1772 | NR_029158 | clade_325 | N | N |
| Staphylococcus saprophyticus | 1773 | NC_007350 | clade_325 | N | N |
| Staphylococcus sp. clone bottae7 | 1777 | AF467424 | clade_325 | N | N |
| Staphylococcus sp. H292 | 1775 | AB177642 | clade_325 | N | N |
| Staphylococcus sp. H780 | 1776 | AB177644 | clade_325 | N | N |
| Staphylococcus succinus | 1778 | NR_028667 | clade_325 | N | N |
| Staphylococcus warneri | 1780 | ACPZ01000009 | clade_325 | N | N |
| Staphylococcus xylosus | 1781 | AY395016 | clade_325 | N | N |
| Cardiobacterium hominis | 490 | ACKY01000036 | clade_326 | N | N |
| Cardiobacterium valvarum | 491 | NR_028847 | clade_326 | N | N |
| Pseudomonas fluorescens | 1593 | AY622220 | clade_326 | N | N |
| Pseudomonas gessardii | 1594 | FJ943496 | clade_326 | N | N |
| Pseudomonas monteilii | 1596 | NR_024910 | clade_326 | N | N |
| Pseudomonas poae | 1597 | GU188951 | clade_326 | N | N |
| Pseudomonas putida | 1599 | AF094741 | clade_326 | N | N |
| Pseudomonas sp. G1229 | 1601 | DQ910482 | clade_326 | N | N |
| Pseudomonas tolaasii | 1604 | AF320988 | clade_326 | N | N |
| Pseudomonas viridiflava | 1605 | NR_042764 | clade_326 | N | N |
| Listeria grayi | 1185 | ACCR02000003 | clade_328 | N | OP |
| Listeria innocua | 1186 | JF967625 | clade_328 | N | N |
| Listeria ivanovii | 1187 | X56151 | clade_328 | N | N |
| Listeria monocytogenes | 1188 | CP002003 | clade_328 | N | Category-B |
| Listeria welshimeri | 1189 | AM263198 | clade_328 | N | OP |
| Capnocytophaga sp. oral clone ASCH05 | 484 | AY923149 | clade_333 | N | N |
| Capnocytophaga sputigena | 489 | ABZV01000054 | clade_333 | N | N |
| Leptotrichia genomosp. C1 | 1166 | AY278621 | clade_334 | N | N |
| Leptotrichia shahii | 1169 | AY029806 | clade_334 | N | N |
| Leptotrichia sp. neutropenicPatient | 1170 | AF189244 | clade_334 | N | N |
| Leptotrichia sp. oral clone GT018 | 1171 | AY349384 | clade_334 | N | N |
| Leptotrichia sp. oral clone GT020 | 1172 | AY349385 | clade_334 | N | N |
| Bacteroides sp. 20_3 | 296 | ACRQ01000064 | clade_335 | N | N |
| Bacteroides sp. 3_1_19 | 307 | ADCJ01000062 | clade_335 | N | N |
| Bacteroides sp. 3_2_5 | 311 | ACIB01000079 | clade_335 | N | N |
| Parabacteroides distasonis | 1416 | CP000140 | clade_335 | N | N |
| Parabacteroides goldsteinii | 1417 | AY974070 | clade_335 | N | N |
| Parabacteroides gordonii | 1418 | AB470344 | clade_335 | N | N |
| Parabacteroides sp. D13 | 1421 | ACPW01000017 | clade_335 | N | N |
| Capnocytophaga genomosp. C1 | 477 | AY278613 | clade_336 | N | N |
| Capnocytophaga ochracea | 480 | AEOH01000054 | clade_336 | N | N |
| Capnocytophaga sp. GEJ8 | 481 | GU561335 | clade_336 | N | N |
| Capnocytophaga sp. oral strain A47ROY | 486 | AY005077 | clade_336 | N | N |
| Capnocytophaga sp. S1b | 482 | U42009 | clade_336 | N | N |
| Paraprevotella clara | 1426 | AFFY01000068 | clade_336 | N | N |
| Bacteroides heparinolyticus | 282 | JN867284 | clade_338 | N | N |
| Prevotella heparinolytica | 1500 | GQ422742 | clade_338 | N | N |
| Treponema genomosp. P4 oral clone MB2_G19 | 1928 | DQ003618 | clade_339 | N | N |
| Treponema genomosp. P6 oral clone MB4_G11 | 1930 | DQ003625 | clade_339 | N | N |
| Treponema sp. oral taxon 254 | 1952 | GU408803 | clade_339 | N | N |
| Treponema sp. oral taxon 508 | 1956 | GU413616 | clade_339 | N | N |
| Treponema sp. oral taxon 518 | 1957 | GU413640 | clade_339 | N | N |
| Chlamydia muridarum | 502 | AE002160 | clade_341 | N | OP |
| Chlamydia trachomatis | 504 | U68443 | clade_341 | N | OP |
| Chlamydia psittaci | 503 | NR_036864 | clade_342 | N | Category-B |
| Chlamydophila pneumoniae | 509 | NC_002179 | clade_342 | N | OP |
| Chlamydophila psittaci | 510 | D85712 | clade_342 | N | OP |
| Anaerococcus octavius | 146 | NR_026360 | clade_343 | N | N |
| Anaerococcus sp. 8405254 | 149 | HM587319 | clade_343 | N | N |
| Anaerococcus sp. 9401487 | 150 | HM587322 | clade_343 | N | N |
| Anaerococcus sp. 9403502 | 151 | HM587325 | clade_343 | N | N |
| Gardnerella vaginalis | 923 | CP001849 | clade_344 | N | N |
| Campylobacter lari | 466 | CP000932 | clade_346 | N | OP |
| Anaerobiospirillum succiniciproducens | 142 | NR_026075 | clade_347 | N | N |
| Anaerobiospirillum thomasii | 143 | AJ420985 | clade_347 | N | N |
| Ruminobacter amylophilus | 1654 | NR_026450 | clade_347 | N | N |
| Succinatimonas hippei | 1897 | AEVO01000027 | clade_347 | N | N |
| Actinomyces europaeus | 54 | NR_026363 | clade_348 | N | N |
| Actinomyces sp. oral clone GU009 | 82 | AY349361 | clade_348 | N | N |
| Moraxella catarrhalis | 1260 | CP002005 | clade_349 | N | N |
| Moraxella lincolnii | 1261 | FR822735 | clade_349 | N | N |
| Moraxella sp. 16285 | 1263 | JF682466 | clade_349 | N | N |
| Psychrobacter sp. 13983 | 1613 | HM212668 | clade_349 | N | N |
| Actinobaculum massiliae | 49 | AF487679 | clade_350 | N | N |
| Actinobaculum schaalii | 50 | AY957507 | clade_350 | N | N |
| Actinobaculum sp. BM#101342 | 51 | AY282578 | clade_350 | N | N |
| Actinobaculum sp. P2P_19 P1 | 52 | AY207066 | clade_350 | N | N |
| Actinomyces sp. oral clone IO076 | 84 | AY349363 | clade_350 | N | N |
| Actinomyces sp. oral taxon 848 | 93 | ACUY01000072 | clade_350 | N | N |
| Actinomyces neuii | 65 | X71862 | clade_352 | N | N |
| Mobiluncus mulieris | 1252 | ACKW01000035 | clade_352 | N | N |
| Blastomonas natatoria | 372 | NR_040824 | clade_356 | N | N |
| Novosphingobium aromaticivorans | 1357 | AAAV03000008 | clade_356 | N | N |
| Sphingomonas sp. oral clone FI012 | 1745 | AY349411 | clade_356 | N | N |
| Sphingopyxis alaskensis | 1749 | CP000356 | clade_356 | N | N |
| Oxalobacter formigenes | 1389 | ACDQ01000020 | clade_357 | N | N |
| Veillonella atypica | 1974 | AEDS01000059 | clade_358 | N | N |
| Veillonella dispar | 1975 | ACIK02000021 | clade_358 | N | N |
| Veillonella genomosp. P1 oral clone MB5_P17 | 1976 | DQ003631 | clade_358 | N | N |
| Veillonella parvula | 1978 | ADFU01000009 | clade_358 | N | N |
| Veillonella sp. 3_1_44 | 1979 | ADCV01000019 | clade_358 | N | N |
| Veillonella sp. 6_1_27 | 1980 | ADCW01000016 | clade_358 | N | N |
| Veillonella sp. ACP1 | 1981 | HQ616359 | clade_358 | N | N |
| Veillonella sp. AS16 | 1982 | HQ616365 | clade_358 | N | N |
| Veillonella sp. BS32b | 1983 | HQ616368 | clade_358 | N | N |
| Veillonella sp. ICM51a | 1984 | HQ616396 | clade_358 | N | N |
| Veillonella sp. MSA12 | 1985 | HQ616381 | clade_358 | N | N |
| Veillonella sp. NVG 100cf | 1986 | EF108443 | clade_358 | N | N |
| Veillonella sp. OK11 | 1987 | JN695650 | clade_358 | N | N |
| Veillonella sp. oral clone ASCG01 | 1990 | AY923144 | clade_358 | N | N |
| Veillonella sp. oral clone ASCG02 | 1991 | AY953257 | clade_358 | N | N |
| Veillonella sp. oral clone OH1A | 1992 | AY947495 | clade_358 | N | N |
| Veillonella sp. oral taxon 158 | 1993 | AENU01000007 | clade_358 | N | N |
| Kocuria marina | 1040 | GQ260086 | clade_365 | N | N |
| Kocuria rhizophila | 1042 | AY030315 | clade_365 | N | N |
| Kocuria rosea | 1043 | X87756 | clade_365 | N | N |
| Kocuria varians | 1044 | AF542074 | clade_365 | N | N |
| Clostridiaceae bacterium END_2 | 531 | EF451053 | clade_368 | N | N |
| Micrococcus antarcticus | 1242 | NR_025285 | clade_371 | N | N |
| Micrococcus luteus | 1243 | NR_075062 | clade_371 | N | N |
| Micrococcus lylae | 1244 | NR_026200 | clade_371 | N | N |
| Micrococcus sp. 185 | 1245 | EU714334 | clade_371 | N | N |
| Lactobacillus brevis | 1072 | EU194349 | clade_372 | N | N |
| Lactobacillus parabrevis | 1104 | NR_042456 | clade_372 | N | N |
| Pediococcus acidilactici | 1436 | ACXB01000026 | clade_372 | N | N |
| Pediococcus pentosaceus | 1437 | NR_075052 | clade_372 | N | N |
| Lactobacillus dextrinicus | 1081 | NR_036861 | clade_373 | N | N |
| Lactobacillus perolens | 1109 | NR_029360 | clade_373 | N | N |
| Lactobacillus rhamnosus | 1113 | ABWJ01000068 | clade_373 | N | N |
| Lactobacillus saniviri | 1118 | AB602569 | clade_373 | N | N |
| Lactobacillus sp. BT6 | 1121 | HQ616370 | clade_373 | N | N |
| Mycobacterium mageritense | 1282 | FR798914 | clade_374 | N | OP |
| Mycobacterium neoaurum | 1286 | AF268445 | clade_374 | N | OP |
| Mycobacterium smegmatis | 1291 | CP000480 | clade_374 | N | OP |
| Mycobacterium sp. HE5 | 1304 | AJ012738 | clade_374 | N | N |
| Dysgonomonas gadei | 775 | ADLV01000001 | clade_377 | N | N |
| Dysgonomonas mossii | 776 | ADLW01000023 | clade_377 | N | N |
| Porphyromonas levii | 1474 | NR_025907 | clade_377 | N | N |
| Porphyromonas somerae | 1476 | AB547667 | clade_377 | N | N |
| Bacteroides barnesiae | 267 | NR_041446 | clade_378 | N | N |
| Bacteroides coprocola | 272 | ABIY02000050 | clade_378 | N | N |
| Bacteroides coprophilus | 273 | ACBW01000012 | clade_378 | N | N |
| Bacteroides dorei | 274 | ABWZ01000093 | clade_378 | N | N |
| Bacteroides massiliensis | 284 | AB200226 | clade_378 | N | N |
| Bacteroides plebeius | 289 | AB200218 | clade_378 | N | N |
| Bacteroides sp. 3_1_33FAA | 309 | ACPS01000085 | clade_378 | N | N |
| Bacteroides sp. 3_1_40A | 310 | ACRT01000136 | clade_378 | N | N |
| Bacteroides sp. 4_3_47FAA | 313 | ACDR02000029 | clade_378 | N | N |
| Bacteroides sp. 9_1_42FAA | 314 | ACAA01000096 | clade_378 | N | N |
| Bacteroides sp. NB_8 | 323 | AB117565 | clade_378 | N | N |
| Bacteroides vulgatus | 331 | CP000139 | clade_378 | N | N |
| Bacteroides ovatus | 287 | ACWH01000036 | clade_38 | N | N |
| Bacteroides sp. 1_1_30 | 294 | ADCL01000128 | clade_38 | N | N |
| Bacteroides sp. 2_1_22 | 297 | ACPQ01000117 | clade_38 | N | N |
| Bacteroides sp. 2_2_4 | 299 | ABZZ01000168 | clade_38 | N | N |
| Bacteroides sp. 3_1_23 | 308 | ACRS01000081 | clade_38 | N | N |
| Bacteroides sp. D1 | 318 | ACAB02000030 | clade_38 | N | N |
| Bacteroides sp. D2 | 321 | ACGA01000077 | clade_38 | N | N |
| Bacteroides sp. D22 | 320 | ADCK01000151 | clade_38 | N | N |
| Bacteroides xylanisolvens | 332 | ADKP01000087 | clade_38 | N | N |
| Treponema lecithinolyticum | 1931 | NR_026247 | clade_380 | N | OP |
| Treponema parvum | 1933 | AF302937 | clade_380 | N | OP |
| Treponema sp. oral clone JU025 | 1940 | AY349417 | clade_380 | N | N |
| Treponema sp. oral taxon 270 | 1954 | GQ422733 | clade_380 | N | N |
| Parascardovia denticolens | 1428 | ADEB01000020 | clade_381 | N | N |
| Scardovia inopinata | 1688 | AB029087 | clade_381 | N | N |
| Scardovia wiggsiae | 1689 | AY278626 | clade_381 | N | N |
| Clostridiales bacterium 9400853 | 533 | HM587320 | clade_384 | N | N |
| Mogibacterium diversum | 1254 | NR_027191 | clade_384 | N | N |
| Mogibacterium neglectum | 1255 | NR_027203 | clade_384 | N | N |
| Mogibacterium pumilum | 1256 | NR_028608 | clade_384 | N | N |
| Mogibacterium timidum | 1257 | Z36296 | clade_384 | N | N |
| Borrelia burgdorferi | 389 | ABGI01000001 | clade_386 | N | OP |
| Borrelia garinii | 392 | ABJV01000001 | clade_386 | N | OP |
| Borrelia sp. NE49 | 397 | AJ224142 | clade_386 | N | OP |
| Caldimonas manganoxidans | 457 | NR_040787 | clade_387 | N | N |
| Comamonadaceae bacterium oral taxon F47 | 667 | HM099651 | clade_387 | N | N |
| Lautropia mirabilis | 1149 | AEQP01000026 | clade_387 | N | N |
| Lautropia sp. oral clone AP009 | 1150 | AY005030 | clade_387 | N | N |
| Peptoniphilus asaccharolyticus | 1441 | D14145 | clade_389 | N | N |
| Peptoniphilus duerdenii | 1442 | EU526290 | clade_389 | N | N |
| Peptoniphilus harei | 1443 | NR_026358 | clade_389 | N | N |
| Peptoniphilus indolicus | 1444 | AY153431 | clade_389 | N | N |
| Peptoniphilus lacrimalis | 1446 | ADDO01000050 | clade_389 | N | N |
| Peptoniphilus sp. gpac077 | 1450 | AM176527 | clade_389 | N | N |
| Peptoniphilus sp. JC140 | 1447 | JF824803 | clade_389 | N | N |
| Peptoniphilus sp. oral taxon 386 | 1452 | ADCS01000031 | clade_389 | N | N |
| Peptoniphilus sp. oral taxon 836 | 1453 | AEAA01000090 | clade_389 | N | N |
| Peptostreptococcaceae bacterium ph1 | 1454 | JN837495 | clade_389 | N | N |
| Dialister pneumosintes | 765 | HM596297 | clade_390 | N | N |
| Dialister sp. oral taxon 502 | 767 | GQ422739 | clade_390 | N | N |
| Cupriavidus metallidurans | 741 | GU230889 | clade_391 | N | N |
| Herbaspirillum seropedicae | 1001 | CP002039 | clade_391 | N | N |
| Herbaspirillum sp. JC206 | 1002 | JN657219 | clade_391 | N | N |
| Janthinobacterium sp. SY12 | 1015 | EF455530 | clade_391 | N | N |
| Massilia sp. CCUG 43427A | 1197 | FR773700 | clade_391 | N | N |
| Ralstonia pickettii | 1615 | NC_010682 | clade_391 | N | N |
| Ralstonia sp. 5_7_47FAA | 1616 | ACUF01000076 | clade_391 | N | N |
| Francisella novicida | 889 | ABSS01000002 | clade_392 | N | N |
| Francisella philomiragia | 890 | AY928394 | clade_392 | N | N |
| Francisella tularensis | 891 | ABAZ01000082 | clade_392 | N | Category-A |
| Ignatzschineria indica | 1009 | HQ823562 | clade_392 | N | N |
| Ignatzschineria sp. NML 95_0260 | 1010 | HQ823559 | clade_392 | N | N |
| Streptococcus mutans | 1814 | AP010655 | clade_394 | N | N |
| Lactobacillus gasseri | 1084 | ACOZ01000018 | clade_398 | N | N |
| Lactobacillus hominis | 1090 | FR681902 | clade_398 | N | N |
| Lactobacillus iners | 1091 | AEKJ01000002 | clade_398 | N | N |
| Lactobacillus johnsonii | 1093 | AE017198 | clade_398 | N | N |
| Lactobacillus senioris | 1119 | AB602570 | clade_398 | N | N |
| Lactobacillus sp. oral clone HT002 | 1135 | AY349382 | clade_398 | N | N |
| Weissella beninensis | 2006 | EU439435 | clade_398 | N | N |
| Sphingomonas echinoides | 1744 | NR_024700 | clade_399 | N | N |
| Sphingomonas sp. oral taxon A09 | 1747 | HM099639 | clade_399 | N | N |
| Sphingomonas sp. oral taxon F71 | 1748 | HM099645 | clade_399 | N | N |
| Zymomonas mobilis | 2032 | NR_074274 | clade_399 | N | N |
| Arcanobacterium haemolyticum | 174 | NR_025347 | clade_400 | N | N |
| Arcanobacterium pyogenes | 175 | GU585578 | clade_400 | N | N |
| Trueperella pyogenes | 1962 | NR_044858 | clade_400 | N | N |
| Lactococcus garvieae | 1144 | AF061005 | clade_401 | N | N |
| Lactococcus lactis | 1145 | CP002365 | clade_401 | N | N |
| Brevibacterium mcbrellneri | 424 | ADNU01000076 | clade_402 | N | N |
| Brevibacterium paucivorans | 425 | EU086796 | clade_402 | N | N |
| Brevibacterium sp. JC43 | 428 | JF824806 | clade_402 | N | N |
| Selenomonas artemidis | 1692 | HM596274 | clade_403 | N | N |
| Selenomonas sp. FOBRC9 | 1704 | HQ616378 | clade_403 | N | N |
| Selenomonas sp. oral taxon 137 | 1715 | AENV01000007 | clade_403 | N | N |
| Desmospora activa | 751 | AM940019 | clade_404 | N | N |
| Desmospora sp. 8437 | 752 | AFHT01000143 | clade_404 | N | N |
| Paenibacillus sp. oral taxon F45 | 1407 | HM099647 | clade_404 | N | N |
| Corynebacterium ammoniagenes | 682 | ADNS01000011 | clade_405 | N | N |
| Corynebacterium aurimucosum | 687 | ACLH01000041 | clade_405 | N | N |
| Corynebacterium bovis | 688 | AF537590 | clade_405 | N | N |
| Corynebacterium canis | 689 | GQ871934 | clade_405 | N | N |
| Corynebacterium casei | 690 | NR_025101 | clade_405 | N | N |
| Corynebacterium durum | 694 | Z97069 | clade_405 | N | N |
| Corynebacterium efficiens | 695 | ACLI01000121 | clade_405 | N | N |
| Corynebacterium falsenii | 696 | Y13024 | clade_405 | N | N |
| Corynebacterium flavescens | 697 | NR_037040 | clade_405 | N | N |
| Corynebacterium glutamicum | 701 | BA000036 | clade_405 | N | N |
| Corynebacterium jeikeium | 704 | ACYW01000001 | clade_405 | N | OP |
| Corynebacterium kroppenstedtii | 705 | NR_026380 | clade_405 | N | N |
| Corynebacterium lipophiloflavum | 706 | ACHJ01000075 | clade_405 | N | N |
| Corynebacterium matruchotii | 709 | ACSH02000003 | clade_405 | N | N |
| Corynebacterium minutissimum | 710 | X82064 | clade_405 | N | N |
| Corynebacterium resistens | 718 | ADGN01000058 | clade_405 | N | N |
| Corynebacterium simulans | 720 | AF537604 | clade_405 | N | N |
| Corynebacterium singulare | 721 | NR_026394 | clade_405 | N | N |
| Corynebacterium sp. 1 ex sheep | 722 | Y13427 | clade_405 | N | N |
| Corynebacterium sp. NML 99_0018 | 726 | GU238413 | clade_405 | N | N |
| Corynebacterium striatum | 727 | ACGE01000001 | clade_405 | N | OP |
| Corynebacterium urealyticum | 732 | X81913 | clade_405 | N | OP |
| Corynebacterium variabile | 734 | NR_025314 | clade_405 | N | N |
| Aerococcus sanguinicola | 98 | AY837833 | clade_407 | N | N |
| Aerococcus urinae | 99 | CP002512 | clade_407 | N | N |
| Aerococcus urinaeequi | 100 | NR_043443 | clade_407 | N | N |
| Aerococcus viridans | 101 | ADNT01000041 | clade_407 | N | N |
| Fusobacterium naviforme | 898 | HQ223106 | clade_408 | N | N |
| Moryella indoligenes | 1268 | AF527773 | clade_408 | N | N |
| Selenomonas genomosp. P5 | 1697 | AY341820 | clade_410 | N | N |
| Selenomonas sp. oral clone IQ048 | 1710 | AY349408 | clade_410 | N | N |
| Selenomonas sputigena | 1717 | ACKP02000033 | clade_410 | N | N |
| Hyphomicrobium sulfonivorans | 1007 | AY468372 | clade_411 | N | N |
| Methylocella silvestris | 1228 | NR_074237 | clade_411 | N | N |
| Legionella pneumophila | 1153 | NC_002942 | clade_412 | N | OP |
| Lactobacillus coryniformis | 1077 | NR_044705 | clade_413 | N | N |
| Arthrobacter agilis | 178 | NR_026198 | clade_414 | N | N |
| Arthrobacter arilaitensis | 179 | NR_074608 | clade_414 | N | N |
| Arthrobacter bergerei | 180 | NR_025612 | clade_414 | N | N |
| Arthrobacter globiformis | 181 | NR_026187 | clade_414 | N | N |
| Arthrobacter nicotianae | 182 | NR_026190 | clade_414 | N | N |
| Mycobacterium abscessus | 1269 | AGQU01000002 | clade_418 | N | OP |
| Mycobacterium chelonae | 1273 | AB548610 | clade_418 | N | OP |
| Bacteroides salanitronis | 291 | CP002530 | clade_419 | N | N |
| Paraprevotella xylaniphila | 1427 | AFBR01000011 | clade_419 | N | N |
| Barnesiella intestinihominis | 336 | AB370251 | clade_420 | N | N |
| Barnesiella viscericola | 337 | NR_041508 | clade_420 | N | N |
| Parabacteroides sp. NS31_3 | 1422 | JN029805 | clade_420 | N | N |
| Porphyromonadaceae bacterium NML 060648 | 1470 | EF184292 | clade_420 | N | N |
| Tannerella forsythia | 1913 | CP003191 | clade_420 | N | N |
| Tannerella sp. 6_1_58FAA_CT1 | 1914 | ACWX01000068 | clade_420 | N | N |
| Mycoplasma amphoriforme | 1311 | AY531656 | clade_421 | N | N |
| Mycoplasma genitalium | 1317 | L43967 | clade_421 | N | N |
| Mycoplasma pneumoniae | 1322 | NC_000912 | clade_421 | N | N |
| Mycoplasma penetrans | 1321 | NC_004432 | clade_422 | N | N |
| Ureaplasma parvum | 1966 | AE002127 | clade_422 | N | N |
| Ureaplasma urealyticum | 1967 | AAYN01000002 | clade_422 | N | N |
| Treponema genomosp. P1 | 1927 | AY341822 | clade_425 | N | N |
| Treponema sp. oral taxon 228 | 1943 | GU408580 | clade_425 | N | N |
| Treponema sp. oral taxon 230 | 1944 | GU408603 | clade_425 | N | N |
| Treponema sp. oral taxon 231 | 1945 | GU408631 | clade_425 | N | N |
| Treponema sp. oral taxon 232 | 1946 | GU408646 | clade_425 | N | N |
| Treponema sp. oral taxon 235 | 1947 | GU408673 | clade_425 | N | N |
| Treponema sp. ovine footrot | 1959 | AJ010951 | clade_425 | N | N |
| Treponema vincentii | 1960 | ACYH01000036 | clade_425 | N | OP |
| Burkholderiales bacterium 1_1_47 | 452 | ADCQ01000066 | clade_432 | N | OP |
| Parasutterella excrementihominis | 1429 | AFBP01000029 | clade_432 | N | N |
| Parasutterella secunda | 1430 | AB491209 | clade_432 | N | N |
| Sutterella morbirenis | 1898 | AJ832129 | clade_432 | N | N |
| Sutterella sanguinus | 1900 | AJ748647 | clade_432 | N | N |
| Sutterella sp. YIT 12072 | 1901 | AB491210 | clade_432 | N | N |
| Sutterella stercoricanis | 1902 | NR_025600 | clade_432 | N | N |
| Sutterella wadsworthensis | 1903 | ADMF01000048 | clade_432 | N | N |
| Propionibacterium freudenreichii | 1572 | NR_036972 | clade_433 | N | N |
| Propionibacterium sp. oral taxon 192 | 1580 | GQ422728 | clade_433 | N | N |
| Tessaracoccus sp. oral taxon F04 | 1917 | HM099640 | clade_433 | N | N |
| Peptoniphilus ivorii | 1445 | Y07840 | clade_434 | N | N |
| Peptoniphilus sp. gpac007 | 1448 | AM176517 | clade_434 | N | N |
| Peptoniphilus sp. gpac018A | 1449 | AM176519 | clade_434 | N | N |
| Peptoniphilus sp. gpac148 | 1451 | AM176535 | clade_434 | N | N |
| Flexispira rappini | 887 | AY126479 | clade_436 | N | N |
| Helicobacter bilis | 993 | ACDN01000023 | clade_436 | N | N |
| Helicobacter cinaedi | 995 | ABQT01000054 | clade_436 | N | N |
| Helicobacter sp. None | 998 | U44756 | clade_436 | N | N |
| Brevundimonas subvibrioides | 429 | CP002102 | clade_438 | N | N |
| Hyphomonas neptunium | 1008 | NR_074092 | clade_438 | N | N |
| Phenylobacterium zucineum | 1465 | AY628697 | clade_438 | N | N |
| Streptococcus downei | 1793 | AEKN01000002 | clade_441 | N | N |
| Streptococcus sp. SHV515 | 1848 | Y07601 | clade_441 | N | N |
| Acinetobacter sp. CIP 53.82 | 40 | JQ638584 | clade_443 | N | N |
| Halomonas elongata | 990 | NR_074782 | clade_443 | N | N |
| Halomonas johnsoniae | 991 | FR775979 | clade_443 | N | N |
| Butyrivibrio fibrisolvens | 456 | U41172 | clade_444 | N | N |
| Roseburia sp. 11SE37 | 1640 | FM954975 | clade_444 | N | N |
| Roseburia sp. 11SE38 | 1641 | FM954976 | clade_444 | N | N |
| Shuttleworthia satelles | 1728 | ACIP02000004 | clade_444 | N | N |
| Shuttleworthia sp. MSX8B | 1729 | HQ616383 | clade_444 | N | N |
| Shuttleworthia sp. oral taxon G69 | 1730 | GU432167 | clade_444 | N | N |
| Bdellovibrio sp. MPA | 344 | AY294215 | clade_445 | N | N |
| Desulfobulbus sp. oral clone CH031 | 755 | AY005036 | clade_445 | N | N |
| Desulfovibrio desulfuricans | 757 | DQ092636 | clade_445 | N | N |
| Desulfovibrio fairfieldensis | 758 | U42221 | clade_445 | N | N |
| Desulfovibrio piger | 759 | AF192152 | clade_445 | N | N |
| Desulfovibrio sp. 3_1_syn3 | 760 | ADDR01000239 | clade_445 | N | N |
| Geobacter bemidjiensis | 941 | CP001124 | clade_445 | N | N |
| Brachybacterium alimentarium | 401 | NR_026269 | clade_446 | N | N |
| Brachybacterium conglomeratum | 402 | AB537169 | clade_446 | N | N |
| Brachybacterium tyrofermentans | 403 | NR_026272 | clade_446 | N | N |
| Dermabacter hominis | 749 | FJ263375 | clade_446 | N | N |
| Aneurinibacillus thermoaerophilus | 171 | NR_029303 | clade_448 | N | N |
| Brevibacillus agri | 409 | NR_040983 | clade_448 | N | N |
| Brevibacillus centrosporus | 411 | NR_043414 | clade_448 | N | N |
| Brevibacillus choshinensis | 412 | NR_040980 | clade_448 | N | N |
| Brevibacillus invocatus | 413 | NR_041836 | clade_448 | N | N |
| Brevibacillus parabrevis | 415 | NR_040981 | clade_448 | N | N |
| Brevibacillus reuszeri | 416 | NR_040982 | clade_448 | N | N |
| Brevibacillus sp. phR | 417 | JN837488 | clade_448 | N | N |
| Brevibacillus thermoruber | 418 | NR_026514 | clade_448 | N | N |
| Lactobacillus murinus | 1100 | NR_042231 | clade_449 | N | N |
| Lactobacillus oeni | 1102 | NR_043095 | clade_449 | N | N |
| Lactobacillus ruminis | 1115 | ACGS02000043 | clade_449 | N | N |
| Lactobacillus vini | 1141 | NR_042196 | clade_449 | N | N |
| Gemella haemolysans | 924 | ACDZ02000012 | clade_450 | N | N |
| Gemella morbillorum | 925 | NR_025904 | clade_450 | N | N |
| Gemella morbillorum | 926 | ACRX01000010 | clade_450 | N | N |
| Gemella sanguinis | 927 | ACRY01000057 | clade_450 | N | N |
| Gemella sp. oral clone ASCE02 | 929 | AY923133 | clade_450 | N | N |
| Gemella sp. oral clone ASCF04 | 930 | AY923139 | clade_450 | N | N |
| Gemella sp. oral clone ASCF12 | 931 | AY923143 | clade_450 | N | N |
| Gemella sp. WAL 1945J | 928 | EU427463 | clade_450 | N | N |
| Sporolactobacillus nakayamae | 1753 | NR_042247 | clade_451 | N | N |
| Gluconacetobacter entanii | 945 | NR_028909 | clade_452 | N | N |
| Gluconacetobacter europaeus | 946 | NR_026513 | clade_452 | N | N |
| Gluconacetobacter hansenii | 947 | NR_026133 | clade_452 | N | N |
| Gluconacetobacter oboediens | 949 | NR_041295 | clade_452 | N | N |
| Gluconacetobacter xylinus | 950 | NR_074338 | clade_452 | N | N |
| Auritibacter ignavus | 193 | FN554542 | clade_453 | N | N |
| Dermacoccus sp. Ellin185 | 750 | AEIQ01000090 | clade_453 | N | N |
| Janibacter limosus | 1013 | NR_026362 | clade_453 | N | N |
| Janibacter melonis | 1014 | EF063716 | clade_453 | N | N |
| Acetobacter aceti | 7 | NR_026121 | clade_454 | N | N |
| Acetobacter fabarum | 8 | NR_042678 | clade_454 | N | N |
| Acetobacter lovaniensis | 9 | NR_040832 | clade_454 | N | N |
| Acetobacter malorum | 10 | NR_025513 | clade_454 | N | N |
| Acetobacter orientalis | 11 | NR_028625 | clade_454 | N | N |
| Acetobacter pasteurianus | 12 | NR_026107 | clade_454 | N | N |
| Acetobacter pomorum | 13 | NR_042112 | clade_454 | N | N |
| Acetobacter syzygii | 14 | NR_040868 | clade_454 | N | N |
| Acetobacter tropicalis | 15 | NR_036881 | clade_454 | N | N |
| Gluconacetobacter azotocaptans | 943 | NR_028767 | clade_454 | N | N |
| Gluconacetobacter diazotrophicus | 944 | NR_074292 | clade_454 | N | N |
| Gluconacetobacter johannae | 948 | NR_024959 | clade_454 | N | N |
| Nocardia brasiliensis | 1351 | AIHV01000038 | clade_455 | N | N |
| Nocardia cyriacigeorgica | 1352 | HQ009486 | clade_455 | N | N |
| Nocardia puris | 1354 | NR_028994 | clade_455 | N | N |
| Nocardia sp. 01_Je_025 | 1355 | GU574059 | clade_455 | N | N |
| Rhodococcus equi | 1623 | ADNW01000058 | clade_455 | N | N |
| Oceanobacillus caeni | 1358 | NR_041533 | clade_456 | N | N |
| Oceanobacillus sp. Ndiop | 1359 | CAER01000083 | clade_456 | N | N |
| Ornithinibacillus bavariensis | 1384 | NR_044923 | clade_456 | N | N |
| Ornithinibacillus sp. 7_10AIA | 1385 | FN397526 | clade_456 | N | N |
| Virgibacillus proomii | 2005 | NR_025308 | clade_456 | N | N |
| Corynebacterium amycolatum | 683 | ABZU01000033 | clade_457 | N | OP |
| Corynebacterium hansenii | 702 | AM946639 | clade_457 | N | N |
| Corynebacterium xerosis | 735 | FN179330 | clade_457 | N | OP |
| Staphylococcaceae bacterium NML 92_0017 | 1756 | AY841362 | clade_458 | N | N |
| Staphylococcus fleurettii | 1766 | NR_041326 | clade_458 | N | N |
| Staphylococcus sciuri | 1774 | NR_025520 | clade_458 | N | N |
| Staphylococcus vitulinus | 1779 | NR_024670 | clade_458 | N | N |
| Stenotrophomonas maltophilia | 1782 | AAVZ01000005 | clade_459 | N | N |
| Stenotrophomonas sp. FG_6 | 1783 | EF017810 | clade_459 | N | N |
| Mycobacterium africanum | 1270 | AF480605 | clade_46 | N | OP |
| Mycobacterium alsiensis | 1271 | AJ938169 | clade_46 | N | OP |
| Mycobacterium avium | 1272 | CP000479 | clade_46 | N | OP |
| Mycobacterium colombiense | 1274 | AM062764 | clade_46 | N | OP |
| Mycobacterium gordonae | 1276 | GU142930 | clade_46 | N | OP |
| Mycobacterium intracellulare | 1277 | GQ153276 | clade_46 | N | OP |
| Mycobacterium kansasii | 1278 | AF480601 | clade_46 | N | OP |
| Mycobacterium lacus | 1279 | NR_025175 | clade_46 | N | OP |
| Mycobacterium leprae | 1280 | FM211192 | clade_46 | N | OP |
| Mycobacterium lepromatosis | 1281 | EU203590 | clade_46 | N | OP |
| Mycobacterium mantenii | 1283 | FJ042897 | clade_46 | N | OP |
| Mycobacterium marinum | 1284 | NC_010612 | clade_46 | N | OP |
| Mycobacterium microti | 1285 | NR_025234 | clade_46 | N | OP |
| Mycobacterium parascrofulaceum | 1287 | ADNV01000350 | clade_46 | N | OP |
| Mycobacterium seoulense | 1290 | DQ536403 | clade_46 | N | OP |
| Mycobacterium sp. 1761 | 1292 | EU703150 | clade_46 | N | N |
| Mycobacterium sp. 1791 | 1295 | EU703148 | clade_46 | N | N |
| Mycobacterium sp. 1797 | 1296 | EU703149 | clade_46 | N | N |
| Mycobacterium sp. B10_07.09.0206 | 1298 | HQ174245 | clade_46 | N | N |
| Mycobacterium sp. NLA001000736 | 1305 | HM627011 | clade_46 | N | N |
| Mycobacterium sp. W | 1306 | DQ437715 | clade_46 | N | N |
| Mycobacterium tuberculosis | 1307 | CP001658 | clade_46 | N | Category-C |
| Mycobacterium ulcerans | 1308 | AB548725 | clade_46 | N | OP |
| Mycobacterium vulneris | 1309 | EU834055 | clade_46 | N | OP |
| Xanthomonas campestris | 2016 | EF101975 | clade_461 | N | N |
| Xanthomonas sp. kmd_489 | 2017 | EU723184 | clade_461 | N | N |
| Dietzia natronolimnaea | 769 | GQ870426 | clade_462 | N | N |
| Dietzia sp. BBDP51 | 770 | DQ337512 | clade_462 | N | N |
| Dietzia sp. CA149 | 771 | GQ870422 | clade_462 | N | N |
| Dietzia timorensis | 772 | GQ870424 | clade_462 | N | N |
| Gordonia bronchialis | 951 | NR_027594 | clade_463 | N | N |
| Gordonia polyisoprenivorans | 952 | DQ385609 | clade_463 | N | N |
| Gordonia sp. KTR9 | 953 | DQ068383 | clade_463 | N | N |
| Gordonia sputi | 954 | FJ536304 | clade_463 | N | N |
| Gordonia terrae | 955 | GQ848239 | clade_463 | N | N |
| Leptotrichia goodfellowii | 1167 | ADAD01000110 | clade_465 | N | N |
| Leptotrichia sp. oral clone IK040 | 1174 | AY349387 | clade_465 | N | N |
| Leptotrichia sp. oral clone P2PB_51 P1 | 1175 | AY207053 | clade_465 | N | N |
| Bacteroidales genomosp. P7 oral clone MB3_P19 | 264 | DQ003623 | clade_466 | N | N |
| Butyricimonas virosa | 454 | AB443949 | clade_466 | N | N |
| Odoribacter laneus | 1363 | AB490805 | clade_466 | N | N |
| Odoribacter splanchnicus | 1364 | CP002544 | clade_466 | N | N |
| Capnocytophaga gingivalis | 478 | ACLQ01000011 | clade_467 | N | N |
| Capnocytophaga granulosa | 479 | X97248 | clade_467 | N | N |
| Capnocytophaga sp. oral clone AH015 | 483 | AY005074 | clade_467 | N | N |
| Capnocytophaga sp. oral strain S3 | 487 | AY005073 | clade_467 | N | N |
| Capnocytophaga sp. oral taxon 338 | 488 | AEXX01000050 | clade_467 | N | N |
| Capnocytophaga canimorsus | 476 | CP002113 | clade_468 | N | N |
| Capnocytophaga sp. oral clone ID062 | 485 | AY349368 | clade_468 | N | N |
| Lactobacillus catenaformis | 1075 | M23729 | clade_469 | N | N |
| Lactobacillus vitulinus | 1142 | NR_041305 | clade_469 | N | N |
| Cetobacterium somerae | 501 | AJ438155 | clade_470 | N | N |
| Fusobacterium gonidiaformans | 896 | ACET01000043 | clade_470 | N | N |
| Fusobacterium mortiferum | 897 | ACDB02000034 | clade_470 | N | N |
| Fusobacterium necrogenes | 899 | X55408 | clade_470 | N | N |
| Fusobacterium necrophorum | 900 | AM905356 | clade_470 | N | N |
| Fusobacterium sp. 12_1B | 905 | AGWJ01000070 | clade_470 | N | N |
| Fusobacterium sp. 3_1_5R | 911 | ACDD01000078 | clade_470 | N | N |
| Fusobacterium sp. D12 | 918 | ACDG02000036 | clade_470 | N | N |
| Fusobacterium ulcerans | 921 | ACDH01000090 | clade_470 | N | N |
| Fusobacterium varium | 922 | ACIE01000009 | clade_470 | N | N |
| Mycoplasma arthritidis | 1312 | NC_011025 | clade_473 | N | N |
| Mycoplasma faucium | 1314 | NR_024983 | clade_473 | N | N |
| Mycoplasma hominis | 1318 | AF443616 | clade_473 | N | N |
| Mycoplasma orale | 1319 | AY796060 | clade_473 | N | N |
| Mycoplasma salivarium | 1324 | M24661 | clade_473 | N | N |
| Mitsuokella jalaludinii | 1247 | NR_028840 | clade_474 | N | N |
| Mitsuokella multacida | 1248 | ABWK02000005 | clade_474 | N | N |
| Mitsuokella sp. oral taxon 521 | 1249 | GU413658 | clade_474 | N | N |
| Mitsuokella sp. oral taxon G68 | 1250 | GU432166 | clade_474 | N | N |
| Selenomonas genomosp. C1 | 1695 | AY278627 | clade_474 | N | N |
| Selenomonas genomosp. P8 oral clone MB5_P06 | 1700 | DQ003628 | clade_474 | N | N |
| Selenomonas ruminantium | 1703 | NR_075026 | clade_474 | N | N |
| Veillonellaceae bacterium oral taxon 131 | 1994 | GU402916 | clade_474 | N | N |
| Alloscardovia omnicolens | 139 | NR_042583 | clade_475 | N | N |
| Alloscardovia sp. OB7196 | 140 | AB425070 | clade_475 | N | N |
| Bifidobacterium urinalis | 366 | AJ278695 | clade_475 | N | N |
| Prevotella loescheii | 1503 | JN867231 | clade_48 | N | N |
| Prevotella sp. oral clone ASCG12 | 1530 | DQ272511 | clade_48 | N | N |
| Prevotella sp. oral clone GU027 | 1540 | AY349398 | clade_48 | N | N |
| Prevotella sp. oral taxon 472 | 1553 | ACZS01000106 | clade_48 | N | N |
| Selenomonas dianae | 1693 | GQ422719 | clade_480 | N | N |
| Selenomonas flueggei | 1694 | AF287803 | clade_480 | N | N |
| Selenomonas genomosp. C2 | 1696 | AY278628 | clade_480 | N | N |
| Selenomonas genomosp. P6 oral clone MB3_C41 | 1698 | DQ003636 | clade_480 | N | N |
| Selenomonas genomosp. P7 oral clone MB5_C08 | 1699 | DQ003627 | clade_480 | N | N |
| Selenomonas infelix | 1701 | AF287802 | clade_480 | N | N |
| Selenomonas noxia | 1702 | GU470909 | clade_480 | N | N |
| Selenomonas sp. oral clone FT050 | 1705 | AY349403 | clade_480 | N | N |
| Selenomonas sp. oral clone GI064 | 1706 | AY349404 | clade_480 | N | N |
| Selenomonas sp. oral clone GT010 | 1707 | AY349405 | clade_480 | N | N |
| Selenomonas sp. oral clone HU051 | 1708 | AY349406 | clade_480 | N | N |
| Selenomonas sp. oral clone IK004 | 1709 | AY349407 | clade_480 | N | N |
| Selenomonas sp. oral clone JI021 | 1711 | AY349409 | clade_480 | N | N |
| Selenomonas sp. oral clone JS031 | 1712 | AY349410 | clade_480 | N | N |
| Selenomonas sp. oral clone OH4A | 1713 | AY947498 | clade_480 | N | N |
| Selenomonas sp. oral clone P2PA_80 P4 | 1714 | AY207052 | clade_480 | N | N |
| Selenomonas sp. oral taxon 149 | 1716 | AEEJ01000007 | clade_480 | N | N |
| Veillonellaceae bacterium oral taxon 155 | 1995 | GU470897 | clade_480 | N | N |
| Agrococcus jenensis | 117 | NR_026275 | clade_484 | N | N |
| Microbacterium gubbeenense | 1232 | NR_025098 | clade_484 | N | N |
| Pseudoclavibacter sp. Timone | 1590 | FJ375951 | clade_484 | N | N |
| Tropheryma whipplei | 1961 | BX251412 | clade_484 | N | N |
| Zimmermannella bifida | 2031 | AB012592 | clade_484 | N | N |
| Legionella hackeliae | 1151 | M36028 | clade_486 | N | OP |
| Legionella longbeachae | 1152 | M36029 | clade_486 | N | OP |
| Legionella sp. D3923 | 1154 | JN380999 | clade_486 | N | OP |
| Legionella sp. D4088 | 1155 | JN381012 | clade_486 | N | OP |
| Legionella sp. H63 | 1156 | JF831047 | clade_486 | N | OP |
| Legionella sp. NML 93L054 | 1157 | GU062706 | clade_486 | N | OP |
| Legionella steelei | 1158 | HQ398202 | clade_486 | N | OP |
| Tatlockia micdadei | 1915 | M36032 | clade_486 | N | N |
| Helicobacter pullorum | 996 | ABQU01000097 | clade_489 | N | N |
| Acetobacteraceae bacterium AT 5844 | 16 | AGEZ01000040 | clade_490 | N | N |
| Roseomonas cervicalis | 1643 | ADVL01000363 | clade_490 | N | N |
| Roseomonas mucosa | 1644 | NR_028857 | clade_490 | N | N |
| Roseomonas sp. NML94_0193 | 1645 | AF533357 | clade_490 | N | N |
| Roseomonas sp. NML97_0121 | 1646 | AF533359 | clade_490 | N | N |
| Roseomonas sp. NML98_0009 | 1647 | AF533358 | clade_490 | N | N |
| Roseomonas sp. NML98_0157 | 1648 | AF533360 | clade_490 | N | N |
| Rickettsia akari | 1627 | CP000847 | clade_492 | N | OP |
| Rickettsia conorii | 1628 | AE008647 | clade_492 | N | OP |
| Rickettsia prowazekii | 1629 | M21789 | clade_492 | N | Category-B |
| Rickettsia rickettsii | 1630 | NC_010263 | clade_492 | N | OP |
| Rickettsia slovaca | 1631 | L36224 | clade_492 | N | OP |
| Rickettsia typhi | 1632 | AE017197 | clade_492 | N | OP |
| Anaeroglobus geminatus | 160 | AGCJ01000054 | clade_493 | N | N |
| Megasphaera genomosp. C1 | 1201 | AY278622 | clade_493 | N | N |
| Megasphaera micronuciformis | 1203 | AECS01000020 | clade_493 | N | N |
| Clostridiales genomosp. BVAB3 | 540 | CP001850 | clade_495 | N | N |
| Tsukamurella paurometabola | 1963 | X80628 | clade_496 | N | N |
| Tsukamurella tyrosinosolvens | 1964 | AB478958 | clade_496 | N | N |
| Abiotrophia para_adiacens | 2 | AB022027 | clade_497 | N | N |
| Carnobacterium divergens | 492 | NR_044706 | clade_497 | N | N |
| Carnobacterium maltaromaticum | 493 | NC_019425 | clade_497 | N | N |
| Enterococcus avium | 800 | AF133535 | clade_497 | N | N |
| Enterococcus caccae | 801 | AY943820 | clade_497 | N | N |
| Enterococcus casseliflavus | 802 | AEWT01000047 | clade_497 | N | N |
| Enterococcus durans | 803 | AJ276354 | clade_497 | N | N |
| Enterococcus faecalis | 804 | AE016830 | clade_497 | N | N |
| Enterococcus faecium | 805 | AM157434 | clade_497 | N | N |
| Enterococcus gallinarum | 806 | AB269767 | clade_497 | N | N |
| Enterococcus gilvus | 807 | AY033814 | clade_497 | N | N |
| Enterococcus hawaiiensis | 808 | AY321377 | clade_497 | N | N |
| Enterococcus hirae | 809 | AF061011 | clade_497 | N | N |
| Enterococcus italicus | 810 | AEPV01000109 | clade_497 | N | N |
| Enterococcus mundtii | 811 | NR_024906 | clade_497 | N | N |
| Enterococcus raffinosus | 812 | FN600541 | clade_497 | N | N |
| Enterococcus sp. BV2CASA2 | 813 | JN809766 | clade_497 | N | N |
| Enterococcus sp. CCRI_16620 | 814 | GU457263 | clade_497 | N | N |
| Enterococcus sp. F95 | 815 | FJ463817 | clade_497 | N | N |
| Enterococcus sp. RfL6 | 816 | AJ133478 | clade_497 | N | N |
| Enterococcus thailandicus | 817 | AY321376 | clade_497 | N | N |
| Fusobacterium canifelinum | 893 | AY162222 | clade_497 | N | N |
| Fusobacterium genomosp. C1 | 894 | AY278616 | clade_497 | N | N |
| Fusobacterium genomosp. C2 | 895 | AY278617 | clade_497 | N | N |
| Fusobacterium periodonticum | 902 | ACJY01000002 | clade_497 | N | N |
| Fusobacterium sp. 1_1_41FAA | 906 | ADGG01000053 | clade_497 | N | N |
| Fusobacterium sp. 11_3_2 | 904 | ACUO01000052 | clade_497 | N | N |
| Fusobacterium sp. 2_1_31 | 907 | ACDC02000018 | clade_497 | N | N |
| Fusobacterium sp. 3_1_27 | 908 | ADGF01000045 | clade_497 | N | N |
| Fusobacterium sp. 3_1_33 | 909 | ACQE01000178 | clade_497 | N | N |
| Fusobacterium sp. 3_1_36A2 | 910 | ACPU01000044 | clade_497 | N | N |
| Fusobacterium sp. AC18 | 912 | HQ616357 | clade_497 | N | N |
| Fusobacterium sp. ACB2 | 913 | HQ616358 | clade_497 | N | N |
| Fusobacterium sp. AS2 | 914 | HQ616361 | clade_497 | N | N |
| Fusobacterium sp. CM1 | 915 | HQ616371 | clade_497 | N | N |
| Fusobacterium sp. CM21 | 916 | HQ616375 | clade_497 | N | N |
| Fusobacterium sp. CM22 | 917 | HQ616376 | clade_497 | N | N |
| Fusobacterium sp. oral clone ASCF06 | 919 | AY923141 | clade_497 | N | N |
| Fusobacterium sp. oral clone ASCF11 | 920 | AY953256 | clade_497 | N | N |
| Granulicatella adiacens | 959 | ACKZ01000002 | clade_497 | N | N |
| Granulicatella elegans | 960 | AB252689 | clade_497 | N | N |
| Granulicatella paradiacens | 961 | AY879298 | clade_497 | N | N |
| Granulicatella sp. oral clone ASC02 | 963 | AY923126 | clade_497 | N | N |
| Granulicatella sp. oral clone ASCA05 | 964 | DQ341469 | clade_497 | N | N |
| Granulicatella sp. oral clone ASCB09 | 965 | AY953251 | clade_497 | N | N |
| Granulicatella sp. oral clone ASCG05 | 966 | AY923146 | clade_497 | N | N |
| Tetragenococcus halophilus | 1918 | NR_075020 | clade_497 | N | N |
| Tetragenococcus koreensis | 1919 | NR_043113 | clade_497 | N | N |
| Vagococcus fluvialis | 1973 | NR_026489 | clade_497 | N | N |
| Chryseobacterium anthropi | 514 | AM982793 | clade_498 | N | N |
| Chryseobacterium gleum | 515 | ACKQ02000003 | clade_498 | N | N |
| Chryseobacterium hominis | 516 | NR_042517 | clade_498 | N | N |
| Treponema refringens | 1936 | AF426101 | clade_499 | N | OP |
| Treponema sp. oral clone JU031 | 1941 | AY349416 | clade_499 | N | N |
| Treponema sp. oral taxon 239 | 1948 | GU408738 | clade_499 | N | N |
| Treponema sp. oral taxon 271 | 1955 | GU408871 | clade_499 | N | N |
| Alistipes finegoldii | 129 | NR_043064 | clade_500 | N | N |
| Alistipes onderdonkii | 131 | NR_043318 | clade_500 | N | N |
| Alistipes putredinis | 132 | ABFK02000017 | clade_500 | N | N |
| Alistipes shahii | 133 | FP929032 | clade_500 | N | N |
| Alistipes sp. HGB5 | 134 | AENZ01000082 | clade_500 | N | N |
| Alistipes sp. JC50 | 135 | JF824804 | clade_500 | N | N |
| Alistipes sp. RMA 9912 | 136 | GQ140629 | clade_500 | N | N |
| Mycoplasma agalactiae | 1310 | AF010477 | clade_501 | N | N |
| Mycoplasma bovoculi | 1313 | NR_025987 | clade_501 | N | N |
| Mycoplasma fermentans | 1315 | CP002458 | clade_501 | N | N |
| Mycoplasma flocculare | 1316 | X62699 | clade_501 | N | N |
| Mycoplasma ovipneumoniae | 1320 | NR_025989 | clade_501 | N | N |
| Arcobacter butzleri | 176 | AEPT01000071 | clade_502 | N | N |
| Arcobacter cryaerophilus | 177 | NR_025905 | clade_502 | N | N |
| Campylobacter curvus | 461 | NC_009715 | clade_502 | N | OP |
| Campylobacter rectus | 467 | ACFU01000050 | clade_502 | N | OP |
| Campylobacter showae | 468 | ACVQ01000030 | clade_502 | N | OP |
| Campylobacter sp. FOBRC14 | 469 | HQ616379 | clade_502 | N | OP |
| Campylobacter sp. FOBRC15 | 470 | HQ616380 | clade_502 | N | OP |
| Campylobacter sp. oral clone BB120 | 471 | AY005038 | clade_502 | N | OP |
| Campylobacter sputorum | 472 | NR_044839 | clade_502 | N | OP |
| Bacteroides ureolyticus | 330 | GQ167666 | clade_504 | N | N |
| Campylobacter gracilis | 463 | ACYG01000026 | clade_504 | N | OP |
| Campylobacter hominis | 464 | NC_009714 | clade_504 | N | OP |
| Dialister invisus | 762 | ACIM02000001 | clade_506 | N | N |
| Dialister micraerophilus | 763 | AFBB01000028 | clade_506 | N | N |
| Dialister microaerophilus | 764 | AENT01000008 | clade_506 | N | N |
| Dialister propionicifaciens | 766 | NR_043231 | clade_506 | N | N |
| Dialister succinatiphilus | 768 | AB370249 | clade_506 | N | N |
| Megasphaera elsdenii | 1200 | AY038996 | clade_506 | N | N |
| Megasphaera genomosp. type_1 | 1202 | ADGP01000010 | clade_506 | N | N |
| Megasphaera sp. BLPYG_07 | 1204 | HM990964 | clade_506 | N | N |
| Megasphaera sp. UPII 199_6 | 1205 | AFIJ01000040 | clade_506 | N | N |
| Chromobacterium violaceum | 513 | NC_005085 | clade_507 | N | N |
| Laribacter hongkongensis | 1148 | CP001154 | clade_507 | N | N |
| Methylophilus sp. ECd5 | 1229 | AY436794 | clade_507 | N | N |
| Finegoldia magna | 883 | ACHM02000001 | clade_509 | N | N |
| Parvimonas micra | 1431 | AB729072 | clade_509 | N | N |
| Parvimonas sp. oral taxon 110 | 1432 | AFII01000002 | clade_509 | N | N |
| Peptostreptococcus micros | 1456 | AM176538 | clade_509 | N | N |
| Peptostreptococcus sp. oral clone FJ023 | 1460 | AY349390 | clade_509 | N | N |
| Peptostreptococcus sp. P4P_31 P3 | 1458 | AY207059 | clade_509 | N | N |
| Helicobacter pylori | 997 | CP000012 | clade_510 | N | OP |
| Anaplasma marginale | 165 | ABOR01000019 | clade_511 | N | N |
| Anaplasma phagocytophilum | 166 | NC_007797 | clade_511 | N | N |
| Ehrlichia chaffeensis | 783 | AAIF01000035 | clade_511 | N | OP |
| Neorickettsia risticii | 1349 | CP001431 | clade_511 | N | N |
| Neorickettsia sennetsu | 1350 | NC_007798 | clade_511 | N | N |
| Pseudoramibacter alactolyticus | 1606 | AB036759 | clade_512 | N | N |
| Veillonella montpellierensis | 1977 | AF473836 | clade_513 | N | N |
| Veillonella sp. oral clone ASCA08 | 1988 | AY923118 | clade_513 | N | N |
| Veillonella sp. oral clone ASCB03 | 1989 | AY923122 | clade_513 | N | N |
| Inquilinus limosus | 1012 | NR_029046 | clade_514 | N | N |
| Sphingomonas sp. oral clone FZ016 | 1746 | AY349412 | clade_514 | N | N |
| Anaerococcus lactolyticus | 145 | ABYO01000217 | clade_515 | N | N |
| Anaerococcus prevotii | 147 | CP001708 | clade_515 | N | N |
| Anaerococcus sp. gpac104 | 152 | AM176528 | clade_515 | N | N |
| Anaerococcus sp. gpac126 | 153 | AM176530 | clade_515 | N | N |
| Anaerococcus sp. gpac155 | 154 | AM176536 | clade_515 | N | N |
| Anaerococcus sp. gpac199 | 155 | AM176539 | clade_515 | N | N |
| Anaerococcus tetradius | 157 | ACGC01000107 | clade_515 | N | N |
| Bacteroides coagulans | 271 | AB547639 | clade_515 | N | N |
| Clostridiales bacterium 9403326 | 534 | HM587324 | clade_515 | N | N |
| Clostridiales bacterium ph2 | 539 | JN837487 | clade_515 | N | N |
| Peptostreptococcus sp. 9succ1 | 1457 | X90471 | clade_515 | N | N |
| Peptostreptococcus sp. oral clone AP24 | 1459 | AB175072 | clade_515 | N | N |
| Tissierella praeacuta | 1924 | NR_044860 | clade_515 | N | N |
| Helicobacter canadensis | 994 | ABQS01000108 | clade_518 | N | N |
| Peptostreptococcus anaerobius | 1455 | AY326462 | clade_520 | N | N |
| Peptostreptococcus stomatis | 1461 | ADGQ01000048 | clade_520 | N | N |
| Bilophila wadsworthia | 367 | ADCP01000166 | clade_521 | N | N |
| Desulfovibrio vulgaris | 761 | NR_074897 | clade_521 | N | N |
| Actinomyces nasicola | 64 | AJ508455 | clade_523 | N | N |
| Cellulosimicrobium funkei | 500 | AY501364 | clade_523 | N | N |
| Lactococcus raffinolactis | 1146 | NR_044359 | clade_524 | N | N |
| Bacteroidales genomosp. P1 | 258 | AY341819 | clade_529 | N | N |
| Bacteroidales genomosp. P2 oral clone MB1_G13 | 259 | DQ003613 | clade_529 | N | N |
| Bacteroidales genomosp. P3 oral clone MB1_G34 | 260 | DQ003615 | clade_529 | N | N |
| Bacteroidales genomosp. P4 oral clone MB2_G17 | 261 | DQ003617 | clade_529 | N | N |
| Bacteroidales genomosp. P5 oral clone MB2_P04 | 262 | DQ003619 | clade_529 | N | N |
| Bacteroidales genomosp. P6 oral clone MB3_C19 | 263 | DQ003634 | clade_529 | N | N |
| Bacteroidales genomosp. P8 oral clone MB4_G15 | 265 | DQ003626 | clade_529 | N | N |
| Bacteroidetes bacterium oral taxon D27 | 333 | HM099638 | clade_530 | N | N |
| Bacteroidetes bacterium oral taxon F31 | 334 | HM099643 | clade_530 | N | N |
| Bacteroidetes bacterium oral taxon F44 | 335 | HM099649 | clade_530 | N | N |
| Flavobacterium sp. NF2_1 | 885 | FJ195988 | clade_530 | N | N |
| Myroides odoratimimus | 1326 | NR_042354 | clade_530 | N | N |
| Myroides sp. MY15 | 1327 | GU253339 | clade_530 | N | N |
| Chlamydiales bacterium NS16 | 507 | JN606076 | clade_531 | N | N |
| Chlamydophila pecorum | 508 | D88317 | clade_531 | N | OP |
| Parachlamydia sp. UWE25 | 1423 | BX908798 | clade_531 | N | N |
| Fusobacterium russii | 903 | NR_044687 | clade_532 | N | N |
| Streptobacillus moniliformis | 1784 | NR_027615 | clade_532 | N | N |
| Eubacteriaceae bacterium P4P_50 P4 | 833 | AY207060 | clade_533 | N | N |
| Abiotrophia defectiva | 1 | ACIN02000016 | clade_534 | N | N |
| Abiotrophia sp. oral clone P4PA_155 P1 | 3 | AY207063 | clade_534 | N | N |
| Catonella genomosp. P1 oral clone MB5_P12 | 496 | DQ003629 | clade_534 | N | N |
| Catonella morbi | 497 | ACIL02000016 | clade_534 | N | N |
| Catonella sp. oral clone FL037 | 498 | AY349369 | clade_534 | N | N |
| Eremococcus coleocola | 818 | AENN01000008 | clade_534 | N | N |
| Facklamia hominis | 879 | Y10772 | clade_534 | N | N |
| Granulicatella sp. M658_99_3 | 962 | AJ271861 | clade_534 | N | N |
| Campylobacter coli | 459 | AAFL01000004 | clade_535 | N | OP |
| Campylobacter concisus | 460 | CP000792 | clade_535 | N | OP |
| Campylobacter fetus | 462 | ACLG01001177 | clade_535 | N | OP |
| Campylobacter jejuni | 465 | AL139074 | clade_535 | N | Category-B |
| Campylobacter upsaliensis | 473 | AEPU01000040 | clade_535 | N | OP |
| Atopobium minutum | 183 | HM007583 | clade_539 | N | N |
| Atopobium parvulum | 184 | CP001721 | clade_539 | N | N |
| Atopobium rimae | 185 | ACFE01000007 | clade_539 | N | N |
| Atopobium sp. BS2 | 186 | HQ616367 | clade_539 | N | N |
| Atopobium sp. F0209 | 187 | EU592966 | clade_539 | N | N |
| Atopobium sp. ICM42b10 | 188 | HQ616393 | clade_539 | N | N |
| Atopobium sp. ICM57 | 189 | HQ616400 | clade_539 | N | N |
| Atopobium vaginae | 190 | AEDQ01000024 | clade_539 | N | N |
| Coriobacteriaceae bacterium BV3Ac1 | 677 | JN809768 | clade_539 | N | N |
| Actinomyces naeslundii | 63 | X81062 | clade_54 | N | N |
| Actinomyces oricola | 67 | NR_025559 | clade_54 | N | N |
| Actinomyces oris | 69 | BABV01000070 | clade_54 | N | N |
| Actinomyces sp. 7400942 | 70 | EU484334 | clade_54 | N | N |
| Actinomyces sp. ChDC B197 | 72 | AF543275 | clade_54 | N | N |
| Actinomyces sp. GEJ15 | 73 | GU561313 | clade_54 | N | N |
| Actinomyces sp. M2231_94_1 | 79 | AJ234063 | clade_54 | N | N |
| Actinomyces sp. oral clone GU067 | 83 | AY349362 | clade_54 | N | N |
| Actinomyces sp. oral clone IO077 | 85 | AY349364 | clade_54 | N | N |
| Actinomyces sp. oral clone IP073 | 86 | AY349365 | clade_54 | N | N |
| Actinomyces sp. oral clone JA063 | 88 | AY349367 | clade_54 | N | N |
| Actinomyces sp. oral taxon 170 | 89 | AFBL01000010 | clade_54 | N | N |
| Actinomyces sp. oral taxon 171 | 90 | AECW01000034 | clade_54 | N | N |
| Actinomyces urogenitalis | 95 | ACFH01000038 | clade_54 | N | N |
| Actinomyces viscosus | 96 | ACRE01000096 | clade_54 | N | N |
| Orientia tsutsugamushi | 1383 | AP008981 | clade_541 | N | OP |
| Megamonas funiformis | 1198 | AB300988 | clade_542 | N | N |
| Megamonas hypermegale | 1199 | AJ420107 | clade_542 | N | N |
| Aeromicrobium marinum | 102 | NR_025681 | clade_544 | N | N |
| Aeromicrobium sp. JC14 | 103 | JF824798 | clade_544 | N | N |
| Luteococcus sanguinis | 1190 | NR_025507 | clade_544 | N | N |
| Propionibacteriaceae bacterium NML 02_0265 | 1568 | EF599122 | clade_544 | N | N |
| Rhodococcus corynebacterioides | 1622 | X80615 | clade_546 | N | N |
| Rhodococcus erythropolis | 1624 | ACNO01000030 | clade_546 | N | N |
| Rhodococcus fascians | 1625 | NR_037021 | clade_546 | N | N |
| Segniliparus rotundus | 1690 | CP001958 | clade_546 | N | N |
| Segniliparus rugosus | 1691 | ACZI01000025 | clade_546 | N | N |
| Exiguobacterium acetylicum | 878 | FJ970034 | clade_547 | N | N |
| Macrococcus caseolyticus | 1194 | NR_074941 | clade_547 | N | N |
| Streptomyces sp. 1 AIP_2009 | 1890 | FJ176782 | clade_548 | N | N |
| Streptomyces sp. SD 524 | 1892 | EU544234 | clade_548 | N | N |
| Streptomyces sp. SD 528 | 1893 | EU544233 | clade_548 | N | N |
| Streptomyces thermoviolaceus | 1895 | NR_027616 | clade_548 | N | N |
| Borrelia afzelii | 388 | ABCU01000001 | clade_549 | N | OP |
| Borrelia crocidurae | 390 | DQ057990 | clade_549 | N | OP |
| Borrelia duttonii | 391 | NC_011229 | clade_549 | N | OP |
| Borrelia hermsii | 393 | AY597657 | clade_549 | N | OP |
| Borrelia hispanica | 394 | DQ057988 | clade_549 | N | OP |
| Borrelia persica | 395 | HM161645 | clade_549 | N | OP |
| Borrelia recurrentis | 396 | AF107367 | clade_549 | N | OP |
| Borrelia spielmanii | 398 | ABKB01000002 | clade_549 | N | OP |
| Borrelia turicatae | 399 | NC_008710 | clade_549 | N | OP |
| Borrelia valaisiana | 400 | ABCY01000002 | clade_549 | N | OP |
| Providencia alcalifaciens | 1586 | ABXW01000071 | clade_55 | N | N |
| Providencia rettgeri | 1587 | AM040492 | clade_55 | N | N |
| Providencia rustigianii | 1588 | AM040489 | clade_55 | N | N |
| Providencia stuartii | 1589 | AF008581 | clade_55 | N | N |
| Treponema pallidum | 1932 | CP001752 | clade_550 | N | OP |
| Treponema phagedenis | 1934 | AEFH01000172 | clade_550 | N | N |
| Treponema sp. clone DDKL_4 | 1939 | Y08894 | clade_550 | N | N |
| Acholeplasma laidlawii | 17 | NR_074448 | clade_551 | N | N |
| Mycoplasma putrefaciens | 1323 | U26055 | clade_551 | N | N |
| Mycoplasmataceae genomosp. P1 oral clone MB1_G23 | 1325 | DQ003614 | clade_551 | N | N |
| Spiroplasma insolitum | 1750 | NR_025705 | clade_551 | N | N |
| Collinsella intestinalis | 660 | ABXH02000037 | clade_553 | N | N |
| Collinsella stercoris | 661 | ABXJ01000150 | clade_553 | N | N |
| Collinsella tanakaei | 662 | AB490807 | clade_553 | N | N |
| Caminicella sporogenes | 458 | NR_025485 | clade_554 | N | N |
| Acidaminococcus fermentans | 21 | CP001859 | clade_556 | N | N |
| Acidaminococcus intestini | 22 | CP003058 | clade_556 | N | N |
| Acidaminococcus sp. D21 | 23 | ACGB01000071 | clade_556 | N | N |
| Phascolarctobacterium faecium | 1462 | NR_026111 | clade_556 | N | N |
| Phascolarctobacterium sp. YIT 12068 | 1463 | AB490812 | clade_556 | N | N |
| Phascolarctobacterium succinatutens | 1464 | AB490811 | clade_556 | N | N |
| Acidithiobacillus ferrivorans | 25 | NR_074660 | clade_557 | N | N |
| Xanthomonadaceae bacterium NML 03_0222 | 2015 | EU313791 | clade_557 | N | N |
| Catabacter hongkongensis | 494 | AB671763 | clade_558 | N | N |
| Christensenella minuta | 512 | AB490809 | clade_558 | N | N |
| Clostridiales bacterium oral clone P4PA_66 P1 | 536 | AY207065 | clade_558 | N | N |
| Clostridiales bacterium oral taxon 093 | 537 | GQ422712 | clade_558 | N | N |
| Heliobacterium modesticaldum | 1000 | NR_074517 | clade_560 | N | N |
| Alistipes indistinctus | 130 | AB490804 | clade_561 | N | N |
| Bacteroidales bacterium ph 8 | 257 | JN837494 | clade_561 | N | N |
| Candidates Sulcia muelleri | 475 | CP002163 | clade_561 | N | N |
| Cytophaga xylanolytica | 742 | FR733683 | clade_561 | N | N |
| Flavobacteriaceae genomosp. C1 | 884 | AY278614 | clade_561 | N | N |
| Gramella forsetii | 958 | NR_074707 | clade_561 | N | N |
| Sphingobacterium faecium | 1740 | NR_025537 | clade_562 | N | N |
| Sphingobacterium mizutaii | 1741 | JF708889 | clade_562 | N | N |
| Sphingobacterium multivorum | 1742 | NR_040953 | clade_562 | N | N |
| Sphingobacterium spiritivorum | 1743 | ACHA02000013 | clade_562 | N | N |
| Jonquetella anthropi | 1017 | ACOO02000004 | clade_563 | N | N |
| Pyramidobacter piscolens | 1614 | AY207056 | clade_563 | N | N |
| Synergistes genomosp. C1 | 1904 | AY278615 | clade_563 | N | N |
| Synergistes sp. RMA 14551 | 1905 | DQ412722 | clade_563 | N | N |
| Synergistetes bacterium ADV897 | 1906 | GQ258968 | clade_563 | N | N |
| Candidates Arthromitus sp. | 474 | NR_074460 | clade_564 | N | N |
| SFB_mouse_Yit | | | | | |
| Gracilibacter thermotolerans | 957 | NR_043559 | clade_564 | N | N |
| Brachyspira aalborgi | 404 | FM178386 | clade_565 | N | N |
| Brachyspira sp. HIS3 | 406 | FM178387 | clade_565 | N | N |
| Brachyspira sp. HIS4 | 407 | FM178388 | clade_565 | N | N |
| Brachyspira sp. HIS5 | 408 | FM178389 | clade_565 | N | N |
| Adlercreutzia equolifaciens | 97 | AB306661 | clade_566 | N | N |
| Coriobacteriaceae bacterium JC110 | 678 | CAEM01000062 | clade_566 | N | N |
| Coriobacteriaceae bacterium phI | 679 | JN837493 | clade_566 | N | N |
| Cryptobacterium curtum | 740 | GQ422741 | clade_566 | N | N |
| Eggerthella sinensis | 779 | AY321958 | clade_566 | N | N |
| Eggerthella sp. 1_3_56FAA | 780 | ACWN01000099 | clade_566 | N | N |
| Eggerthella sp. HGA1 | 781 | AEXR01000021 | clade_566 | N | N |
| Eggerthella sp. YY7918 | 782 | AP012211 | clade_566 | N | N |
| Gordonibacter pamelaeae | 680 | AM886059 | clade_566 | N | N |
| Gordonibacter pamelaeae | 956 | FP929047 | clade_566 | N | N |
| Slackia equolifaciens | 1732 | EU377663 | clade_566 | N | N |
| Slackia exigua | 1733 | ACUX01000029 | clade_566 | N | N |
| Slackia faecicanis | 1734 | NR_042220 | clade_566 | N | N |
| Slackia heliotrinireducens | 1735 | NR_074439 | clade_566 | N | N |
| Slackia isoflavoniconvertens | 1736 | AB566418 | clade_566 | N | N |
| Slackia piriformis | 1737 | AB490806 | clade_566 | N | N |
| Slackia sp. NATTS | 1738 | AB505075 | clade_566 | N | N |
| Chlamydiales bacterium NS13 | 506 | JN606075 | clade_567 | N | N |
| Victivallaceae bacterium NML 080035 | 2003 | FJ394915 | clade_567 | N | N |
| Victivallis vadensis | 2004 | ABDE02000010 | clade_567 | N | N |
| Streptomyces griseus | 1889 | NR_074787 | clade_573 | N | N |
| Streptomyces sp. SD 511 | 1891 | EU544231 | clade_573 | N | N |
| Streptomyces sp. SD 534 | 1894 | EU544232 | clade_573 | N | N |
| Cloacibacillus evryensis | 530 | GQ258966 | clade_575 | N | N |
| Deferribacteres sp. oral clone JV001 | 743 | AY349370 | clade_575 | N | N |
| Deferribacteres sp. oral clone JV023 | 745 | AY349372 | clade_575 | N | N |
| Synergistetes bacterium LBVCM1157 | 1907 | GQ258969 | clade_575 | N | N |
| Synergistetes bacterium oral taxon 362 | 1909 | GU410752 | clade_575 | N | N |
| Synergistetes bacterium oral taxon D48 | 1910 | GU430992 | clade_575 | N | N |
| Peptococcus sp. oral clone JM048 | 1439 | AY349389 | clade_576 | N | N |
| Helicobacter winghamensis | 999 | ACDO01000013 | clade_577 | N | N |
| Wolinella succinogenes | 2014 | BX571657 | clade_577 | N | N |
| Olsenella genomosp. C1 | 1368 | AY278623 | clade_578 | N | N |
| Olsenella profusa | 1369 | FN178466 | clade_578 | N | N |
| Olsenella sp. F0004 | 1370 | EU592964 | clade_578 | N | N |
| Olsenella sp. oral taxon 809 | 1371 | ACVE01000002 | clade_578 | N | N |
| Olsenella uli | 1372 | CP002106 | clade_578 | N | N |
| Nocardiopsis dassonvillei | 1356 | CP002041 | clade_579 | N | N |
| Peptococcus niger | 1438 | NR_029221 | clade_580 | N | N |
| Peptococcus sp. oral taxon 167 | 1440 | GQ422727 | clade_580 | N | N |
| Akkermansia muciniphila | 118 | CP001071 | clade_583 | N | N |
| Opitutus terrae | 1373 | NR_074978 | clade_583 | N | N |
| Clostridiales bacterium oral taxon F32 | 538 | HM099644 | clade_584 | N | N |
| Leptospira borgpetersenii | 1161 | NC_008508 | clade_585 | N | OP |
| Leptospira broomii | 1162 | NR_043200 | clade_585 | N | OP |
| Leptospira interrogans | 1163 | NC_005823 | clade_585 | N | OP |
| Methanobrevibacter gottschalkii | 1213 | NR_044789 | clade_587 | N | N |
| Methanobrevibacter millerae | 1214 | NR_042785 | clade_587 | N | N |
| Methanobrevibacter oralis | 1216 | HE654003 | clade_587 | N | N |
| Methanobrevibacter thaueri | 1219 | NR_044787 | clade_587 | N | N |
| Methanobrevibacter smithii | 1218 | ABYV02000002 | clade_588 | N | N |
| Deinococcus radiodurans | 746 | AE000513 | clade_589 | N | N |
| Deinococcus sp. R_43890 | 747 | FR682752 | clade_589 | N | N |
| Thermus aquaticus | 1923 | NR_025900 | clade_589 | N | N |
| Actinomyces sp. c109 | 81 | AB167239 | clade_590 | N | N |
| Syntrophomonadaceae genomosp. P1 | 1912 | AY341821 | clade_590 | N | N |
| Anaerobaculum hydrogeniformans | 141 | ACJX02000009 | clade_591 | N | N |
| Microcystis aeruginosa | 1246 | NC_010296 | clade_592 | N | N |
| Prochlorococcus marinus | 1567 | CP000551 | clade_592 | N | N |
| Methanobrevibacter acididurans | 1208 | NR_028779 | clade_593 | N | N |
| Methanobrevibacter arboriphilus | 1209 | NR_042783 | clade_593 | N | N |
| Methanobrevibacter curvatus | 1210 | NR_044796 | clade_593 | N | N |
| Methanobrevibacter cuticularis | 1211 | NR_044776 | clade_593 | N | N |
| Methanobrevibacter filiformis | 1212 | NR_044801 | clade_593 | N | N |
| Methanobrevibacter woesei | 1220 | NR_044788 | clade_593 | N | N |
| Roseiflexus castenholzii | 1642 | CP000804 | clade_594 | N | N |
| Methanobrevibacter olleyae | 1215 | NR_043024 | clade_595 | N | N |
| Methanobrevibacter ruminantium | 1217 | NR_042784 | clade_595 | N | N |
| Methanobrevibacter wolinii | 1221 | NR_044790 | clade_595 | N | N |
| Methanosphaera stadtmanae | 1222 | AY196684 | clade_595 | N | N |
| Chloroflexi genomosp. P1 | 511 | AY331414 | clade_596 | N | N |
| Halorubrum lipolyticum | 992 | AB477978 | clade_597 | N | N |
| Methanobacterium formicicum | 1207 | NR_025028 | clade_597 | N | N |
| Acidilobus saccharovorans | 24 | AY350586 | clade_598 | N | N |
| Hyperthermus butylicus | 1006 | CP000493 | clade_598 | N | N |
| Ignicoccus islandicus | 1011 | X99562 | clade_598 | N | N |
| Metallosphaera sedula | 1206 | D26491 | clade_598 | N | N |
| Thermofilum pendens | 1922 | X14835 | clade_598 | N | N |
| Prevotella melaninogenica | 1506 | CP002122 | clade_6 | N | N |
| Prevotella sp. ICM1 | 1520 | HQ616385 | clade_6 | N | N |
| Prevotella sp. oral clone FU048 | 1535 | AY349393 | clade_6 | N | N |
| Prevotella sp. oral clone GI030 | 1537 | AY349395 | clade_6 | N | N |
| Prevotella sp. SEQ116 | 1526 | JN867246 | clade_6 | N | N |
| Streptococcus anginosus | 1787 | AECT01000011 | clade_60 | N | N |
| Streptococcus milleri | 1812 | X81023 | clade_60 | N | N |
| Streptococcus sp. 16362 | 1829 | JN590019 | clade_60 | N | N |
| Streptococcus sp. 69130 | 1832 | X78825 | clade_60 | N | N |
| Streptococcus sp. AC15 | 1833 | HQ616356 | clade_60 | N | N |
| Streptococcus sp. CM7 | 1839 | HQ616373 | clade_60 | N | N |
| Streptococcus sp. OBRC6 | 1847 | HQ616352 | clade_60 | N | N |
| Burkholderia ambifaria | 442 | AAUZ01000009 | clade_61 | N | OP |
| Burkholderia cenocepacia | 443 | AAHI01000060 | clade_61 | N | OP |
| Burkholderia cepacia | 444 | NR_041719 | clade_61 | N | OP |
| Burkholderia mallei | 445 | CP000547 | clade_61 | N | Category-B |
| Burkholderia multivorans | 446 | NC_010086 | clade_61 | N | OP |
| Burkholderia oklahomensis | 447 | DQ108388 | clade_61 | N | OP |
| Burkholderia pseudomallei | 448 | CP001408 | clade_61 | N | Category-B |
| Burkholderia rhizoxinica | 449 | HQ005410 | clade_61 | N | OP |
| Burkholderia sp. 383 | 450 | CP000151 | clade_61 | N | OP |
| Burkholderia xenovorans | 451 | U86373 | clade_61 | N | OP |
| Prevotella buccae | 1488 | ACRB01000001 | clade_62 | N | N |
| Prevotella genomosp. P8 oral clone MB3_P13 | 1498 | DQ003622 | clade_62 | N | N |
| Prevotella sp. oral clone FW035 | 1536 | AY349394 | clade_62 | N | N |
| Prevotella bivia | 1486 | ADFO01000096 | clade_63 | N | N |
| Prevotella disiens | 1494 | AEDO01000026 | clade_64 | N | N |
| Bacteroides faecis | 276 | GQ496624 | clade_65 | N | N |
| Bacteroides fragilis | 279 | AP006841 | clade_65 | N | N |
| Bacteroides nordii | 285 | NR_043017 | clade_65 | N | N |
| Bacteroides salyersiae | 292 | EU136690 | clade_65 | N | N |
| Bacteroides sp. 1_1_14 | 293 | ACRP01000155 | clade_65 | N | N |
| Bacteroides sp. 1_1_6 | 295 | ACIC01000215 | clade_65 | N | N |
| Bacteroides sp. 2_1_56FAA | 298 | ACWI01000065 | clade_65 | N | N |
| Bacteroides sp. AR29 | 316 | AF139525 | clade_65 | N | N |
| Bacteroides sp. B2 | 317 | EU722733 | clade_65 | N | N |
| Bacteroides thetaiotaomicron | 328 | NR_074277 | clade_65 | N | N |
| Actinobacillus minor | 45 | ACFT01000025 | clade_69 | N | N |
| Haemophilias parasuis | 978 | GU226366 | clade_69 | N | N |
| Vibrio cholerae | 1996 | AAUR01000095 | clade_71 | N | Category-B |
| Vibrio fluvialis | 1997 | X76335 | clade_71 | N | Category-B |
| Vibrio furnissii | 1998 | CP002377 | clade_71 | N | Category-B |
| Vibrio mimicus | 1999 | ADAF01000001 | clade_71 | N | Category-B |
| Vibrio parahaemolyticus | 2000 | AAWQ01000116 | clade_71 | N | Category-B |
| Vibrio sp. RC341 | 2001 | ACZT01000024 | clade_71 | N | Category-B |
| Vibrio vulnificus | 2002 | AE016796 | clade_71 | N | Category-B |
| Lactobacillus acidophilus | 1067 | CP000033 | clade_72 | N | N |
| Lactobacillus amylolyticus | 1069 | ADNY01000006 | clade_72 | N | N |
| Lactobacillus amylovorus | 1070 | CP002338 | clade_72 | N | N |
| Lactobacillus crispatus | 1078 | ACOG01000151 | clade_72 | N | N |
| Lactobacillus delbrueckii | 1080 | CP002341 | clade_72 | N | N |
| Lactobacillus helveticus | 1088 | ACLM01000202 | clade_72 | N | N |
| Lactobacillus kalixensis | 1094 | NR_029083 | clade_72 | N | N |
| Lactobacillus kefiranofaciens | 1095 | NR_042440 | clade_72 | N | N |
| Lactobacillus leichmannii | 1098 | JX986966 | clade_72 | N | N |
| Lactobacillus sp. 66c | 1120 | FR681900 | clade_72 | N | N |
| Lactobacillus sp. KLDS 1.0701 | 1122 | EU600905 | clade_72 | N | N |
| Lactobacillus sp. KLDS 1.0712 | 1130 | EU600916 | clade_72 | N | N |
| Lactobacillus sp. oral clone HT070 | 1136 | AY349383 | clade_72 | N | N |
| Lactobacillus ultunensis | 1139 | ACGU01000081 | clade_72 | N | N |
| Prevotella intermedia | 1502 | AF414829 | clade_81 | N | N |
| Prevotella nigrescens | 1511 | AFPX01000069 | clade_81 | N | N |
| Prevotella pallens | 1515 | AFPY01000135 | clade_81 | N | N |
| Prevotella sp. oral taxon 310 | 1551 | GQ422737 | clade_81 | N | N |
| Prevotella genomosp. C1 | 1495 | AY278624 | clade_82 | N | N |
| Prevotella sp. CM38 | 1519 | HQ610181 | clade_82 | N | N |
| Prevotella sp. oral taxon 317 | 1552 | ACQH01000158 | clade_82 | N | N |
| Prevotella sp. SG12 | 1527 | GU561343 | clade_82 | N | N |
| Prevotella denticola | 1493 | CP002589 | clade_83 | N | N |
| Prevotella genomosp. P7 oral clone MB2_P31 | 1497 | DQ003620 | clade_83 | N | N |
| Prevotella histicola | 1501 | JN867315 | clade_83 | N | N |
| Prevotella multiformis | 1508 | AEWX01000054 | clade_83 | N | N |
| Prevotella sp. JCM 6330 | 1522 | AB547699 | clade_83 | N | N |
| Prevotella sp. oral clone GI059 | 1539 | AY349397 | clade_83 | N | N |
| Prevotella sp. oral taxon 782 | 1555 | GQ422745 | clade_83 | N | N |
| Prevotella sp. oral taxon G71 | 1559 | GU432180 | clade_83 | N | N |
| Prevotella sp. SEQ065 | 1524 | JN867234 | clade_83 | N | N |
| Prevotella veroralis | 1565 | ACVA01000027 | clade_83 | N | N |
| Bacteroides acidifaciens | 266 | NR_028607 | clade_85 | N | N |
| Bacteroides cellulosilyticus | 269 | ACCH01000108 | clade_85 | N | N |
| Bacteroides clarus | 270 | AFBM01000011 | clade_85 | N | N |
| Bacteroides eggerthii | 275 | ACWG01000065 | clade_85 | N | N |
| Bacteroides oleiciplenus | 286 | AB547644 | clade_85 | N | N |
| Bacteroides pyogenes | 290 | NR_041280 | clade_85 | N | N |
| Bacteroides sp. 315_5 | 300 | FJ848547 | clade_85 | N | N |
| Bacteroides sp. 31SF15 | 301 | AJ583248 | clade_85 | N | N |
| Bacteroides sp. 31SF18 | 302 | AJ583249 | clade_85 | N | N |
| Bacteroides sp. 35AE31 | 303 | AJ583244 | clade_85 | N | N |
| Bacteroides sp. 35AE37 | 304 | AJ583245 | clade_85 | N | N |
| Bacteroides sp. 35BE34 | 305 | AJ583246 | clade_85 | N | N |
| Bacteroides sp. 35BE35 | 306 | AJ583247 | clade_85 | N | N |
| Bacteroides sp. WH2 | 324 | AY895180 | clade_85 | N | N |
| Bacteroides sp. XB12B | 325 | AM230648 | clade_85 | N | N |
| Bacteroides stercoris | 327 | ABFZ02000022 | clade_85 | N | N |
| Actinobacillus pleuropneumoniae | 46 | NR_074857 | clade_88 | N | N |
| Actinobacillus ureae | 48 | AEVG01000167 | clade_88 | N | N |
| Haemophilus aegyptius | 969 | AFBC01000053 | clade_88 | N | N |
| Haemophilus ducreyi | 970 | AE017143 | clade_88 | N | OP |
| Haemophilus haemolyticus | 973 | JN175335 | clade_88 | N | N |
| Haemophilus influenzae | 974 | AADP01000001 | clade_88 | N | OP |
| Haemophilus parahaemolyticus | 975 | GU561425 | clade_88 | N | N |
| Haemophilus parainfluenzae | 976 | AEWU01000024 | clade_88 | N | N |
| Haemophilus paraphrophaemolyticus | 977 | M75076 | clade_88 | N | N |
| Haemophilus somnus | 979 | NC_008309 | clade_88 | N | N |
| Haemophilus sp. 70334 | 980 | HQ680854 | clade_88 | N | N |
| Haemophilus sp. HK445 | 981 | FJ685624 | clade_88 | N | N |
| Haemophilus sp. oral clone ASCA07 | 982 | AY923117 | clade_88 | N | N |
| Haemophilus sp. oral clone ASCG06 | 983 | AY923147 | clade_88 | N | N |
| Haemophilus sp. oral clone BJ021 | 984 | AY005034 | clade_88 | N | N |
| Haemophilus sp. oral clone BJ095 | 985 | AY005033 | clade_88 | N | N |
| Haemophilus sp. oral taxon 851 | 987 | AGRK01000004 | clade_88 | N | N |
| Haemophilus sputorum | 988 | AFNK01000005 | clade_88 | N | N |
| Histophilus somni | 1003 | AF549387 | clade_88 | N | N |
| Mannheimia haemolytica | 1195 | ACZX01000102 | clade_88 | N | N |
| Pasteurella bettyae | 1433 | L06088 | clade_88 | N | N |
| Moellerella wisconsensis | 1253 | JN175344 | clade_89 | N | N |
| Morganella morganii | 1265 | AJ301681 | clade_89 | N | N |
| Morganella sp. JB_T16 | 1266 | AJ781005 | clade_89 | N | N |
| Proteus mirabilis | 1582 | ACLE01000013 | clade_89 | N | N |
| Proteus penneri | 1583 | ABVP01000020 | clade_89 | N | N |
| Proteus sp. HS7514 | 1584 | DQ512963 | clade_89 | N | N |
| Proteus vulgaris | 1585 | AJ233425 | clade_89 | N | N |
| Oribacterium sinus | 1374 | ACKX1000142 | clade_90 | N | N |
| Oribacterium sp. ACB1 | 1375 | HM120210 | clade_90 | N | N |
| Oribacterium sp. ACB7 | 1376 | HM120211 | clade_90 | N | N |
| Oribacterium sp. CM12 | 1377 | HQ616374 | clade_90 | N | N |
| Oribacterium sp. ICM51 | 1378 | HQ616397 | clade_90 | N | N |
| Oribacterium sp. OBRC12 | 1379 | HQ616355 | clade_90 | N | N |
| Oribacterium sp. oral taxon 108 | 1382 | AFIH01000001 | clade_90 | N | N |
| Actinobacillus actinomycetemcomitans | 44 | AY362885 | clade_92 | N | N |
| Actinobacillus succinogenes | 47 | CP000746 | clade_92 | N | N |
| Aggregatibacter actinomycetemcomitans | 112 | CP001733 | clade_92 | N | N |
| Aggregatibacter aphrophilus | 113 | CP001607 | clade_92 | N | N |
| Aggregatibacter segnis | 114 | AEPS01000017 | clade_92 | N | N |
| Averyella dalhousiensis | 194 | DQ481464 | clade_92 | N | N |
| Bisgaard Taxon | 368 | AY683487 | clade_92 | N | N |
| Bisgaard Taxon | 369 | AY683489 | clade_92 | N | N |
| Bisgaard Taxon | 370 | AY683491 | clade_92 | N | N |
| Bisgaard Taxon | 371 | AY683492 | clade_92 | N | N |
| Buchnera aphidicola | 440 | NR_074609 | clade_92 | N | N |
| Cedecea davisae | 499 | AF493976 | clade_92 | N | N |
| Citrobacter amalonaticus | 517 | FR870441 | clade_92 | N | N |
| Citrobacter braakii | 518 | NR_028687 | clade_92 | N | N |
| Citrobacter farmeri | 519 | AF025371 | clade_92 | N | N |
| Citrobacter freundii | 520 | NR_028894 | clade_92 | N | N |
| Citrobacter gillenii | 521 | AF025367 | clade_92 | N | N |
| Citrobacter koseri | 522 | NC_009792 | clade_92 | N | N |
| Citrobacter murliniae | 523 | AF025369 | clade_92 | N | N |
| Citrobacter rodentium | 524 | NR_074903 | clade_92 | N | N |
| Citrobacter sedlakii | 525 | AF025364 | clade_92 | N | N |
| Citrobacter sp. 30_2 | 526 | ACDJ01000053 | clade_92 | N | N |
| Citrobacter sp. KMSI_3 | 527 | GQ468398 | clade_92 | N | N |
| Citrobacter werkmanii | 528 | AF025373 | clade_92 | N | N |
| Citrobacter youngae | 529 | ABWL02000011 | clade_92 | N | N |
| Cronobacter malonaticus | 737 | GU122174 | clade_92 | N | N |
| Cronobacter sakazakii | 738 | NC_009778 | clade_92 | N | N |
| Cronobacter turicensis | 739 | FN543093 | clade_92 | N | N |
| Enterobacter aerogenes | 786 | AJ251468 | clade_92 | N | N |
| Enterobacter asburiae | 787 | NR_024640 | clade_92 | N | N |
| Enterobacter cancerogenus | 788 | Z96078 | clade_92 | N | N |
| Enterobacter cloacae | 789 | FP929040 | clade_92 | N | N |
| Enterobacter cowanii | 790 | NR_025566 | clade_92 | N | N |
| Enterobacter hormaechei | 791 | AFHR01000079 | clade_92 | N | N |
| Enterobacter sp. 247BMC | 792 | HQ122932 | clade_92 | N | N |
| Enterobacter sp. 638 | 793 | NR_074777 | clade_92 | N | N |
| Enterobacter sp. JC163 | 794 | JN657217 | clade_92 | N | N |
| Enterobacter sp. SCSS | 795 | HM007811 | clade_92 | N | N |
| Enterobacter sp. TSE38 | 796 | HM156134 | clade_92 | N | N |
| Enterobacteriaceae bacterium 9_2_54FAA | 797 | ADCU01000033 | clade_92 | N | N |
| Enterobacteriaceae bacterium CF01Ent_1 | 798 | AJ489826 | clade_92 | N | N |
| Enterobacteriaceae bacterium Smarlab 3302238 | 799 | AY538694 | clade_92 | N | N |
| Escherichia albertii | 824 | ABKX01000012 | clade_92 | N | N |
| Escherichia coli | 825 | NC_008563 | clade_92 | N | Category-B |
| Escherichia fergusonii | 826 | CU928158 | clade_92 | N | N |
| Escherichia hermannii | 827 | HQ407266 | clade_92 | N | N |
| Escherichia sp. 1_1_43 | 828 | ACID01000033 | clade_92 | N | N |
| Escherichia sp. 4_1_40B | 829 | ACDM02000056 | clade_92 | N | N |
| Escherichia sp. B4 | 830 | EU722735 | clade_92 | N | N |
| Escherichia vulneris | 831 | NR_041927 | clade_92 | N | N |
| Ewingella americana | 877 | JN175329 | clade_92 | N | N |
| Haemophilus genomosp. P2 oral clone MB3_C24 | 971 | DQ003621 | clade_92 | N | N |
| Haemophilus genomosp. P3 oral clone MB3_C38 | 972 | DQ003635 | clade_92 | N | N |
| Haemophilus sp. oral clone JM053 | 986 | AY349380 | clade_92 | N | N |
| Hafnia alvei | 989 | DQ412565 | clade_92 | N | N |
| Klebsiella oxytoca | 1024 | AY292871 | clade_92 | N | OP |
| Klebsiella pneumoniae | 1025 | CP000647 | clade_92 | N | OP |
| Klebsiella sp. AS10 | 1026 | HQ616362 | clade_92 | N | N |
| Klebsiella sp. Co9935 | 1027 | DQ068764 | clade_92 | N | N |
| Klebsiella sp. enrichment culture clone SRC_DSD25 | 1036 | HM195210 | clade_92 | N | N |
| Klebsiella sp. OBRC7 | 1028 | HQ616353 | clade_92 | N | N |
| Klebsiella sp. SP_BA | 1029 | FJ999767 | clade_92 | N | N |
| Klebsiella sp. SRC_DSD1 | 1033 | GU797254 | clade_92 | N | N |
| Klebsiella sp. SRC_DSD11 | 1030 | GU797263 | clade_92 | N | N |
| Klebsiella sp. SRC_DSD12 | 1031 | GU797264 | clade_92 | N | N |
| Klebsiella sp. SRC_DSD15 | 1032 | GU797267 | clade_92 | N | N |
| Klebsiella sp. SRC_DSD2 | 1034 | GU797253 | clade_92 | N | N |
| Klebsiella sp. SRC_DSD6 | 1035 | GU797258 | clade_92 | N | N |
| Klebsiella variicola | 1037 | CP001891 | clade_92 | N | N |
| Kluyvera ascorbata | 1038 | NR_028677 | clade_92 | N | N |
| Kluyvera cryocrescens | 1039 | NR_028803 | clade_92 | N | N |
| Leminorella grimontii | 1159 | AJ233421 | clade_92 | N | N |
| Leminorella richardii | 1160 | HF558368 | clade_92 | N | N |
| Pantoea agglomerans | 1409 | AY335552 | clade_92 | N | N |
| Pantoea ananatis | 1410 | CP001875 | clade_92 | N | N |
| Pantoea brenneri | 1411 | EU216735 | clade_92 | N | N |
| Pantoea citrea | 1412 | EF688008 | clade_92 | N | N |
| Pantoea conspicua | 1413 | EU216737 | clade_92 | N | N |
| Pantoea septica | 1414 | EU216734 | clade_92 | N | N |
| Pasteurella dagmatis | 1434 | ACZR01000003 | clade_92 | N | N |
| Pasteurella multocida | 1435 | NC_002663 | clade_92 | N | N |
| Plesiomonas shigelloides | 1469 | X60418 | clade_92 | N | N |
| Raoultella ornithinolytica | 1617 | AB364958 | clade_92 | N | N |
| Raoultella planticola | 1618 | AF129443 | clade_92 | N | N |
| Raoultella terrigena | 1619 | NR_037085 | clade_92 | N | N |
| Salmonella bongori | 1683 | NR_041699 | clade_92 | N | Category-B |
| Salmonella enterica | 1672 | NC_011149 | clade_92 | N | Category-B |
| Salmonella enterica | 1673 | NC_011205 | clade_92 | N | Category-B |
| Salmonella enterica | 1674 | DQ344532 | clade_92 | N | Category-B |
| Salmonella enterica | 1675 | ABEH02000004 | clade_92 | N | Category-B |
| Salmonella enterica | 1676 | ABAK02000001 | clade_92 | N | Category-B |
| Salmonella enterica | 1677 | NC_011080 | clade_92 | N | Category-B |
| Salmonella enterica | 1678 | EU118094 | clade_92 | N | Category-B |
| Salmonella enterica | 1679 | NC_011094 | clade_92 | N | Category-B |
| Salmonella enterica | 1680 | AE014613 | clade_92 | N | Category-B |
| Salmonella enterica | 1682 | ABFH02000001 | clade_92 | N | Category-B |
| Salmonella enterica | 1684 | ABEM01000001 | clade_92 | N | Category-B |
| Salmonella enterica | 1685 | ABAM02000001 | clade_92 | N | Category-B |
| Salmonella typhimurium | 1681 | DQ344533 | clade_92 | N | Category-B |
| Salmonella typhimurium | 1686 | AF170176 | clade_92 | N | Category-B |
| Serratia fonticola | 1718 | NR_025339 | clade_92 | N | N |
| Serratia liquefaciens | 1719 | NR_042062 | clade_92 | N | N |
| Serratia marcescens | 1720 | GU826157 | clade_92 | N | N |
| Serratia odorifera | 1721 | ADBY01000001 | clade_92 | N | N |
| Serratia proteamaculans | 1722 | AAUN01000015 | clade_92 | N | N |
| Shigella boydii | 1724 | AAKA01000007 | clade_92 | N | Category-B |
| Shigella dysenteriae | 1725 | NC_007606 | clade_92 | N | Category-B |
| Shigella flexneri | 1726 | AE005674 | clade_92 | N | Category-B |
| Shigella sonnei | 1727 | NC_007384 | clade_92 | N | Category-B |
| Tatumella ptyseos | 1916 | NR_025342 | clade_92 | N | N |
| Trabulsiella guamensis | 1925 | AYS73830 | clade_92 | N | N |
| Yersinia aldovae | 2019 | AJ871363 | clade_92 | N | OP |
| Yersinia aleksiciae | 2020 | AJ627597 | clade_92 | N | OP |
| Yersinia bercovieri | 2021 | AF366377 | clade_92 | N | OP |
| Yersinia enterocolitica | 2022 | FR729477 | clade_92 | N | Category-B |
| Yersinia frederiksenii | 2023 | AF366379 | clade_92 | N | OP |
| Yersinia intermedia | 2024 | AF366380 | clade_92 | N | OP |
| Yersinia kristensenii | 2025 | ACCA01000078 | clade_92 | N | OP |
| Yersinia mollaretii | 2026 | NR_027546 | clade_92 | N | OP |
| Yersinia pestis | 2027 | AE013632 | clade_92 | N | Category-A |
| Yersinia pseudotuberculosis | 2028 | NC_009708 | clade_92 | N | OP |
| Yersinia rohdei | 2029 | ACCD01000071 | clade_92 | N | OP |
| Yokenella regensburgei | 2030 | AB273739 | clade_92 | N | N |
| Conchiformibius kuhniae | 669 | NR_041821 | clade_94 | N | N |
| Morococcus cerebrosus | 1267 | JN175352 | clade_94 | N | N |
| Neisseria bacilliformis | 1328 | AFAY01000058 | clade_94 | N | N |
| Neisseria cinerea | 1329 | ACDY01000037 | clade_94 | N | N |
| Neisseria flavescens | 1331 | ACQV01000025 | clade_94 | N | N |
| Neisseria gonorrhoeae | 1333 | CP002440 | clade_94 | N | OP |
| Neisseria lactamica | 1334 | ACEQ01000095 | clade_94 | N | N |
| Neisseria macacae | 1335 | AFQE01000146 | clade_94 | N | N |
| Neisseria meningitidis | 1336 | NC_003112 | clade_94 | N | OP |
| Neisseria mucosa | 1337 | ACDX01000110 | clade_94 | N | N |
| Neisseria pharyngis | 1338 | AJ239281 | clade_94 | N | N |
| Neisseria polysaccharea | 1339 | ADBE01000137 | clade_94 | N | N |
| Neisseria sicca | 1340 | ACKO02000016 | clade_94 | N | N |
| Neisseria sp. KEM232 | 1341 | GQ203291 | clade_94 | N | N |
| Neisseria sp. oral clone AP132 | 1344 | AY005027 | clade_94 | N | N |
| Neisseria sp. oral strain B33KA | 1346 | AY005028 | clade_94 | N | N |
| Neisseria sp. oral taxon 014 | 1347 | ADEA01000039 | clade_94 | N | N |
| Neisseria sp. TM10_1 | 1343 | DQ279352 | clade_94 | N | N |
| Neisseria subflava | 1348 | ACEO01000067 | clade_94 | N | N |
| Okadaella gastrococcus | 1365 | HQ699465 | clade_98 | N | N |
| Streptococcus agalactiae | 1785 | AAJO01000130 | clade_98 | N | N |
| Streptococcus alactolyticus | 1786 | NR_041781 | clade_98 | N | N |
| Streptococcus australis | 1788 | AEQR01000024 | clade_98 | N | N |
| Streptococcus bovis | 1789 | AEEL01000030 | clade_98 | N | N |
| Streptococcus canis | 1790 | AJ413203 | clade_98 | N | N |
| Streptococcus constellatus | 1791 | AY277942 | clade_98 | N | N |
| Streptococcus cristatus | 1792 | AEVC01000028 | clade_98 | N | N |
| Streptococcus dysgalactiae | 1794 | AP010935 | clade_98 | N | N |
| Streptococcus equi | 1795 | CP001129 | clade_98 | N | N |
| Streptococcus equinus | 1796 | AEVB01000043 | clade_98 | N | N |
| Streptococcus gallolyticus | 1797 | FR824043 | clade_98 | N | N |
| Streptococcus genomosp. C1 | 1798 | AY278629 | clade_98 | N | N |
| Streptococcus genomosp. C2 | 1799 | AY278630 | clade_98 | N | N |
| Streptococcus genomosp. C3 | 1800 | AY278631 | clade_98 | N | N |
| Streptococcus genomosp. C4 | 1801 | AY278632 | clade_98 | N | N |
| Streptococcus genomosp. C5 | 1802 | AY278633 | clade_98 | N | N |
| Streptococcus genomosp. C6 | 1803 | AY278634 | clade_98 | N | N |
| Streptococcus genomosp. C7 | 1804 | AY278635 | clade_98 | N | N |
| Streptococcus genomosp. C8 | 1805 | AY278609 | clade_98 | N | N |
| Streptococcus gordonii | 1806 | NC_009785 | clade_98 | N | N |
| Streptococcus infantarius | 1807 | ABJK02000017 | clade_98 | N | N |
| Streptococcus infantis | 1808 | AFNN01000024 | clade_98 | N | N |
| Streptococcus intermedius | 1809 | NR_028736 | clade_98 | N | N |
| Streptococcus lutetiensis | 1810 | NR_037096 | clade_98 | N | N |
| Streptococcus massiliensis | 1811 | AY769997 | clade_98 | N | N |
| Streptococcus mitis | 1813 | AM157420 | clade_98 | N | N |
| Streptococcus oligofermentans | 1815 | AY099095 | clade_98 | N | N |
| Streptococcus oralis | 1816 | ADMV01000001 | clade_98 | N | N |
| Streptococcus parasanguinis | 1817 | AEKM01000012 | clade_98 | N | N |
| Streptococcus pasteurianus | 1818 | AP012054 | clade_98 | N | N |
| Streptococcus peroris | 1819 | AEVF01000016 | clade_98 | N | N |
| Streptococcus pneumoniae | 1820 | AE008537 | clade_98 | N | N |
| Streptococcus porcinus | 1821 | EF121439 | clade_98 | N | N |
| Streptococcus pseudopneumoniae | 1822 | FJ827123 | clade_98 | N | N |
| Streptococcus pseudoporcinus | 1823 | AENS01000003 | clade_98 | N | N |
| Streptococcus pyogenes | 1824 | AE006496 | clade_98 | N | OP |
| Streptococcus ratti | 1825 | X58304 | clade_98 | N | N |
| Streptococcus sanguinis | 1827 | NR_074974 | clade_98 | N | N |
| Streptococcus sinensis | 1828 | AF432857 | clade_98 | N | N |
| Streptococcus sp. 2_1_36FAA | 1831 | ACOI01000028 | clade_98 | N | N |
| Streptococcus sp. 2285_97 | 1830 | AJ131965 | clade_98 | N | N |
| Streptococcus sp. ACS2 | 1834 | HQ616360 | clade_98 | N | N |
| Streptococcus sp. AS20 | 1835 | HQ616366 | clade_98 | N | N |
| Streptococcus sp. BS35a | 1836 | HQ616369 | clade_98 | N | N |
| Streptococcus sp. C150 | 1837 | ACRI01000045 | clade_98 | N | N |
| Streptococcus sp. CM6 | 1838 | HQ616372 | clade_98 | N | N |
| Streptococcus sp. ICM10 | 1840 | HQ616389 | clade_98 | N | N |
| Streptococcus sp. ICM12 | 1841 | HQ616390 | clade_98 | N | N |
| Streptococcus sp. ICM2 | 1842 | HQ616386 | clade_98 | N | N |
| Streptococcus sp. ICM4 | 1844 | HQ616387 | clade_98 | N | N |
| Streptococcus sp. ICM45 | 1843 | HQ616394 | clade_98 | N | N |
| Streptococcus sp. M143 | 1845 | ACRK01000025 | clade_98 | N | N |
| Streptococcus sp. M334 | 1846 | ACRL01000052 | clade_98 | N | N |
| Streptococcus sp. oral clone ASB02 | 1849 | AY923121 | clade_98 | N | N |
| Streptococcus sp. oral clone ASCA03 | 1850 | DQ272504 | clade_98 | N | N |
| Streptococcus sp. oral clone ASCA04 | 1851 | AY923116 | clade_98 | N | N |
| Streptococcus sp. oral clone ASCA09 | 1852 | AY923119 | clade_98 | N | N |
| Streptococcus sp. oral clone ASCB04 | 1853 | AY923123 | clade_98 | N | N |
| Streptococcus sp. oral clone ASCB06 | 1854 | AY923124 | clade_98 | N | N |
| Streptococcus sp. oral clone ASCC04 | 1855 | AY923127 | clade_98 | N | N |
| Streptococcus sp. oral clone ASCC05 | 1856 | AY923128 | clade_98 | N | N |
| Streptococcus sp. oral clone ASCC12 | 1857 | DQ272507 | clade_98 | N | N |
| Streptococcus sp. oral clone ASCD01 | 1858 | AY923129 | clade_98 | N | N |
| Streptococcus sp. oral clone ASCD09 | 1859 | AY923130 | clade_98 | N | N |
| Streptococcus sp. oral clone ASCD10 | 1860 | DQ272509 | clade_98 | N | N |
| Streptococcus sp. oral clone ASCE03 | 1861 | AY923134 | clade_98 | N | N |
| Streptococcus sp. oral clone ASCE04 | 1862 | AY953253 | clade_98 | N | N |
| Streptococcus sp. oral clone ASCE05 | 1863 | DQ272510 | clade_98 | N | N |
| Streptococcus sp. oral clone ASCE06 | 1864 | AY923135 | clade_98 | N | N |
| Streptococcus sp. oral clone ASCE09 | 1865 | AY923136 | clade_98 | N | N |
| Streptococcus sp. oral clone ASCE10 | 1866 | AY923137 | clade_98 | N | N |
| Streptococcus sp. oral clone ASCE12 | 1867 | AY923138 | clade_98 | N | N |
| Streptococcus sp. oral clone ASCF05 | 1868 | AY923140 | clade_98 | N | N |
| Streptococcus sp. oral clone ASCF07 | 1869 | AY953255 | clade_98 | N | N |
| Streptococcus sp. oral clone ASCF09 | 1870 | AY923142 | clade_98 | N | N |
| Streptococcus sp. oral clone ASCG04 | 1871 | AY923145 | clade_98 | N | N |
| Streptococcus sp. oral clone BW009 | 1872 | AY005042 | clade_98 | N | N |
| Streptococcus sp. oral clone CH016 | 1873 | AY005044 | clade_98 | N | N |
| Streptococcus sp. oral clone GK051 | 1874 | AY349413 | clade_98 | N | N |
| Streptococcus sp. oral clone GM006 | 1875 | AY349414 | clade_98 | N | N |
| Streptococcus sp. oral clone P2PA_41 P2 | 1876 | AY207051 | clade_98 | N | N |
| Streptococcus sp. oral clone P4PA_30 P4 | 1877 | AY207064 | clade_98 | N | N |
| Streptococcus sp. oral taxon 071 | 1878 | AEEP01000019 | clade_98 | N | N |
| Streptococcus sp. oral taxon G59 | 1879 | GU432132 | clade_98 | N | N |
| Streptococcus sp. oral taxon G62 | 1880 | GU432146 | clade_98 | N | N |
| Streptococcus sp. oral taxon G63 | 1881 | GU432150 | clade_98 | N | N |
| Streptococcus suis | 1882 | FM252032 | clade_98 | N | N |
| Streptococcus thermophilus | 1883 | CP000419 | clade_98 | N | N |
| Streptococcus salivarius | 1826 | AGBV01000001 | clade_98 | N | N |
| Streptococcus uberis | 1884 | HQ391900 | clade_98 | N | N |
| Streptococcus urinalis | 1885 | DQ303194 | clade_98 | N | N |
| Streptococcus vestibularis | 1886 | AEKO01000008 | clade_98 | N | N |
| Streptococcus viridans | 1887 | AF076036 | clade_98 | N | N |
| Synergistetes bacterium oral clone 03 5 D05 | 1908 | GU227192 | clade_98 | N | N |

List of Operational Taxonomic Units (OTU) with taxonomic assignments made to Genus, Species, and Phylogenetic Clade. Clade membership of bacterial OTUs is based on 16S sequence data. Clades are defined based on the topology of a phylogenetic tree that is constructed from full-length 16S sequences using maximum likelihood methods familiar to individuals with ordinary skill in the art of phylogenetics. Clades are constructed to ensure that all OTUs in a given clade are: (i) within a specified number of bootstrap supported nodes from one another, and (ii) within 5% genetic similarity. OTUs that are within the same clade can be distinguished as genetically and phylogenetically distinct from OTUs in a different clade based on 16S-V4 sequence data, while OTUs falling within the same clade are closely related. OTUs falling within the same clade are evolutionarily closely related and may or may not be distinguishable from one another using 16S-V4 sequence data. Members of the same clade, due to their evolutionary relatedness, play similar functional roles in a microbial ecology such as that found in the human gut. Compositions substituting one species with another from the same clade are likely to have conserved ecological function and therefore are useful in the present invention. All OTUs are denoted as to their putative capacity to form spores and whether they are a Pathogen or Pathobiont (see Definitions for description of “Pathobiont”). NIAID Priority Pathogens are denoted as ‘Category-A’, ‘Category-B’ or ‘Category-C’, and Opportunistic Pathogens are denoted as ‘OP’. OTUs that are not pathogenic or for which their ability to exist as a pathogen is unknown are denoted as ‘N’. The ‘SEQ ID Number’ denotes the identifier of the OTU in the Sequence Listing File and ‘Public DB Accession’ denotes the identifier of the OTU in a public sequence repository.

TABLE 2
Spore quantitation for ethanol treated spore preparations
using spore CFU (SCFU) assay and DPA assay

| | SCFU/30 | DPA SEq/30 | Ratio |
|---|---|---|---|
| Preparation | capsules | capsules | SCFU/DPA |
| Preparation 1 | 4.0 × 105 | 6.8 × 107 | 5.9 × 10−3 |
| Preparation 2 | 2.1 × 107 | 9.2 × 108 | 0.023 |
| Preparation 3 | 6.9 × 109 | 9.6 × 109 | 0.72 |

TABLE 3
DPA doses in Table 2 when normalized to 4 × 105 SCFU per dose

| | SCFU/30 | DPA SEq/30 | Fraction of |
|---|---|---|---|
| Preparation | capsules | capsules | Preparation 1 Dose |
| Preparation 1 | 4.0 × 105 | 6.8 × 107 | 1.0 |
| Preparation 2 | 4.0 × 105 | 1.8 × 107 | 0.26 |
| Preparation 3 | 4.0 × 105 | 5.6 × 105 | 0.0082 |

TABLE 4
Interpretation of Results from USP <62>
Table 2. Interpretation of Results

| Results for Each Quantity of Product | | | Probable Number |
|---|---|---|---|
| 0.1 g or | 0.01 g or | 0.001 g or | of Bacteria per g |
| 0.1 mL | 0.01 mL | 0.001 mL | or mL of Product |
| + | + | + | more than 10³ |
| + | + | − | less than 10³ and |
| | | | more than 10² |
| + | − | − | less than 10² and |
| | | | more than 10 |
| − | − | − | less than 10 |

TABLE 5

Clostridium_paraputrificum
Clostridium_disporicum
Clostridium_glycolicum
Clostridium_bartlettii
Clostridium_butyricum
Ruminococcus_bromii
Lachnospiraceae_bacterium_2_1_58FAA
Eubacterium_hadrum
Turicibacter_sanguinis
Lachnospiraceae_bacterium_oral_taxon_F15
Clostridium_perfringens
Clostridium_bifermentans
Roseburia_sp_11SE37
Clostridium_quinii
Ruminococcus_lactaris
Clostridium_botulinum
Clostridium_tyrobutyricum
Blautia_hansenii
Clostridium_kluyveri
Clostridium_sp_JC122
Clostridium_hylemonae
Clostridium_celatum
Clostridium_straminisolvens
Clostridium_orbiscindens
Roseburia_cecicola
Eubacterium_tenue
Clostridium_sp_7_2_43FAA
Lachnospiraceae_bacterium_4_1_37FAA
Eubacterium_rectale
Clostridium_viride
Ruminococcus_sp_K_1
Clostridium_symbiosum
Ruminococcus_torques
Clostridium_algidicarnis

TABLE 6

Clostridium_paraputrificum
Clostridium_bartlettii
Lachnospiraceae_bacterium_2_1_58FAA
Clostridium_disporicum
Ruminococcus_bromii
Eubacterium_hadrum
Clostridium_butyricum
Roseburia_sp_11SE37
Clostridium_perfringens
Clostridium_glycolicum
Clostridium_hylemonae
Clostridium_orbiscindens
Ruminococcus_lactaris
Clostridium_symbiosum
Lachnospiraceae_bacterium_oral_taxon_F15
Blautia_hansenii
Turicibacter_sanguinis
Clostridium_straminisolvens
Clostridium_botulinum
Lachnospiraceae_bacterium_4_1_37FAA
Roseburia_cecicola
Ruminococcus_sp_K_1
Clostridium_bifermentans
Eubacterium_rectale
Clostridium_quinii
Clostridium_viride
Clostridium_kluyveri
Clostridium_tyrobutyricum
Oscillibacter_sp_G2
Clostridium_sp_JC122
Lachnospiraceae_bacterium_3_1_57FAA
Clostridium_aldenense
Ruminococcus_torques
Clostridium_sp_7_2_43FAA
Clostridium_celatum
Eubacterium_sp_WAL_14571
Eubacterium_tenue
Lachnospiraceae_bacterium_5_1_57FAA
Clostridium_clostridioforme
Clostridium_sp_YIT_12070
Blautia_sp_M25
Anaerostipes_caccae
Roseburia_inulinivorans
Clostridium_sp_D5
Clostridium_asparagiforme
Coprobacillus_sp_D7
Clostridium_sp_HGF2
Clostridium_citroniae
Clostridium_difficile
Oscillibacter_valericigenes
Clostridium_algidicarnis

TABLE 7

| | | GAM + | | Sweet B + | Sweet | |
|---|---|---|---|---|---|---|
| OTU | BBA | FOS/inulin | M2GSC | FOS/Inulin | GAM | Total |
| Blautia producta | 1 | | | | | 1 |
| Clostridium bartlettii | 4 | | 1 | | | 5 |
| Clostridium bolteae | 2 | | | 5 | 1 | 8 |
| Clostridium botulinum | | | | 5 | | 5 |
| Clostridium butyricum | 37 | 43 | 8 | 1 | 33 | 122 |
| Clostridium celatum | 4 | | | | 1 | 5 |
| Clostridium clostridioforme | 1 | | | | 1 | 2 |
| Clostridium disporicum | 26 | 26 | 22 | 33 | 50 | 157 |
| Clostridium glycolicum | 4 | 9 | 14 | | | 27 |
| Clostridium mayombei | 2 | 2 | | | | 4 |
| Clostridium paraputrificum | 8 | 8 | 33 | 16 | 6 | 71 |
| Clostridium sordellii | | | 14 | | | 14 |
| Clostridium sp. 7_2_43FAA | | 1 | | | | 1 |
| Clostridium symbiosum | 3 | | | | | 3 |
| Clostridium tertium | | 1 | | 1 | | 2 |
| (blank) | | 2 | | 31 | | 33 |
| Totals | 92 | 92 | 92 | 92 | 92 | 460 |

TABLE 8
Results of the prophylaxis mouse model and dosing
information for the germinable, and sporulatable fractions

| | | | Average | |
|---|---|---|---|---|
| | | | Weight on | Average |
| | | | Day 3 | Clinical |
| | | # Deaths | Relative to | Score |
| Test Article | Dose | by Day 6 | Day −1 | on Day 3 |
| Vehicle | NA | 10 | 0.72 | NA |
| Naive | NA | 0 | 1.03 | 0 |
| Donor B fecal | 0.2 mL of 10% | 1 | 0.91 | 0.11 |
| suspension | suspension | | | |
| Donor A | 8.99 * 10\hat{ }7 Spore | 0 | 1.02 | 0 |
| Spore Prep | Equivalents/dose | | | |
| germinable | | | | |
| Donor A | 7.46 * 10\hat{ }7 Spore | 0 | 0.99 | 0 |
| Spore Prep | Equivalents/dose | | | |
| Sporulatable | | | | |

TABLE 9
16s rDNA identification of colony picks from plating a
20% fecal suspension or ethanol treated preparation to selective
media (number of colony picks matching each species in parentheses).

| | ethanol treated feces | 20% Suspension of feces |
|---|---|---|
| | (# of colonies) | (# of colonies) |
| Raffinose | Ruminococcus albus (5) | Bifidobacterium adolescentis (3) |
| Bifidobacterium | Clostridium sp. D5 (7) | Bifidobacterium longum (6) |
| Agar | Lachnospiraceae bacterium | Streptococcus bovis (1) |
| | 3_1_57FAA_CT1 (1) | Escherichia coli (4) |
| | Clostridium bolteae (3) | |
| | Robinsoniella peoriensis (1) | |
| | Ruminococcus lactaris (1) | |
| | Eubacterium fissicatena (1) | |
| | Eubacterium contortum | |
| | Eubacterium xylanophilum (1) | |
| | Clostridium clostridiiformes (1) | |
| Enterococcosel | no colonies observed | Streptococcous bovis (4) |
| Agar | | Streptococcus pasteurianus (1) |
| Mitis Salivarius | Bacillus subtilis (1) | Streptococcus vestibularis (3) |
| Agar | Bacillus sonorensis (1) | Streptococcus bovis (4) |
| | | Streptococcus salivarius (1) |

TABLE 10
16s rDNA identification of colony picks from plating a
20% fecal suspension or ethanol treated preparation to selective
media (number of colony picks matching each species in parentheses)

| | ethanol treated feces | 20% Suspension of feces |
|---|---|---|
| | (# of colonies) | (# of colonies) |
| Raffinose | Ruminococcus sp. 5_1_39BFAA (12) | Bifidobacterium adolescentis (3) |
| Bifidobacterium Agar | Dorea longicatena (3) | Bifidobacterium longum (10) |
| | Eubacterium contortum (4) | Enterococcus faecium (1) |
| | Clostridium sp. D5 (5) | |
| | Clostridium sp. 7_2_43FAA (1) | |
| | Bryantella formatexigens (1) | |
| | Clostridium orbiscindens (1) | |
| Enterococcosel Agar | no colonies observed | Enterococcus faecium (5) |
| Mitis Salivarius Agar | Bacillus sp. BT1B_CT2 (2) | Streptococcus vestibularis (1) |
| | Bacillus sp. B27(2008) (1) | Enterococcus faecium (4) |
| | Bacillus sonorensis (1) | Streptococcus salivarius (1) |

TABLE 11
Colony counts in Log CFU/mL determined from a 20%
fecal suspension and ethanol treated spore composition
Table 11 depicts the estimated concentration of a 20% fecal suspension
and the ethanol treated spore composition Colonies were counted
from plating a 20% feces suspension (Sample1) or ethanol treated
suspension to selective media and used to back-calculate the
concentration of presumptive cells in each sample (Log CFU/mL).

| | Log CFU/mL of | Log CFU/mL of | |
|---|---|---|---|
| | 20% | Ethanol-treated | Log |
| Selective Media | Suspension | spore composition | Reduction |
| Mitis Salivarius | 4.92 | 1 (1) | 3.92 |
| Enterococcosel | 4.75 | 1 Limit of Detection | 3.75 |
| | | (0) | |
| Raffinose | 6.65 | 2.70 | 3.95 |
| Bifidobacterium | | | |

TABLE 12
Colony counts in Log CFU/mL determined from a 20%
fecal suspension and ethanol treated spore composition.
Table 12 depicts the estimated concentration of a 20% fecal suspension
and the ethanol treated spore composition Colonies were counted from
plating a 20% feces suspension (Sample2) or ethanol treated
suspension to selective media and used to back-calculate the
concentration of presumptive cells in each sample (Log CFU/mL).

| | Log CFU/mL of | Log CFU/mL of | |
|---|---|---|---|
| | 20% | Ethanol-treated | Log |
| Selective Media | Suspension | spore composition | Reduction |
| Mitis Salivarius | 5.25 | 1.90 (8) | 3.34 |
| Enterococcosel | 5.14 | 1 Limit of Detection | 4.14 |
| | | (0) | |
| BIFIDO | 7.22 | 4.60 | 2.62 |
| Raffinose | 6.36 | 3.18 | 3.19 |
| Bifidobacterium | | | |

TABLE 13
Results of Plating Ethanol-treated fecal suspensions on
BBE and MacConkey II lactose agar

| | | MacConkey II Lactose |
|---|---|---|
| Donor | BBE Agar Results* | Result* |
| Donor A Sample 1 | No Colonies Observed | No Colonies Observed |
| Donor A Sample 2 | No Colonies Observed | No Colonies Observed |
| Donor B Sample 1 | No Colonies Observed | No Colonies Observed |
| Donor B Sample 2 | No Colonies Observed | No Colonies Observed |
| Donor B Sample 3 | No Colonies Observed | No Colonies Observed |
| Donor B Sample 4 | No Colonies Observed | No Colonies Observed |
| Donor B Sample 5 | No Colonies Observed | No Colonies Observed |
| Donor C Sample 1 | No Colonies Observed | No Colonies Observed |
| Donor C Sample 1 | No Colonies Observed | No Colonies Observed |
| Donor C Sample 1 | No Colonies Observed | No Colonies Observed |
| Donor C Sample 1 | No Colonies Observed | No Colonies Observed |
| Donor C Sample 1 | No Colonies Observed | No Colonies Observed |
| Donor C Sample 1 | No Colonies Observed | No Colonies Observed |
| Donor C Sample 1 | No Colonies Observed | No Colonies Observed |
| Donor D Sample 1 | No Colonies Observed | No Colonies Observed |
| Donor D Sample 2 | No Colonies Observed | No Colonies Observed |
| Donor E Sample 1 | No Colonies Observed | No Colonies Observed |
| Donor E Sample 2 | No Colonies Observed | No Colonies Observed |
| Donor F Sample 1 | No Colonies Observed | No Colonies Observed |
| Donor F Sample 2 | No Colonies Observed | No Colonies Observed |

*Note: The limit of detection for these results is 10 colony forming units per mL of sample.

TABLE 14
Results from Sabouraud Dextrose agar plating of fecal
suspensions before and after treatment with 50% Ethanol

| | CFU/mL | CFU/mL |
|---|---|---|
| Donor | Pre-Ethanol Inactivation | Post-Ethanol Inactivation |
| A | 2.00 × 10⁵ cfu/mL | No Colonies Detected* |
| B | 1.80 × 10⁶ cfu/mL | No Colonies Detected* |
| C | 2.00 × 10³ cfu/mL | 2.00 × 10³ cfu/mL |
| D | 2.00 × 10³ cfu/mL | No Colonies Detected* |

*Note: The limit of detection for this experiment was 2.00 × 10³ cfu/mL.

TABLE 15
Mortality and weight change in mice challenged with C. difficile
with or without ethanol treated, spore product treatment

| | mortality | % weight change |
|---|---|---|
| Test article | (n = 10) | on Day 3 |
| vehicle (negative | 20% | −10.5% |
| control) | | |
| Donor feces (positive | 0 | −0.1% |
| control) | | |
| EtOH-treated feces 1x | 0 | 2.3% |
| EtOH-treated feces 0.1x | 0 | 2.4% |
| EtOH-treated feces | 0 | −3% |
| 0.01x | | |
| heat-treated feces | 0 | 0.1% |

TABLE 16
16S rDNA identified spore forming species from picked colony plates

| Treatment | Species | No. isolates |
|---|---|---|
| 70 deg 1 h | Clostridium_celatum | 4 |
| 70 deg 1 h | Clostridium_clostridioform | 1 |
| 70 deg 1 h | Clostridium_hylemonae | 1 |
| 70 deg 1 h | Clostridium_paraputrificum | 3 |
| 70 deg 1 h | Clostridium_sp_D5 | 1 |
| 70 deg 1 h | Clostridium_symbiosum | 1 |
| 80 deg 1 h | Clostridium_bartlettii | 6 |
| 80 deg 1 h | Clostridium_butyricum | 1 |
| 80 deg 1 h | Clostridium_paraputrificum | 5 |
| 80 deg 1 h | Coprobacillus_sp_D7 | 1 |
| 80 deg 1 h | Eubacterium_sp_WAL_14571 | 1 |
| 80 deg 1 h | Ruminococcus_bromii | 1 |
| 90 deg 1 h | Clostridium_butyricum | 1 |
| 90 deg 10 min | Ruminococcus_bromii | 1 |
| 90 deg 10 min | Anaerotruncus_colihominis | 2 |
| 90 deg 10 min | Clostridium_bartlettii | 1 |
| 100 deg 10 min | Ruminococcus_bromii | 1 |

TABLE 17
Spore-forming species identified in ethanol treated or
heat treated samples and not identified in untreated samples

| | isolated from | isolated from | isolated from |
|---|---|---|---|
| Species | untreated | EtOH-treated | heat-treated |
| Acetivibrio ethanolgignens | | x | |
| Anaerofustis stercorihominis | | x | |
| Bacillus anthracis | | x | |
| Bacillus horti | | x | |
| Bacillus licheniformis | | | x |
| Bacillus nealsonii | | x | |
| Bacillus pumilus | | | x |
| Bacillus sp. BT1B_CT2 | | x | |
| Bacillus thuringiensis | | x | |
| Bacteroides galacturonicus | | x | |
| (phylogenetically in | | | |
| Clostridiales) | | | |
| Bacteroides pectinophilus | | x | |
| (phylogenetically in | | | |
| Clostridiales) | | | |
| Blautia wexlerae | | x | x |
| Brachyspira pilosicoli | | x | |
| Brevibacillus parabrevis | | | x |
| Clostridium aldenense | | x | |
| Clostridium beijerinckii | | x | |
| Clostridium carnis | | x | |
| Clostridium celatum | | x | |
| Clostridium favososporum | | x | |
| Clostridium hylemonae | | | x |
| Clostridium irregulare | | x | |
| Clostridium methylpentosum | | | x |
| Clostridium sp. D5 | | x | x |
| Clostridium sp. L2-50 | | x | |
| Clostridium sp. MT4 E | | x | |
| Clostridium sp. NML 04A032 | | x | |
| Clostridium sp. SS2/1 | | x | |
| Clostridium sp. YIT 12069 | | | x |
| Clostridium stercorarium | | x | |
| Clostridium xylanolyticum | | x | |
| Coprococcus sp. ART55/1 | | x | |
| Deferribacteres sp. oral clone | | x | |
| JV006 | | | |
| Desulfitobacterium frappieri | | x | |
| Eubacterium callanderi | | x | |
| Eubacterium siraeum | | x | |
| Exiguobacterium acetylicum | | x | |
| Gemmiger formicilis | | x | |
| Lachnospira multipara | | x | |
| Lachnospira pectinoschiza | | x | |
| Roseburia faecalis | | x | |
| Ruminococcus albus | | x | |

TABLE 18
Donor A, 45 species in 374 EtOH-resistant colonies sequenced

OTU
Anaerostipes_sp_3_2_56FAA
Bacillus_anthracis
Bacillus_cereus
Bacillus_thuringiensis
Blautia_producta
Blautia_sp_M25
Clostridiales_sp_SSC_2
Clostridium_aldenense
Clostridium_bartlettii
Clostridium_bolteae
Clostridium_celatum
Clostridium_disporicum
Clostridium_ghonii
Clostridium_hathewayi
Clostridium_lactatifermentans
Clostridium_mayombei
Clostridium_orbiscindens
Clostridium_paraputrificum
Clostridium_perfringens
Clostridium_sordellii
Clostridium_stercorarium
Clostridium_straminisolvens
Clostridium_tertium
Coprobacillus_sp_D7
Coprococcus_catus
Deferribacteres_sp_oral_clone_JV006
Dorea_formicigenerans
Eubacterium_rectale
Eubacterium_siraeum
Eubacterium_sp_WAL_14571
Eubacterium_ventriosum
Flexistipes_sinusarabici
Fulvimonas_sp_NML_060897
Lachnospiraceae_bacterium_2_1_58FAA
Lachnospiraceae_bacterium_3_1_57FAA
Lachnospiraceae_bacterium_A4
Lachnospiraceae_bacterium_oral_taxon_F15
Moorella_thermoacetica
Roseburia_faecalis
Roseburia_hominis
Ruminococcus_albus
Ruminococcus_bromii
Ruminococcus_gnavus
Ruminococcus_sp_5_1_39BFAA
Ruminococcus_torques

TABLE 19
Donor B, 26 species in 195 EtOH-resistant
colonies sequenced
OTU
Bacillus_horti
Blautia_wexlerae
Chlamydiales_bacterium_NS11
Clostridiales_sp_SSC_2
Clostridium_bartlettii
Clostridium_celatum
Clostridium_disporicum
Clostridium_ghonii
Clostridium_oroticum
Clostridium_paraputrificum
Clostridium_perfringens
Clostridium_sordellii
Clostridium_sp_L2_50
Clostridium_sp_MT4_E
Clostridium_straminisolvens
Coprococcus_sp_ART55_1
Eubacterium_callanderi
Eubacterium_rectale
Eubacterium_ruminantium
Gemmiger_formicilis
Lachnospira_pectinoschiza
Ruminococcus_albus
Ruminococcus_gnavus
Ruminococcus_obeum
Ruminococcus_sp_5_1_39BFAA
Ruminococcus_sp_K _1

TABLE 20
Donor C, 39 species in 416 EtOH-resistant
colonies sequenced
OTU
Bacteroides_galacturonicus
Bacteroides_pectinophilus
Blautia_producta
Blautia_sp_M25
Blautia_wexlerae
Clostridiales_sp_SS3_4
Clostridiales_sp_SSC_2
Clostridium_bartlettii
Clostridium_citroniae
Clostridium_disporicum
Clostridium_indolis
Clostridium_orbiscindens
Clostridium_paraputrificum
Clostridium_sordellii
Clostridium_sp_NML_04A032
Clostridium_sp_SS2_1
Clostridium_straminisolvens
Clostridium_viride
Clostridium_xylanolyticum
Coprobacillus_sp_D7
Dorea_longicatena
Eubacterium_rectale
Eubacterium_ventriosum
Hydrogenoanaerobacterium_saccharovorans
Lachnospira_multipara
Lachnospira_pectinoschiza
Lachnospiraceae_bacterium_A4
Oscillibacter_sp_G2
Pseudoflavonifractor_capillosus
Roseburia_hominis
Roseburia_intestinalis
Ruminococcus_albus
Ruminococcus_lactaris
Ruminococcus_obeum
Ruminococcus_sp_5_1_39BFAA
Ruminococcus_sp_K _1
Ruminococcus_torques
Syntrophococcus_sucromutans

TABLE 21
Donor D, 12 species in 118 EtOH-resistant
colonies sequenced
OTU
Blautia_luti
Blautia_wexlerae
Brachyspira_pilosicoli
Clostridium_paraputrificum
Collinsella_aerofaciens
Coprobacillus_sp_D7
Desulfitobacterium_frappieri
Eubacterium_rectale
Moorella_thermoacetica
Ruminococcus_gnavus
Ruminococcus_obeum
Ruminococcus_sp_K_1

TABLE 22
Donor E, 11 species in 118 EtOH-resistant colonies
sequenced
OTU
Blautia_luti
Blautia_wexlerae
Brachyspira_pilosicoli
Clostridium_paraputrificum
Coprobacillus_sp_D7
Desulfitobacterium_frappieri
Eubacterium_rectale
Moorella_thermoacetica
Ruminococcus_gnavus
Ruminococcus_obeum
Ruminococcus_sp_K_1

TABLE 23
Donor F, 54 OTUs in 768 EtOH-resistant colonies
sequenced
OTU
Anaerofustis_stercorihominis
Anaerostipes_sp_3_2_56FAA
Bacillus_nealsonii
Bacillus_sp_BT1B_CT2
Blautia_producta
Butyrivibrio_crossotus
Clostridiales_bacterium_SY8519
Clostridiales_sp_1_7_47
Clostridium_aldenense
Clostridium_bartlettii
Clostridium_bolteae
Clostridium_butyricum
Clostridium_citroniae
Clostridium_clostridioforme
Clostridium_disporicum
Clostridium_favososporum
Clostridium_glycolicum
Clostridium_hathewayi
Clostridium_indolis
Clostridium_leptum
Clostridium_mayombei
Clostridium_nexile
Clostridium_orbiscindens
Clostridium_sordellii
Clostridium_sp_7_2_43FAA
Clostridium_sp_D5
Clostridium_sp_M62_1
Clostridium_sp_NML_04A032
Clostridium_spiroforme
Clostridium_symbiosum
Clostridium_tertium
Coprobacillus_sp_29_1
Coprobacillus_sp_D7
Eubacterium_contortum
Eubacterium_desmolans
Eubacterium_ramulus
Exiguobacterium_acetylicum
Faecalibacterium_prausnitzii
Lachnospiraceae_bacterium_2_1_58FAA
Lachnospiraceae_bacterium_3_1_57FAA
Lachnospiraceae_bacterium_5_1_57FAA
Lachnospiraceae_bacterium_6_1_63FAA
Lachnospiraceae_bacterium_oral_taxon_F15
Marvinbryantia_formatexigens
Mycoplasma_amphoriforme
Oscillibacter_sp_G2
Pseudoflavonifractor_capillosus
Ruminococcus_gnavus
Ruminococcus_hansenii
Ruminococcus_obeum
Ruminococcus_sp_5_1_39BFAA
Ruminococcus_sp_ID8
Turicibacter_sanguinis

TABLE 24
Organisms crown from ethanol treated spore population
on various media (See Example 7 for full media names and
references).

| | total number | unique | % unique |
|---|---|---|---|
| Media | reads | OTUs | OTUs |
| M2GSC | 93 | 33 | 0.35 |
| M-BHI | 66 | 26 | 0.39 |
| Sweet B | 74 | 23 | 0.31 |
| GAM fructose | 44 | 18 | 0.41 |
| M2 mannitol | 39 | 17 | 0.44 |
| M2 soluble starch | 62 | 16 | 0.26 |
| M2 lactate | 43 | 14 | 0.33 |
| GAM FOS/Inulin | 52 | 14 | 0.27 |
| EYA | 29 | 13 | 0.45 |
| Mucin | 19 | 12 | 0.63 |
| M2 lactose | 32 | 12 | 0.38 |
| BHIS az1/ge2 | 35 | 12 | 0.34 |
| BHIS CInM az1/ge2 | 24 | 11 | 0.46 |
| GAM mannitol | 41 | 11 | 0.27 |
| BBA | 29 | 10 | 0.34 |
| Sulfite-polymyxin milk | 48 | 9 | 0.19 |
| Noack-Blaut Eubacterium | 12 | 4 | 0.33 |
| agar | | | |
| | 742 total analyzed | | |

TABLE 25
Species identified as germinable and sporulatable by colony picking

| | | GAM + | | Sweet B + | Sweet | |
|---|---|---|---|---|---|---|
| OTU | BBA | FOS/inulin | M2GSC | FOS/Inulin | GAM | Total |
| Blautia producta | 1 | | | | | 1 |
| Clostridium bartlettii | 4 | | 1 | | | 5 |
| Clostridium bolteae | 2 | | | 5 | 1 | 8 |
| Clostridium botulinum | | | | 5 | | 5 |
| Clostridium butyricum | 37 | 43 | 8 | 1 | 33 | 122 |
| Clostridium celatum | 4 | | | | 1 | 5 |
| Clostridium clostridioforme | 1 | | | | 1 | 2 |
| Clostridium disporicum | 26 | 26 | 22 | 33 | 50 | 157 |
| Clostridium glycolicum | 4 | 9 | 14 | | | 27 |
| Clostridium mayombei | 2 | 2 | | | | 4 |
| Clostridium paraputrificum | 8 | 8 | 33 | 16 | 6 | 71 |
| Clostridium sordellii | | | 14 | | | 14 |
| Clostridium sp. 7_2_43FAA | | 1 | | | | 1 |
| Clostridium symbiosum | 3 | | | | | 3 |
| Clostridium tertium | | 1 | | 1 | | 2 |
| (blank) | | 2 | | 31 | | 33 |
| Totals | 92 | 92 | 92 | 92 | 92 | 460 |

| | | | Dominant |
|---|---|---|---|
| | | Spore | OTU in |
| | Phylogenetic | Forming | Augmented |
| OTU | Clade | OTU | Ecology |
| Bacteroides sp. 2_1_22 | clade38 | N | Y |
| Streptococcus anginosus | clade60 | N | |
| Prevotella intermedia | clade8l | N | |
| Prevotella nigrescens | clade8l | N | |
| Oribacterium sp. ACB7 | clade90 | N | |
| Prevotella salivae | clade104 | N | |
| Bacteroides intestinalis | clade171 | N | Y |
| Bifidobacterium dentium | clade172 | N | |
| Alcaligenes faecalis | clade183 | N | |
| Rothia dentocariosa | clade194 | N | |
| Peptoniphilus lacrimalis | clade291 | N | |
| Anaerococcus sp. gpac155 | clade294 | N | |
| Sutterella stercoricanis | clade302 | N | Y |
| Bacteroides sp. 3_1_19 | clade335 | N | Y |
| Parabacteroides goldsteinii | clade335 | N | |
| Bacteroides dorei | clade378 | N | Y |
| Bacteroides massiliensis | clade378 | N | |
| Lactobacillus iners | clade398 | N | |
| Granulicatella adiacens | clade460 | N | |
| Eggerthella sp. 1_3_56FAA | clade477 | N | |
| Gordonibacter pamelaeae | clade477 | N | |
| Finegoldia magna | clade509 | N | |
| Actinomyces nasicola | clade523 | N | |
| Streptobacillus moniliformis | clade532 | N | |
| Oscillospira guilliermondii | clade540 | N | |
| Orientia tsutsugamushi | clade541 | N | |
| Christensenella minuta | clade558 | N | |
| Clostridium oroticum | clade96 | Y | |
| Clostridium sp. D5 | clade96 | Y | |
| Clostridium glycyrrhizinilyticum | clade147 | Y | |
| Coprococcus comes | clade147 | Y | |
| Ruminococcus lactaris | clade147 | Y | |
| Ruminococcus torques | clade147 | Y | Y |
| Clostridiales sp. SS3/4 | clade246 | Y | |
| Clostridium hylemonae | clade260 | Y | |
| Clostridium aerotolerans | clade269 | Y | |
| Clostridium asparagiforme | clade300 | Y | Y |
| Clostridium sp. M62/1 | clade300 | Y | |
| Clostridium symbiosum | clade300 | Y | |
| Lachnospiraceae genomosp. C1 | clade300 | Y | |
| Blautia sp. M25 | clade304 | Y | Y |
| Blautia stercoris | clade304 | Y | |
| Ruminococcus hansenii | clade304 | Y | |
| Ruminococcus obeum | clade304 | Y | |
| Ruminococcus sp. 5_1_39BFAA | clade304 | Y | |
| Bryantella formatexigens | clade309 | Y | |
| Eubacterium cellulosolvens | clade309 | Y | |
| Clostridium sp. HGF2 | clade351 | Y | |
| Clostridium bartlettii | clade354 | Y | |
| Clostridium bifermentans | clade354 | Y | |
| Clostridium glycolicum | clade354 | Y | |
| Eubacterium tenue | clade354 | Y | |
| Dorea formicigenerans | clade360 | Y | |
| Dorea longicatena | clade360 | Y | |
| Lachnospiraceae bacterium | clade360 | Y | |
| 2_1_46FAA | | | |
| Lachnospiraceae bacterium | clade360 | Y | Y |
| 9_1_43BFAA | | | |
| Ruminococcus gnavus | clade360 | Y | |
| Clostridium hathewayi | clade362 | Y | |
| Blautia hydrogenotrophica | clade368 | Y | |
| Clostridiaceae bacterium END-2 | clade368 | Y | |
| Roseburia faecis | clade369 | Y | |
| Roseburia hominis | clade370 | Y | |
| Roseburia intestinalis | clade370 | Y | |
| Eubacterium sp. WAL 14571 | clade384 | Y | |
| Erysipelotrichaceae bacterium | clade385 | Y | |
| 5_2_54FAA | | | |
| Eubacterium biforme | clade385 | Y | |
| Eubacterium dolichum | clade385 | Y | |
| Coprococcus catus | clade393 | Y | |
| Acetivibrio ethanolgignens | clade396 | Y | |
| Anaerosporobacter mobilis | clade396 | Y | |
| Bacteroides pectinophilus | clade396 | Y | |
| Eubacterium hallii | clade396 | Y | |
| Eubacterium xylanophilum | clade396 | Y | |
| Anaerostipes caccae | clade408 | Y | |
| Clostridiales bacterium 1_7_47FAA | clade408 | Y | |
| Clostridium aldenense | clade408 | Y | |
| Clostridium citroniae | clade408 | Y | |
| Eubacterium hadrum | clade408 | Y | Y |
| Acetanaerobacterium elongatum | clade439 | Y | |
| Faecalibacterium prausnitzii | clade478 | Y | |
| Gemmiger formicilis | clade478 | Y | Y |
| Eubacterium ramulus | clade482 | Y | |
| Lachnospiraceae bacterium | clade483 | Y | |
| 3_1_57FAA_CT1 | | | |
| Lachnospiraceae bacterium A4 | clade483 | Y | Y |
| Lachnospiraceae bacterium DJF | clade483 | Y | |
| VP30 | | | |
| Holdemania filiformis | clade485 | Y | |
| Clostridium orbiscindens | clade494 | Y | |
| Pseudoflavonifractor capillosus | clade494 | Y | |
| Ruminococcaceae bacterium D16 | clade494 | Y | |
| Acetivibrio cellulolyticus | clade495 | Y | |
| Eubacterium limosum | clade512 | Y | |
| Anaerotruncus colihominis | clade516 | Y | |
| Clostridium methylpentosum | clade516 | Y | |
| Clostridium sp. YIT 12070 | clade516 | Y | |
| Hydrogenoanaerobacterium | clade516 | Y | |
| saccharovorans | | | |
| Eubacterium ventriosum | clade519 | Y | |
| Eubacterium eligens | clade522 | Y | |
| Lachnospira pectinoschiza | clade522 | Y | |
| Lactobacillus rogosae | clade522 | Y | Y |
| Clostridium leptum | clade537 | Y | |
| Eubacterium coprostanoligenes | clade537 | Y | |
| Ruminococcus bromii | clade537 | Y | |
| Clostridium viride | clade540 | Y | |
| Butyrivibrio crossotus | clade543 | Y | |
| Coprococcus eutactus | clade543 | Y | |
| Eubacterium ruminantium | clade543 | Y | |
| Eubacterium rectale | clade568 | Y | Y |
| Roseburia inulinivorans | clade568 | Y | |
| Butyricicoccus pullicaecorum | clade572 | Y | |
| Eubacterium desmolans | clade572 | Y | |
| Papillibacter cinnamivorans | clade572 | Y | |
| Sporobacter termitidis | clade572 | Y | |
| Clostridium lactatifermentans | clade576 | Y | |

Bacterial OTUs associated with engraftment and ecological augmentation and establishment of a more diverse microbial ecology in patients treated with an ethanol treated spore preparation. OTUs that comprise an augmented ecology are not present in the patient prior to treatment and/or exist at extremely low frequencies such that they do not comprise a significant fraction of the total microbial carriage and are not detectable by genomic and/or microbiological assay methods. OTUs that are members of the engrafting and augmented ecologies were identified by characterizing the OTUs that increase in their relative abundance post treatment and that respectively are: (i) present in the ethanol treated spore preparation and absent in the patient pretreatment, or (ii) absent in the ethanol treated spore preparation, but increase in their relative abundance through time post treatment with the preparation due to the formation of favorable growth conditions by the treatment. Notably, the latter OTUs can grow from low frequency reservoirs in the patient, or be introduced from exogenous sources such as diet. OTUs that comprise a “core” augmented or engrafted ecology can be defined by the percentage of total patients in which they are observed to engraft and/or augment; the greater this percentage the more likely they are to be part of a core ecology responsible for catalyzing a shift away from a dysbiotic ecology. The dominant OTUs in an ecology can be identified using several methods including but not limited to defining the OTUs that have the greatest relative abundance in either the augmented or engrafted ecologies and defining a total relative abundance threshold. As example, the dominant OTUs in the augmented ecology of Patient-1 were identified by defining the OTUs with the greatest relative abundance, which together comprise 60% of the microbial carriage in this patient's augmented ecology.

TABLE 27
Reduction in the opportunistic pathogen or pathobiont
load by ethanol treated spores.

| | Pretreatment | Day 5 | Day 14 | Day 25 |
|---|---|---|---|---|
| Klebsiella (% of total reads) | 20.27% | 1.32% | 7.62% | 0.00% |
| Fusobacterium (% total of | 19.14% | 3.01% | 0.01% | 0.00% |
| reads) | | | | |

TABLE 28
Changes in Enterobacteria as a function of treatment measured
on Simmons Citrate Agar

| | | Pretreatment | Day 25 |
|---|---|---|---|
| Patient | Organism | titer (cfu/g) | titer (cfu/g) |
| 1 | Klebsiella pneumoniae | 9 × 10⁶ | 1 × 10³ |
| 1 | Klebsiella sp. Co9935 | 4 × 10⁶ | 1 × 10³ |
| 1 | Escherichia coli | 7 × 10⁶ | 1 × 10⁶ |
| 2 | Klebsiella sp. Co9935 | 4 × 10⁶ | 1 × 10³ |
| 4 | Klebsiella pneumoniae | 3 × 10⁸ | <1 × 10⁴ |
| 4 | Klebsiella sp. Co9935 | 6 × 10⁷ | <1 × 10⁴ |
| 5 | Klebsiella pneumoniae | 1 × 10⁶ | <1 × 10⁴ |

TABLE 29
Augmentation of Bacteroides as a function of bacterial
composition treatment of Patient 1.

| | Bacteroides | Pretreatment titer | Day 25 |
|---|---|---|---|
| Media | species | (cfu/g) | titer (cfu/g) |
| BBE | B. fragilis group | <2 × 10⁴ | 3 × 10⁸ |
| PFA | All Bacteroides | <2 × 10⁷ | 2 × 10¹⁰ |

TABLE 30
Bacteroides spp. in Patient 1 post-treatment
with the ethanol treated spore preparation
based full-length 16S rDNA sequences of isolated strains.

| | % of total Bacteroides |
|---|---|
| Species | cfu (1.58E10 cfu/g) |
| Bacteroides sp. 4_1_36 | 63% |
| Bacteroides cellulosilyticus | 14% |
| Bacteroides sp. 1_1_30 | 14% |
| Bacteroides uniformis | 4.8% |
| Bacteroides ovatus | 1.7% |
| Bacteroides dorei | 0.91% |
| Bacteroides xylanisolvens | 0.83% |
| Bacteroides sp. 3_1_19 | 0.23% |

TABLE 31
Titers (in cfu/g) of imipenem-resistant M. morganii,
P. rettgeri and P. pennerii from Patients 2, 4 & 5

| Patient | Organism | Pretreatment titer | Day 28 titer * |
|---|---|---|---|
| Patient 2 | M. morganii | 1 × 10⁴ | 6 × 10² |
| Patient 2 | P. rettgeri | 9 × 10³ | <5 × 10¹ |
| Patient 4 | M. morganii | 2 × 10⁴ | <5 × 10¹ |
| Patient 4 | P. pennerii | 2 × 10⁴ | <5 × 10¹ |
| Patient 5 | M. morganii | 5 × 10³ | <5 × 10¹ |

* Limit of detection based on plating 200 uL of 10% wt/vol suspension is 5 × 10¹

TABLE 32
OTUs detected by a minimum of ten 16S-V4 sequence reads
in at least a one ethanol treated spore preparation
(pan-microbiome). OTUs that engraft in a treated patients
and the percentage of patients in which they
engraft are denoted, as are the clades, spore forming status,
and Keystone OTU status. Starred OTUs occur in ≧80% of the
ethanol preps and engraft in ≧50% of the treated patients.

| | | % of | | | |
|---|---|---|---|---|---|
| | | Spore | % of | | |
| | | Preps | Patients | Spore | Key- |
| | | with | OTU | For- | stone |
| OTU | clade | OTU | Engrafts | mer | OTU |
| Prevotella_maculosa | clade_104 | 10% | 0% | N | N |
| Prevotella_copri | clade_168 | 20% | 0% | N | N |
| Bacteroides_caccae | clade_170 | 30% | 0% | N | Y |
| Bifidobacterium_sp_TM_7* | clade_172 | 90% | 60% | N | N |
| Bifidobacterium_gallicum | clade_172 | 70% | 20% | N | N |
| Bifidobacterium_dentium | clade_172 | 50% | 0% | N | N |
| Lactobacillus_casei | clade_198 | 20% | 10% | N | N |
| Actinomyces_odontolyticus | clade_212 | 20% | 30% | N | N |
| Clostridium_colicanis | clade_223 | 10% | 10% | Y | N |
| Clostridiales_sp_SS3_4* | clade_246 | 100% | 70% | Y | N |
| Clostridium_sporogenes | clade_252 | 40% | 40% | Y | N |
| Clostridium_butyricum | clade_252 | 20% | 20% | Y | N |
| Clostridium_disporicum | clade_253 | 40% | 30% | Y | N |
| Clostridium_hylemonae* | clade_260 | 100% | 50% | Y | N |
| Clostridium_scindens | clade_260 | 10% | 60% | Y | N |
| Coprococcus_comes* | clade_262 | 90% | 80% | Y | Y |
| Lachnospiraceae_ | clade_262 | 90% | 80% | Y | Y |
| bacterium_1_4_56FAA* | | | | | |
| Ruminococcus_torques | clade_262 | 30% | 70% | Y | Y |
| Parabacteroides_merdae | clade_286 | 30% | 20% | N | Y |
| Bifidobacterium_bifidum | clade_293 | 10% | 0% | N | N |
| Johnsonella_ignava | clade_298 | 10% | 10% | N | N |
| Blautia_glucerasea* | clade_309 | 100% | 80% | Y | N |
| Blautia_sp_M25* | clade_309 | 100% | 70% | Y | Y |
| Lachnospiraceae_ | clade_309 | 100% | 60% | Y | N |
| bacterium_6_1_63FAA* | | | | | |
| Eubacterium_cellulosolvens | clade_309 | 10% | 30% | Y | Y |
| Lactobacillus_fermentum | clade_313 | 10% | 0% | N | N |
| Sarcina_ventriculi | clade_353 | 10% | 10% | Y | N |
| Clostridium_bartlettii* | clade_354 | 90% | 70% | Y | N |
| Clostridium_bifermentans | clade_354 | 70% | 70% | Y | N |
| Clostridium_mayombei | clade_354 | 50% | 50% | Y | N |
| Dorea_longicatena* | clade_360 | 100% | 60% | Y | Y |
| Lachnospiraceae_ | clade_360 | 100% | 30% | Y | N |
| bacterium_9_1_43BFAA | | | | | |
| Lachnospiraceae_ | clade_360 | 80% | 80% | Y | N |
| bacterium_2_1_58FAA* | | | | | |
| Lachnospiraceae_ | clade_360 | 50% | 50% | Y | N |
| bacterium_2_1_46FAA | | | | | |
| Lactobacillus_perolens | clade_373 | 10% | 0% | N | N |
| Bacteroides_dorei | clade_378 | 60% | 50% | N | Y |
| Eubacterium_biforme | clade_385 | 10% | 0% | Y | N |
| Peptoniphilus_sp_gpac077 | clade_389 | 10% | 20% | N | N |
| Coprococcus_catus* | clade_393 | 100% | 70% | Y | Y |
| Eubacterium_hallii* | clade_396 | 90% | 60% | Y | Y |
| Anaerosporobacter_mobilis | clade_396 | 40% | 60% | Y | N |
| Bacteroides_pectinophilus | clade_396 | 10% | 60% | Y | N |
| Lactobacillus_hominis | clade_398 | 10% | 0% | N | N |
| Lactococcus_lactis | clade_401 | 40% | 40% | N | N |
| Ruminococcus_ | clade_406 | 80% | 50% | Y | N |
| champanellensis* | | | | | |
| Ruminococcus_callidus | clade_406 | 10% | 10% | Y | N |
| Clostridium_clostridioforme* | clade_408 | 100% | 60% | Y | Y |
| Eubacterium_hadrum* | clade_408 | 100% | 90% | Y | Y |
| Clostridium_symbiosum | clade_408 | 30% | 50% | Y | Y |
| Anaerostipes_caccae | clade_408 | 10% | 50% | Y | N |
| Parasutterella_ | clade_432 | 10% | 0% | N | N |
| excrementihominis | | | | | |
| Sutterella_stercoricanis | clade_432 | 10% | 0% | N | N |
| Eubacterium_rectale* | clade_444 | 100% | 80% | Y | Y |
| Lachnobacterium_bovis* | clade_444 | 100% | 80% | Y | N |
| Desulfovibrio_desulfuricans | clade_445 | 10% | 0% | N | Y |
| Eubacterium_sp_oral_ | clade_476 | 80% | 70% | Y | N |
| clone_JS001* | | | | | |
| Faecalibacterium_prausnitzii* | clade_478 | 100% | 60% | Y | Y |
| Subdoligranulum_variabile* | clade_478 | 100% | 80% | Y | Y |
| Coprobacillus_sp_D7* | clade_481 | 90% | 60% | Y | N |
| Clostridium_cocleatum | clade_481 | 60% | 20% | Y | N |
| Clostridium_spiroforme | clade_481 | 40% | 50% | Y | N |
| Eubacterium_ramulus* | clade_482 | 80% | 60% | Y | N |
| Flavonifractor_plautii | clade_494 | 70% | 60% | Y | Y |
| Pseudoflavonifractor_capillosus | clade_494 | 60% | 60% | Y | Y |
| Ruminococcaceae_ | clade_494 | 30% | 50% | Y | Y |
| bacterium_D16 | | | | | |
| Acetivibrio_cellulolyticus* | clade_495 | 70% | 80% | Y | N |
| Clostridium_stercorarium | clade_495 | 40% | 50% | Y | N |
| Enterococcus_durans | clade_497 | 10% | 10% | N | N |
| Enterococcus_ faecium | clade_497 | 10% | 10% | N | N |
| Dialister_invisus | clade_506 | 50% | 10% | N | N |
| Eubacterium_limosum | clade_512 | 20% | 0% | Y | N |
| Ruminococcus_flavefaciens | clade_516 | 60% | 60% | Y | N |
| Eubacterium_ventriosum | clade_519 | 30% | 60% | Y | Y |
| Bilophila_wadsworthia | clade_521 | 90% | 0% | N | Y |
| Lachnospira_pectinoschiza | clade_522 | 40% | 60% | Y | N |
| Eubacterium_eligens | clade_522 | 30% | 50% | Y | Y |
| Catonella_morbi | clade_534 | 20% | 0% | N | N |
| Clostridium_sporosphaeroides* | clade_537 | 100% | 80% | Y | N |
| Ruminococcus_bromii | clade_537 | 60% | 30% | Y | Y |
| Clostridium_leptum | clade_537 | 40% | 70% | Y | Y |
| Clostridium_sp_YIT_12069 | clade_537 | 40% | 60% | Y | N |
| Clostridium_viride | clade_540 | 10% | 10% | Y | N |
| Megamonas_funiformis | clade_542 | 50% | 0% | N | N |
| Eubacterium_ruminantium* | clade_543 | 80% | 90% | Y | N |
| Coprococcus_eutactus | clade_543 | 20% | 20% | Y | N |
| Collinsella_aerofaciens | clade_553 | 50% | 10% | Y | Y |
| Alkaliphilus_metalliredigenes | clade_554 | 40% | 10% | Y | N |
| Turicibacter_sanguinis | clade_555 | 80% | 40% | Y | N |
| Phascolarctobacterium_faecium | clade_556 | 20% | 0% | N | N |
| Clostridiales_bacterium_ | clade_558 | 80% | 50% | N | N |
| oral_clone_P4PA* | | | | | |
| Lutispora_thermophila | clade_564 | 100% | 0% | Y | N |
| Coriobacteriaceae_ | clade_566 | 70% | 0% | N | N |
| bacterium_JC110 | | | | | |
| Eggerthella_sp_1_3_56FAA | clade_566 | 70% | 30% | N | N |
| Adlercreutzia_equolifaciens | clade_566 | 40% | 0% | N | N |
| Gordonibacter_pamelaeae | clade_566 | 30% | 0% | N | Y |
| Slackia_isoflavoniconvertens | clade_566 | 10% | 0% | N | N |
| Eubacterium_desmolans* | clade_572 | 90% | 70% | Y | N |
| Papillibacter_cinnamivorans* | clade_572 | 90% | 80% | Y | N |
| Clostridium_colinum | clade_576 | 30% | 30% | Y | N |
| Akkermansia_muciniphila | clade_583 | 60% | 10% | N | Y |
| Clostridiales_bacterium_ | clade_584 | 60% | 30% | N | N |
| oral_taxon_F32 | | | | | |
| Prochlorococcus_marinus | clade_592 | 30% | 0% | N | N |
| Methanobrevibacter_wolinii | clade_595 | 30% | 0% | N | N |
| Bacteroides_fragilis | clade_65 | 20% | 30% | N | Y |
| Lactobacillus_delbrueckii | clade_72 | 10% | 0% | N | N |
| Escherichia_coli | clade_92 | 50% | 0% | N | Y |
| Clostridium_sp_D5 | clade_96 | 80% | 60% | Y | N |
| Streptococcus_thermophilus | clade_98 | 90% | 20% | N | Y |
| Streptococcus_sp_CM6 | clade_98 | 20% | 10% | N | N |
| Streptococcus_sp_oral_ | clade_98 | 10% | 0% | N | N |
| clone_ASCE05 | | | | | |

TABLE 33
Top 20 OTUs ranked by CES

| | | | Spore | Keystone |
|---|---|---|---|---|
| OTU | Clade | CES | Former | OTU |
| Eubacterium_hadrum | clade_408 | 4.2 | Y | Y |
| Eubacterium_rectale | clade_444 | 4.2 | Y | Y |
| Subdoligranulum_variabile | clade_478 | 4.2 | Y | Y |
| Blautia_sp_M25 | clade_309 | 4.2 | Y | Y |
| Coprococcus_catus | clade_393 | 4.2 | Y | Y |
| Lachnospiraceae_bacterium_ | clade_262 | 4.2 | Y | Y |
| 1_4_56FAA | | | | |
| Coprococcus_comes | clade_262 | 4.2 | Y | Y |
| Blautia_glucerasea | clade_309 | 4.0 | Y | N |
| Lachnobacterium_bovis | clade_444 | 4.0 | Y | N |
| Clostridium_sporosphaeroides | clade_537 | 4.0 | Y | N |
| Clostridiales_sp_SS3_4 | clade_246 | 4.0 | Y | N |
| Papillibacter_cinnamivorans | clade_572 | 4.0 | Y | N |
| Clostridium_bartlettii | clade_354 | 4.0 | Y | N |
| Eubacterium_desmolans | clade_572 | 4.0 | Y | N |
| Clostridium_clostridioforme | clade_408 | 3.2 | Y | Y |
| Dorea_longicatena | clade_360 | 3.2 | Y | Y |
| Faecalibacterium_prausnitzii | clade_478 | 3.2 | Y | Y |
| Eubacterium_hallii | clade_396 | 3.2 | Y | Y |
| Clostridium_leptum | clade_537 | 3.2 | Y | Y |
| Lachnospiraceae_bacterium_ | clade_309 | 3.0 | Y | N |
| 6_1_63FAA | | | | |

TABLE 34
Subsets of the Core Ecology tested in the C. difficile mouse model

| | | Substitute For OTU in |
|---|---|---|
| Subset | OTU | Table 1 (Clade) |
| Subset 1 | Collinsella aerofaciens | none (Clade_553) |
| | Clostridium tertium | C. sporogenes (Clade_252) |
| | Clostridium disporicum | none (Clade_253) |
| | Clostridium innocuum | Clostridium_sp_HGF2 |
| | | (Clade_351) |
| | Clostridium mayombei | none (Clade_354) |
| | Clostridium butyricum | none (Clade_252) |
| | Coprococcus comes | none (Clade_262) |
| | Clostridium hylemonae | none (Clade_260) |
| | Clostridium bolteae | E. hadrum (Clade_408) |
| | Clostridium symbiosum | C. clostridioforme |
| | | (Clade_408) |
| | Clostridium orbiscindens | R._bacterium_D16 |
| | | (Clade_494) |
| | Lachnospiraceae | C. scindens (Clade_260) |
| | bacterium_5_1_57FAA | |
| | Blautia producta | Blautia_sp_M25 |
| | | (Clade_309) |
| | Ruminococcus gnavus | D. longicatena (Clade_360) |
| | Ruminococcus bromii | none (Clade_537) |
| Subset 2 | Collinsella aerofaciens | none (Clade_553) |
| | Clostridium butyricum | none (Clade_252) |
| | Clostridium hylemonae | none (Clade_260) |
| | Blautia producta | Blautia_sp_M25 |
| | | (Clade_309) |
| Subset 3 | Collinsella aerofaciens | none (Clade_553) |
| | Clostridium innocuum | Clostridium_sp_HGF2 |
| | | (Clade_351) |
| | Coprococcus comes | none (Clade_262) |
| | Ruminococcus bromii | none (Clade_537) |
| Subset 4 | Clostridium butyricum | none (Clade_252) |
| | Clostridium hylemonae | none (Clade_260) |
| | Blautia producta | Blautia_sp_M25 |
| | | (Clade_309) |
| Subset 5 | Clostridium butyricum | none (Clade_252) |
| | Clostridium hylemonae | none (Clade_260) |
| Subset 6 | Blautia producta | Blautia_sp_M25 |
| | | (Clade_309) |
| | Clostridium butyricum | none (Clade_252) |
| Subset 7 | Clostridium orbiscindens | R._bacterium_D16 |
| | | (Clade_494) |
| | Lachnospiraceae | C. scindens (Clade_260) |
| | bacterium_5_1_57FAA | |
| | Eubacterium rectale | none (Clade_444) |

TABLE 35
Results of bacterial compositions tested in a C. difficile mouse model

| | | | | Avg. |
|---|---|---|---|---|
| | | | Avg. | Maximum |
| | | Cumulative | Minimum | Clinical |
| | | Mortality | Relative | Score |
| Group | Dose | (%) | Weight | (Death = 4) |
| Vehicle | — | 40 | 0.87 | 2.8 |
| Control | | | | |
| Feces | 5.8e8 cfu | 0 | 0.99 | 0 |
| Control | total | | | |
| Subset 1 | 1e8 | 0 | 0.98 | 0 |
| | cfu/OTU | | | |
| Subset 2 | 1e8 | 10 | 0.84 | 2.1 |
| | cfu/OTU | | | |
| Subset 3 | 1e8 | 10 | 0.84 | 2.2 |
| | cfu/OTU | | | |
| Subset 4 | 1e8 | 0 | 0.87 | 2 |
| | cfu/OTU | | | |
| Subset 5 | 1e8 | 20 | 0.91 | 1.7 |
| | cfu/OTU | | | |
| Subset 6 | 1e8 | 40 | 0.82 | 2.8 |
| | cfu/OTU | | | |
| Subset 7 | 1e8 | 0 | 0.90 | 1 |
| | cfu/OTU | | | |

TABLE 36
OTUs and their clade assignments tested in ternary combinations with results in the in
vitro inhibition assay

| OTU1 | Clade1 | OTU2 | Clade2 |
|---|---|---|---|
| Clostridium_bolteae | clade_408 | Blautia_producta | clade_309 |
| Clostridium_bolteae | clade_408 | Clostridium_symbiosum | clade_408 |
| Clostridium_bolteae | clade_408 | Clostridium_symbiosum | clade_408 |
| Clostridium_bolteae | clade_408 | Clostridium_symbiosum | clade_408 |
| Clostridium_bolteae | clade_408 | Clostridium_symbiosum | clade_408 |
| Clostridium_bolteae | clade_408 | Faecalibacterium_prausnitzii | clade_478 |
| Clostridium_bolteae | clade_408 | Faecalibacterium_prausnitzii | clade_478 |
| Clostridium_bolteae | clade_408 | Faecalibacterium_prausnitzii | clade_478 |
| Clostridium_bolteae | clade_408 | Lachnospiraceae_bacterium_5_1_57FAA | clade_260 |
| Clostridium_bolteae | clade_408 | Lachnospiraceae_bacterium_5_1_57FAA | clade_260 |
| Clostridium_symbiosum | clade_408 | Blautia_producta | clade_309 |
| Clostridium_symbiosum | clade_408 | Faecalibacterium_prausnitzii | clade_478 |
| Clostridium_symbiosum | clade_408 | Faecalibacterium_prausnitzii | clade_478 |
| Clostridium_symbiosum | clade_408 | Faecalibacterium_prausnitzii | clade_478 |
| Clostridium_symbiosum | clade_408 | Lachnospiraceae_bacterium_5_1_57FAA | clade_260 |
| Clostridium_symbiosum | clade_408 | Lachnospiraceae_bacterium_5_1_57FAA | clade_260 |
| Collinsella_aerofaciens | clade_553 | Blautia_producta | clade_309 |
| Collinsella_aerofaciens | clade_553 | Clostridium_bolteae | clade_408 |
| Collinsella_aerofaciens | clade_553 | Clostridium_bolteae | clade_408 |
| Collinsella_aerofaciens | clade_553 | Clostridium_bolteae | clade_408 |
| Collinsella_aerofaciens | clade_553 | Clostridium_bolteae | clade_408 |
| Collinsella_aerofaciens | clade_553 | Clostridium_bolteae | clade_408 |
| Collinsella_aerofaciens | clade_553 | Clostridium_symbiosum | clade_408 |
| Collinsella_aerofaciens | clade_553 | Clostridium_symbiosum | clade_408 |
| Collinsella_aerofaciens | clade_553 | Clostridium_symbiosum | clade_408 |
| Collinsella_aerofaciens | clade_553 | Clostridium_symbiosum | clade_408 |
| Collinsella_aerofaciens | clade_553 | Coprococcus_comes | clade_262 |
| Collinsella_aerofaciens | clade_553 | Coprococcus_comes | clade_262 |
| Collinsella_aerofaciens | clade_553 | Coprococcus_comes | clade_262 |
| Collinsella_aerofaciens | clade_553 | Coprococcus_comes | clade_262 |
| Collinsella_aerofaciens | clade_553 | Coprococcus_comes | clade_262 |
| Collinsella_aerofaciens | clade_553 | Coprococcus_comes | clade_262 |
| Collinsella_aerofaciens | clade_553 | Faecalibacterium_prausnitzii | clade_478 |
| Collinsella_aerofaciens | clade_553 | Faecalibacterium_prausnitzii | clade_478 |
| Collinsella_aerofaciens | clade_553 | Faecalibacterium_prausnitzii | clade_478 |
| Collinsella_aerofaciens | clade_553 | Lachnospiraceae_bacterium_5_1_57FAA | clade_260 |
| Collinsella_aerofaciens | clade_553 | Lachnospiraceae_bacterium_5_1_57FAA | clade_260 |
| Coprococcus_comes | clade_262 | Blautia_producta | clade_309 |
| Coprococcus_comes | clade_262 | Clostridium_bolteae | clade_408 |
| Coprococcus_comes | clade_262 | Clostridium_bolteae | clade_408 |
| Coprococcus_comes | clade_262 | Clostridium_bolteae | clade_408 |
| Coprococcus_comes | clade_262 | Clostridium_bolteae | clade_408 |
| Coprococcus_comes | clade_262 | Clostridium_bolteae | clade_408 |
| Coprococcus_comes | clade_262 | Clostridium_symbiosum | clade_408 |
| Coprococcus_comes | clade_262 | Clostridium_symbiosum | clade_408 |
| Coprococcus_comes | clade_262 | Clostridium_symbiosum | clade_408 |
| Coprococcus_comes | clade_262 | Clostridium_symbiosum | clade_408 |
| Coprococcus_comes | clade_262 | Faecalibacterium_prausnitzii | clade_478 |
| Coprococcus_comes | clade_262 | Faecalibacterium_prausnitzii | clade_478 |
| Coprococcus_comes | clade_262 | Faecalibacterium_prausnitzii | clade_478 |
| Coprococcus_comes | clade_262 | Lachnospiraceae_bacterium_5_1_57FAA | clade_260 |
| Coprococcus_comes | clade_262 | Lachnospiraceae_bacterium_5_1_57FAA | clade_260 |
| Faecalibacterium_prausnitzii | clade_478 | Blautia_producta | clade_309 |
| Faecalibacterium_prausnitzii | clade_478 | Lachnospiraceae_bacterium_5_1_57FAA | clade_260 |
| Faecalibacterium_prausnitzii | clade_478 | Lachnospiraceae_bacterium_5_1_57FAA | clade_260 |
| Lachnospiraceae_bacterium_5_1_57FAA | clade_260 | Blautia_producta | clade_309 |

| OTU3 | Clade3 | Results |
|---|---|---|
| Eubacterium_rectale | clade_444 | ++++ |
| Blautia_producta | clade_309 | ++++ |
| Eubacterium_rectale | clade_444 | − |
| Faecalibacterium_prausnitzii | clade_478 | − |
| Lachnospiraceae_bacterium_5_1_57FAA | clade_260 | |
| Blautia_producta | clade_309 | ++++ |
| Eubacterium_rectale | clade_444 | |
| Lachnospiraceae_bacterium_5_1_57FAA | clade_260 | ++++ |
| Blautia_producta | clade_309 | ++++ |
| Eubacterium_rectale | clade_444 | + |
| Eubacterium_rectale | clade_444 | ++++ |
| Blautia_producta | clade_309 | ++++ |
| Eubacterium_rectale | clade_444 | |
| Lachnospiraceae_bacterium_5_1_57FAA | clade_260 | + |
| Blautia_producta | clade_309 | ++++ |
| Eubacterium_rectale | clade_444 | |
| Eubacterium_rectale | clade_444 | ++++ |
| Blautia_producta | clade_309 | ++++ |
| Clostridium_symbiosum | clade_408 | ++++ |
| Eubacterium_rectale | clade_444 | ++++ |
| Faecalibacterium_prausnitzii | clade_478 | ++++ |
| Lachnospiraceae_bacterium_5_1_57FAA | clade_260 | ++++ |
| Blautia_producta | clade_309 | ++++ |
| Eubacterium_rectale | clade_444 | |
| Faecalibacterium_prausnitzii | clade_478 | |
| Lachnospiraceae_bacterium_5_1_57FAA | clade_260 | + |
| Blautia_producta | clade_309 | ++++ |
| Clostridium_bolteae | clade_408 | ++++ |
| Clostridium_symbiosum | clade_408 | +++ |
| Eubacterium_rectale | clade_444 | +++ |
| Faecalibacterium_prausnitzii | clade_478 | ++++ |
| Lachnospiraceae_bacterium_5_1_57FAA | clade_260 | +++ |
| Blautia_producta | clade_309 | ++++ |
| Eubacterium_rectale | clade_444 | +++ |
| Lachnospiraceae_bacterium_5_1_57FAA | clade_260 | +++ |
| Blautia_producta | clade_309 | ++++ |
| Eubacterium_rectale | clade_444 | ++++ |
| Eubacterium_rectale | clade_444 | ++++ |
| Blautia_producta | clade_309 | ++++ |
| Clostridium_symbiosum | clade_408 | |
| Eubacterium_rectale | clade_444 | −− |
| Faecalibacterium_prausnitzii | clade_478 | +++ |
| Lachnospiraceae_bacterium_5_1_57FAA | clade_260 | +++ |
| Blautia_producta | clade_309 | ++++ |
| Eubacterium_rectale | clade_444 | −−− |
| Faecalibacterium_prausnitzii | clade_478 | |
| Lachnospiraceae_bacterium_5_1_57FAA | clade_260 | |
| Blautia_producta | clade_309 | ++++ |
| Eubacterium_rectale | clade_444 | − |
| Lachnospiraceae_bacterium_5_1_57FAA | clade_260 | |
| Blautia_producta | clade_309 | ++++ |
| Eubacterium_rectale | clade_444 | |
| Eubacterium_rectale | clade_444 | ++++ |
| Blautia_producta | clade_309 | ++++ |
| Eubacterium_rectale | clade_444 | |
| Eubacterium_rectale | clade_444 | ++++ |

What is claimed is:

1. A method of characterizing a therapeutic composition, comprising the steps of:
(a) providing a therapeutic composition comprising at least one desired bacterial strain and optionally comprising at least one undesired bacterial strain;
(b) subjecting the therapeutic composition to a first detection step and a second detection step,
wherein the first detection step comprises attempting to culture at least one undesired bacterial strain, and wherein the second detection step comprises attempting to amplify at least one target nucleic acid sequence not present in the desired bacterial strain, thereby characterizing the therapeutic composition.

2. The method of claim 1, wherein the desired bacterial strain comprises a plurality of desired bacterial strains.

3. The method of claim 1, wherein the result of the attempt to culture the at least one undesired bacterial strain is that the undesired bacterial strain is not detectably cultured.

4. The method of claim 1, wherein the undesired bacterial strain is not known to be present in the therapeutic composition.

5. The method of claim 1, wherein the undesired bacterial strain is a contaminating bacterial strain derived from the manufacturing environment or process.

6. The method of claim 1, wherein the result of the attempt to amplify the at least one target nucleic acid sequence is that the target nucleic acid sequence is not detectably amplified.

7. The method of claim 6, wherein the target nucleic acid sequence is present in i) a bacterial strain derived from a fecal culture, and/or ii) a fecal material.

8. The method of claim 1, wherein the first detection step has a sensitivity for the undesired bacterial strain of at least 1×10⁻³, and wherein the second detection step has a sensitivity for the undesired bacterial strain of at least 1×10⁻³.

9. The method of claim 1, wherein the first detection step has a sensitivity for the undesired bacterial strain of at least 1×10⁻⁴, and wherein the second detection step has a sensitivity for the undesired bacterial strain of at least 1×10⁻⁴.

10. The method of claim 1, wherein the first detection step has a sensitivity for the undesired bacterial strain of at least 1×10⁻⁵, and wherein the second detection step has a sensitivity for the undesired bacterial strain of at least 1×10⁻⁵.

11. The method of claim 1, further comprising the step of detecting, or attempting to detect, a non-bacterial microbial contaminant in the therapeutic composition.

12. The method of claim 11, wherein the non-bacterial microbial contaminant comprises a phage, virus, or eukaryotic contaminant.

13. The method of claim 1, wherein the first detection step is performed prior to the second detection step.

14. The method of claim 1, wherein the first detection step is performed after the second detection step.

15. The method of claim 1, wherein the first detection step and the second detection step are performed concurrently.

16. The method of any of claims 1-15, wherein the second detection step is carried out using a product of the first detection step.

17. The method of any of claims 1-15, wherein the first detection step is carried out using a product of the second detection step.

18. The method of claim 1, wherein the therapeutic composition is validated to detect a contaminant in a background of 1×10⁵ CFU of the product bacteria.

19. The method of claim 1, further comprising the step of attempting to enrich at least one undesired bacterial strain in the therapeutic composition.

20. The validated therapeutic composition provided by the method of claim 1.

21. A method of characterizing a therapeutic composition, comprising the steps of:
(a) providing a therapeutic composition comprising at least one desired entity and optionally comprising at least one undesired entity;
(b) subjecting the therapeutic composition to an enrichment step wherein the at least one undesired entity or component thereof, if present in the therapeutic composition, is enriched; and
(c) subjecting the enriched therapeutic composition to a first detection step and a second detection step, wherein the first detection step comprises attempting to detect the undesired entity at a concentration of about less than or equal to 1×10⁻³ the concentration of the desired entity, and wherein the second detection step comprises attempting to detect the undesired entity at a concentration of about less than or equal to 1×10⁻³ the concentration of the desired entity, wherein the first detection step and the second detection step are not identical, thereby characterizing the therapeutic composition.

22. The method of claim 21, wherein the first detection step comprises attempting to detect the undesired entity at a concentration of about less than or equal to 1×10⁻⁴ the concentration of the desired entity, and wherein the second detection step comprises attempting to detect the undesired entity at a concentration of about less than or equal to 1×10⁻⁴ the concentration of the desired entity.

23. The method of claim 21, wherein the first detection step comprises attempting to detect the undesired entity at a concentration of about less than or equal to 1×10⁻⁵ the concentration of the desired entity, and wherein the second detection step comprises attempting to detect the undesired entity at a concentration of about less than or equal to 1×10⁻⁵ the concentration of the desired entity.

24. The method of claim 21, wherein the desired entity comprises a plurality of desired entities.

25. The method of claim 21, wherein the at least one desired entity comprises a bacteria.

26. The method of claim 21, wherein the at least one undesired entity comprises a bacterium, yeast, virus or combination thereof.

27. The method of claim 21, wherein the first detection step and the second detection step are performed simultaneously.

28. The method of claim 21, wherein the first detection step and the second detection step are performed sequentially.

29. The method of claim 21, wherein the second detection step detects a product of the first detection step.

30. The method of claim 21, wherein the undesired entity is not detectably present in the characterized therapeutic composition at a concentration of about greater than or equal to 1×10⁻⁷ the concentration of the desired entity.

31. The method of claim 21, wherein the component of the undesired entity comprises a nucleic acid.

32. A method of characterizing a bacterial composition, comprising the steps of:
(a) providing a composition comprising at least one desired bacterial species and optionally comprising at least one undesired entity;
(b) subjecting the therapeutic composition to a first detection step and a second detection step, wherein the first detection step comprises attempting to detect the at least one undesired entity and the first detection step has a sensitivity for the undesired entity of at least 1×10-3, and wherein the second detection step comprises attempting to detect the at least one undesired entity and the second detection step has a sensitivity for the undesired entity of at least 1×10-3, wherein the first and second detection steps are not identical and have a combined sensitivity for the undesired entity of at least 1×10-6.

33. The method of claim 32, wherein the first detection step comprises attempting to detect the at least one undesired entity and the first detection step has a sensitivity for the undesired entity of at least 1×10⁻⁴, and wherein the second detection step comprises attempting to detect the at least one undesired entity and the second detection step has a sensitivity for the undesired entity of at least 1×10⁻⁴.

34. The method of claim 32, wherein the first detection step comprises attempting to detect the at least one undesired entity and the first detection step has a sensitivity for the undesired entity of at least 1×10⁻⁵, and wherein the second detection step comprises attempting to detect the at least one undesired entity and the second detection step has a sensitivity for the undesired entity of at least 1×10⁻⁵.

35. The method of claim 32, wherein the at least one desired bacterial species comprises a plurality of desired bacterial species.

36. The method of claim 32, wherein the first detection step is performed prior to the second detection step.

37. The method of claim 32, wherein the first detection step and the second detection step are performed concurrently.

38. The method of claim 32, wherein the first detection step is carried out using a product of the second detection step.

39. The method of claim 32, wherein the second detection step is carried out using a product of the first detection step.

40. A method of characterizing a spore population present in a composition comprising the steps of:
(a) purifying the spore population present in a composition from a fecal donation; and
(b) deriving the spore population present in a composition through culture methods.

41. The method of claim 40, wherein the spore population present in a composition is purified via solvent, acid, detergent, or heat treatment, or a density gradient separation, filtration, or any combination of methods.

42. The method of claim 40, wherein the purifying increases the purity, potency, and/or concentration of spores in a sample.

43. The method of claim 40, wherein the spore population is derived starting from isolated spore former species or spore former OTUs or from a mixture of such species.

44. The method of claim 40, wherein the spore population is in vegetative or spore form.

45. The method of claim 40, wherein the spores can be purified from natural sources including but not limited to feces, soil, and water.

46. The method of claim 40, wherein the spore population is a non-limiting subset of a microbial composition.

47. The method of claim 40, wherein ethanol treated fecal suspensions are a non-limiting additional subset of a microbial composition enriched for spores and spore formers.

48. The method of claim 40, wherein the spore population comprises spore forming species wherein residual non-spore forming species have been inactivated by chemical or physical treatments.

49. The method of claim 48, wherein the chemical or physical treatments include ethanol, detergent, heat or sonication.

50. The method of claim 40, wherein the non-spore forming species have been removed from the spore preparation by various separation steps.

51. The method of claim 50, wherein the separation steps include density gradients, centrifugation, filtration and chromatography.

52. The method of claim 40, wherein inactivation and separation methods are combined to make the spore preparation.

53. The method of claim 40, wherein the spore preparation comprises spore forming species that are enriched over viable non-spore formers or vegetative forms of spore formers.

54. The method of claim 53, wherein the spores are enriched by 2-fold, 5-fold, 10-fold, 50-fold, 100-fold, 1000-fold, 10,000 fold or greater than 10,000-fold compared to all vegetative forms of bacteria.

55. The method of claim 40, wherein the spores in the spore preparation undergo partial germination during processing and formulation such that the final composition comprises spores and vegetative bacteria derived from spore forming species.
